SEQUENCE LISTING (131 sequences)

SEQ ID NO: 1 - DNA, 20 bp, synthetic construct
  source 1..20: mol_type = other DNA
gatggtcagacatttccaga

SEQ ID NO: 2 - DNA, 24 bp, synthetic construct
  source 1..24: mol_type = other DNA
ctggctcaacctcgtctttaaatg

SEQ ID NO: 3 - DNA, 24 bp, synthetic construct
  source 1..24: mol_type = other DNA
ttctgatggtcagacatttccaga

SEQ ID NO: 4 - DNA, 24 bp, synthetic construct
  source 1..24: mol_type = other DNA
aggtagatgatccagagtcaggag

SEQ ID NO: 5 - DNA, 21 bp, synthetic construct
  source 1..21: mol_type = other DNA
tgatggacagacattgcgaga

SEQ ID NO: 6 - DNA, 21 bp, synthetic construct
  source 1..21: mol_type = other DNA
gcatgagtagcccgtggcttc

SEQ ID NO: 7 - DNA, 21 bp, synthetic construct
  source 1..21: mol_type = other DNA
tcagagagaggggttggaagc

SEQ ID NO: 8 - DNA, 21 bp, synthetic construct
  source 1..21: mol_type = other DNA
agatgatccaaagtcagagag

SEQ ID NO: 9 - DNA, 20 bp, synthetic construct
  source 1..20: mol_type = other DNA
tctatgtcctctttcccatc

SEQ ID NO: 10 - DNA, 20 bp, synthetic construct
  source 1..20: mol_type = other DNA
tctacgtcctcttccccatc

SEQ ID NO: 11 - DNA, 20 bp, synthetic construct
  source 1..20: mol_type = other DNA
ttgctgatctcctctctata

SEQ ID NO: 12 - DNA, 20 bp, synthetic construct
  source 1..20: mol_type = other DNA
gtggtaagaaccatatagag

SEQ ID NO: 13 - DNA, 20 bp, synthetic construct
  source 1..20: mol_type = other DNA
gaggagatcagcaagaaaag

SEQ ID NO: 14 - DNA, 20 bp, synthetic construct
  source 1..20: mol_type = other DNA
agatcagcaagaaaagaggc

SEQ ID NO: 15 - DNA, 20 bp, synthetic construct
  source 1..20: mol_type = other DNA
ctggggaaagcaacttctga

SEQ ID NO: 16 - DNA, 1182 bp, synthetic construct
  source 1..1182: mol_type = other DNA
tagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtc

SEQ ID NO: 17 - DNA, 1125 bp, synthetic construct
  source 1..1125: mol_type = other DNA
tcgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtc

SEQ ID NO: 18 - DNA, 1074 bp, synthetic construct
  source 1..1074: mol_type = other DNA
atggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaa

SEQ ID NO: 19 - DNA, 1074 bp, synthetic construct
  source 1..1074: mol_type = other DNA
atggaggaaggaatggatgtgctccatgactttggaatccagtcaacgcactacctccaggtgaattaccaggactctcaggattggttcatcttggtgtccgtgattgcagacctcaggaatgccttctatgtcctctttcccatctggttccatctgcgtgaagctgtgggcatcaaacttctctgggtagctgtgattggagactggctcaacctcgtctttaaatggattctgtttggacagcgtccatactggtgggtcatggacaccgactactatagcaacacctctgtgccactgataaagcaatttccagttacctgtgaaactggaccagggagtccctctggtcatgccatgggtacagcaggtgtatactacgtgatggtcacatctaccctttctatctttcgggggagaaaaaggccaacctacagatttcggtgcttgaatatccttctgtggttgggattctgggctgtgcagctgaacgtctgtctgtcccgaatctaccttgctgctcatttcccccatcaggttgttgctggagtcctgtcaggcattgctgttgctgaaactttccgccacatccagagcatctacaatgccagcctcaagaaatattttctcattactttcttcctgttcagttttgccattggattttacctgctgctcaaggggctgggtgtggacctcctgtggacactggaaaaagccaggagatggtgtgagcggccggaatgggttcacattgacaccacaccctttgccagccttctcaagaacgtggggaccctctttggcctgggggtggctctcaactccagcatgtacagggaaagctgcaagggcaagcttagcaagtggttcccattccgcctcagctgcattgtggtgtctctcatcctcctgcacctctttgactctttgaaacccccatcccaaactgagctgatcttctacaccttgtccttctgcaagagtgcagcagtgcccctggcatctgtcagcctcatcccctactgccttgcccgggtcttcgaccagccagacaagaagtctttgtaa

SEQ ID NO: 20 - DNA, 293 bp, synthetic construct
  source 1..293: mol_type = other DNA
cctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactca

SEQ ID NO: 21 - DNA, 303 bp, synthetic construct
  source 1..303: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagc

SEQ ID NO: 22 - DNA, 356 bp, synthetic construct
  source 1..356: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 23 - DNA, 1284 bp, synthetic construct
  source 1..1284: mol_type = other DNA
agctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 24 - DNA, 1475 bp, synthetic construct
  source 1..1475: mol_type = other DNA
cctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtc

SEQ ID NO: 25 - DNA, 911 bp, synthetic construct
  source 1..911: mol_type = other DNA
cactcagtttgttcccaaatcacttttattctgtgtgtggagagaaacaggaggtcagaggtatcccttcctcttcgtcagagcaaatggttctattacactagggctctgtctttatggtctcctttaagttatacttttaacttcttttggcttaaagtacctatctccaactgtgatccacgtgaacttggtgaaagtccaagagagtgagcatttccaggacaacaaagccctactgctgtgtattttgtatattttaaattaataacttaaaaggtcacttccggcagtagcaaacttccctttgtcttcaaatagatataattgcagacttcaaaaaacagatgcccttgcaagagtcatggttgaaacagactctgccctgggcctctggcctggcttcaaggaccaggaaggagggcagcctctagcactgtcaagcagtgtgcccaagttaataattggctctgccaatggcgatcaggctgtttttgtgtgcctgttttgctattttacgtaaatcaccctgaacatgtttgcatcaacctactgatgatgcacctttgatcaatagattttagacaaaagtggttttttgagtccaaagatcagggctgggttgacctacagactgaatccagggcatataaaacaggggcaaggcacagactgatagcagagggatcaagccaaccgggcttggactcactgcacgggctctgctggcagcttcctgaggtaccaagggaggaagctcgagggaaggaatgaacattctccatgactttgggatccagtcgactcgctatctccaagtgaattaccaagactcccaggactggttcatccttgtgtctgtgattgctgacctgaggaacgccttctatgtcgactttcccatctagatc

SEQ ID NO: 26 - DNA, 1002 bp, synthetic construct
  source 1..1002: mol_type = other DNA
tctagatcacgcgtaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatg

SEQ ID NO: 27 - DNA, 758 bp, synthetic construct
  source 1..758: mol_type = other DNA
cactcagtttgttcccaaatcacttttattctgtgtgtggagagaaacaggaggtcagaggtatcccttcctcttcgtcagagcaaatggttctattacactagggctctgtctttatggtctcctttaagttatacttttaacttcttttggcttaaagtacctatctccaactgtgatccacgtgaacttggtgaaagtccaagagagtgagcatttccaggacaacaaagccctactgctgtgtattttgtatattttaaattaataacttaaaaggtcacttccggcagtagcaaacttccctttgtcttcaaatagatataattgcagacttcaaaaaacagatgcccttgcaagagtcatggttgaaacagactctgccctgggcctctggcctggcttcaaggaccaggaaggagggcagcctctagcactgtcaagcagtgtgcccaagttaataattggctctgccaatggcgatcaggctgtttttgtgtgcctgttttgctattttacgtaaatcaccctgaacatgtttgcatcaacctactgatgatgcacctttgatcaatagattttagacaaaagtggttttttgagtccaaagatcagggctgggttgacctacagactgaatccagggcatataaaacaggggcaaggcacagactgatagcagagggatcaagccaaccgggcttggactcactgcacgggctctgctggcagcttcctgaggtaccaagggaggaagc

SEQ ID NO: 28 - DNA, 1010 bp, synthetic construct
  source 1..1010: mol_type = other DNA
ctttcccatctagatccatcttaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatg

SEQ ID NO: 29 - DNA, 1675 bp, synthetic construct
  source 1..1675: mol_type = other DNA
atgctaagtgaaataagacagaaaaagacaaatatcatatggtttcacttatatgtagggtctaaaaagcgaaacaaatgagtaaacaaacacaaaaagcagactcaaacctactatgtggagaacaaattgatacttgcccaagaggaggagcagtgagaggatggggaaaatgggtgaaggggaatgagagatacaggcttccagacctcatggaatgagtaagtcactggaatgaaaggcacagcacagggaatacagtcaatggtattgtaacagtgttgtacagtgacaggtggtagctatactgtggtgaacgaagcataatgcacagagatgttgaataactatgttgtacacctgaaactaatgttagattagtttcattatgtaacatcagtttcagctacattctaatacactttaaaataatacagtagtcctttccagagtccccagtcaccaatttgctcgtgtttttatttagaaccccatattcattacattgtttttgcatgaacacacattcattctactgcttaaacttatgtgttaaaaccccaaaggtcctaacttggaggacattgcatgaattaatcagtgcattcaaagatgattacttcctggccctaagatatatagaatcaacactaggaagaaggtttgatctcccaaaccagaggcacttcacagacactatgcagttaagaatacacagcaaggtaggtgatggttcccagtacaatagaacttagcgaatatgtttttttgagattttatttatgagagacacacagagagaggcagagacacaggtagaaggagaagcaggctccctgaagggagcccgatgccggactcaatcccaggaccctgagatcacaacctgagccaaaggtagatgctcatccactgagccatgcaggtaccccgaacttagcatattttaaaagtaatatgtttaaaacatgctcactagggtaccaagacttttctttgtcctcaaataagtgcacttgcagacagaaaatcaggatctttgcaagaaccatggcggatttagactctgtcctgtgtctccggcccagtttcggagccaggagggcagcccttgcactatcaagtagcatgccaaagttaatcattggccctgccaagtacatggcagatcaggctgtttttgtgtgcctgtttttctattttacgtaaatcaccctgaacatgtttgcatcaacctcctggtgatgtacctttgatcaatagattttagacaaaagtggtttttgagtccaaagatcagggctgggttgacctgcagactggatacagagtgtataaaaacaggggcaaggcgcaggctgatagcagagcaatcgccaagtcggtaccgagctcggaaagggcgaattccagcacactggcggccgttactagtggatccaaggatggaggaaggaatggatgtgctccatgactttggaatccagtcaacgcactacctccaggtgaattaccaggactctcaggattggttcatcttggtgtccgtgattgcagacctcaggaatgccttctatgtcctctttcccatctggttccatctgcgtgaagctgtgggcatcaaacttctctgggtagctgtgattggagactggctcaacctcgtctttaaatg

SEQ ID NO: 30 - DNA, 1445 bp, synthetic construct
  source 1..1445: mol_type = other DNA
atgctaagtgaaataagacagaaaaagacaaatatcatatggtttcacttatatgtagggtctaaaaagcgaaacaaatgagtaaacaaacacaaaaagcagactcaaacctactatgtggagaacaaattgatacttgcccaagaggaggagcagtgagaggatggggaaaatgggtgaaggggaatgagagatacaggcttccagacctcatggaatgagtaagtcactggaatgaaaggcacagcacagggaatacagtcaatggtattgtaacagtgttgtacagtgacaggtggtagctatactgtggtgaacgaagcataatgcacagagatgttgaataactatgttgtacacctgaaactaatgttagattagtttcattatgtaacatcagtttcagctacattctaatacactttaaaataatacagtagtcctttccagagtccccagtcaccaatttgctcgtgtttttatttagaaccccatattcattacattgtttttgcatgaacacacattcattctactgcttaaacttatgtgttaaaaccccaaaggtcctaacttggaggacattgcatgaattaatcagtgcattcaaagatgattacttcctggccctaagatatatagaatcaacactaggaagaaggtttgatctcccaaaccagaggcacttcacagacactatgcagttaagaatacacagcaaggtaggtgatggttcccagtacaatagaacttagcgaatatgtttttttgagattttatttatgagagacacacagagagaggcagagacacaggtagaaggagaagcaggctccctgaagggagcccgatgccggactcaatcccaggaccctgagatcacaacctgagccaaaggtagatgctcatccactgagccatgcaggtaccccgaacttagcatattttaaaagtaatatgtttaaaacatgctcactagggtaccaagacttttctttgtcctcaaataagtgcacttgcagacagaaaatcaggatctttgcaagaaccatggcggatttagactctgtcctgtgtctccggcccagtttcggagccaggagggcagcccttgcactatcaagtagcatgccaaagttaatcattggccctgccaagtacatggcagatcaggctgtttttgtgtgcctgtttttctattttacgtaaatcaccctgaacatgtttgcatcaacctcctggtgatgtacctttgatcaatagattttagacaaaagtggtttttgagtccaaagatcagggctgggttgacctgcagactggatacagagtgtataaaaacaggggcaaggcgcaggctgatagcagagcaatcgccaagtcggtaccgagctcggaaagggcgaattccagcacactggcggccgttactagtggatccaagg

SEQ ID NO: 31 - DNA, 1033 bp, synthetic construct
  source 1..1033: mol_type = other DNA
ttctctgggtagctgtgattggagactggctcaacctcgtctttaaatggtgagtaagagccagacagagaggagatcggcaagggaagaggctggtatagtctctggaaatgtctgacatcagaaattgccttctccgtgctagtcgggaagccacaggttactcccctcctgactctggatcatctacctaaagagggaagacagcgaaaatcttgttgatgaattgaagatacaggcaaggctatttcatatggatggaaggccagccggacttagtgaaaagtctaaagtatataggctataagcaaagaagcacagttgtattactttgaagcagaataggtaacataaagataggtgtatgaaagatgatgtatgaaaacaggaaacaagtatataatttaaaaacccactgcatggagaaagaagaagtaacccagagaactgggctctaacccttgttcggccacctaccagctgtatgtcctgggaaagtcagagcaactctctgagtttctgtcttacaatctgtaaacttaaattatccttgggagcttaataataattctagttaacatttattgaacatttagtaggtactaggccatgtgccaagtgcttttctgtgaggtagttactactgtattatagatgaagagacagagccattacgataagggaccagaactagatccacatggtggtcaaaggcagaggcagcattttaaggccagacaggctagtttcagaggcaaagctcttaaggattatgctgtattcattgctgacaaattgactttttcagggagcaggaggaagccaaattttaatgtttgtcaatttctctggtgtatatattcctattggggccacttttgatgggtcaagcttgcaaaaatcctgaatattttacaattgtcagccaataccatatgattctagctcattagtggcttggcctctatttcctcatctagaaaataaagagcttgggccagctaaaacctttaccatccctaaactatctatg

SEQ ID NO: 32 - DNA, 1537 bp, synthetic construct
  source 1..1537: mol_type = other DNA
gagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtg

SEQ ID NO: 33 - DNA, 1307 bp, synthetic construct
  source 1..1307: mol_type = other DNA
gagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgagg

SEQ ID NO: 34 - DNA, 1007 bp, synthetic construct
  source 1..1007: mol_type = other DNA
gtaagaaccatatagagaggagatcagcaagaaaagaggctggcattcgctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcgg

SEQ ID NO: 35 - DNA, 1042 bp, synthetic construct
  source 1..1042: mol_type = other DNA
gtaagaaccatatagagaggagatcagcaagaaaagaggctggcataggctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcggcaatgctcttatgaactatgttacgtccagtgctg

SEQ ID NO: 36 - DNA, 3694 bp, synthetic construct
  source 1..3694: mol_type = other DNA
cactcagtttgttcccaaatcacttttattctgtgtgtggagagaaacaggaggtcagaggtatcccttcctcttcgtcagagcaaatggttctattacactagggctctgtctttatggtctcctttaagttatacttttaacttcttttggcttaaagtacctatctccaactgtgatccacgtgaacttggtgaaagtccaagagagtgagcatttccaggacaacaaagccctactgctgtgtattttgtatattttaaattaataacttaaaaggtcacttccggcagtagcaaacttccctttgtcttcaaatagatataattgcagacttcaaaaaacagatgcccttgcaagagtcatggttgaaacagactctgccctgggcctctggcctggcttcaaggaccaggaaggagggcagcctctagcactgtcaagcagtgtgcccaagttaataattggctctgccaatggcgatcaggctgtttttgtgtgcctgttttgctattttacgtaaatcaccctgaacatgtttgcatcaacctactgatgatgcacctttgatcaatagattttagacaaaagtggttttttgagtccaaagatcagggctgggttgacctacagactgaatccagggcatataaaacaggggcaaggcacagactgatagcagagggatcaagccaaccgggcttggactcactgcacgggctctgctggcagcttcctgaggtaccaagggaggaagctcgagggaaggaatgaacattctccatgactttgggatccagtcgactcgctatctccaagtgaattaccaagactcccaggactggttcatccttgtgtctgtgattgctgacctgaggaacgccttctatgtcgactttcccatctagatcacgcgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcgaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggaattctgcagatctcgagtctagatcacgcgtaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatg

SEQ ID NO: 37 - DNA, 3280 bp, synthetic construct
  source 1..3280: mol_type = other DNA
cactcagtttgttcccaaatcacttttattctgtgtgtggagagaaacaggaggtcagaggtatcccttcctcttcgtcagagcaaatggttctattacactagggctctgtctttatggtctcctttaagttatacttttaacttcttttggcttaaagtacctatctccaactgtgatccacgtgaacttggtgaaagtccaagagagtgagcatttccaggacaacaaagccctactgctgtgtattttgtatattttaaattaataacttaaaaggtcacttccggcagtagcaaacttccctttgtcttcaaatagatataattgcagacttcaaaaaacagatgcccttgcaagagtcatggttgaaacagactctgccctgggcctctggcctggcttcaaggaccaggaaggagggcagcctctagcactgtcaagcagtgtgcccaagttaataattggctctgccaatggcgatcaggctgtttttgtgtgcctgttttgctattttacgtaaatcaccctgaacatgtttgcatcaacctactgatgatgcacctttgatcaatagattttagacaaaagtggttttttgagtccaaagatcagggctgggttgacctacagactgaatccagggcatataaaacaggggcaaggcacagactgatagcagagggatcaagccaaccgggcttggactcactgcacgggctctgctggcagcttcctgaggtaccaagggaggaagctcgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcgaattctgcagatctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcaaaaacgtcgactttcccatctagatccatcttaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatg

SEQ ID NO: 38 - DNA, 4278 bp, synthetic construct
  source 1..4278: mol_type = other DNA
atgctaagtgaaataagacagaaaaagacaaatatcatatggtttcacttatatgtagggtctaaaaagcgaaacaaatgagtaaacaaacacaaaaagcagactcaaacctactatgtggagaacaaattgatacttgcccaagaggaggagcagtgagaggatggggaaaatgggtgaaggggaatgagagatacaggcttccagacctcatggaatgagtaagtcactggaatgaaaggcacagcacagggaatacagtcaatggtattgtaacagtgttgtacagtgacaggtggtagctatactgtggtgaacgaagcataatgcacagagatgttgaataactatgttgtacacctgaaactaatgttagattagtttcattatgtaacatcagtttcagctacattctaatacactttaaaataatacagtagtcctttccagagtccccagtcaccaatttgctcgtgtttttatttagaaccccatattcattacattgtttttgcatgaacacacattcattctactgcttaaacttatgtgttaaaaccccaaaggtcctaacttggaggacattgcatgaattaatcagtgcattcaaagatgattacttcctggccctaagatatatagaatcaacactaggaagaaggtttgatctcccaaaccagaggcacttcacagacactatgcagttaagaatacacagcaaggtaggtgatggttcccagtacaatagaacttagcgaatatgtttttttgagattttatttatgagagacacacagagagaggcagagacacaggtagaaggagaagcaggctccctgaagggagcccgatgccggactcaatcccaggaccctgagatcacaacctgagccaaaggtagatgctcatccactgagccatgcaggtaccccgaacttagcatattttaaaagtaatatgtttaaaacatgctcactagggtaccaagacttttctttgtcctcaaataagtgcacttgcagacagaaaatcaggatctttgcaagaaccatggcggatttagactctgtcctgtgtctccggcccagtttcggagccaggagggcagcccttgcactatcaagtagcatgccaaagttaatcattggccctgccaagtacatggcagatcaggctgtttttgtgtgcctgtttttctattttacgtaaatcaccctgaacatgtttgcatcaacctcctggtgatgtacctttgatcaatagattttagacaaaagtggtttttgagtccaaagatcagggctgggttgacctgcagactggatacagagtgtataaaaacaggggcaaggcgcaggctgatagcagagcaatcgccaagtcggtaccgagctcggaaagggcgaattccagcacactggcggccgttactagtggatccaaggatggaggaaggaatggatgtgctccatgactttggaatccagtcaacgcactacctccaggtgaattaccaggactctcaggattggttcatcttggtgtccgtgattgcagacctcaggaatgccttctatgtcctctttcccatctggttccatctgcgtgaagctgtgggcatcaaacttctctgggtagctgtgattggagactggctcaacctcgtctttaaatggattctgtttggacagcgtccatactggtgggtcatggacaccgactactatagcaacacctctgtgccactgataaagcaatttccagttacctgtgaaactggaccagggagtccctctggtcatgccatgggtacagcaggtgtatactacgtgatggtcacatctaccctttctatctttcgggggagaaaaaggccaacctacagatttcggtgcttgaatatccttctgtggttgggattctgggctgtgcagctgaacgtctgtctgtcccgaatctaccttgctgctcatttcccccatcaggttgttgctggagtcctgtcaggcattgctgttgctgaaactttccgccacatccagagcatctacaatgccagcctcaagaaatattttctcattactttcttcctgttcagttttgccattggattttacctgctgctcaaggggctgggtgtggacctcctgtggacactggaaaaagccaggagatggtgtgagcggccggaatgggttcacattgacaccacaccctttgccagccttctcaagaacgtggggaccctctttggcctgggggtggctctcaactccagcatgtacagggaaagctgcaagggcaagcttagcaagtggttcccattccgcctcagctgcattgtggtgtctctcatcctcctgcacctctttgactctttgaaacccccatcccaaactgagctgatcttctacaccttgtccttctgcaagagtgcagcagtgcccctggcatctgtcagcctcatcccctactgccttgcccgggtcttcgaccagccagacaagaagtctttgtaaggcgaattctgcagatatccatcacactggcggccgctcgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagtcttgtcctaataaaattaagttgcatcattttgtctgactaggtgtccttctataatattatggggtggaggggggtggtatggagcaagggaatagattctctgggtagctgtgattggagactggctcaacctcgtctttaaatggtgagtaagagccagacagagaggagatcggcaagggaagaggctggtatagtctctggaaatgtctgacatcagaaattgccttctccgtgctagtcgggaagccacaggttactcccctcctgactctggatcatctacctaaagagggaagacagcgaaaatcttgttgatgaattgaagatacaggcaaggctatttcatatggatggaaggccagccggacttagtgaaaagtctaaagtatataggctataagcaaagaagcacagttgtattactttgaagcagaataggtaacataaagataggtgtatgaaagatgatgtatgaaaacaggaaacaagtatataatttaaaaacccactgcatggagaaagaagaagtaacccagagaactgggctctaacccttgttcggccacctaccagctgtatgtcctgggaaagtcagagcaactctctgagtttctgtcttacaatctgtaaacttaaattatccttgggagcttaataataattctagttaacatttattgaacatttagtaggtactaggccatgtgccaagtgcttttctgtgaggtagttactactgtattatagatgaagagacagagccattacgataagggaccagaactagatccacatggtggtcaaaggcagaggcagcattttaaggccagacaggctagtttcagaggcaaagctcttaaggattatgctgtattcattgctgacaaattgactttttcagggagcaggaggaagccaaattttaatgtttgtcaatttctctggtgtatatattcctattggggccacttttgatgggtcaagcttgcaaaaatcctgaatattttacaattgtcagccaataccatatgattctagctcattagtggcttggcctctatttcctcatctagaaaataaagagcttgggccagctaaaacctttaccatccctaaactatctatg

SEQ ID NO: 39 - DNA, 4149 bp, synthetic construct
  source 1..4149: mol_type = other DNA
gagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaaggcgaattctgcagatatccatcacactggcggccgctcgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagtcttgtcctaataaaattaagttgcatcattttgtctgactaggtgtccttctataatattatggggtggaggggggtggtatggagcaagggaatagagtaagaaccatatagagaggagatcagcaagaaaagaggctggcataggctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcggcaatgctcttatgaactatgttacgtccagtgctg

SEQ ID NO: 40 - DNA, 4114 bp, synthetic construct
  source 1..4114: mol_type = other DNA
gagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaaggcgaattctgcagatatccatcacactggcggccgctcgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagtcttgtcctaataaaattaagttgcatcattttgtctgactaggtgtccttctataatattatggggtggaggggggtggtatggagcaagggaatagagtaagaaccatatagagaggagatcagcaagaaaagaggctggcattcgctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcgg

SEQ ID NO: 41 - DNA, 7027 bp, synthetic construct
  source 1..7027: mol_type = other DNA
atgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccctgaaaacacaaatggcgtgttttggttggcgtaaggcgcctgtcagttaaccactcagtttgttcccaaatcacttttattctgtgtgtggagagaaacaggaggtcagaggtatcccttcctcttcgtcagagcaaatggttctattacactagggctctgtctttatggtctcctttaagttatacttttaacttcttttggcttaaagtacctatctccaactgtgatccacgtgaacttggtgaaagtccaagagagtgagcatttccaggacaacaaagccctactgctgtgtattttgtatattttaaattaataacttaaaaggtcacttccggcagtagcaaacttccctttgtcttcaaatagatataattgcagacttcaaaaaacagatgcccttgcaagagtcatggttgaaacagactctgccctgggcctctggcctggcttcaaggaccaggaaggagggcagcctctagcactgtcaagcagtgtgcccaagttaataattggctctgccaatggcgatcaggctgtttttgtgtgcctgttttgctattttacgtaaatcaccctgaacatgtttgcatcaacctactgatgatgcacctttgatcaatagattttagacaaaagtggttttttgagtccaaagatcagggctgggttgacctacagactgaatccagggcatataaaacaggggcaaggcacagactgatagcagagggatcaagccaaccgggcttggactcactgcacgggctctgctggcagcttcctgaggtaccaagggaggaagctcgagggaaggaatgaacattctccatgactttgggatccagtcgactcgctatctccaagtgaattaccaagactcccaggactggttcatccttgtgtctgtgattgctgacctgaggaacgccttctatgtcgactttcccatctagatcacgcgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcgaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggaattctgcagatctcgagtctagatcacgcgtaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatgccgcggcagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctatgtcctctttcccatcgttttagagctagaaatagcaagttaaaataaggctagtccgttatcaacttgaaaaagtggcaccgagtcggtgcttttttgccgcggtggggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac

SEQ ID NO: 42 - DNA, 6569 bp, synthetic construct
  source 1..6569: mol_type = other DNA
atgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccagttaaccactcagtttgttcccaaatcacttttattctgtgtgtggagagaaacaggaggtcagaggtatcccttcctcttcgtcagagcaaatggttctattacactagggctctgtctttatggtctcctttaagttatacttttaacttcttttggcttaaagtacctatctccaactgtgatccacgtgaacttggtgaaagtccaagagagtgagcatttccaggacaacaaagccctactgctgtgtattttgtatattttaaattaataacttaaaaggtcacttccggcagtagcaaacttccctttgtcttcaaatagatataattgcagacttcaaaaaacagatgcccttgcaagagtcatggttgaaacagactctgccctgggcctctggcctggcttcaaggaccaggaaggagggcagcctctagcactgtcaagcagtgtgcccaagttaataattggctctgccaatggcgatcaggctgtttttgtgtgcctgttttgctattttacgtaaatcaccctgaacatgtttgcatcaacctactgatgatgcacctttgatcaatagattttagacaaaagtggttttttgagtccaaagatcagggctgggttgacctacagactgaatccagggcatataaaacaggggcaaggcacagactgatagcagagggatcaagccaaccgggcttggactcactgcacgggctctgctggcagcttcctgaggtaccaagggaggaagctcgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcgaattctgcagatctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcaaaaacgtcgactttcccatctagatccatcttaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatgccgcggcagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctatgtcctctttcccatcgttttagagctagaaatagcaagttaaaataaggctagtccgttatcaacttgaaaaagtggcaccgagtcggtgcttttttgccgcggtggggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac

SEQ ID NO: 43 - DNA, 6753 bp, synthetic construct
  source 1..6753: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgctctagaatgctaagtgaaataagacagaaaaagacaaatatcatatggtttcacttatatgtagggtctaaaaagcgaaacaaatgagtaaacaaacacaaaaagcagactcaaacctactatgtggagaacaaattgatacttgcccaagaggaggagcagtgagaggatggggaaaatgggtgaaggggaatgagagatacaggcttccagacctcatggaatgagtaagtcactggaatgaaaggcacagcacagggaatacagtcaatggtattgtaacagtgttgtacagtgacaggtggtagctatactgtggtgaacgaagcataatgcacagagatgttgaataactatgttgtacacctgaaactaatgttagattagtttcattatgtaacatcagtttcagctacattctaatacactttaaaataatacagtagtcctttccagagtccccagtcaccaatttgctcgtgtttttatttagaaccccatattcattacattgtttttgcatgaacacacattcattctactgcttaaacttatgtgttaaaaccccaaaggtcctaacttggaggacattgcatgaattaatcagtgcattcaaagatgattacttcctggccctaagatatatagaatcaacactaggaagaaggtttgatctcccaaaccagaggcacttcacagacactatgcagttaagaatacacagcaaggtaggtgatggttcccagtacaatagaacttagcgaatatgtttttttgagattttatttatgagagacacacagagagaggcagagacacaggtagaaggagaagcaggctccctgaagggagcccgatgccggactcaatcccaggaccctgagatcacaacctgagccaaaggtagatgctcatccactgagccatgcaggtaccccgaacttagcatattttaaaagtaatatgtttaaaacatgctcactagggtaccaagacttttctttgtcctcaaataagtgcacttgcagacagaaaatcaggatctttgcaagaaccatggcggatttagactctgtcctgtgtctccggcccagtttcggagccaggagggcagcccttgcactatcaagtagcatgccaaagttaatcattggccctgccaagtacatggcagatcaggctgtttttgtgtgcctgtttttctattttacgtaaatcaccctgaacatgtttgcatcaacctcctggtgatgtacctttgatcaatagattttagacaaaagtggtttttgagtccaaagatcagggctgggttgacctgcagactggatacagagtgtataaaaacaggggcaaggcgcaggctgatagcagagcaatcgccaagtcggtaccgagctcggaaagggcgaattccagcacactggcggccgttactagtggatccaaggatggaggaaggaatggatgtgctccatgactttggaatccagtcaacgcactacctccaggtgaattaccaggactctcaggattggttcatcttggtgtccgtgattgcagacctcaggaatgccttctatgtcctctttcccatctggttccatctgcgtgaagctgtgggcatcaaacttctctgggtagctgtgattggagactggctcaacctcgtctttaaatggattctgtttggacagcgtccatactggtgggtcatggacaccgactactatagcaacacctctgtgccactgataaagcaatttccagttacctgtgaaactggaccagggagtccctctggtcatgccatgggtacagcaggtgtatactacgtgatggtcacatctaccctttctatctttcgggggagaaaaaggccaacctacagatttcggtgcttgaatatccttctgtggttgggattctgggctgtgcagctgaacgtctgtctgtcccgaatctaccttgctgctcatttcccccatcaggttgttgctggagtcctgtcaggcattgctgttgctgaaactttccgccacatccagagcatctacaatgccagcctcaagaaatattttctcattactttcttcctgttcagttttgccattggattttacctgctgctcaaggggctgggtgtggacctcctgtggacactggaaaaagccaggagatggtgtgagcggccggaatgggttcacattgacaccacaccctttgccagccttctcaagaacgtggggaccctctttggcctgggggtggctctcaactccagcatgtacagggaaagctgcaagggcaagcttagcaagtggttcccattccgcctcagctgcattgtggtgtctctcatcctcctgcacctctttgactctttgaaacccccatcccaaactgagctgatcttctacaccttgtccttctgcaagagtgcagcagtgcccctggcatctgtcagcctcatcccctactgccttgcccgggtcttcgaccagccagacaagaagtctttgtaaggcgaattctgcagatatccatcacactggcggccgctcgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagtcttgtcctaataaaattaagttgcatcattttgtctgactaggtgtccttctataatattatggggtggaggggggtggtatggagcaagggaatagattctctgggtagctgtgattggagactggctcaacctcgtctttaaatggtgagtaagagccagacagagaggagatcggcaagggaagaggctggtatagtctctggaaatgtctgacatcagaaattgccttctccgtgctagtcgggaagccacaggttactcccctcctgactctggatcatctacctaaagagggaagacagcgaaaatcttgttgatgaattgaagatacaggcaaggctatttcatatggatggaaggccagccggacttagtgaaaagtctaaagtatataggctataagcaaagaagcacagttgtattactttgaagcagaataggtaacataaagataggtgtatgaaagatgatgtatgaaaacaggaaacaagtatataatttaaaaacccactgcatggagaaagaagaagtaacccagagaactgggctctaacccttgttcggccacctaccagctgtatgtcctgggaaagtcagagcaactctctgagtttctgtcttacaatctgtaaacttaaattatccttgggagcttaataataattctagttaacatttattgaacatttagtaggtactaggccatgtgccaagtgcttttctgtgaggtagttactactgtattatagatgaagagacagagccattacgataagggaccagaactagatccacatggtggtcaaaggcagaggcagcattttaaggccagacaggctagtttcagaggcaaagctcttaaggattatgctgtattcattgctgacaaattgactttttcagggagcaggaggaagccaaattttaatgtttgtcaatttctctggtgtatatattcctattggggccacttttgatgggtcaagcttgcaaaaatcctgaatattttacaattgtcagccaataccatatgattctagctcattagtggcttggcctctatttcctcatctagaaaataaagagcttgggccagctaaaacctttaccatccctaaactatctatggaagggtctctcctgtagggcctgcggggtctattgggaaccaagctggagtgcagtggcacaatcttggctcactgcaatctccgcctcctgggttcaagcgattctcctgcctcagcctcccgagttgttgggattccaggcatcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 44 - DNA, 6624 bp, synthetic construct
  source 1..6624: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgctctagagagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaaggcgaattctgcagatatccatcacactggcggccgctcgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagtcttgtcctaataaaattaagttgcatcattttgtctgactaggtgtccttctataatattatggggtggaggggggtggtatggagcaagggaatagagtaagaaccatatagagaggagatcagcaagaaaagaggctggcataggctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcggcaatgctcttatgaactatgttacgtccagtgctggaagggtctctcctgtagggcctgcggggtctattgggaaccaagctggagtgcagtggcacaatcttggctcactgcaatctccgcctcctgggttcaagcgattctcctgcctcagcctcccgagttgttgggattccaggcatcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 45 - DNA, 6764 bp, synthetic construct
  source 1..6764: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgctctagagagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaaggcgaattctgcagatatccatcacactggcggccgctcgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagtcttgtcctaataaaattaagttgcatcattttgtctgactaggtgtccttctataatattatggggtggaggggggtggtatggagcaagggaatagagtaagaaccatatagagaggagatcagcaagaaaagaggctggcattcgctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcggcagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctacgtcctcttccccatcgttttagagctagaaatagcaagttaaaataaggctagtccgttatcaacttgaaaaagtggcaccgagtcggtgcttttttgccgcgggaagggtctctcctgtagggccttcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 46 - DNA, 7758 bp, synthetic construct
  source 1..7758: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaattcgagctcggtaccctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagctctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagtgagtcgacaataaaatatctttattttcattacatctgtgtgttggttttttgtgtgtgcatgctggggacggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 47 - DNA, 7499 bp, synthetic construct
  source 1..7499: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggatccgaattcgagctcggtaccaagcttgccgagctcaggtacccgctaatctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatggtcagacatttccagagttttagtactctggaaacagaatctactaaaacaaggcaaaatgccgtgtttatctcgtcaacttgttggcgagattttttctcgaggtcggaggatccacaaaggctagccggtctatgcatacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacaccatccacgctgttttgacctccatagaagacaccgggaccgatccagcctaagcttgccaccatgaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaagattacaaagacgatgacgataagtgagtcgaccgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 48 - DNA, 7501 bp, synthetic construct
  source 1..7501: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctcgaggatccgaattcgagctcggtaccaagcttgccgagctcaggtacccgctaatctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgatggacagacattgcgagagttttagtactctggaaacagaatctactaaaacaaggcaaaatgccgtgtttatctcgtcaacttgttggcgagattttttctcgaggtcggaggatccacaaaggctagccggtctatgcatacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacaccatccacgctgttttgacctccatagaagacaccgggaccgatccagcctaagcttgccaccatgaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaagattacaaagacgatgacgataagtgagtcgaccgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 49 - DNA, 4034 bp, synthetic construct
  source 1..4034: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcgaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 50 - DNA, 114 bp, synthetic construct
  source 1..114: mol_type = other DNA
gggaatagattctctgggtagctgtgattggagactggctcaacctcgtctttaaatggtgagtaagagccagacagagaggagatcggcaagggaagaggctggtatagtctc

SEQ ID NO: 51 - DNA, 26 bp, synthetic construct
  source 1..26: mol_type = other DNA
ataaagagcttgggccagctaaaacc

SEQ ID NO: 52 - DNA, 1206 bp, synthetic construct
  source 1..1206: mol_type = other DNA
ctcctggccctggaagttgccactccagtgcccaccagtcttgtcctaataaaattaagttgcatcattttgtctgactaggtgtccttctataatattatggggtggaggggggtggtatggagcaagggaatagattctctgggtagctgtgattggagactggctcaacctcgtctttaaatggtgagtaagagccagacagagaggagatcggcaagggaagaggctggtatagtctctggaaatgtctgacatcagaaattgccttctccgtgctagtcgggaagccacaggttactcccctcctgactctggatcatctacctaaagagggaagacagcgaaaatcttgttgatgaattgaagatacaggcaaggctatttcatatggatggaaggccagccggacttagtgaaaagtctaaagtatataggctataagcaaagaagcacagttgtattactttgaagcagaataggtaacataaagataggtgtatgaaagatgatgtatgaaaacaggaaacaagtatataatttaaaaacccactgcatggagaaagaagaagtaacccagagaactgggctctaacccttgttcggccacctaccagctgtatgtcctgggaaagtcagagcaactctctgagtttctgtcttacaatctgtaaacttaaattatccttgggagcttaataataattctagttaacatttattgaacatttagtaggtactaggccatgtgccaagtgcttttctgtgaggtagttactactgtattatagatgaagagacagagccattacgataagggaccagaactagatccacatggtggtcaaaggcagaggcagcattttaaggccagacaggctagtttcagaggcaaagctcttaaggattatgctgtattcattgctgacaaattgactttttcagggagcaggaggaagccaaattttaatgtttgtcaatttctctggtgtatatattcctattggggccacttttgatgggtcaagcttgcaaaaatcctgaatattttacaattgtcagccaataccatatgattctagctcattagtggcttggcctctatttcctcatctagaaaataaagagcttgggccagctaaaacctttaccatccctaaactatctatgtctactgtcaagtgattctgaagacccttctagctc

SEQ ID NO: 53 - protein, 1368 aa, Streptococcus pyogenes [sequence omitted]

SEQ ID NO: 54 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 55 - DNA, 4152 bp, synthetic construct
  source 1..4152: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaa

SEQ ID NO: 56 - DNA, 3159 bp, synthetic construct
  source 1..3159: mol_type = other DNA
atgaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaaggga

SEQ ID NO: 57 - DNA, 21 bp, synthetic construct
  source 1..21: mol_type = other DNA
gccttctatgtcctctttccc

SEQ ID NO: 58 - DNA, 24 bp, synthetic construct
  source 1..24: mol_type = other DNA
ttagagcccagttctctgggttac

SEQ ID NO: 59 - DNA, 25 bp, synthetic construct
  source 1..25: mol_type = other DNA
gccagacaagaagtctttgtaaggc

SEQ ID NO: 60 - DNA, 26 bp, synthetic construct
  source 1..26: mol_type = other DNA
ggacatggacaaggtcgagacattcc

SEQ ID NO: 61 - DNA, 23 bp, synthetic construct
  source 1..23: mol_type = other DNA
ccaaagaatattagagctagaag

SEQ ID NO: 62 - DNA, 27 bp, synthetic construct
  source 1..27: mol_type = other DNA
gctgttgaataggggacattacagacg

SEQ ID NO: 63 - DNA, 27 bp, synthetic construct
  source 1..27: mol_type = other DNA
cgtctgtaatgtcccctattcaacagc

SEQ ID NO: 64 - DNA, 27 bp, synthetic construct
  source 1..27: mol_type = other DNA
aagtacctagaacagtgtctggcacag

SEQ ID NO: 65 - DNA, 20 bp, synthetic construct
  source 1..20: mol_type = other DNA
tcttcgaccagccagacaag

SEQ ID NO: 66 - DNA, 20 bp, synthetic construct
  source 1..20: mol_type = other DNA
ggtcctttaggaggtcatag

SEQ ID NO: 67 - DNA, 22 bp, synthetic construct
  source 1..22: mol_type = other DNA
gcagttccctgtaacctgtgag

SEQ ID NO: 68 - DNA, 21 bp, synthetic construct
  source 1..21: mol_type = other DNA
ggtcggctttatctttccctg

SEQ ID NO: 69 - DNA, 22 bp, synthetic construct
  source 1..22: mol_type = other DNA
gttggtatacacggtgtgcctg

SEQ ID NO: 70 - DNA, 20 bp, synthetic construct
  source 1..20: mol_type = other DNA
ctgacgccagcgtcaatcac

SEQ ID NO: 71 - DNA, 22 bp, synthetic construct
  source 1..22: mol_type = other DNA
atggaatcctgcggcatccatg

SEQ ID NO: 72 - DNA, 21 bp, synthetic construct
  source 1..21: mol_type = other DNA
cagggtacatggtggttccac

SEQ ID NO: 73 - DNA, 24 bp, synthetic construct
  source 1..24: mol_type = other DNA
tgatagcagagcaatcgccaagtc

SEQ ID NO: 74 - DNA, 24 bp, synthetic construct
  source 1..24: mol_type = other DNA
agggtagatgtgaccatcacgtag

SEQ ID NO: 75 - DNA, 22 bp, synthetic construct
  source 1..22: mol_type = other DNA
tgtccgtgattgcagacctcag

SEQ ID NO: 76 - DNA, 22 bp, synthetic construct
  source 1..22: mol_type = other DNA
agatgatccagagtcaggaggg

SEQ ID NO: 77 - DNA, 24 bp, synthetic construct
  source 1..24: mol_type = other DNA
ctgttcgaagagtctgcatgtaag

SEQ ID NO: 78 - DNA, 22 bp, synthetic construct
  source 1..22: mol_type = other DNA
gaggggttttaggaagtgctgg

SEQ ID NO: 79 - DNA, 22 bp, synthetic construct
  source 1..22: mol_type = other DNA
cagcttgttcacaaccagactc

SEQ ID NO: 80 - DNA, 22 bp, synthetic construct
  source 1..22: mol_type = other DNA
cagttttctccaggctcctaac

SEQ ID NO: 81 - DNA, 22 bp, synthetic construct
  source 1..22: mol_type = other DNA
tgggacattttagggagccaag

SEQ ID NO: 82 - DNA, 22 bp, synthetic construct
  source 1..22: mol_type = other DNA
cattctcgtcttggaacgtgtg

SEQ ID NO: 83 - DNA, 24 bp, synthetic construct
  source 1..24: mol_type = other DNA
gcaagataagagtcggtcttcgtg

SEQ ID NO: 84 - DNA, 22 bp, synthetic construct
  source 1..22: mol_type = other DNA
acactagcagtcatacggactc

SEQ ID NO: 85 - DNA, 19 bp, synthetic construct
  source 1..19: mol_type = other DNA
actggggctcgtatctcgg

SEQ ID NO: 86 - DNA, 22 bp, synthetic construct
  source 1..22: mol_type = other DNA
caagggagaggcttcagaatcg

SEQ ID NO: 87 - DNA, 21 bp, synthetic construct
  source 1..21: mol_type = other DNA
tacagggagcctgcttctgcc

SEQ ID NO: 88 - DNA, 22 bp, synthetic construct
  source 1..22: mol_type = other DNA
cacaatcccttgtgggaagggc

SEQ ID NO: 89 - DNA, 22 bp, synthetic construct
  source 1..22: mol_type = other DNA
agcacatcattcattcctgggc

SEQ ID NO: 90 - DNA, 22 bp, synthetic construct
  source 1..22: mol_type = other DNA
ccagttcacttcgagcgtcttc

SEQ ID NO: 91 - DNA, 20 bp, synthetic construct
  source 1..20: mol_type = other DNA
gcatccctgggatccttagg

SEQ ID NO: 92 - DNA, 20 bp, synthetic construct
  source 1..20: mol_type = other DNA
tagctgagcgtgagaagggg

SEQ ID NO: 93 - DNA, 22 bp, synthetic construct
  source 1..22: mol_type = other DNA
ctgtttagctggcattgactcc

SEQ ID NO: 94 - DNA, 22 bp, synthetic construct
  source 1..22: mol_type = other DNA
gcttttgtctggtgtcatcccc

SEQ ID NO: 95 - DNA, 24 bp, synthetic construct
  source 1..24: mol_type = other DNA
ctctggaagagctacatagtcatc

SEQ ID NO: 96 - DNA, 24 bp, synthetic construct
  source 1..24: mol_type = other DNA
gtaccatgaacttttaggacggtc

SEQ ID NO: 97 - DNA, 20 bp, synthetic construct
  source 1..20: mol_type = other DNA
aatggtcagacatttccaaa

SEQ ID NO: 98 - DNA, 20 bp, synthetic construct
  source 1..20: mol_type = other DNA
aatggtgagagatttccaga

SEQ ID NO: 99 - DNA, 20 bp, synthetic construct
  source 1..20: mol_type = other DNA
gatgctcagacatttcaaaa

SEQ ID NO: 100 - DNA, 20 bp, synthetic construct
  source 1..20: mol_type = other DNA
aatgtttagacatttccaga

SEQ ID NO: 101 - DNA, 20 bp, synthetic construct
  source 1..20: mol_type = other DNA
gatgggctggcatttccaga

SEQ ID NO: 102 - DNA, 20 bp, synthetic construct
  source 1..20: mol_type = other DNA
gagtgtcagacacttccaga

SEQ ID NO: 103 - DNA, 20 bp, synthetic construct
  source 1..20: mol_type = other DNA
gatggtcataaatttcctga

SEQ ID NO: 104 - DNA, 20 bp, synthetic construct
  source 1..20: mol_type = other DNA
tatagtcagacttttccaga

SEQ ID NO: 105 - DNA, 20 bp, synthetic construct
  source 1..20: mol_type = other DNA
gattttcagacatttccaca

SEQ ID NO: 106 - DNA, 20 bp, synthetic construct
  source 1..20: mol_type = other DNA
catggtgagacatttccagt

SEQ ID NO: 107 - DNA, 20 bp, synthetic construct
  source 1..20: mol_type = other DNA
agtacagcatcggcctggac

SEQ ID NO: 108 - DNA, 20 bp, synthetic construct
  source 1..20: mol_type = other DNA
gggctccgatcaggttcttc

SEQ ID NO: 109 - DNA, 20 bp, synthetic construct
  source 1..20: mol_type = other DNA
ggctgtattcccctccatcg

SEQ ID NO: 110 - DNA, 22 bp, synthetic construct
  source 1..22: mol_type = other DNA
ccagttggtaacaatgccatgt

SEQ ID NO: 111 - DNA, 22 bp, synthetic construct
  source 1..22: mol_type = other DNA
tgacctacagactgaatccagg

SEQ ID NO: 112 - DNA, 22 bp, synthetic construct
  source 1..22: mol_type = other DNA
taacatctgtgctcaggagctg

SEQ ID NO: 113 - DNA, 21 bp, synthetic construct
  source 1..21: mol_type = other DNA
cagccgcacaagaagtcgttg

SEQ ID NO: 114 - DNA, 20 bp, synthetic construct
  source 1..20: mol_type = other DNA
ccactcccactgtcctttcc

SEQ ID NO: 115 - DNA, 23 bp, synthetic construct
  source 1..23: mol_type = other DNA
ggctcagtagatcaagtgcctgc

SEQ ID NO: 116 - DNA, 20 bp, synthetic construct
  source 1..20: mol_type = other DNA
tctgggaatcagggactggg

SEQ ID NO: 117 - RNA, 20 nt, synthetic construct
  source 1..20: mol_type = other RNA
gatggtcagacatttccaga

SEQ ID NO: 118 - RNA, 24 nt, synthetic construct
  source 1..24: mol_type = other RNA
ctggctcaacctcgtctttaaatg

SEQ ID NO: 119 - RNA, 24 nt, synthetic construct
  source 1..24: mol_type = other RNA
ttctgatggtcagacatttccaga

SEQ ID NO: 120 - RNA, 24 nt, synthetic construct
  source 1..24: mol_type = other RNA
aggtagatgatccagagtcaggag

SEQ ID NO: 121 - RNA, 21 nt, synthetic construct
  source 1..21: mol_type = other RNA
tgatggacagacattgcgaga

SEQ ID NO: 122 - RNA, 21 nt, synthetic construct
  source 1..21: mol_type = other RNA
gcatgagtagcccgtggcttc

SEQ ID NO: 123 - RNA, 21 nt, synthetic construct
  source 1..21: mol_type = other RNA
tcagagagaggggttggaagc

SEQ ID NO: 124 - RNA, 21 nt, synthetic construct
  source 1..21: mol_type = other RNA
agatgatccaaagtcagagag

SEQ ID NO: 125 - RNA, 20 nt, synthetic construct
  source 1..20: mol_type = other RNA
tctatgtcctctttcccatc

SEQ ID NO: 126 - RNA, 20 nt, synthetic construct
  source 1..20: mol_type = other RNA
tctacgtcctcttccccatc

SEQ ID NO: 127 - RNA, 20 nt, synthetic construct
  source 1..20: mol_type = other RNA
ttgctgatctcctctctata

SEQ ID NO: 128 - RNA, 20 nt, synthetic construct
  source 1..20: mol_type = other RNA
gtggtaagaaccatatagag

SEQ ID NO: 129 - RNA, 20 nt, synthetic construct
  source 1..20: mol_type = other RNA
gaggagatcagcaagaaaag

SEQ ID NO: 130 - RNA, 20 nt, synthetic construct
  source 1..20: mol_type = other RNA
agatcagcaagaaaagaggc

SEQ ID NO: 131 - RNA, 20 nt, synthetic construct
  source 1..20: mol_type = other RNA
ctggggaaagcaacttctga

(19) United States
(12) Patent Application Publication
(10) Pub. No.: US 2025/0376698 A1
(43) Pub. Date: Dec. 11, 2025

(54) COMPOSITIONS AND METHODS FOR PROMOTING LIVER REGENERATION BY GENE EDITING IN METABOLIC LIVER DISEASE

(71) Applicant: Duke University, Durham, NC (US)

(72) Inventors: Dwight Koeberl, Durham, NC (US); Hye Ri Kang, Durham, NC (US); Benjamin Arnson, Durham, NC (US); Bryan Cullen, Durham, NC (US)

(21) Appl. No.: 18/854,346

(22) PCT Filed: Apr. 6, 2023
(86) PCT No.: PCT/US2023/065442

§ 371 Date: Oct. 4, 2024

Related U.S. Application Data
(60) Provisional application No. 63/328,482, filed on Apr. 7, 2022.
(60) Provisional application No. 63/329,561, filed on Apr. 11, 2022.

(51) Int. Cl. C12N 15/864; A61K 48/00; A61P 3/08; C12N 9/22; C12N 15/113; C12N 15/52

(52) U.S. Cl. CPC C12N 15/8645; A61K 48/0058; A61P 3/08; C12N 9/226; C12N 15/113; C12N 15/52; C12Y 301/03009; C12N 2750/14143

(57) ABSTRACT

Embodiments of the instant disclosure relate to novel gene editing vectors, compositions, and methods for editing a G6PC gene to treat glucose storage diseases (e.g., GSD Ia). In certain embodiments, vectors described herein comprise one or more CRISPR/Cas9 components to allow for integration of a G6PC transgene into a target gene locus in a subject in need thereof, thereby allowing for stable expression of a therapeutic protein (e.g., glucose-6-phosphatase) and reversal and/or treatment of disease symptoms in the subject.

### CROSS REFERENCE TO RELATED APPLICATIONS

This application claims priority to U.S. Provisional Patent Application Ser. No. 63/329,561, filed Apr. 11, 2022, and U.S. Provisional Patent Application Ser. No. 63/328,482, filed Apr. 7, 2022, the contents of each are hereby incorporated by reference in their entirety.

### FEDERAL FUNDING LEGEND

This invention was made with Government support under Federal Grant No. R01DK105434 awarded by the National Institutes of Health. The Federal Government has certain rights to this invention.

### INCORPORATION BY REFERENCE OF SEQUENCE LISTING

This application contains a sequence listing that has been submitted via PatentCenter in a computer readable format and is hereby incorporated by reference in its entirety. The computer readable file, created on Apr. 5, 2023 is named 109726-754012_23-2071-WO_SequenceListing.xml and is about 223,000 bytes in size.

### BACKGROUND OF THE INVENTION

Fatty liver disease has been linked with impaired (macro) autography and enhanced apoptosis, which leads to progressive hepatosteatosis and an increased risk for hepatocellular carcinoma. Hepatosteatosis occurs in liver metabolic diseases from single gene defects, including the glycogenosis or glycogen storage diseases (GSD). GSD Ia (von Gierke disease) results from pathogenic variants in the G6PC gene that causes glucose-6-phosphatase (G6Pase) deficiency in liver. G6Pase deficiency leads to the accumulation of glycogen in the liver due to accumulated glucose-6-phosphate, accompanied by hepatosteatosis. GSD Ia can be treated with gene therapy, however, the effect of gene therapy wanes quickly due to the loss of non-integrating viral vectors under clinical development, including adeno-associated virus (AAV) vectors.

### BRIEF SUMMARY OF THE DISCLOSURE

The present disclosure provides, in part, nucleic acids, gene editing vectors, compositions, and methods for the treatment and management of various glycogen storage diseases (GSD).

Disclosed herein is an isolated nucleic acid comprising (i) a nucleotide sequence encoding a glucose-6-phosphatase, (ii) a nucleotide sequence with homology with a region located 5′ of a target site in a G6PC gene locus, and (iii) a nucleotide sequence with sequence homology with a region located 3′ of the target site in a G6PC gene locus, wherein (i) is flanked by (ii) and (iii).

In some aspects, the nucleotide sequence of (i) comprises a human, canine, or murine G6PC coding sequence, or a codon optimized sequence thereof. For example, in some aspects, the nucleotide sequence of (i) can have at least 70%, at least 75%, at least 80%, at least 85%, at least 90%, at least 95%, at least 96%, at least 97%, at least 98%, at least 99% or 100% sequence identity to any one of SEQ ID NOs: 16 to 19. In some aspects, the nucleotide sequence of (i) comprises any one of SEQ ID NOs: 16 to 19.

In some aspects, the nucleotide sequence of (i) comprises a human G6PC or codon optimized sequence thereof. For example, in some aspects, the nucleotide sequence of (i) has at least 70%, at least 75%, at least 80%, at least 85%, at least 90%, at least 95%, at least 96%, at least 97%, at least 98%, at least 99% or 100% sequence identity to any one of SEQ ID NOs: 16 to 18. In some aspects, the nucleotide sequence of (i) comprises any one of SEQ ID NOs: 16 to 18. In some further aspects, the nucleotide sequence of (i) comprises SEQ ID NO: 18.

In some aspects, the nucleotide sequence of (i) further comprises a promoter sequence operably linked to the nucleotide sequence encoding the glucose-6-phosphatase. In some aspects, the promoter sequence comprises a human G6PC promoter.

In any of the foregoing or related aspects, the nucleotide sequence of (ii) can have sequence homology to a region located 5′ to the target site in a murine, canine, or human G6PC gene locus. For example, in some aspects the nucleotide sequence of (ii) has at least 70%, at least 75%, at least 80%, at least 85%, at least 90%, at least 95%, at least 96%, at least 97%, at least 98%, at least 99% or 100% sequence identity to any one of SEQ ID NOs: 25, 27, 29, 30, 32, or 33. For example, in some aspects, the the nucleotide sequence of (ii) comprises any one of SEQ ID NO: 25, 27, 29, 30, 32, or 33.

In some aspects, the nucleotide sequence of (ii) has sequence homology to a region located 5′ upstream of the target site in a human G6PC gene locus. For example, in some aspects, the nucleotide sequence of (ii) may have at least 70%, at least 75%, at least 80%, at least 85%, at least 90%, at least 95%, at least 96%, at least 97%, at least 98%, at least 99% or 100% sequence identity to SEQ ID NO 32 or SEQ ID NO: 33. In some aspects, the nucleotide sequence of (ii) comprises SEQ ID NO: 32 or SEQ ID NO: 33.

In any of the foregoing or related aspects, the nucleotide sequence of (iii) may have sequence homology to a region located 3′ to the target site in a murine, canine, or human G6PC gene locus. For example, in some aspects, the nucleotide sequence of (iii) has at least 70%, at least 75%, at least 80%, at least 85%, at least 90%, at least 95%, at least 96%, at least 97%, at least 98%, at least 99% or 100% sequence identity to any one of SEQ ID NOs: 26, 28, 31, 34, or 35. In some aspects, the nucleotide sequence of (iii) comprises SEQ ID NO: 26, 28, 31, 34, or 35.

In further aspects, the nucleotide sequence of (iii) may have sequence homology to a region located 3′ to the target site in a human G6PC gene locus. For example, in some aspects, the nucleotide sequence of (iii) may have at least 70%, at least 75%, at least 80%, at least 85%, at least 90%, at least 95%, at least 96%, at least 97%, at least 98%, at least 99% or 100% sequence identity to SEQ ID NOs: 34 or 35. In some aspects, the nucleotide sequence of (iii) comprises SEQ ID NO: 34 or 35.

In any of the foregoing or related aspects, the nucleotide sequence of the isolated nucleic acid provided herein may have at least 70%, at least 75%, at least 80%, at least 85%, at least 90%, at least 95%, at least 96%, at least 97%, at least 98%, at least 99% or 100% sequence identity to any one of SEQ ID NO: 36 to 40. For example, in some aspects the nucleotide sequence of the isolated nucleic acid provided herein may comprise any one of SEQ ID NOs: 36 to 40. In some aspects, a nucleotide sequence of an isolated nucleic acid provided herein has at least 70%, at least 75%, at least 80%, at least 85%, at least 90%, at least 95%, at least 96%, at least 97%, at least 98%, at least 99% or 100% sequence identity to SEQ ID NO: 39 or 40. For example, in some aspects, a nucleotide sequence of an isolated nucleic acid provided herein comprises SEQ ID NO: 39 or 40.

Also disclosed are vectors comprising any of the isolated nucleic acids provided herein.

In further aspects, disclosed herein are vector systems for stably integrating a therapeutic G6PC transgene in a cell, the system comprising (a) a first vector comprising the isolated nucleic acid disclosed herein; and a second vector comprising a nucleotide sequence encoding a Cas9 endonuclease; wherein either the first vector or the second vector further comprises a nucleotide sequence encoding a small guide RNA (gRNA) targeting the target site in the G6PC gene locus.

In various aspects, the Cas9 endonuclease encoded by the vector system comprises a Staphylococcus aureus Cas9 (SaCas9) or a Streptococcus pyogenes Cas9 (SpCas9). In some aspects, the Cas9 endonuclease comprises a SaCas9 endonuclease and the target site in the G6PC gene locus comprises any one of SEQ ID NOs: 1 to 8. For example, in some aspects, the target site in the G6PC gene locus may comprise any one of SEQ ID NOs: 5 to 8. In further aspects, the Cas9 endonuclease comprises a SpCas9 endonuclease and the target site in the G6PC gene locus comprises any one of SEQ ID NOs: 9 to 15. For example, in some aspects, the target site in the G6PC gene locus may comprise or consist of any one of SEQ ID NOs: 10 to 15.

In various aspects, the nucleotide sequence encoding the gRNA is operably linked to an exogenous promoter and/or enhancer. In various aspects, the nucleotide sequence encoding the Cas9 endonuclease is operably linked to an exogenous promoter and/or enhancer. In various aspects, the exogenous promoter and/or enhancer can be a U6 promoter, a CMV enhancer or a human G6PC promoter.

In any of the vector systems provided herein, the first and second vector can be viral vectors. For example, in some aspects, the the first and the second vector comprise adeno-associated virus (AAV) vectors, lentivirus vectors, adenovirus vectors, retrovirus vectors, herpesvirus vectors, and combinations thereof. In some aspects, the first and second vectors are AAV vectors.

In any of the vector systems provided herein, the first vector can comprise a nucleic acid sequence of any one of SEQ ID NOs: 41 to 45. In any of the vector system provided herein, the second vector can comprise a nucleic acid sequence of any one of SEQ ID NOs: 46 to 48. In some aspects, the first vector of a vector system provided herein comprises a nucleic acid sequence of SEQ ID NO: 41 or 42 and the second vector comprises a nucleic acid sequence of SEQ ID NO: 46. In some aspects, the first vector of a vector system provided herein comprises a nucleic acid sequence of SEQ ID NO: 43 and the second vector comprises a nucleic acid sequence of SEQ ID NO: 47. In some aspects, the first vector of a vector system provided herein comprises a nucleic acid sequence of SEQ ID NO: 44 and the second vector comprises a nucleic acid sequence of SEQ ID NO: 48. In still further aspects, the first vector of a vector system provided herein comprises a nucleic acid sequence of any one of SEQ ID NOs: 45 and the second vector comprises a nucleic acid sequence of SEQ ID NOs: 46.

Also disclosed herein are pharmaceutical compostions comprising any of the first and/or second vector of a vector system provided herein and a pharmaceutically acceptable diluent, carrier and/or excipient.

In additional aspects, disclosed herein are methods of stably integrating a therapeutic G6PC transgene into a cell, the method comprising delivering the vector system disclosed herein to the cell, the vector system comprising the therapeutic G6PC transgene, wherein the cell stably integrates the therapeutic transgene into its genomic DNA.

Also disclosed herein are methods of expressing a G6PC transgene in a subject, the method comprising administering to the subject a therapeutically effective amount of the vector system disclosed herein, wherein at least one cell of the subject stably integrates and expresses the G6PC transgene into its genomic DNA.

Also disclosed herein are methods of treating, slowing and/or preventing progression of a glycogen storage disease in a subject by stably integrating a G6PC transgene into genomic DNA of at least one cell of a subject in need thereof. In some aspects, stably integrating the G6PC transgene comprises delivering one or more nucleic acid vectors to the subject, the nucleic acid vectors encoding for a site-directed endonuclease, a guide RNA targeting a target site in a G6PC gene locus, and the G6PC transgene. In some aspects, the site directed endonuclease generates a double stranded break at or near the target site in the G6PC gene locus and the G6PC transgene is integrated at the site of the double stranded break via homologous recombination. In further aspects, the cell can stably express the integrated G6PC transgene. In still further aspects, the method of treating, slowing and/or preventing progression of a glycogen storage disease can comprise administering to the subject a therapeutically effect amount of a vector system disclosed herein.

In various aspects, delivering or administering the vector system in any of the methods herein can comprise administering or delivering the first and second vectors separately. For example, in some aspects, the the first vector can be administered or delivered before the second vector. In other aspects, the first vector is administered or delivered after the second vector. In still other aspects, the first vector and the second vector are administered or delivered concurrently.

In various aspects, a ratio of the first vector to the second vector delivered to the cell or administered to the subject is from about 10:1 to about 1:1, from about 8:1 to about 1:1, from about 5:1 to about 1:1, or from about 4:1 to about 1:1. For example, in some aspects, the ratio of the first vector to the second vector can be about 10:1, about 9:1, about 8:1, about 7:1, about 6:1, about 5:1, about 4:1, about 3:1, about 2:1, or about 1:1.

In an aspect, a disclosed method can comprise measuring and/or determining one or more liver enzymes and/or metabolites. Liver enzyme and/or metabolites can comprise Alanine transaminase (ALT), Aspartate transaminase (AST), Alkaline phosphatase (ALP), Albumin and total protein, Bilirubin, Gamma-glutamyltransferase (GGT), L-lactate dehydrogenase (LD), Prothrombin time (PT), or any combination thereof. In an aspect, a disclosed method can comprise measuring and/or determining one or more urine enzymes and/or metabolites (such as, for example, glucotetrasaccharides (HEX4)).

In any of the foregoing or related methods of treating a subject, the method can further comprise administering one or more additional therapeutic agent(s) to the subject.

In some aspects, the one or more additional therapeutic agent(s) can comprise a gene replacement vector comprising a G6PC transgene operably linked to a promoter. In some aspects, the gene replacement vector is an AAV vector. In some aspects, the gene replacement vector expresses the G6PC transgene episomally in at least one cell of the subject.

In some aspects, the one or more additional therapeutic agent(s) comprises an antilipemic agent, an mTOR inhibitor that induces autophagy and/or an agent that improves transduction. For example, in some aspects, the one or more additional therapeutic agent(s) can comprise cholestryramine, colesevelam, colestipol, clofibrate, fenofibrate, gemfibrozil, benzafibrate, alirocumab, evinacumab, evolocumab, niacin, icosapent theyl, omedga-3-acid ethyl esters, omega-3 carboxylic acids, atorvastatin, fluvastatin, lovastatin, pitavastatin, pravastatin, rosuvastatin, simvastatin, exetimibe, lomitapide, mipomoersen, resveratrol, rapamycin, CC1-779, RAD001, Torin 1, KU-0063794, WYE-354, AZD8055, metformin or any combination thereof.

In any of the foregoing or related aspects herein, the glycogen storage disease can comprise a GSD I. For example, the glycogen storage disease can comprise GSD Ia.

In any of the foregoing or related aspects, treating and/or slowing and/or preventing progression of the glycogen storage disease in the subject can comprise restoring one or more aspects of cellular homeostasis and/or cellular functionality and/or metabolic dysregulation in at least one cell of the subject. In various aspects, the subject in any of the methods herein may be a neonate or infant that is 2 or 3 months of age. In various aspects, the subject in any of the methods herein may be an adult.

Also provided herein is a kit for prevention and/or treatment of a GSD disease (e.g., GSD type 1a) in a subject, the kit comprising a vector system described herein and instructions for use.

These and other features and advantages of the disclosure will be fully understood from the following detailed description and the accompanying drawings.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A is a representative schematic depicting integration of a G6PC transgene in the canine G6PC locus.

FIG. 1B is a representative agarose gel showing cleaved DNA that reflect indels in the G6PC locus with the Surveyor assay, and a representative immunoblot showing Cas9 protein expression in transfected dog fibroblasts.

FIG. 1C is a schematic of a corrected cG6PC locus containing an integrated transgene and a representative agarose gel showing PCR of the integrated transgene in fibroblasts transfected with both CRISPR and donor vectors.

FIG. 1D is a sequencing output of a PCR product confirming integrated transgene in a canine G6PC locus in transfected dog fibroblasts.

FIG. 2A is a diagram of an experimental protocol where GSD Ia dogs are treated initially with gene replacement (AAV-G6Pase/AAV9, AAV-G6Pase/AAV10, and AAV-G6Pase/AAV8) followed by gene editing (AAV-CRISPR/Cas9+AAV-cG6PC/AAV7).

FIG. 2B is a representative agarose gel showing PCR of the integrated transgene in dogs 4 months (4M) and 16 months (16M) after gene editing vector treatment as adults.

FIG. 2C-2D are histograms depicting the quantification of AAV-G6Pase (FIG. 2C), AAV-cG6PC (Donor) (FIG. 2D), and AAV-CRISPR/Cas9 (FIG. 2E) vector genomes in adult dogs before CRISPR (BC), and 4 and 16 months after treatment as adults with gene editing vectors described herein.

FIG. 2F-2G depict levels of G6Pase activity (FIG. 2F) and glycogen content (FIG. 2G) in livers of dogs 4 and 16 months after treatment as adults with gene editing vectors described herein.

FIG. 2H is a line plot showing results from an 8-hour fasting test in dogs before and after treatment as adults with gene editing vectors described herein (AAV-CRISPR/Cas9+AAV-cG6PC, arrow).

FIG. 3A-3B are plots showing IgG response determined by ELISA for anti-AAV7 (FIG. 3A) and anti-Cas9 (FIG. 3B) antibodies in dogs treated as adults with gene editing vectors described herein.

FIG. 4A is a diagram of an experimental protocol where GSD Ia puppies are treated as neonates with gene editing vectors (AAV-CRISPR/Cas9+AAV-cG6PC/AAV7) and then treated at 2 and 3 months with gene therapy vectors (AAV-G6Pase/AAV10, AAV-G6Pase/AAV9, and AAV-G6Pase/AAV8).

FIG. 4B is a representative agarose gel showing PCR of the integrated transgene in puppies 4 months (4M) and 16 months (16M) after treatment as neonates with gene editing vectors.

FIG. 4C-4E plots depicting the quantification of AAV-G6Pase (FIG. 4C), AAV-cG6PC (Donor) (FIG. 4D), and AAV-CRISPR/Cas9 (FIG. 4E) vector genomes in puppies 4 and 16 months after treatment as neonates with gene editing vectors described herein.

FIG. 4F-4G depict levels of G6Pase activity (FIG. 4F) and glycogen content (FIG. 4G) in livers of puppies 4 and 16 months after treatment as neonates with gene editing vectors described herein, as well as normal controls (wt/c) and untreated puppies with GSD Ia (affected).

FIG. 4H line plot showing results from 8-hour fasting tests performed from 0 to 20 months in puppies treated as neonates with the gene editing vectors described herein.

FIG. 5A is a representative agarose gel for the standard curve for the integration PCR showing serial dilutions of a starting template representing an integrated transgene.

FIG. 5B is a representative agarose gel showing the integration PCR for quantification of transgene integration in dogs treated with gene editing vectors as adults.

FIG. 5C is a representative agarose gel showing the integration PCR for quantification of transgene integration in puppies treated with gene editing vectors as neonates.

FIG. 5D-5E are plots showing the level of transgene integration 4 and 16 months after gene editing treatment in livers of dogs treated as adults (FIG. 5D) or puppies treated as neonates (FIG. 5E).

FIG. 6A-6B are plots quantifying hG6PC transgene expression 4 and 16 months after gene editing treatment in livers of dogs treated as adults (FIG. 6A) or puppies treated as neonates (FIG. 6B).

FIG. 6C-6D are plots depicting CRISPR/Cas9 nuclease activity quantified as modified allele percentage at 4 and 16 months after gene editing treatment in dogs treated as adults (FIG. 6C) or puppies treated as neonates (FIG. 6D).

FIG. 7 shows representative photomicrographs of hepatic sections of three GSD Ia dogs before gene editing treatment (pre-treatment (BC)), and at 4 months after gene editing treatment (4M). Also shown are photomicrographs of a control untreated dog (GSD Ia UT) and a GSD Ia carrier (GSD Ia carrier). The latter represents a normal dog liver.

FIG. 8A-8B show representative plots quantifying analytes in blood before (T=0) and immediately following CRISPR treatment in adult (FIG. 8A) and neonatal (FIG. 8B) GSD Ia dogs.

FIG. 9A is a representative agarose gel from a Surveyor assay demonstrating no on-target cleavage detected on dog and puppy liver samples at 4 and 16 months following AAV vector administration.

FIG. 9B depicts representative immunoblots showing SaCas9 protein in liver obtained 4 months after administration of gene editing vectors.

FIG. 10 depicts a schematic of an illustrative gene editing vector plasmid (AAV-cG5PgRNACas9 DOG CRISPR) for packaging the AAV-CRISPR/Cas9 vector according to various aspects of the present disclosure.

FIG. 11 depicts a schematic of an illustrative gene editing vector plasmid (AAV-2xG6P Donor DOG DONOR) for packaging the AAV-cG6PC (Donor) vector according to various aspects of the present disclosure.

FIG. 12 depicts a schematic of an illustrative gene editing vector plasmid (AAV-G6Pcmin 303 SpCas9 Final MOUSE CRISPR) for packaging the CRISPR vector according to various aspects of the present disclosure.

FIG. 13 depicts a schematic of an illustrative gene editing vector plasmid (AAV-mouseG6pcdonorbGHPolyA+SpCas9gRNA Final MOUSE DONOR) for packaging the Donor vector according to various aspects of the present disclosure.

FIG. 14A depicts illustrative schematics of two murine gene editing constructs according to various aspects of the present disclosure.

FIG. 14B depicts a schematic of murine transgene integration into a G6PC locus in a target mouse according to various aspects of the present disclosure.

FIG. 15A is a plot depicting levels of blood glucose after an 8 hour fast two weeks after treatment with low, medium or high doses of gene editing vectors.

FIG. 15B-15C depict plots of blood glucose levels at baseline (FIG. 15B) and after 120 minutes (FIG. 15C) during a glucose tolerance test (GTT) administered 4 weeks after treatment with low, medium, or high doses of gene editing vectors.

FIG. 16A-16B depict levels of G6Pase activity (FIG. 16A) and glycogen content (FIG. 16B) in livers of mice 4 weeks after treatment with different concentrations of gene editing vectors described herein.

FIG. 17A-17B depict quantification of hG6PC vector copy number (FIG. 17A) and donor transcripts (FIG. 17B) in mice four weeks after treatment with gene editing vectors described herein, at three different doses.

FIG. 18A-18B depicts quantification of CRISPR vector copy number (SpCas9 DNA, FIG. 18A) or CRISPR transcript levels (SpCas9 RNA, FIG. 18B) in mice four weeks after treatment with gene editing vectors described herein.

FIG. 19 is a Kaplan Meier Survival curve of mice treated with low or high concentrations of gene editing vectors (Donor+/−CRISPR), optionally with bezafibrate (+drug).

FIG. 20A-20B are bar plots quantifying blood glucose levels after an 8 hour fast in mice two weeks (FIG. 20A) or eleven weeks (FIG. 20B) after treatment with indicated doses of gene editing vectors (with or without bezafibrate).

FIG. 21A-21B are bar plots quantifying results from a glucose tolerance test (GTT) and show blood glucose levels at baseline (FIG. 21A) or 120 minutes after administration of dextrose (FIG. 21B) in mice 12 weeks after treatment with indicated doses of gene editing vectors (with or without bezafibrate).

FIG. 22A-22B are bar plots quantifying G6Pase activity (FIG. 22A) and glycogen content (FIG. 22B) in livers obtained from mice 12 weeks after treatment with indicated doses of gene editing vectors (with or without bezafibrate).

FIG. 23A-23B depict quantification of hG6PC vector copy number (FIG. 23A) and donor transcripts (FIG. 23B) in mice 12 weeks after treatment with indicated doses of gene editing vectors (with or without bezafibrate).

FIG. 24A-24B are bar plots quantifying levels of spCas9 DNA (vector copy number, FIG. 24A), or spCas9 RNA (transcript levels, FIG. 24B) in mice 12 weeks after treatment with indicated doses of gene editing vectors (with or without bezafibrate).

FIG. 25 depicts representative immunoblots and quantification of results from a Surveyor Assay showing indel formation in liver samples obtained from mice after treatment with gene editing vectors described herein.

FIG. 26 depicts representative agarose gels and quantification thereof showing results from a G6PC transgene integration PCR assay in samples from mice treated with gene editing vectors with bezafibrate or without bezafibrate treatment (no drug) as described herein.

FIG. 27 is a schematic of an illustrative gene editing vector plasmid (New Donor W/hG6PC MRW/EI) for packaging a new Donor vector for editing in mice with GSD Ia according to various aspects of the present disclosure.

FIG. 28 depicts a schematic of an illustrative gene editing vector plasmid (AAV-SaCas9 Human Do DONOR) for packaging the AAV-cG6PC (Donor) vector according to various aspects of the present disclosure.

FIG. 29 depicts a schematic of an illustrative gene editing vector plasmid (AAV-SaCas9 Human CRISPR) for packaging the AAV-CRISPR/Cas9 vector according to various aspects of the present disclosure.

FIG. 30 depicts a schematic of an illustrative gene editing vector plasmid (AAV-AAV-SpCas9 Human DONOR) for packaging the AAV-cG6PC (Donor) vector according to various aspects of the present disclosure.

### DETAILED DESCRIPTION

Glucose Phosphatases, including glucose-6-phosphatase plays a crucial role in glycogen storage. GSD Ia (von Gierke disease) results from pathogenic variants in the G6PC gene that causes glucose-6-phosphatase (G6Pase) deficiency in liver. G6Pase deficiency leads to the accumulation of glycogen in the liver due to accumulated glucose-6-phosphate, accompanied by hepatosteatosis. GSD Ia can be treated with gene therapy, however, the effect of gene therapy wanes quickly due to the loss of non-integrating viral vectors under clinical development, including adeno-associated virus (AAV) vectors.

The present disclosure is based, in part, on the discovery of gene editing systems that allow for stable integration of a therapeutic G6PC transgene in the genome of a subject to allow for endogenous and persistent expression of a functional glucose-6-phosphatase in a patient for a therapeutic effect. Accordingly, disclosed herein are novel nucleic acids, vectors, and compositions that can be used in gene editing methods for treating glycogen storage diseases.

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition (e.g., a GSD) manifested by a patient or to which a patient may be susceptible. The aim of treatment includes the alleviation or prevention of symptoms, slowing or stopping the progression or worsening of a disease, disorder, or condition and/or the remission of the disease, disorder or condition (e.g., a GSD).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, the terms “prevent,” “preventing,” “prevention,” “prophylactic treatment” and the like refer to reducing the probability of developing a disease, disorder or condition (e.g., GSD) in a subject, who does not have, but is at risk of or susceptible to developing a disease, disorder or condition. The term “effective amount” or “therapeutically effective amount” refers to an amount sufficient to effect beneficial or desirable biological and/or clinical results. In other words, in an aspect, preventing glycogen storage disruption or and/or restoring glycogen storage homeostasis is intended. The words “prevent” and “preventing” and “prevention” also refer to prophylactic or preventative measures for protecting or precluding a subject (e.g., an individual) not having glycogen storage dysfunction and/or a given glycogen storage dysfunction related complication from progressing to that complication.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample can be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It can be caused by an external factor, such as an infectious disease, or by internal dysfunctions, such as cancer, cancer metastasis, and the like.

As used herein, the term “glycogen storge disease” or “GSD” or “GSD-mediated disease” is broadly defined and refers to those disorders associated with glycogen storage disorders. Examples include, but are not limited to, glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, glycogenosis due to AMP-activated protein kinase gamma subunit 2-deficiency (PRKAG2), or cardiac glycogenosis due to AMP-activated protein kinase gamma subunit 2 deficiency. In some embodiments, GSD I can be selected from GSD Ia, GSD Ib, or GSD Ic. In some embodiments, GSD I is GSD Ia. In some embodiments, GSD-III can be selected from GSD-type IIIa, type IIIb, type IIIc, or type IIId.

“Contacting” as used herein, e.g., as in “contacting a sample” refers to contacting a sample directly or indirectly in vitro, ex vivo, or in vivo (i.e., within a subject as defined herein). Contacting a sample can include addition of a compound (e.g., a nucleic acid and/or vector as provided herein) to a sample, or administration to a subject. Contacting encompasses administration to a solution, cell, tissue, mammal, subject, patient, or human. Further, contacting a cell includes adding an agent to a cell culture.

As used herein, the term “therapeutic agent” means an agent utilized to treat, combat, ameliorate, prevent or improve an unwanted condition or disease of a subject, such as glycogen storage disorders. In part, embodiments described herein can be directed to the treatment of various cytoplasmic glycogen storage disorders, including, but not limited to glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, glycogenosis due to AMP-activated protein kinase gamma subunit 2-deficiency (PRKAG2), or cardiac glycogenosis due to AMP-activated protein kinase gamma subunit 2 deficiency. In some embodiments, GSD I can be selected from GSD Ia, GSD Ib, or GSD Ic. In some embodiments, GSD I is GSD Ia. In some embodiments, GSD-III can be selected from GSD-type IIIa, type IIIb, type IIIc, or type IIId.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient).

As used herein, the term “sequence identity” refers to the number of identical or similar residues (i.e., nucleotide bases or amino acid) on a comparison between a test and reference nucleotide or amino acid sequence. Sequence identity can be determined by sequence alignment of nucleic acid to identify regions of similarity or identity. As described herein, sequence identity is generally determined by alignment to identify identical residues. Matches, mismatches, and gaps can be identified between compared sequences. Alternatively, sequence identity can be determined without taking into account gaps as the number of identical positions/length of the total aligned sequence×100. In one non-limiting embodiment, the term “at least 90% sequence identity to” refers to percent identities from 90 to 100%, relative to the reference nucleotide or amino acid sequence. Identity at a level of 90% or more is indicative of the fact that, assuming for exemplary purposes a test and reference oligonucleotide or length of 100 nucleotides are compared, no more than 10% (i.e., 10 out of 100) of the nucleotides in the test oligonucleotide differ from those of the reference oligonucleotide. Differences are defined as nucleic acid or amino acid substitutions, insertions, or deletions.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, a “regulatory element” can refer to promoters, enhancers, internal ribosomal entry sites (IRES), and other expression control elements (e.g., transcription termination signals, such as polyadenylation signals and poly-U sequences). Regulatory elements are discussed infra and can include those that direct constitutive expression of a nucleotide sequence in many types of host cells and those that direct expression of the nucleotide sequence only in certain host cells (e.g., tissue-specific regulatory sequences).

As used herein, “recombinant” is used herein to refer to new combinations of genetic material as a result of genetic engineering. For instance, a recombinant organism (e.g., bacteria) can be an organism that contains different genetic material from either of its parents as a result of genetic modification, recombinant DNA can be a form of artificial DNA, a recombinant protein or enzyme can be an artificially produced and purified form of the protein or enzyme, and a recombinant virus can be a virus formed by recombining genetic material.

As used herein, the term “open reading frame (ORF)” refers to the parts of a reading frame that has the ability to be translated. An ORF can be a continuous chain of codons that begins with a start codon (e.g., ATG) and ends at a stop codon (e.g., TAA, TAG, TGA). A reading frame is a sequence of nucleotides that are read as codons specifying amino acids.

As used herein, the term “endogenous promoter/enhancer” refers to a disclosed promoter or disclosed promoter/enhancer that is naturally linked with its gene. In an aspect, a disclosed endogenous promoter can generally be obtained from a non-coding region upstream of a transcription initiation site of a gene (such as, for example, a disclosed phosphorylase kinase, phosphorylase, or some other enzyme involved in the glycogen metabolic pathway). In an aspect, a disclosed endogenous promoter can be used for constitutive and efficient expression of a disclosed transgene (e.g., a nucleic acid sequence encoding a polypeptide capable of preventing glycogen accumulation and/or degrading accumulated glycogen). In an aspect, a disclosed endogenous promoter can be an endogenous promoter/enhancer.

As used herein, the term “exogenous promoter” or “heterologous promoter” refers to a disclosed promoter or a disclosed promoter/enhancer that can be placed in juxtaposition to a gene by means of molecular biology techniques such that the transcription of that gene can be directed by the linked promoter or linked promoter/enhancer.

Unless otherwise defined, all technical terms used herein have the same meaning as commonly understood by one of ordinary skill in the art to which this disclosure belongs.

### II. Gene Editing Systems

The present disclosure is based, in part, on the discovery of gene editing systems that allow for stable integration of a therapeutic G6PC transgene in the genome of a cell to allow for endogenous correction of a gene defect and expression of a functional protein for a therapeutic effect. As described further below, the gene editing systems of the present disclosure are intended to correct a G6PC gene which encodes for glucose-6-phosphatase. In certain glycogen storage diseases (e.g., GSD Ia), the G6PC gene has a mutation that prevents expression of functional glucose-6-phosphatase. Accordingly, the present disclosure provides novel nucleic acids, vectors and vector systems and pharmaceutical compositions thereof that allow for stable integration of a G6PC transgene into a cell such that the cell expresses a functional glucose-6-phosphatase protein.

As used herein, “genome editing” generally refers to the process of modifying the nucleotide sequence of a genome, preferably in a precise or pre-determined manner, such that the modified nucleic acid comprises a nucleic acid insertion that encodes a therapeutic protein. Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as described in Cox et al., Nature Medicine, 2015, 21 (2), 121-31. These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, that has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature, 2015, 518, 174-76; Kent et al., Nature Structural and Molecular Biology, 2015, 22 (3): 230-7; Mateos-Gomez et al., Nature, 2015, 518, 254-57; Ceccaldi et al., Nature, 2015, 528, 258-62. In some instances, it may be possible to predict likely repair outcomes based on analysis of potential microhomologies at the site of the DNA break. Each of these genome editing mechanisms can be used to create desired genetic modifications. A step in the genome editing process can be to create one or two DNA breaks, the latter as double-strand breaks or as two single-stranded breaks, in the target locus as near the site of intended mutation. This can be achieved via the use of endonucleases, as described and illustrated herein

In certain aspects, the gene editing methods herein comprise inserting a therapeutic transgene into a target location in a genome using homologous dependent recombination (HDR). This method of gene editing therefore allows for endogenous, stable expression of the therapeutic protein and is contrasted with “gene therapy” which herein refers to a method of delivering an exogenous nucleic acid to a cell such that the exogenous nucleic acid can be expressed but remains episomal and is not integrated into the genome of the cell via a gene editing system described herein (e.g., an AAV vector encoding a G6PC gene alone).

Accordingly, in some embodiments, a CRISPR-endonuclease system is provided herein that can be used to genetically modify a cell having a mutation in a G6PC gene (e.g., to insert a G6PC transgene within or near the G6PC gene locus) and thereby increasing expression of a therapeutic protein (glucose-6-phosphatase) in the cell.

The CRISPR-endonuclease system is a naturally occurring defense mechanism in prokaryotes that has been repurposed as a RNA-guided DNA-targeting platform used for gene editing. CRISPR systems include Types I, II, III, IV, V, and VI systems. In some aspects, the CRISPR system is a Type II CRISPR/Cas9 system. In various aspects, the CRISPR-endonuclease systems (e.g., Type II CRISPR/Cas9 systems) used herein comprise three primary components: a site directed (RNA-guided) endonuclease, a guide RNA that directs the site-directed endonuclease to a target location in a genome, and a donor nucleic acid that can be incorporated into the genome at the target location. Each of these components are described in more detail below.

### (a) Site-Directed (RNA-Guided) Endonucleases

In various aspects, the gene editing system herein comprises one or more site-directed endonuclease. In various aspects, the site directed endonuclease is from a Type II CRISPR system. In some embodiments, the site directed endonuclease is a Cas9 (CRISPR associated protein 9). In some embodiments, the Cas9 endonuclease is from Streptococcus pyogenes (SpCas9) or Staphylococcus aureus (SaCas9), although other Cas9 homologs can be used, e.g., N. meningitidis Cas9, S. thermophilus CRISPR 1 Cas9, S. thermophilus CRISPR 3 Cas9, or T. denticola Cas9.

Exemplary CRISPR/Cas polypeptides include the Cas9 polypeptides as published in Fonfara et al., Nucleic Acids Research, 2014, 42:2577-2590. The CRISPR/Cas gene naming system has undergone extensive rewriting since the Cas genes were discovered. Fonfara et al. also provides PAM sequences for the Cas9 polypeptides from various species.

The RNA-guided endonuclease systems as used herein can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary endonuclease, e.g., a Cas9 from S. pyogenes or a Cas9 from S. aureus provided below. The endonuclease can comprise at least 70, 75, 80, 85, 90, 95, 97, 99, or 100% identity to a wild-type endonuclease (e.g., Cas9 from S. pyogenes or S. aureus) over 10 contiguous amino acids. The endonuclease can comprise at most: 70, 75, 80, 85, 90, 95, 97, 99, or 100% identity to a wild-type endonuclease (e.g., Cas9 from S. pyogenes or S. aureus) over 10 contiguous amino acids. The endonuclease can comprise at least: 70, 75, 80, 85, 90, 95, 97, 99, or 100% identity to a wild-type endonuclease (e.g., Cas9 from S. pyogenes or S. aureus) over 10 contiguous amino acids in a HNH nuclease domain of the endonuclease. The endonuclease can comprise at most: 70, 75, 80, 85, 90, 95, 97, 99, or 100% identity to a wild-type endonuclease (e.g., Cas9 from S. pyogenes or S. aureus) over 10 contiguous amino acids in a HNH nuclease domain of the endonuclease. The endonuclease can comprise at least: 70, 75, 80, 85, 90, 95, 97, 99, or 100% identity to a wild-type endonuclease (e.g., Cas9 from S. pyogenes or S. aureus) over 10 contiguous amino acids in a RuvC nuclease domain of the endonuclease. The endonuclease can comprise at most: 70, 75, 80, 85, 90, 95, 97, 99, or 100% identity to a wild-type endonuclease (e.g., Cas9 from S. pyogenes or S. aureus) over 10 contiguous amino acids in a RuvC nuclease domain of the endonuclease. Exemplary sequences of Cas9 from S. pyogenes or S. aureus are provided in Table 7 at the end of this application along with illustrative nucleic acids that can be used to encode them according to various aspects herein.

In any of the preceding embodiments, the CRISPR endonuclease can be linked to at least one nuclear localization signal (NLS). The at least one NLS can be located at or within 50 amino acids of the amino-terminus of the CRISPR nuclease and/or at least one NLS can be located at or within 50 amino acids of the carboxy-terminus of the CRISPR nuclease.

Other site-directed endonucleases are contemplated in this disclosure. For example, the site-directed endonuclease can comprise a zinc-finger nuclease or Transcription Activator-Like Effector Nucleases (TALENs), which are described further below.

Zinc finger nucleases (ZFNs) are modular proteins comprised of an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZEN is typically comprised of 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers. In most applications of ZFNs, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96 (6): 2758-63; Dreier B et al., J Mol Biol., 2000, 303 (4): 489-502; Liu Q et al., J Biol Chem., 2002, 277 (6): 3850-6; Dreier et al., J Biol Chem., 2005, 280 (42): 35588-97; and Dreier et al., J Biol Chem. 2001, 276 (31): 29466-78.

TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9 or CRISPR/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science, 2009 326 (5959): 1509-12; Mak et al., Science, 2012, 335 (6069): 716-9; and Moscou et al., Science, 2009, 326 (5959): 1501. The use of TALENs based on the “Golden Gate” platform, or cloning scheme, has been described by multiple groups; see, e.g., Cermak et al., Nucleic Acids Res., 2011, 39 (12): e82; Li et al., Nucleic Acids Res., 2011, 39 (14): 6315-25; Weber et al., PLOS One., 2011, 6 (2): e16765; Wang et al., J Genet Genomics, 2014, 41 (6): 339-47.; and Cermak T et al., Methods Mol Biol., 2015 1239:133-59.

### (b) Guide RNAs

The present disclosure provides a guide RNAs (gRNAs) that can direct the activities of an associated endonuclease to a specific target site within a polynucleotide. A guide RNA can comprise at least a spacer sequence that hybridizes to a target nucleic acid sequence of interest, and a CRISPR repeat sequence. In CRISPR Type II systems, the gRNA also comprises a second RNA called the tracrRNA sequence. In the CRISPR Type II guide RNA (gRNA), the CRISPR repeat sequence and tracrRNA sequence hybridize to each other to form a duplex. In some embodiments, a gRNA can bind an endonuclease, such that the gRNA and endonuclease form a complex. The gRNA can provide target specificity to the complex by virtue of its association with the endonuclease. The genome-targeting nucleic acid thus can direct the activity of the endonuclease.

Exemplary guide RNAs include a spacer sequence that comprises 15-200 nucleotides wherein the gRNA targets a genome location based on the GRCh38 human genome assembly. As is understood by the person of ordinary skill in the art, each gRNA can be designed to include a spacer sequence complementary to its genomic target site or region. See Jinek et al., Science, 2012, 337, 816-821 and Deltcheva et al., Nature, 2011, 471, 602-60.

The gRNA can be a double-molecule guide RNA. The gRNA can be a single-molecule guide RNA.

A double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

In some embodiments, a sgRNA comprises a 20-nucleotide spacer sequence at the 5′ end of the sgRNA sequence. In some embodiments, a sgRNA comprises a less than a 20-nucleotide spacer sequence at the 5′ end of the sgRNA sequence. In some embodiments, a sgRNA comprises a more than 20 nucleotide spacer sequence at the 5′ end of the sgRNA sequence. In some embodiments, a sgRNA comprises a variable length spacer sequence with 17-30 nucleotides at the 5′ end of the sgRNA sequence. In some embodiments, a sgRNA comprises a spacer extension sequence with a length of more than 1, 5, 10, 15, 20, 25, 30, 35, 40, 45, 50, 60, 70, 80, 90, 100, 120, 140, 160, 180, or 200 nucleotides. In some embodiments, a sgRNA comprises a spacer extension sequence with a length of less than 3, 5, 10, 15, 20, 25, 30, 35, 40, 45, 50, 60, 70, 80, 90, or 100 nucleotides.

In some embodiments, a sgRNA comprises a spacer extension sequence that comprises another moiety (e.g., a stability control sequence, an endoribonuclease binding sequence, or a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

In some embodiments, a sgRNA comprises a spacer sequence that hybridizes to a sequence in a target polynucleotide. The spacer of a gRNA can interact with a target polynucleotide in a sequence-specific manner via hybridization (i.e., base pairing). The nucleotide sequence of the spacer can vary depending on the sequence of the target nucleic acid of interest.

In a CRISPR-endonuclease system, a spacer sequence can be designed to hybridize to a target polynucleotide that is located 5′ of a PAM of the endonuclease used in the system. The spacer may perfectly match the target sequence or may have mismatches. Each endonuclease, e.g., Cas9 nuclease, has a particular PAM sequence that it recognizes in a target DNA. For example, S. pyogenes Cas9 recognizes a PAM that comprises the sequence 5′-NRG-3′, where R comprises either A or G, where N is any nucleotide and N is immediately 3′ of the target nucleic acid sequence targeted by the spacer sequence. S. aureus Cas9 recognizes a PAM that comprises the sequence 5′-NNGRRT-3′ (where R represents A or G) an NN is immediately 3′ of the target nucleic acid sequence targeted by the spacer sequence.

A target polynucleotide sequence can comprise 20 nucleotides. The target polynucleotide can comprise less than 20 nucleotides. The target polynucleotide can comprise more than 20 nucleotides. The target polynucleotide can comprise at least: 5, 10, 15, 16, 17, 18, 19, 20, 21, 22, 23, 24, 25, 30 or more nucleotides. The target polynucleotide can comprise at most: 5, 10, 15, 16, 17, 18, 19, 20, 21, 22, 23, 24, 25, 30 or more nucleotides. The target polynucleotide sequence can comprise 20 bases immediately 5′ of the first nucleotide of the PAM.

A spacer sequence that hybridizes to a target polynucleotide can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides. In some examples, the spacer can comprise 18 nucleotides. In some examples, the spacer can comprise 22 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges. In some aspects, the gRNA spacer sequence is the full length of the “target sequence” and is 100% identical to the “target sequence”—that is, it is an RNA version of the DNA “target sequence”

A tracrRNA sequence can comprise nucleotides that hybridize to a minimum CRISPR repeat sequence in a cell. A minimum tracrRNA sequence and a minimum CRISPR repeat sequence may form a duplex, i.e., a base-paired double-stranded structure. Together, the minimum tracrRNA sequence and the minimum CRISPR repeat can bind to an RNA-guided endonuclease. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., supra.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides.

The duplex can comprise a mismatch (i.e., the two strands of the duplex are not 100% complementary). The duplex can comprise at least about 1, 2, 3, 4, or 5 or mismatches. The duplex can comprise at most about 1, 2, 3, 4, or 5 or mismatches. The duplex can comprise no more than 2 mismatches.

In some embodiments, a tracrRNA may be a 3′ tracrRNA. In some embodiments, a 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, a gRNA may comprise a tracrRNA extension sequence. A tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, or 200 nucleotides. The tracrRNA extension sequence can have a length from about 20 to about 5000 or more nucleotides. The tracrRNA extension sequence can have a length of less than 5, 10, 15, 20, 25, 30, 35, 40, 45, 50, 60, 70, 80, 90, or 100 nucleotides. The tracrRNA extension sequence can comprise less than 10 nucleotides in length. The tracrRNA extension sequence can be 10-30 nucleotides in length. The tracrRNA extension sequence can be 30-70 nucleotides in length.

The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt.

In some embodiments, a sgRNA may comprise a linker sequence with a length from about 3 nucleotides to about 100 nucleotides. In Jinek et al., supra, for example, a simple 4 nucleotide “tetraloop” (-GAAA-) was used (Jinek et al., Science, 2012, 337 (6096): 816-821). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker will not comprise sequences that have extensive regions of homology with other portions of the guide RNA, which might cause intramolecular binding that could interfere with other functional regions of the guide. In Jinek et al., supra, a simple 4 nucleotide sequence-GAAA- was used (Jinek et al., Science, 2012, 337 (6096): 816-821), but numerous other sequences, including longer sequences can likewise be used.

The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

In some embodiments, a sgRNA does not comprise a uracil, e.g., at the 3′end of the sgRNA sequence. In some embodiments, a sgRNA does comprise one or more uracils, e.g., at the 3′end of the sgRNA sequence. In some embodiments, a sgRNA comprises 1, 2, 3, 4, 5, 6, 7, 8, 9, or 10 uracils (U) at the 3′ end of the sgRNA sequence.

A sgRNA may be chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art.

In some embodiments, a modified gRNA may comprise a modified backbone, for example, phosphorothioates, phosphotriesters, morpholinos, methyl phosphonates, short chain alkyl or cycloalkyl intersugar linkages or short chain heteroatomic or heterocyclic intersugar linkages.

Morpholino-based compounds are described in Braasch and David Corey, Biochemistry, 2002, 41 (14): 4503-4510; Genesis, 2001, Volume 30, Issue 3; Heasman, Dev. Biol., 2002, 243:209-214; Nasevicius et al., Nat. Genet., 2000, 26:216-220; Lacerra et al., Proc. Natl. Acad. Sci., 2000, 97:9591-9596.; and U.S. Pat. No. 5,034,506, issued Jul. 23, 1991.

Cyclohexenyl nucleic acid oligonucleotide mimetics are described in Wang et al., J. Am. Chem. Soc., 2000, 122:8595-8602.

In some embodiments, a modified gRNA may comprise one or more substituted sugar moieties, e.g., one of the following at the 2′ position: OH, SH, SCH₃, F, OCN, OCH₃, OCH₃ O (CH₂)n CH₃, O(CH₂) n NH₂, or O(CH₂) n CH₃, where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O—CH₃); 2′-propoxy (2′—OCH₂ CH₂CH₃); and 2′-fluoro (2′-F). Similar modifications may also be made at other positions on the gRNA, particularly the 3′ position of the sugar on the 3′ terminal nucleotide and the 5′ position of 5′ terminal nucleotide. In some examples, both a sugar and an internucleoside linkage, i.e., the backbone, of the nucleotide units can be replaced with novel groups.

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine, and 2,6-diaminopurine. Kornberg, A., DNA Replication, W. H. Freeman & Co., San Francisco, pp 75-77, 1980; Gebeyehu et al., Nucl. Acids Res. 1997, 15:4513. A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine. In accord with any of the foregoing, the present disclosure provides gRNAs that target specific locations in a G6PC gene locus. In accordance with further aspects, the gRNAs provided herein can be used with various CRISPR associated (Cas) endonucleases as described herein. For example, in some aspects, exemplary gRNAs are provided in Tables 1A and 1B, below, which are designed to work with saCas9 or spCas9 endonucleases, respectively. Further, different gRNAs are provided to target the murine, canine or human G6PC gene locus, as desired. In any of these aspects, the target sequence in the G6PC gene locus can comprise or consist of any one of SEQ ID NOs: 1 to 15, as provided in Tables 1A and 1B below. In some aspects, such as when a SaCas9 endonuclease is used, the target sequence in the G6PC gene locus can comprise or consist of any one of SEQ ID NOs: 1 to 8 as provided in Table 1A. In some aspects, such as when an SpCas9 endonuclease is used, the target sequence in the G6PC gene locus can comprise or consist of any one of SEQ ID NOs: 9 to 15 as provided in Table 1B below. Note that SEQ ID NOs 1 to 15 represent the DNA sequence of the genomic target, but as understood in the art and described above, these gRNAs may also be provided in RNA nucleotides to represent an illustrative spacer sequence that can target these DNA targets. These RNA sequences are provided as SEQ ID NOs 117-131 and are understood to correspond to SEQ ID NOs 1-15, respectively.

TABLE 1A
gRNAs for use with SaCas9

| | | | | gRNA |
|---|---|---|---|---|
| | | | | spacer |
| gRNA | | | | sequence |
| Target | | Sequence | | (SEQ ID |
| Species | Cas9 | (SEQ ID NO) | PAM | NO) |
| Canine | SaCas9 | GATGGTCAGACATTTCCAGA | GAGAAT | 117 |
| | | (SEQ ID NO: 1) | | |
| Canine | SaCas9 | CTGGCTCAACCTCGTCTTTAAATG | GTGAGT | 118 |
| | | (SEQ ID NO: 2) | | |
| Canine | SaCas9 | TTCTGATGGTCAGACATTTCCAGA | GAGAAT | 119 |
| | | (SEQ ID NO: 3) | | |
| Canine | SaCas9 | AGGTAGATGATCCAGAGTCAGGAG | GGGAGT | 120 |
| | | (SEQ ID NO: 4) | | |
| Human | SaCas9 | TGATGGACAGACATTGCGAGA | GCGAAT | 121 |
| | | (SEQ ID NO: 5) | | |
| Human | SaCas9 | GCATGAGTAGCCCGTGGCTTC | CTGAAT | 122 |
| | | (SEQ ID NO: 6) | | |
| Human | SaCas9 | TCAGAGAGAGGGGTTGGAAGC | ATGAGT | 123 |
| | | (SEQ ID NO: 7) | | |
| Human | SaCas9 | AGATGATCCAAAGTCAGAGAG | AGGGGT | 124 |
| | | (SEQ ID NO: 8) | | |

TABLE 1B
gRNAs for use with SpCas9

| | | | | gRNA |
|---|---|---|---|---|
| | | | | spacer |
| gRNA | | | | sequence |
| Target | | Sequence | | (SEQ ID |
| Species | Cas9 | (SEQ ID NO) | PAM | NO) |
| Mouse | SpCas9 | TCTATGTCCTCTTTCCCATC | TGG | 125 |
| | | (SEQ ID NO: 9) | | |
| Human | SpCas9 | TCTACGTCCTCTTCCCCATC | TGG | 126 |
| | | (SEQ ID NO: 10) | | |
| Human | SpCas9 | TTGCTGATCTCCTCTCTATA | TGG | 127 |
| | | (SEQ ID NO: 11) | | |
| Human | SpCas9 | GTGGTAAGAACCATATAGAG | AGG | 128 |
| | | (SEQ ID NO: 12) | | |
| Human | SpCas9 | GAGGAGATCAGCAAGAAAAG | AGG | 129 |
| | | (SEQ ID NO: 13) | | |
| Human | SpCas9 | AGATCAGCAAGAAAAGAGGC | TGG | 130 |
| | | (SEQ ID NO: 14) | | |
| Human | SpCas9 | CTGGGGAAAGCAACTTCTGA | TGG | 131 |
| | | (SEQ ID NO: 15) | | |

### (c) Donor Nucleic Acids

In various aspects, the gene editing system herein comprises one or more donor nucleic acids. The donor nucleic acids herein comprise (i) a nucleotide sequence encoding a therapeutic protein (e.g., glucose-6-phosphatase), (ii) a nucleotide sequence having sequence homology with a sequence 5′ upstream to a site targeted by the gRNA/Cas9 endonuclease described above, and (iii) a nucleotide sequence having sequence homology with a sequence 3′ downstream to a site targeted by the gRNA/Cas9 endonuclease described above, where (i) is flanked by (ii) and (iii).

In accordance with various aspects, the nucleotide sequence of (i) that encodes a therapeutic protein (e.g., glucose-6-phosphatase) above is referred to as a transgene (e.g., a G6PC transgene). As used herein, the term “transgene” refers to exogenous nucleic acid sequences that encode a polypeptide to be expressed in a cell into which the transgene is introduced. A transgene can include a heterologous nucleic acid sequence that is not naturally found in the cell into which it has been introduced, a nucleic acid sequence that is a mutant form of a nucleic acid sequence naturally found in the cell into which it has been introduced, or a nucleic acid sequence that is the same as a naturally occurring nucleic in the cell into which it has been introduced. A transgene can include genes from the same organism into which it is introduced or from a different organism. A transgene of the present disclosure includes, but is not limited to, G6PC1, G6PC2, G6PC3, or any gene encoding a G6PC. In certain aspects, the nucleic acid encoding glucose-6-phosphatase encodes for a murine, human, or canine glucose-6-phosphatase (and is therefore referred to as a human, murine or canine G6PC transgene respectively). In various aspects, the nucleotide sequence of (i) has at least 80%, at least 85%, at least 90%, at least 95%, at least 96%, at least 97%, at least 98%, at least 99% or 100% sequence homology to any one of SEQ ID NOs: 16-19. In some aspects, the nucleotide sequence of (i) comprises any one of SEQ ID NOs: 16-19. In further aspects, the nucleotide sequence of (i) consists of any one of SEQ ID NO: 16-19. For example, the nucleotide sequence of (i) can comprise or consist of SEQ ID NO: 16. For example, the nucleotide sequence of (i) can comprise or consist of SEQ ID NO: 17. For example, the nucleotide sequence of (i) can comprise or consist of SEQ ID NO: 18. For example, the nucleotide sequence of (i) can comprise or consist of SEQ ID NO: 19. In some aspects, the target nucleotide sequence in the G6PC locus is within an exon of the G6PC gene locus. In these cases, the nucleotide sequence of (i), which is inserted into that location of the G6PC gene locus, can optionally comprise a mutation (e.g., an A>G mutation) such that the PAM used by the Cas endonuclease is mutated in the edited gene and cannot be the basis for further editing. Accordingly, some of SEQ ID NOs 16-19 comprise this A>G mutation (e.g., SEQ ID NO: 19), but it would be appreciated by one of skill in the art that this mutation is optional and a native G6PC gene can be used instead. For ease of reference, SEQ ID NOs: 16-19 are presented in Table 7 at end of this application.

In various aspects, the nucleotide sequence of (i) can further comprise a regulatory sequence (e.g., a promoter or enhancer) that is operably linked to the nucleotide sequence encoding the therapeutic protein (e.g., glucose-6-phosphatase). In some aspects, the regulatory sequence can comprise a promoter sequence. In some aspects, the promoter is a G6PC promoter. In some aspects, the regulatory sequence is obtained from the same species as the G6PC transgene. For example, if a human G6PC transgene (e.g., any of SEQ ID NOs: 16-18) is selected, the nucleotide sequence of (i) can further comprise a human G6PC promoter. The full length human G6PC promoter is provided herein as SEQ ID NO: 23 (see Table 7). Optionally, a smaller minimal human G6PC promoter can be used as required by the size of a desired vector or construct delivering the donor nucleic acid. Illustrative smaller minimal G6PC promoters that can be incorporated into the nucleotide sequence of (i) are provided as SEQ ID NOs: 20-22, herein. Other promoters or regulatory sequences can be envisioned by one of skill in the art and are provided, for example, in Schmoll et al. (Biochem J (1999) 338, 457-463) which is incorporated herein by reference in its entirety. Accordingly, in some aspects, the additional regulatory sequence can comprise a nucleotide sequence having at least 80%, at least 85%, at least 90%, at least 95%, at least 96%, at least 97%, at least 98%, at least 99%, or 100% sequence homology with any one of SEQ ID NOs: 20-23. In some aspects, the additional regulatory sequence can comprise any one of SEQ ID NOs: 20-22. In some aspects, the additional regulatory sequence can consist of any one of SEQ ID NOs: 20-22. In some aspects, the additional regulatory sequence can comprise SEQ ID NO: 20. In some aspects, the additional regulatory sequence can consist of a SEQ ID NOs: 20. In some aspects, the additional regulatory sequence can comprise SEQ ID NO: 21. In some aspects, the additional regulatory sequence can consist of a SEQ ID NOs: 21. In some aspects, the additional regulatory sequence can comprise SEQ ID NO: 22. In some aspects, the additional regulatory sequence can consist of a SEQ ID NOs: 22. For ease of reference, SEQ ID NOs: 20-23 are presented in Table 7 at the end of this application.

In accordance with the foregoing, the nucleotide sequence of (i) can comprise at least 80%, at least 85%, at least 90%, at least 95%, at least 96%, at least 97%, at least 98%, at least 99%, or 100% sequence homology with SEQ ID NO: 24. For example, in some aspects, the nucleotide sequence of (i) can comprise or consist of SEQ ID NO: 24. For ease of reference, SEQ ID NO: 24 is provided in Table 7 at the end of this application.

In accord with the foregoing, the present disclosure can refer to a “G6PC transgene,” “a therapeutic G6PC transgene” or “nucleotide sequence of (i).” Unless otherwise specified, all three terms are used interchangeably to refer to a nucleic acid that encodes a therapeutic glucose-6-phosphatase and can or cannot comprise further regulatory sequences as provided herein.

In accordance with various aspects, the nucleotide sequences of (ii) and (iii) above are referred to herein as “homology arms”. In certain aspects, the homology arms provided herein can be designed according to the G6PC gene locus targeted by the gene editing systems as well as the overall intended insertion. For example, in some aspects, a gene editing system herein provides a donor nucleic acid that inserts a functional G6PC transgene into a G6PC gene locus wherein the G6PC transgene further comprises an exogenous promoter. In this aspect, the G6PC transgene is integrated and expressed in a genome but is expressed under control of an exogenous promoter that is also integrated/inserted into the genome (e.g., as in SEQ ID NO: 24, described above). In other aspects, a gene editing system herein provides a donor nucleic acid that inserts a functional G6PC transgene into a G6PC locus where the G6PC transgene is integrated/inserted “in-frame” with a native promoter in the genome. In these aspects, the inserted G6PC transgene is expressed by a native promoter (e.g., the native G6PC promoter in the gene edited cell). Accordingly, the homology arms of the donor nucleic acids are chosen carefully to allow for in frame or out of frame insertion of the transgene according to whichever promoter system is chosen for its expression.

In various aspects, the nucleotide sequence of (ii) can have at least 70%, at least 75%, at least 80%, at least 85%, at least 90%, at least 95%, at least 96%, at least 97%, at least 98%, at least 99% or 100% sequence homology with any one of SEQ ID NOs: 25, 27, 29, 30, 32, or 33. In some aspects, the nucleotide sequence of (ii) comprises any one of SEQ ID NOs: 25, 27, 29, 30, 32, or 33. In some aspects, the nucleotide sequence of (ii) consists of any one of SEQ ID NOs: 25, 27, 29, 30, 32, or 33. In various aspects, the nucleotide sequence of (iii) can have at least 70%, at least 75%, at least 80%, at least 85%, at least 90%, at least 95%, at least 96%, at least 97%, at least 98%, at least 99% or 100% sequence homology with any one of SEQ ID NOs: 26, 28, 31, 34, or 35. In some aspects, the nucleotide sequence of (iii) comprises any one of SEQ ID NOs: 26, 28, 31, 34, or 35. In some aspects, the nucleotide sequence of (iii) consists of any one of SEQ ID NOs: 26, 28, 31, 34, or 35. Various combinations of the nucleotide sequence of (ii) (e.g., nucleotide sequences having at least 70%, at least 75%, at least 80%, at least 85%, at least 90%, at least 95%, at least 96%, at least 97%, at least 98%, at least 99% or 100% sequence homology with any one of SEQ ID NOs: 25, 27, 29, 30, 32, or 33) and nucleotide sequences of (iii) (e.g., nucleotide sequences having at least 70%, at least 75%, at least 80%, at least 85%, at least 90%, at least 95%, at least 96%, at least 97%, at least 98%, at least 99% or 100% sequence homology with any one of SEQ ID NOs: 26, 28, 31, 34, or 35) are envisioned, according to the target G6PC gene locus. Illustrative combinations are described further below but further combinations or variations can be envisioned by one of skill in the art.

In certain aspects, the homology arms of the donor nucleic acid sequences can have homology to a mouse G6PC gene locus. In some aspects, the homology arms of the donor nucleic acid sequences can have homology to a mouse G6PC gene locus such that the inserted nucleotide sequence of (i) is not operably linked to an endogenous mouse promoter for G6PC.

For example, in some aspects, the nucleotide sequence of (ii) (e.g., the 5′ homology arm) can have at least 70%, at least 75%, at least 80%, at least 85%, at least 90%, at least 95%, at least 96%, at least 97%, at least 98%, at least 99% or 100% sequence homology with SEQ ID NO: 25. In some aspects, the nucleotide sequence of (ii) (e.g., the 5′ homology arm) can comprise SEQ ID NO: 25. In further aspects, the nucleotide sequence of (ii) (e.g., the 5′ homology arm) can consist of SEQ ID NO: 25. In further aspects, the nucleotide sequence of (iii) (e.g., the 3′ homology arm) can have at least 70%, at least 75%, at least 80%, at least 85%, at least 90%, at least 95%, at least 96%, at least 97%, at least 98%, at least 99% or 100% sequence homology with SEQ ID NO: 26. In some aspects, the nucleotide sequence of (iii) (e.g., the 3′ homology arm) can comprise SEQ ID NO: 26. In further aspects, the nucleotide sequence of (iii) (e.g., the 3′ homology arm) can consist of SEQ ID NO: 26.

In certain aspects, the homology arms of the donor nucleic acid sequences can have homology to a mouse G6PC gene locus. In certain aspects, the homology arms of the donor nucleic acid sequences can have homology to a mouse G6PC gene locus such that the inserted nucleic acid (the nucleotide sequence of (i) is inserted in frame (is operably linked) with a native mouse promoter for G6PC. For example, in some aspects, the nucleotide sequence of (ii) (e.g., the 5′ homology arm) can have at least 70%, at least 75%, at least 80%, at least 85%, at least 90%, at least 95%, at least 96%, at least 97%, at least 98%, at least 99% or 100% sequence homology with SEQ ID NO: 27. In some aspects, the nucleotide sequence of (ii) (e.g., the 5′ homology arm) can comprise SEQ ID NO: 27. In further aspects, the nucleotide sequence of (ii) (e.g., the 5′ homology arm) can consist of SEQ ID NO: 27. In further aspects, the nucleotide sequence of (iii) (e.g., the 3′ homology arm) can have at least 70%, at least 75%, at least 80%, at least 85%, at least 90%, at least 95%, at least 96%, at least 97%, at least 98%, at least 99% or 100% sequence homology with SEQ ID NO: 28. In some aspects, the nucleotide sequence of (iii) (e.g., the 3′ homology arm) can comprise SEQ ID NO: 28. In further aspects, the nucleotide sequence of (iii) (e.g., the 3′ homology arm) can consist of SEQ ID NO: 28.

In certain aspects, the homology arms of the donor nucleic acid sequences can have homology to a canine G6PC gene locus. In certain aspects, the homology arms of the donor nucleic acid sequences can have homology to a canine G6PC gene locus such that the inserted nucleic acid (the nucleotide sequence of (i) is inserted in frame (is operably linked) with a native canine promoter for G6PC. When the nucleotide sequence of (i) comprises a native canine G6PC transgene, this can lead to an overlap between the terminal 5′ portion of the transgene and the terminal 3′ end of the 5′ homology arm. For example, the 5′ homology arm can be designed to include the first exon of the G6PC transgene. Therefore, in accordance with the understanding of one skilled in the art, the 5′ homology arm can be provided as a full sequence containing the first exon of the G6PC transgene, or the 5′ homology arm can be provided as a shorter sequence that terminates immediately before the first exon of the G6PC transgene. For completeness, two 5′ homology arms for use with a canine G6PC gene locus are provided with the understanding that it is within the normal skill in the art to select a suitable sequence based on the corresponding transgene selected. Accordingly, in some aspects, the nucleotide sequence of (ii) (e.g., the 5′ homology arm) can have at least 70%, at least 75%, at least 80%, at least 85%, at least 90%, at least 95%, at least 96%, at least 97%, at least 98%, at least 99% or 100% sequence homology with SEQ ID NO: 29 (including exon 1 of canine G6PC). In some aspects, the nucleotide sequence of (ii) (e.g., the 5′ homology arm) can have at least 70%, at least 75%, at least 80%, at least 85%, at least 90%, at least 95%, at least 96%, at least 97%, at least 98%, at least 99% or 100% sequence homology with SEQ ID NO: 30 (excluding exon 1 of canine G6PC). In some aspects, the nucleotide sequence of (ii) (e.g., the 5′ homology arm) can comprise SEQ ID NO: 29. In some aspects, the nucleotide sequence of (ii) (e.g., the 5′ homology arm) can comprise SEQ ID NO: 30. In further aspects, the nucleotide sequence of (ii) (e.g., the 5′ homology arm) can consist of SEQ ID NO: 29. In further aspects, the nucleotide sequence of (ii) (e.g., the 5′ homology arm) can consist of SEQ ID NO: 30. In further aspects, the nucleotide sequence of (iii) (e.g., the 3′ homology arm) can have at least 70%, at least 75%, at least 80%, at least 85%, at least 90%, at least 95%, at least 96%, at least 97%, at least 98%, at least 99% or 100% sequence homology with SEQ ID NO: 31. In some aspects, the nucleotide sequence of (iii) (e.g., the 3′ homology arm) can comprise SEQ ID NO: 31. In further aspects, the nucleotide sequence of (iii) (e.g., the 3′ homology arm) can consist of SEQ ID NO: 31.

In certain aspects, the homology arms of the donor nucleic acid sequences can have homology to a human G6PC gene locus. In certain aspects, the homology arms of the donor nucleic acid sequences can have homology to a human G6PC gene locus such that the inserted nucleic acid (the nucleotide sequence of (i) is inserted in frame (is operably linked) with a native human promoter for G6PC. As described above for illustrative canine homology arms, this can result in an overlap between the 5′ homology arm (e.g., nucleotide sequence of (ii)) and the human G6PC transgene (e.g., nucleotide sequence of (i). As above, illustrative 5′ homology arms (e.g., nucleotide sequences of (ii)) are provided herein in both short and long forms—where the short form excludes the first exon from the G6PC transgene and the long form includes it. For example, in some aspects, the nucleotide sequence of (ii) (e.g., the 5′ homology arm) can have at least 70%, at least 75%, at least 80%, at least 85%, at least 90%, at least 95%, at least 96%, at least 97%, at least 98%, at least 99% or 100% sequence homology with SEQ ID NO: 32 (5′ homology arm including exon 1 of human G6PC transgene). In some aspects, the nucleotide sequence of (ii) (e.g., the 5′ homology arm) can have at least 70%, at least 75%, at least 80%, at least 85%, at least 90%, at least 95%, at least 96%, at least 97%, at least 98%, at least 99% or 100% sequence homology with SEQ ID NO: 33 (5′ homology arm not including exon 1 of human G6PC transgene). In some aspects, the nucleotide sequence of (ii) (e.g., the 5′ homology arm) can comprise SEQ ID NO: 32. In some aspects, the nucleotide sequence of (ii) (e.g., the 5′ homology arm) can comprise SEQ ID NO: 33. In further aspects, the nucleotide sequence of (ii) (e.g., the 5′ homology arm) can consist of SEQ ID NO: 32. In further aspects, the nucleotide sequence of (ii) (e.g., the 5′ homology arm) can consist of SEQ ID NO: 33. In further aspects, the nucleotide sequence of (iii) (e.g., the 3′ homology arm) can have at least 70%, at least 75%, at least 80%, at least 85%, at least 90%, at least 95%, at least 96%, at least 97%, at least 98%, at least 99% or 100% sequence homology with SEQ ID NO: 34. In further aspects, the nucleotide sequence of (iii) (e.g., the 3′ homology arm) can have at least 70%, at least 75%, at least 80%, at least 85%, at least 90%, at least 95%, at least 96%, at least 97%, at least 98%, at least 99% or 100% sequence homology with SEQ ID NO: 35. In some aspects, the nucleotide sequence of (iii) (e.g., the 3′ homology arm) can comprise SEQ ID NO: 34. In some aspects, the nucleotide sequence of (iii) (e.g., the 3′ homology arm) can comprise SEQ ID NO: 35. In further aspects, the nucleotide sequence of (iii) (e.g., the 3′ homology arm) can consist of SEQ ID NO: 34. In further aspects, the nucleotide sequence of (iii) (e.g., the 3′ homology arm) can consist of SEQ ID NO: 35. SEQ ID NOs 34 and 35 differ by a single GA>CT antisense mutation (present in SEQ ID NO: 35 but not in SEQ ID NO: 34) that allows for removal of a PAM sequence when used with saCas9 endonucleases.

In any of the foregoing aspects, any of the nucleotide sequence of (i), (ii) or (iii) (e.g., the transgene, the 5′ homology arm or the 3′ homology arm) can optionally further comprise a mutation to remove a target PAM located in the corresponding location of the target G6PC gene locus. This allows insertion of a donor nucleic acid into a target site in the genome, without risk of further editing at that site. When the provided sequences of (i), (ii) or (iii) includes these mutations, they are described above. However, it would be of routine skill to remove, alter, or add mutations, as needed depending on the chosen Cas9 and PAM sequence used in the process.

For ease of reference, SEQ ID NOs: 25-35, corresponding to exemplary homology arms that can be used as nucleotide sequences (ii) or (iii), are provided in annotatd format in Table 7 at the end of this application.

In view of the foregoing, a “donor nucleic acid” is provide comprising at least three nucleotide sequences (e.g., (i), (ii) and (iii)) as provided above, where (i) is flanked by (ii) and (iii). In some aspects, these donor nucleic acids can comprise a nucleotide sequence having at least 70%, at least 75%, at least 80%, at least 85%, at least 90%, at least 95%, at least 96%, at least 97%, at least 98%, at least 99% or 100% sequence homology with any one of SEQ ID NOs: 36-40. For example, the donor nucleic acid can comprise a nucleotide sequence comprising or consisting of SEQ ID NO: 36. As another example, the donor nucleic acid can comprise a nucleotide sequence comprising or consisting of SEQ ID NO: 37. As still another example, the donor nucleic acid can comprise a nucleotide sequence comprising or consisting of SEQ ID NO: 38. As still another example, the donor nucleic acid can comprise a nucleotide sequence comprising or consisting of SEQ ID NO: 39. As still another example, the donor nucleic acid can comprise a nucleotide sequence comprising or consisting of SEQ ID NO: 40. For ease of reference, illustrative donor nucleic acids (e.g., SEQ ID NOs: 36-40) are provided in Table 7 at the end of this application.

### (d) Nucleic Acids Encoding System Components (Nucleic Acid Expression Cassettes and Vectors)

In accordance with various aspects of the present disclosure, the CRISPR-Cas9 gene editing components (e.g., the Cas9 endonuclease and gRNA) can be provided in one or more nucleic acids encoding the endonuclease and/or gRNA. The nucleic acids encoding the endonuclease and/or gRNA can further comprise the donor nucleic acid as provided herein. Accordingly, the complete CRISPR-Cas9 gene editing system can be packaged into one or more nucleic acid expression cassettes and/or vectors that allow for delivery into a cell or organism, expression of the encoded components, and gene editing in vitro or in vivo.

The present disclosure provides a nucleic acid comprising a nucleotide sequence encoding a genome-targeting nucleic acid of the disclosure, an endonuclease of the disclosure, and/or any nucleic acid or proteinaceous molecule necessary to carry out the aspects of the methods of the disclosure. The term “nucleic acid sequence,” “nucleic acid molecule,” “polynucleotide,” and “oligonucleotide” are used interchangeably and refer to a polymeric form of nucleotides that may have various lengths, either deoxyribonucleotides or ribonucleotides, or analogs thereof. Nucleic acid molecules can be deoxyribonucleic acid (DNA) or ribonucleic acid (RNA) fragments generated, for example, by a polymerase chain reaction (PCR) or by in vitro translation, and fragments generated by any one or more of ligation, scission, endonuclease action, or exonuclease action. Nucleic acid molecules can be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination thereof. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, morpholino, or the like. Nucleic acid molecules can be either single stranded or double stranded (e.g., ssDNA, dsDNA, ssRNA, or dsRNA).

The term “nucleotide” refers to sequences with conventional nucleotide bases, sugar residues and internucleotide phosphate linkages, but also to those that contain modifications of any or all of these moieties. The term “nucleotide” as used herein includes those moieties that contain not only the natively found purine and pyrimidine bases adenine (A), guanine (G), thymine (T), cytosine (C), and uracil (U), but also modified or analogous forms thereof. Polynucleotides include RNA and DNA sequences of more than one nucleotide in a single chain. Modified RNA or modified DNA, as used herein, refers to a nucleic acid molecule in which one or more of the components of the nucleic acid, namely sugars, bases, and phosphate moieties, are different from that which occurs in nature.

As used herein, the term “isolated” nucleic acid molecule (e.g., an isolated DNA, isolated cDNA, or an isolated vector genome) means a nucleic acid molecule separated or substantially free from at least some of the other components of the naturally occurring organism or virus, for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

Accordingly, in certain aspects of the disclosure, an isolated nucleic acid is provided wherein the isolated nucleic acid comprises at least one of: (a) a nucleic acid encoding an RNA-guided endonuclease provided herein (e.g., a Cas9 nuclease), (b) a nucleic acid encoding a gRNA provided herein (e.g., a gRNA comprising a spacer sequence targeting any one of SEQ ID NOs: 1 to 15) and/or (c) a donor nucleic acid as provided herein. In some aspects, the isolated nucleic acid comprises the donor nucleic acid (e.g., comprising nucleotide sequences (i), (ii) and (iii) as defined above) and a nucleic acid encoding the gRNA. In other aspects the isolated nucleic acid comprises a nucleic acid encoding an RNA-guided endonuclease (e.g., S. pyogenes Cas9 or S. aureus Cas9 as provided herein) and a nucleic acid encoding the gRNA. Exemplary nucleic acids encoding S. pyogenes Cas9 or S. aureus Cas9 are provided in Table 7 at the end of the application. In some aspects, a pair of isolated nucleic acids are provided wherein a first nucleic acid comprises the donor nucleic acid and the second nucleic acid comprises the nucleic acid encoding the RNA-guided endonuclease, and wherein one of the first or second nucleic acids further comprise the nucleic acid encoding the gRNA.

In any of the aspects of the present disclosure, the nucleic acid encoding a gRNA of the disclosure, an endonuclease of the disclosure, any donor nucleic acid, and/or any nucleic acid or proteinaceous molecule necessary to carry out the aspects of the methods of the disclosure can comprise a nucleic acid expression cassette. As used herein, the term “nucleic acid expression cassette” refers to an isolated nucleic acid molecule that includes one or more transcriptional control elements (e.g., promoters, enhancers, and/or regulatory elements, polyadenylation sequences, and introns) that are operably linked to and direct gene expression in one or more desired cell types, tissues or organs. A nucleic acid expression cassette can contain a transgene, although it is also envisaged that a nucleic acid expression cassette directs expression of an endogenous gene in a cell into which the nucleic acid sequence is inserted.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology, 1990, 185, Academic Press, San Diego, CA. Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the nucleic acid expression cassette can depend on such factors as the choice of the target cell, the level of expression desired, and the like.

In some examples, a nucleic acid expression cassette provided herein can comprise one or more transcription and/or translation control elements. Depending on the host and system utilized, any of a number of suitable transcription and translation control elements, including constitutive and inducible promoters, transcription enhancer elements, transcription terminators, etc. can be used in the expression vector. The transcription and translation control element can be tissue-specific or ubiquitous and can be constitutive or inducible, depending on the pattern of the gene expression desired. The transcription and translation control element can be native or foreign and can be a natural or a synthetic sequence. By foreign, it is intended that the transcriptional initiation region is not found in the wild-type host into which the transcriptional initiation region is introduced.

Suitable transcription and translation control elements include promoters, enhancers, and/or transcriptional termination signals.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter, CAG promoter). In some cases, the promoter can be a spatially restricted and/or temporally restricted promoter (e.g., a tissue specific promoter, a cell type specific promoter, etc.).

The promoter can be chosen so that it will function in the target cell(s) of interest. Tissue-specific promoters refer to promoters that have activity in only certain cell types. The use of a tissue-specific promoter in a nucleic acid expression cassette can restrict unwanted transgene expression in the unaffected tissues as well as facilitate persistent transgene expression by escaping from transgene induced host immune responses. Tissue specific promoters include, but are not limited to, neuron-specific promoters, muscle-specific promoters, liver-specific promoters, skeletal muscle-specific promoters, and heart-specific promoters. Examples of liver-specific promoters include, but are not limited to, the .alpha.1-microglobulin/bikunin enhancer/thyroid hormone-binding globulin promoter, the human albumin (hALB) promoter, the thyroid hormone-binding globulin promoter, the α-1-antitrypsin promoter, the bovine albumin (bAlb) promoter, the murine albumin (mAlb) promoter, the human a1-antitrypsin (hAAT) promoter, the ApoEhAAT promoter composed of the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the al-microglobulin enhancer, the DC190 promoter containing the human albumin promoter and the prothrombin enhancer, and other natural and synthetic liver-specific promoters. In one embodiment, the promoter comprises a human G6PC promoter provided herein as SEQ ID NO: 23 or a minimal functional portion thereof (e.g., any of SEQ ID NOs 20, 21, or 22). In another embodiment, the promoter comprises a U6 promoter. In yet other embodiments, the promotor comprises a glutamate rRNA.

In other aspects, the promoter can be a constitutive promoter. Constitutive promoters refer to promoters that allow for continual transcription of its associated gene. Constitutive promoters are always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells and fibroblasts. Examples of constitutive promoters include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PyK promoter, a human ubiquitin C gene (Ubc) promoter, a MFG promoter, a human beta actin promoter, a CAG promoter, a EGRI promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous promoters. In some embodiments, the constitutively active promoter is selected from the group consisting of human β-actin, human elongation factor-la, chicken β-actin combined with cytomegalovirus early enhancer, cytomegalovirus (CMV), simian virus 40, or herpes simplex virus thymidine kinase.

Inducible promoters refer to promoters that can be regulated by positive or negative control. Factors that can regulate an inducible promoter include, but are not limited to, chemical agents (e.g., the metallothionein promoter or a hormone inducible promoter), temperature, and light.

The tissue-specific promoters can be operably linked to one or more (e.g., 2, 3, 4, 5, 6, 7, or 8) enhancer elements (e.g., a neuron-specific promoter fused to a cytomegalovirus enhancer) or combined to form a tandem promoter (e.g., neuron-specific/constitutive tandem promoter). When two or more tissue-specific promoters are present, the isolated nucleic acid can be targeted to two or more different tissues at the same time.

As discussed above, a disclosed promoter can be an endogenous promoter. Endogenous refers to a disclosed promoter or disclosed promoter/enhancer that is naturally linked with its gene. In an aspect, a disclosed endogenous promoter can generally be obtained from a non-coding region upstream of a transcription initiation site of a gene (such as, for example, a disclosed phosphorylase kinase, phosphorylase, or some other enzyme involved in the glycogen metabolic pathway). In an aspect, a disclosed endogenous promoter can be used for constitutive and efficient expression of a disclosed transgene (e.g., a nucleic acid sequence encoding a polypeptide capable of preventing glycogen accumulation and/or degrading accumulated glycogen). In an aspect, a disclosed endogenous promoter can be an endogenous promoter/enhancer.

As discussed above, a disclosed promoter can be an exogenous promoter. Exogenous (or heterologous) refers to a disclosed promoter or a disclosed promoter/enhancer that can be placed in juxtaposition to a gene by means of molecular biology techniques such that the transcription of that gene can be directed by the linked promoter or linked promoter/enhancer.

An enhancer element is a nucleic acid sequence that functions to enhance transcription. As used herein, the terms “enhance” and “enhancement” with respect to nucleic acid expression or polypeptide production, refers to an increase and/or prolongation of steady-state levels of the indicated nucleic acid or polypeptide, e.g., by at least about 2%, 5%, 10%, 15%, 20%, 25%, 40%, 50%, 60%, 75%, 2-fold, 2.5-fold, 3-fold, 5-fold, 10-fold, 15-fold, 20-fold, 30-fold, 50-fold, 100-fold or more. As used herein, the term “intron” refers to nucleic acid sequences that can enhance transgene expression. An intron can also be a part of the nucleic acid expression cassette or positioned downstream or upstream of the expression cassette in the expression vector. Introns can include, but are not limited to, the SV40 intron, EF-1alpha gene intron 1, or the MVM intron. In some embodiments, the nucleic acid expression cassettes do not contain an intron. Representative enhancer elements that can be used herein include any enhancer elements normally associated with a G6PC gene.

In other aspects, the nucleic acid expression cassettes according to the present disclosure can further comprise a transcriptional termination signal. A transcriptional termination signal is a nucleic acid sequence that marks the end of a gene during transcription. Examples of a transcriptional termination signal include, but are not limited to, bovine growth hormone polyadenylation signal (BGHpA), Simian virus 40 polyadenylation signal (Sv40 PolyA), and a synthetic polyadenylation signal. A polyadenylation sequence can comprise the nucleic acid sequence AATAAA. In some embodiments, the nucleic acid encoding the therapeutic protein (e.g., the nucleic acid encoding glucose-6-phosphatase) comprises a FLAG tag at the C-terminus.

In any of the foregoing or related aspects, the nucleic acids disclosed herein may be “codon optimized” to ensure expression in a target cell or organism. As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

### (e) Vectors

In any of the aspects of the present disclosure, the isolated nucleic acids and/or nucleic acid expression cassettes as provided herein may be packaged or provided in a vector (e.g., a recombinant expression vector). The term “vector” refers to a nucleic acid molecule capable of transporting another nucleic acid to which it has been linked. It will be apparent to those skilled in the art that any suitable vector can be used to deliver the isolated nucleic acids of the disclosure to the target cell(s) or subject of interest. The choice of delivery vector can be made based on a number of factors known in the art, including age and species of the target host, in vitro vs. in vivo delivery, level and persistence of expression desired, intended purpose (e.g., for therapy or enzyme production), the target cell or organ, route of delivery, size of the isolated nucleic acid, safety concerns, and the like.

Accordingly, in some aspects of the present disclosure a vector system is provided comprising (a) a first vector comprising a nucleic acid (e.g., an isolated nucleic acid and/or the nucleic acid expression cassette described herein) that comprises the donor nucleic acid provided herein, and (b) a second vector comprising a nucleic acid (e.g., an isolated nucleic acid and/or the nucleic acid expression cassette described herein) that encodes for a site-directed endonuclease (e.g., a Cas9 endonuclease), wherein at least one of (a) or (b) further comprises a nucleic acid encoding for a gRNA as described herein. The vector system herein can be used for stable integration of a G6PC transgene into the genome of a target cell or organism.

In some aspects, the first vector comprises a nucleotide sequence having at least 70%, at least 75%, at least 80%, at least 85%, at least 90%, at least 95%, at least 96%, at least 97%, at least 98%, at least 99% or 100% to any one of SEQ ID NOs: 41 to 45. For example, in some aspects, the first vector comprises a nucleic acid having a nucleotide sequence comprising any one of SEQ ID NOs: 41 to 45. For example, in some aspects, the first vector comprises a nucleic acid having a nucleotide sequence consisting of any one of SEQ ID NOs: 41 to 45. In some aspects, the first vector consists of any one of SEQ ID NOs: 41 to 45.

In some aspects, the second vector comprises a nucleotide sequence having at least 70%, at least 75%, at least 80%, at least 85%, at least 90%, at least 95%, at least 96%, at least 97%, at least 98%, at least 99% or 100% to any one of SEQ ID NOs: 46 to 48. For example, in some aspects, the second vector comprises a nucleic acid having a nucleotide sequence comprising any one of SEQ ID NOs: 46 to 48. For example, in some aspects, the second vector comprises a nucleic acid having a nucleotide sequence consisting of any one of SEQ ID NOs: 46 to 48. In some aspects, the second vector consists of any one of SEQ ID NOs: 46 to 48.

In some aspects, the vector system provided herein comprises a first vector comprising a nucleic acid sequence of SEQ ID NO: 41 and a second vector comprising a nucleic acid sequence of SEQ ID NO: 46. In some aspects, the vector system provided herein comprises a first vector consisting of a nucleic acid sequence of SEQ ID NO: 41 and a second vector consisting of a nucleic acid sequence of SEQ ID NO: 46.

In some aspects, the vector system provided herein comprises a first vector comprising a nucleic acid sequence of SEQ ID NO: 42 and a second vector comprising a nucleic acid sequence of SEQ ID NO: 46. In some aspects, the vector system provided herein comprises a first vector consisting of a nucleic acid sequence of SEQ ID NO: 42 and a second vector consisting of a nucleic acid sequence of SEQ ID NO: 46.

In some aspects, the vector system provided herein comprises a first vector comprising a nucleic acid sequence of SEQ ID NO: 43 and a second vector comprising a nucleic acid sequence of SEQ ID NO: 47. In some aspects, the vector system provided herein comprises a first vector consisting of a nucleic acid sequence of SEQ ID NO: 43 and a second vector consisting of a nucleic acid sequence of SEQ ID NO: 47.

In some aspects, the vector system provided herein comprises a first vector comprising a nucleic acid sequence of SEQ ID NO: 44 and a second vector comprising a nucleic acid sequence of SEQ ID NO: 48. In some aspects, the vector system provided herein comprises a first vector consisting of a nucleic acid sequence of SEQ ID NO: 44 and a second vector consisting of a nucleic acid sequence of SEQ ID NO: 48.

In some aspects, the vector system provided herein comprises a first vector comprising a nucleic acid sequence of SEQ ID NO: 45 and a second vector comprising a nucleic acid sequence of SEQ ID NO: 46. In some aspects, the vector system provided herein comprises a first vector consisting of a nucleic acid sequence of SEQ ID NO: 45 and a second vector consisting of a nucleic acid sequence of SEQ ID NO: 46.

For ease of reference, exemplary full sequences for vectors that can be used in vector systems provided herein are described in Table 7 at the end of this application.

In accord with any of the foregoing or related aspects, the vectors can comprise one or more further elements (e.g., transcription and/or translation control elements described above) that enable expression of nucleic acids of interest in a target cell or organism. The vectors can be viral or non-viral as described further below. Suitable vectors that are known in the art and that can be used to deliver, and optionally, express the isolated nucleic acids of the disclosure (e.g., viral and non-viral vectors), including, virus vectors (e.g., retrovirus, adenovirus, AAV, lentiviruses, or herpes simplex virus), lipid vectors, poly-lysine vectors, synthetic polyamino polymer vectors that are used with nucleic acid molecules, such as a plasmid, and the like. In some embodiments, the non-viral vector can be a polymer-based vector (e.g., polyethyleimine (PEI), chitosan, poly (DL-Lactide) (PLA), or poly (DL-lactidie-co-glycoside) (PLGA), dendrimers, polymethacrylate) a peptide-based vector, a lipid nanoparticle, a solid lipid nanoparticle, or a cationic lipid based vector.

Other types of vectors include “plasmids”, which are circular double-stranded DNA loops into which additional nucleic acid segments can be ligated and viral vectors wherein additional nucleic acid segments can be ligated into the viral genome and which comprises the vector genome (e.g., viral DNA) packaged within a virion. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In some examples, the vectors, like the nucleic acid expression cassettes above, can be capable of directing the expression of nucleic acids to which they are operatively linked. Such vectors are referred to herein as “recombinant expression vectors”, or more simply “expression vectors”, which serve equivalent functions.

In some embodiments, the nucleic acid expression cassettes and/or transgenes (e.g., G6PC and variants thereof) can be incorporated into a recombinant viral vector. As used herein, the term “viral vector” refers to a virus (e.g., AAV) particle that functions as a nucleic acid delivery vehicle, and which comprises the vector genome (e.g., viral DNA) packaged within a virion. Alternatively, in some contexts, the term “vector” is used to refer to the vector genome/viral DNA alone.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia).

In some aspects, the vector is a recombinant viral vector suitable for gene therapy. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and plant virus satellites.

In some embodiments, the recombinant viral vector is selected from the group consisting of adenoviruses, Adeno-associated viruses (AAV) (e.g., AAV serotypes and genetically modified AAV variants), a herpes simplex viruses (e.g., e.g., HSV-1, HSV), a retrovirus vector (e.g., MMSV, MSCV), a lentivirus vector (HIV-1, HIV-2), and alphavirus vector (e.g., SFV, SIN, VEE, M1), a flavivirus vector (e.g., Kunjin, West Nile, Dengue virus), a rhabdovirus vector (e.g., Rabies, VSV), a measles virus vector (e.g., MV-Edm), a Newcastle disease virus vector, a poxvirus vector (VV), or a picornavirus vector (e.g., Coxsackievirus). The recombinant viral vector of the present disclosure includes any type of viral vector that is capable of packaging and delivering the G6PC transgene or viral vectors that can be designed engineered and generated by methods known in the art.

In some embodiments, the delivery vector is an adenovirus vector. The term “adenovirus” as used herein encompasses all adenoviruses, including the Mastadenovirus and Aviadenovirus genera.

The various regions of the adenovirus genome have been mapped and are understood by those skilled in the art. The genomic sequences of the various Ad serotypes, as well as the nucleotide sequence of the particular coding regions of the Ad genome, are known in the art and may be accessed from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

A recombinant adenovirus (rAd) vector genome can comprise the adenovirus terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small transgene, “stutter DNA” can be used to maintain the total size of the vector within the desired range by methods known in the art.

The genome of an adenovirus can be manipulated such that it encodes and expresses a gene product of interest but is inactivated in terms of its ability to replicate in a normal lytic viral life cycle. Suitable adenoviral vectors derived from the adenovirus strain Ad type 5 (Ad5) or other strains of adenovirus (e.g., Ad2, Ad3, Ad7, etc.) are known to those skilled in the art.

In some embodiments, the viral vector comprises a recombinant Adeno-Associated Viruses (AAV). AAV are parvoviruses and have small icosahedral virions and can contain a single stranded DNA molecule about 4.7 kb (e.g., about 4.5 kb, 4.6 kb, 4.8 kb, 4.9 kb, or 5.0 kb) or less in size. The viruses contain either the sense or antisense strand of the DNA molecule and either strand is incorporated into the virion. Two open reading frames encode a series of Rep and Cap polypeptides. Rep polypeptides (e.g., Rep50, Rep52, Rep68 and Rep78) are involved in replication, rescue and integration of the AAV genome, although significant activity may be observed in the absence of all four Rep polypeptides. The Cap proteins (e.g., VP1, VP2, VP3) form the virion capsid. Flanking the rep and cap open reading frames at the 5′ and 3′ ends of the genome are inverted terminal repeats (ITRs). Typically, in recombinant AAV (rAAV) vectors, the entire rep and cap coding regions are excised and replaced with a transgene of interest.

Recombinant AAV vectors generally require only the inverted terminal repeat(s) (ITR(s)) in cis to generate virus. All other viral sequences are dispensable and may be supplied in trans. Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the present disclosure, the rAAV vector genome comprises at least one terminal repeat (ITR) sequence (e.g., AAV TR sequence), optionally two ITRs (e.g., two AAV ITRs), which typically will be at the 5′ and 3′ ends of the vector genome and flank the heterologous nucleic acid sequence, but need not be contiguous thereto. The ITRs can be the same or different from each other.

The term “inverted terminal repeat” or “ITR” is used equivalently herein with the term “terminal repeat” or “TR” and includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a ITR, which can further be modified by truncation, substitution, deletion, insertion and/or addition. Further, the ITR can be partially or completely synthetic, such as the “double-D sequence.”

An “AAV inverted terminal repeat” or “AAV ITR” may be from any AAV, including but not limited to serotypes 1, 2, 3, 4, 5, 6, 7, 8, 9, 10 or 11 or any other AAV now known or later discovered. An AAV terminal repeat need not have the native terminal repeat sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like. In some embodiments, the vector comprises flanking ITRs derived from the AAV2 genome.

Wild-type AAV can integrate their DNA into non-dividing cells and exhibit a high frequency of stable integration into human chromosome 19. A rAAV vector genome will typically comprise the AAV terminal repeat sequences and packaging signal.

An “AAV particle” or “rAAV particle” comprises an AAV vector genome or rAAV vector genome, respectively, packaged within an AAV capsid. The AAV rep/cap genes can be expressed on a single plasmid. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extrachromosomal elements, designated as an “EBV based nuclear episome,” see Margolski (1992) Curr. Top. Microbiol. Immun. 158:67). The AAV rep and cap genes may be from any AAV serotype for which recombinant virus can be derived and may be from a different AAV serotype than the rAAV genome ITRs.

However, the rAAV vector itself need not contain AAV genes encoding the capsid (cap) and Rep proteins. In particular embodiments of the disclosure, the rep and/or cap genes are deleted from the AAV genome. In a representative embodiment, the rAAV vector retains only the terminal AAV sequences (ITRs) necessary for integration, excision, and replication.

Sources for the AAV capsid genes can include naturally isolated serotypes, including but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7, as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In particular embodiments, the AAV capsids are chimeras either created by capsid evolution or by rational capsid engineering from the naturally isolated AAV variants to capture desirable serotype features such as enhanced or specific tissue tropism and host immune response escape, including but not limited to AAV-DJ, AAV-HAEI, AAV-HAE2, AAVM41, AAV-1829, AAV2 Y/F, AAV2 T/V, AAV218, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS., AAV-PHP.B, AAV-PHP.eB, and AAV-PHP.S.

Accordingly, when referring herein to a specific AAV capsid protein (e.g., an AAV2, AAV3, AAV4, AAV5, AAV6, AAV7, AAV8, AAV9, AAV10, AAVI or AAV12 capsid protein) it is intended to encompass the native capsid protein as well as capsid proteins that have alterations other than the modifications of the invention. Such alterations include substitutions, insertions and/or deletions.

In some embodiments, the recombinant AAV vectors are selected from the group consisting of AAV7, AAVI, AAV10, AAV8, or AAV9. In certain embodiments, the recombinant AAV vector comprises AAV9 due to its ability to easily cross the blood-brain barrier.

In some embodiments, the recombinant viral vectors (e.g., rAAV) according to the present disclosure generally comprise, consist of, or consist essentially of one or more of the following elements: (1) an Inverted Terminal Repeat sequence (ITR); (2) a promoter (e.g., a liver-specific promoter); (3) a transgene (e.g., a nucleic acid sequence encoding G6PC, a fragment thereof, an isoform thereof, or a homologue thereof); (4) a transcription terminator (e.g., a polyadenylation signal); and (5) a flanking Inverted Terminal Repeat sequence (ITR).

In some embodiments, the recombinant viral vectors can comprise a linker sequence. The term “linker sequence” as used herein refers to a nucleic acid sequence that encodes a short polypeptide sequence. A linker sequence can comprise at least 6 nucleotide sequences, at least 15 nucleotides, 27 nucleotides, or at least 30 nucleotides. In some embodiments, the linker sequence has 6 to 27 nucleotides. In other embodiments, the linker sequence has 6 nucleotides, 15 nucleotides, and/or 27 nucleotides. A linker sequence can be used to connect various encoded elements in the vector constructs. For example, a transgene and Myc tag can be operably linked via a linker, or a Myc tag and FLAG can be operably linked via a linker or a FLAG tag and mCherry tag can be operably linked via a linker. Alternatively, the vector elements can be directly linked (e.g., not via a linker).

In some embodiments, the AAV vectors are pseudotyped, which refers to the practice of creating hybrids of certain AAV strains to be able to refine the interaction with desired target cells. The hybrid AAV can be created by taking a capsid from one strain and the genome from another strain. For example, AAV2/5, a hybrid with the genome of AAV2 and the capsid of AAV5, can be used to achieve more accuracy and range in brain cells than AAV2 would be able to achieve unhybridized. Production of pseudotyped rAAV is disclosed in, for example, WO01/83692.

Other types of rAAV variants, for example rAAV with capsid mutations, are also contemplated. See, for example, Marsic et al., Molecular Therapy, 22 (11): 1900-1909 (2014). It is understood that the nucleotide sequences of the genomes of various AAV serotypes are known in the art.

Examples of recombinant AAV that can be constructed to comprise the nucleic acid molecules of the disclosure are set out in International Patent Application No. PCT/US2012/047999 (WO 2013/016352) incorporated by reference herein in its entirety.

Any suitable method known in the art can be used to produce AAV vectors. In one particular method, AAV stocks can be produced by co-transfection of a rep/cap vector plasmid encoding AAV packaging functions and the vector plasmid containing the recombinant AAV genome into human cells infected with the helper adenovirus. General principles of recombinant AAV production are reviewed in, for example, Carter, 1992, Current Opinions in Biotechnology, 1533-539; and Muzyczka, (1992) Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. Nos. 5,173,414; 5,658,776; WO 95/13392; WO 96/17947; WO 97/09441; WO 97/08298; WO 97/21825; WO 97/06243; WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to recombinant AAV production.

The recombinant viral vectors (e.g., rAAV) may be purified by methods standard in the art such as by column chromatography or cesium chloride gradients. Methods for purifying recombinant viral vectors from helper virus are known in the art.

The nucleic acid encoding G6PC and/or CRISPR/Cas9 can be provided to the cell using any method known in the art. For example, the template can be supplied by a non-viral (e.g., plasmid) or viral vector.

The AAV rep and/or cap genes can alternatively be provided by a packaging cell that stably expresses the genes. A method of generating a packaging cell is to create a cell line that stably expresses all the necessary components for viral (e.g., AAV) particle production. For example, in one embodiment, a plasmid (or multiple plasmids) comprising a viral rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

In one embodiment, packaging cells can be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In still further embodiments, the delivery vectors are a hybrid Ad-AAV delivery vector. Briefly, the hybrid Ad-AAV vector comprises an adenovirus vector genome comprising adenovirus (i) 5′ and 3′ cis-elements for viral replication and encapsidation and, further, (ii) a recombinant AAV vector genome comprising the AAV 5′ and 3′ inverted terminal repeats (ITRs), an AAV packaging sequence, and a heterologous sequence(s) flanked by the AAV ITRs, where the recombinant AAV vector genome is flanked by the adenovirus 5′ and 3′ cis-elements. The adenovirus vector genome can further be deleted, as described above.

Another vector for use in the present disclosure comprises Herpes Simplex Virus (HSV). HSV can be modified for the delivery of transgenes to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express transgenes for a long period of time in the central nervous system as long as the lytic cycle does not occur.

Herpes virus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al. (1999) Gene Therapy 6:986 and WO 00/17377.

In other embodiments of the present disclosure, the delivery vector of interest is a retrovirus. Retroviruses normally bind to a species-specific cell surface receptor, e.g., CD4 (for HIV); CAT (for MLV-E; ecotropic Murine leukemic virus E); RAMI/GLVR2 (for murine leukemic virus-A; MLV-A); GLVRI (for Gibbon Ape leukemia virus (GALV) and Feline leukemia virus B (FeLV-B)). The development of specialized cell lines (termed “packaging cells”) which produce only replication-defective retroviruses has increased the utility of retroviruses for gene therapy, and defective retroviruses are characterized for use in gene transfer for gene therapy purposes. A replication-defective retrovirus can be packaged into virions which can be used to infect a target cell through the use of a helper virus by standard techniques.

Yet another suitable vector is a lentiviral vector. Lentiviruses are a subtype of retroviruses but they have the unique ability to infect non-dividing cells, and therefore can have a ride range of potential applications.

Yet another suitable vector is a poxvirus vector. These viruses contain more than 100 proteins. Extracellular forms of the virus have two membranes while intracellular particles only have an inner membrane. The outer surface of the virus is made up of lipids and proteins that surround the biconcave core. Poxviruses are very complex antigenically, inducing both specific and cross-reacting antibodies after infection. Poxvirus can infect a wide range of cells. Poxvirus gene expression is well studied due to the interest in using vaccinia virus as a vector for expression of transgenes.

In another representative embodiment, the nucleic acid sequence encoding G6PC is provided by a replicating rAAV virus. In still other embodiments, an AAV provirus comprising the nucleic acid sequence encoding G6PC and/or CRISPR/Cas9 can be stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

In addition to viral transfer methods, such as those illustrated above, non-viral methods can also be employed. Many non-viral methods of gene transfer rely on normal mechanisms used by mammalian cells for the uptake and intracellular transport of macromolecules. In particular embodiments, non-viral delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present disclosure. Naked plasmids can be introduced into cells by injection into the tissue. Expression can extend over many months. Cationic lipids can aid in introduction of DNA into some cells in culture. Injection of cationic lipid plasmid DNA complexes into the circulation of mice can result in expression of the DNA in organs (e.g., the lung). One advantage of plasmid DNA is that it can be introduced into non-replicating cells.

In a representative embodiment, a nucleic acid molecule (e.g., a plasmid) can be entrapped in a lipid particle bearing positive changes on its surface and, optionally, tagged with antibodies against cell surface antigens of the target tissue.

Liposomes that consist of amphiphilic cationic molecules are useful non-viral vectors for nucleic acid delivery in vitro and in vivo. The positively charged liposomes are believed to complex with negatively charged nucleic acids via electrostatic interactions to form lipid: nucleic acid complexes. The lipid: nucleic acid complexes have several advantages as gene transfer vectors. Unlike viral vectors, the lipid: nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency.

Amphiphilic cationic lipid: nucleic acid complexes can be used for in vivo transfection both in animals and in humans and can be prepared to have a long shelf-life.

In addition, vectors according to the present disclosure can be used in diagnostic and screening methods, whereby a nucleic acid encoding G6PC is transiently or stably expressed in a cell culture system, or alternatively, a transgenic animal model screening method, whereby a nucleic acid of interest is transiently or stably expressed in a cell culture system, or alternatively, a transgenic animal model.

The vectors of the present disclosure can also be used for various non-therapeutic purposes, including but not limited to use in protocols to assess gene targeting, clearance, transcription, translation, etc., as would be apparent to one skilled in the art. The vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

### (f) Non-Gene Editing AAV Vectors

In certain aspects herein, non-gene editing AAV vectors can be administered to a subject that has received, is receiving, or will receive gene editing treatment as described herein. These non-gene editing AAV vectors comprise an AAV vector containing a G6PC transgene operably linked to a promoter. They do not comprise any gene editing components (e.g., sequences encoding a side-directed nuclease or targeting molecule). Treatments using these types of vectors are known as “gene replacement therapy” and allow for exogenous expression in a cell. Exemplary “gene replacement” vectors are described in, for example, Luo, X., et al., (2011). Mol Ther. 19, 1961-1970, which is incorporated herein by reference in its entirety. The non-gene editing vectors can be, optionally, be prepared as AAV vectors and can comprise any serotypes or additional components standard to these vectors, as described above.

In some aspects, the non-gene editing AAV vectors disclosed herein can comprise a nucleic acid sequence having at least 70%, at least 75%, at least 80%, at least 85%, at least 90%, at least 95%, at least 96%, at least 97%, at least 98%, at least 99% or 100% sequence homology to SEQ ID NO: 49. In various aspects, the non-gene editing AAV vectors disclosed herein can comprise a nucleic acid sequence comprising SEQ ID NO: 49. In further aspects, the non-gene editing AAV vectors disclosed herein can consist of a nucleic acid sequence of SEQ ID NO: 49. For ease of reference, SEQ ID NO: 49 is provided in Table 7 at the end of this application.

### III. Pharmaceutical Formulations

Another aspect of the present disclosure provides a composition and/or pharmaceutical formulation comprising, consisting, or consisting essentially of a nucleic acid, a nucleic acid expression cassete, a vector and/or the vector system provided herein. As the gene editing systems herein comprise, in some embodiments, at least two separate nucleic acids (e.g., a nucleic acid comprising the donor nucleic acid and a second nucleic acid encoding for one or more CRISPR elements like Cas9 and/or gRNA), the compositions and/or pharmaceutical formulations can comprise nucleic acids, nucleic acid expression cassettes and/or vectors separately (e.g., two separate compositions) or they can be together as one in a single formulation.

In some embodiments, compositions of the present disclosure comprise, consist of, or consist essentially of a recombinant viral vector (e.g., rAAV) and/or a pharmaceutically acceptable carrier and/or excipient, and, optionally, other medicinal agents, pharmaceutical agents, stabilizing agents, buffers, carriers, adjuvants, diluents, etc. For injection, the carrier will typically be a liquid. For other methods of administration, the carrier can be either solid or liquid. For inhalation administration, the carrier will be respirable, and optionally can be in solid or liquid particulate form.

By “pharmaceutically acceptable” it is meant a material that is not biologically or otherwise undesirable, i.e., the material can be administered to a subject along with the isolated nucleic acid or vector without causing any undesirable biological effects such as toxicity. Thus, such a pharmaceutical composition can be used, for example, in transfection of a cell ex vivo or in administering an isolated nucleic acid or vector directly to a subject.

The compositions can also comprise other ingredients such as diluents and adjuvants. Acceptable carriers, diluents and adjuvants are nontoxic to recipients and are preferably inert at the dosages and concentrations employed, and can include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

The pharmaceutical carriers, diluents or excipients suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some embodiments, sterile injectable solutions are prepared by incorporating the recombinant viral vector (e.g., rAAV) in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of recombinant viral vector (e.g., rAAV) as a free acid (DNA contains acidic phosphate groups) or a pharmacologically acceptable salt can be prepared in water suitably mixed with a surfactant such as hydroxpropylcellulose. A dispersion of recombinant viral vector (e.g., rAAV) can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

In an aspect, a disclosed pharmaceutical formulation can regulate, restore, normalize, and/or maintain one or more liver enzymes and/or metabolites. Liver enzyme and/or metabolites can comprise Alanine transaminase (ALT), Aspartate transaminase (AST), Alkaline phosphatase (ALP), Albumin and total protein, Bilirubin, Gamma-glutamyltransferase (GGT), L-lactate dehydrogenase (LD), Prothrombin time (PT), or any combination thereof. In an aspect, a disclosed pharmaceutical formulation can regulate, restore, normalize, and/or maintain one or more urine enzymes and/or metabolites (such as, for example, glucotetrasaccharides (HEX4)).

Pharmaceutical compositions can be prepared as injectable formulations or as topical formulations to be delivered to the subject by transdermal transport. Numerous formulations for both intramuscular injection and transdermal transport have been previously developed and can be used in the practice of the invention. The recombinant viral vector can be used with any pharmaceutically acceptable carrier and/or excipient for ease of administration and handling.

### III. Gene Editing Methods and Modes of Administration

In accordance with various aspects of the present disclosure, gene editing methods are provided wherein one or more nucleic acids are delivered to a cell, the one or more nucleic acids encoding for a site directed endonuclease as provided herein, a gRNA as provided herein, and a donor nucleic acid as provided herein. Once delivered, the site directed endonuclease and gRNA can be expressed by the cell, effecting a double stranded break at a location in the G6PC gene locus targeted by the gRNA and allowing for insertion of the donor nucleic acid via homologous directed repair (HDR). Accordingly, the gene editing methods can in some aspects provide for stably integrating a G6PC transgene into a cell. Additionally, the gene editing methods can in some aspects, provide for expressing a G6PC transgene in a cell (where the target cell is a cell in the subject). In still other aspects, the gene editing methods provide for treating or preventing a glycogen storage disease in a subject.

The nucleic acids can be delivered as viral vectors (e.g., recombinant viral vectors) as described herein. Accordingly, in certain embodiments, a titer of a recombinant viral vector comprising one or more of the nucleic acids described above is delivered to the cell or subject.

Titers of recombinant viral vectors (e.g., rAAV) to be administered according to the methods of the present disclosure will vary depending, for example, on the particular recombinant viral vector, the mode of administration, the treatment goal, the individual, and the cell type(s) being targeted, and can be determined by methods standard in the art.

In the case of a viral vector(s), virus particles can be contacted with the cells at the appropriate multiplicity of infection according to standard transduction methods appropriate for the particular target cells. Titers of virus to administer can vary, depending upon the target cell type and the particular virus vector, and can be determined by those of skill in the art. Typically, at least about 10³ virus particles, at least about 10⁵ particles, at least about 10⁷ particles, at least about 10⁹ particles, at least about 10¹¹ particles, or at least about 10¹² particles are administered to the cell. In exemplary embodiments, about 10⁷ to about 10¹⁵ particles, about 10⁷ to about 10¹³ particles, about 10⁸ to about 10¹² particles, about 10¹⁰ to about 10¹⁵ particles, about 10¹¹ to about 10¹⁵ particles, about 10¹² to about 10¹⁴ particles, or about 10¹² to about 10¹³ particles are administered. Dosages may also be expressed in units of viral genomes (vg).

The cell to be administered the vectors of the disclosure can be of any type, including but not limited to neuronal cells (including cells of the peripheral and central nervous systems), retinal cells, epithelial cells (including dermal, gut, respiratory, bladder, pulmonary, peritoneal and breast tissue epithelium), muscle (including cardiac, smooth muscle, including pulmonary smooth muscle cells, skeletal muscle, and diaphragm muscle), pancreatic cells (including islet cells), kidney cells, hepatic cells (including parenchyma), cells of the intestine, fibroblasts (e.g., skin fibroblasts such as human skin fibroblasts), fibroblast-derived cells, endothelial cells, intestinal cells, germ cells, lung cells (including bronchial cells and alveolar cells), prostate cells, stem cells, progenitor cells, dendritic cells, and the like. Moreover, the cells can be from any species of origin, as indicated above.

Methods of transducing a target cell with a vector according to the present disclosure are also contemplated by the present disclosure. The term “transduction” is used herein to refer to the administration/delivery of an G6PC transgene to a recipient cell either in vivo or in vitro, via a replication-deficient recombinant viral vector (e.g., rAAV) of the present disclosure thereby resulting in expression of an G6PC by the recipient cell. Thus, the present disclosure provides methods of administering an effective dose (or doses, administered essentially simultaneously or doses given at intervals) of a recombinant viral vector (e.g., rAAV) that encodes G6PC and/or CRISPR/Cas9/gRNA to a subject in need thereof.

The in vivo transduction methods comprise the step of administering an effective dose, or effective multiple doses, of a nucleic acid expression cassette or composition comprising a recombinant viral vector of the present disclosure to an animal (including a human being) in need thereof. If the dose is administered prior to development of a disorder/disease, the administration is prophylactic. If the dose is administered after the development of a disorder/disease, the administration is therapeutic. In embodiments of the present disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the present disclosure is a glycogen storage disease such as but not limited to glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, glycogenosis due to AMP-activated protein kinase gamma subunit 2-deficiency (PRKAG2), or cardiac glycogenosis due to AMP-activated protein kinase gamma subunit 2 deficiency. In some aspects, the disease contemplated for prevention or treatment with methods of the present disclosure is a GSD type I disease selected from GSD Ia, GSD Ib, or GSD Ic. For example, in some aspects, the disease is GSD Ia.

Transduction with a recombinant viral vector(s) (e.g., rAAV) can also be carried out in vitro. In one embodiment, desired target cells are removed from the subject, transduced with recombinant viral vector (e.g., rAAV) and reintroduced into the subject. Alternatively, syngeneic or xenogeneic target cells can be used where those cells will not generate an inappropriate immune response in the subject.

Suitable methods for the transduction of a recombinant viral vector(s) (e.g., rAAV) or the reintroduction of transduced cells into a subject are known in the art. In one embodiment, cells can be transduced in vitro by combining the recombinant viral vector (e.g., rAAV) with target cells, e.g., in appropriate media, and screening for those cells harboring the DNA of interest using conventional techniques such as Southern blots and/or PCR, or by using selectable markers. A recombinant viral vector (e.g., rAAV) or transduced cells can then be formulated into pharmaceutical compositions, and the composition introduced into the subject by various techniques, such as by intramuscular, intravenous, subcutaneous and intraperitoneal injection, by injection into smooth and cardiac muscle, using e.g., a catheter, intrathecal, intracisternal, intraventricular or intraparenchymal into the brain.

Transduction of cells with recombinant viral vector(s) (e.g., rAAV) of the present disclosure can result in the in sustained expression of G6PC and/or CRISPR/Cas9 (e.g., Cas9 endonuclease and gRNA). The present disclosure thus provides methods of administering/delivering a recombinant viral vector (e.g., rAAV) that expresses, for example, G6PC and/or CRISPR/Cas9 to a subject (e.g., a human patient). These methods include transducing tissues (including, but not limited to, tissues such as nervous system and muscle, organs such as brain, heart, liver, and glands such as salivary glands) with one or more recombinant viral vector (e.g., rAAV) of the present disclosure. Transduction can be carried out with gene cassettes comprising tissue specific control elements as described herein.

In any of the gene editing methods herein, the gene editing vectors (e.g., the “first vector” and “second vector” that together form the gene editing vector system) can be delivered separately or concurrently. If delivered separately, the first vector can be delivered before or after the second vector. If done concurrently, the first vector and second vector can be delivered in a single composition or in separate compositions. Likewise, delivery of the two vectors can occur via the same or different routes of administration (described below).

In any of the gene editing methods herein, the gene editing vectors (e.g., the “first vector” and “second vector” that together form the gene ediing vector system) can be delivered in a ratio (e.g., “first vector” to “second vector”). In some aspects, a ratio of the first vector to the second vector js from about 10:1 to about 1:1, from about 9:1 to about 1:1, from about 8:1 to about 1:1, from about 7:1 to about 1:1, from about 6:1 to about 1:1, from about 5:1 to about 1:1, from about 4:1 to about 1:1, from about 3:1 to about 1:1, from about 2:1 to about 1:1. In some aspects, a ratio of the first vector to the second vector js from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1. For example, in some aspects, the ratio of the first vector to the second vector is about 10:1, about 9:1, about 8:1, about 7:1, about 6:1, about 5:1, about 4:1, about 3:1, about 2:1, or about 1:1. In other aspects, the ratio of the first vector to the second vector is 10:1, 9:1, 8:1, 7:1, 6:1, 5:1, 4:1, 3:1, 2:1, or 1:1. For example, in some aspects, the ratio of the first vector is about 4:1, about 2:1, or about 1:1. In further aspects, the ratio of the first vector to the second vector is 4:1, 2:1 or 1:1.

### IV. Use of Gene Editing Compositions in Methods of Treatment

Another aspect of the present disclosure provides a method of treating and/or preventing disease progression of a GSD-mediated disease in a subject, the method comprising, consisting of, or consisting essentially of administering to the subject a therapeutically effective amount of one or more nucleic acid expression cassettes, vectors, compositions, or pharmaceutical compositions comprising a nucleic acid encoding glucose-6-phosphatase (e.g., the “donor nucleic acid”), a nucleic acid encoding a Cas9 endonuclease, and a nucleic acid encoding a gRNA described in the present disclosure. In various aspects, in the methods of treating/preventing disease progression herein, at least one cell in the subject stably integrates the nucleic acid encoding glucose 6 phosphatase into its genome and stably expresses glucose-6-phosphatase. In various aspects, the GSD-mediated disease is treated and/or its progression is slowed following administration of the therapeutically effective amount.

In some aspects, a method of treating and/or preventing disease progression herein comprises restoring one or more aspects of cellular homeostasis and/or cellular functionality in at least one cell of the subject in need thereof. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In any of the therapeutic gene editing methods herein (e.g., methods of treatment), the gene editing nucleic acid expression cassettes, vectors, compositions, and/or pharmaceutical compositions can be administered separately or concurrently. If delivered separately, a gene editing nucleic acid expression cassettes, vectors, compositions, and/or pharmaceutical compositions comprising a first nucleic acid comprising the donor nucleic acid can be delivered before a gene editing nucleic acid expression cassette, vector, composition, and/or pharmaceutical composition comprising a second nucleic acid encoding one or more CRISPR components (e.g., Cas9 endonuclease and/or gRNA). Equally likely, a gene editing nucleic acid expression cassette, vector, composition, and/or pharmaceutical composition comprising a first nucleic acid comprising the donor nucleic acid can be delivered after a gene editing nucleic acid expression cassette, vector, composition, and/or pharmaceutical composition comprising a second nucleic acid encoding one or more CRISPR components (e.g., Cas9 endonuclease and/or gRNA). In still other aspects, if delivered concurrently, the first and second gene editing nucleic acid expression cassette, vector, composition and/or pharmaceutical compositions can be delivered in a single composition or in separate compositions (administered simultaneously). Likewise, delivery of the two components (gene editing nucleic acid expression cassette, vector, composition and/or pharmaceutical compositions) can occur via the same or different routes of administration (described below).

In an aspect, a disclosed method can comprise repeating an administering step one or more times. In an aspect, a disclosed method can comprise monitoring the subject for adverse effects. In an aspect, in the absence of adverse effects, the method can comprise continuing to treat the subject and/or continuing to monitor the subject. In an aspect, in the presence of adverse effects, the method can comprise modifying one or more steps of the method.

As used herein, “modifying the method” can comprise modifying or changing one or more features or aspects of one or more steps of a disclosed method. For example, in an aspect, a method can be altered by changing the amount of one or more of the disclosed compounds, disclosed compositions, disclosed pharmaceutical formulations, or any combination thereof administered to a subject, or by changing the frequency of administration of one or more of the disclosed compounds, disclosed compositions, disclosed pharmaceutical formulations, or any combination thereof to a subject, by changing the duration of time one or more of the disclosed compounds, disclosed compositions, disclosed pharmaceutical formulations, or any combination thereof are administered to a subject, or by substituting for one or more of the disclosed components and/or reagents with a similar or equivalent component and/or reagent.

The same applies to all the disclosed compounds, disclosed compositions, disclosed pharmaceutical formulations, or any combination thereof.

In an aspect, a disclosed method can further comprise administering one or more “gene replacement vectors” to the subject. Gene replacement vectors are described above and refer to vectors delivering a nucleic acid encoding a protein of interest (i.e., glucose-6-phosphatase) operably linked to a promoter or enhancer to allow for expression in a host cell. They are distinguished from “gene editing vectors” provided herein in that they do not contain any CRISPR or other gene editing machinery or components. In some aspects, the disclosed methods comprise administering the gene replacement vectors before the gene editing vectors disclosed herein. For example, in some aspects, a subject can be treated with gene replacement vectors as a neonate and then treated with gene editing vectors as an adult. In other aspects, the disclosed methods comprise administering the gene replacement vectors after the gene editing vectors disclosed herein. For example, in some aspects, a subject can be treated with gene editing vectors as a neonate and gene replacement vectors as needed later (e.g., as an adult). Additional treatment and administration protocols can be derived according to those of skill in the art.

In an aspect, a disclosed method can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a person skilled in the art can determine the appropriate number of cycles. In an aspect, a disclosed immune modulator can be administered as many times as necessary to achieve a desired clinical effect.

In an aspect, a disclosed method can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can comprise reducing the pathological phenotype associated with a disease, condition, or disorder caused by, related to, and/or exacerbated by the presence of a mutated glucose-6-phosphatase, a deficiency and/or absence in normal glucose-6-phosphatase expression or any combination thereof.

In an aspect, a disclosed method can comprise diagnosing the subject as having a disease, condition, or disorder caused by, related to, and/or exacerbated by the presence of a mutated glucose-6-phosphatase, a deficiency and/or absence in normal glucose-6-phosphatase expression or any combination thereof. In an aspect, a disclosed method can further treat one or more symptoms of the subject.

In an aspect, a disclosed method can restore one or more aspects of cellular homeostasis and/or cellular functionality and/or metabolic dysregulation. In an aspect, restoring one or more aspects of cellular homeostasis and/or cellular functionality and/or metabolic dysregulation can comprise reducing the expression and/or activity level of one or more mutated glucose-6-phosphatase and/or increasing expression and/or activity level of one or more wildtype glucose 6-phosphatase or any combination thereof that causes, relates to, elicits, and/or exacerbated a disease, disorder, and/or condition in the subject.

As used herein, the term “subject” and “patient” are used interchangeably and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject can be a human patient that is at risk for, or suffering from, a glycogen storage disease (e.g., glycogen storage disease type I (GSD I), glycogen storage disease III (GSD III), glycogen storage disease IV (GSD IV), glycogen storage disease V (GSD V), glycogen storage disease VI (GSD VI), glycogen storage disease VII (GSD VII), glycogen storage disease IX (GSD IX), glycogen storage disease XI (GSD XI), glycogen storage disease XII (GSD XII), glycogen storage disease XIII (GSD XIII), glycogen storage disease XIV (GSD XIV) (phosphoglucomutase deficiency), Danon disease (GSD 2B, LAMP-2 deficiency), Lafora disease, glycogenosis due to AMP-activated protein kinase gamma subunit 2-deficiency (PRKAG2), or cardiac glycogenosis due to AMP-activated protein kinase gamma subunit 2 deficiency). In some aspects, the subject may be at risk for or suffering from a GSD type I disease such as GSD Ia, GSD Ib, or GSD Ic. For example, in some aspects, the subject is at risk for or suffering from GSD Ia. In some aspects, the subject may be at risk for or suffering from a GSD type III disease such as GSD-type IIIa, GSD-type IIIB, GSD-type IIIc, or GSD-type IIId. The subject can also be a human patient that is at risk for, or suffering from, a disease caused by a mutation in the G6PC gene. In some aspects, the mutation may result in partial or complete loss of expression of native, normal, glucose-6-phosphatase. The human patient can be of any age (e.g., an infant, child, or adult).

As used herein, “treatment” or “treating” refers to the clinical intervention made in response to a disease, disorder or physiological condition manifested by a patient or to which a patient may be susceptible. The aim of treatment includes the alleviation or prevention of symptoms, slowing or stopping (i.e., alleviating) the progression or worsening of a disease, disorder, or condition and/or the remission of the disease, disorder or condition (e.g., a GSD). Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

An “effective amount” or “therapeutically effective amount” as used herein means an amount which provides a therapeutic or prophylactic benefit. Effective amounts of the nucleic acid molecules and/or compositions and/or pharmaceutical compositions can be determined by a physician with consideration of individual differences in age, weight, and condition of the patient (subject).

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The term “administration” or “administering” as it applies to a human, primate, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like.

“Administration” can refer, e.g., to therapeutic, pharmacokinetic, diagnostic, research, placebo, and experimental methods. Treatment of a cell encompasses exposure of the cell to a reagent (e.g., a nucleic acid molecule), as well as contact of a reagent to a fluid, where the fluid is in contact with the cell. “Administering” also encompasses in vitro and ex vivo treatments, e.g., of a cell, by a reagent, diagnostic, binding composition, or by another cell.

Administration of an effective dose of the isolated nucleic acids, vectors, and compositions can be by routes standard in the art including, but not limited to, intravenous (e.g., via portal vein, hepatic artery or renal artery injection), intrarenal, intramuscular, intra-cistern magna (ICM), or parenteral. In some aspects, administration of an effective dose of the isolated nucleic acids, vectors and compositions can be intravenous, intrarenal, intramuscular, or perenteral administration. In some aspects, administration of the effective dose can comprise portal vein injection, hepatic artery injection, renal artery injection, or intra-cistern magna (ICM) administration.

Route(s) of administration and serotype(s) of viral (e.g., AAV) components of the recombinant viral vector(s) (e.g., rAAV, and in particular, the AAV ITRs and capsid protein) of the present disclosure can be chosen and/or matched by those skilled in the art taking into account the disease state being treated and the target cells/tissue(s) that are to express the G6PC.

The present disclosure further provides for local administration and systemic administration of an effective dose of rAAV and compositions of the present disclosure including combination therapy as provided herein. For example, systemic administration is administration into the circulatory system so that the entire body is affected. Systemic administration includes enteral administration such as absorption through the gastrointestinal tract and parenteral administration through injection, infusion or implantation.

In particular, actual administration of a vector (e.g., rAAV) of the present disclosure can be accomplished by using any physical method that will transport the vector into the target tissue of the subject. In certain aspects, the target tissue can comprise the liver, heart, skeletal muscle, smooth muscle, CNS, or PNS of the subject, or any combinaition thereof. The nucleic acid molecules, vectors, and/or compositions can be administered to the desired region(s) by any route known in the art, including but not limited to, intravenous (e.g., via portal vein, hepatic artery or renal artery injection), intrarenal, intramuscular, intra-cistern magna (ICM), or parenteral, intracerebroventricular, intraparenchymal, intracranial, intrathecal, intra-ocular, intracerebral, intraventricular administration, or a combination of any thereof. In an aspect, a disclosed vector can be concurrently and/or serially administered to a subject via multiple routes of administration.

In other embodiments, the nucleic acid molecules, vectors, and/or compositions can be administered in a liquid formulation by direct injection (e.g., stereotactic injection) to the desired region or compartment in the liver, heart, skeletal muscle, CNS or PNS. As a further alternative, the virus vector and/or capsid can be administered as a solid, slow-release formulation.

In other embodiments, more than one route of administration can be utilized (e.g., ICV and ICM administration). For example, resuspending the recombinant viral vector (e.g., rAAV) in phosphate buffered saline (PBS) can be sufficient to provide a vehicle useful for muscle tissue expression, and there are no known restrictions on the carriers or other components that can be co-administered with the recombinant viral vector (e.g., rAAV, although compositions that degrade DNA should be avoided in the normal manner with rAAV). In cases where the recombinant viral vector comprises rAAV, the capsid proteins of a rAAV can be modified so that the rAAV is targeted to a particular target tissue of interest such as muscle.

Dosages will depend upon the mode of administration, the severity of the disease or condition to be treated, the individual subject's condition, the particular vector, and the gene to be delivered, and can be determined in a routine manner. In some embodiments, the isolated nucleic acid molecule or vector is administered to the subject in a therapeutically effective amount, as that term is defined above.

The dose of vector(s) (e.g., rAAV) to be administered in methods disclosed herein will vary depending, for example, on the particular recombinant viral vector, the mode of administration, the treatment goal, the individual, and the cell type(s) being targeted, and can be determined by methods standard in the art. Titers of each recombinant viral vector (e.g., rAAV) administered can range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more per ml. Dosages can also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ respectively). Dosages can also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively).

In another aspect, a therapeutically effective amount of disclosed vector can be delivered via intravenous (IV) administration and can comprise a range of about 1×10¹⁰ vg/kg to about 2×10¹⁴.vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹ no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a therapeutically effective amount of disclosed vector can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, restoring the expression of G6Pase). Methods for titering viral vectors such as AAV are described in Clark et al., Hum. Gene Ther., 10:1031-1039 (1999).

In some embodiments, more than one administration (e.g., two, three, four or more administrations) can be employed to achieve the desired level of gene expression over a period of various intervals, e.g., daily, weekly, monthly, or yearly. In some aspects, the methods herein comprise administering the vectors, nucleic acids or pharmaceutical compositions herein to a subject during a neonatal or infant period (e.g., within the first year of life), during early childhood (e.g., from 1 year to 5 years after birth), during later childhood (e.g., from 6 years to 10 years after birth), during pre-adolescence (e.g, 11 years to 12 years after birth), during adolescence (e.g., 13 years to 18 years after birth), or as an adult (e.g., after age 18). In some aspects, the vectors, nucleic acids and/or pharmaceutical compositiins are delivered during a neonatal or infant period (e.g., at birth, at 1 week after birth, at 2 weeks after birth, at 3 weeks after birth, at 4 weeks after birth, at 1 month after birth, at 2 months after birth, at 3 months after birth, at 4 months after birth, at 5 months after birth, at 6 months after birth, at 7 months after birth, at 8 months after birth, at 9 months after birth, at 10 months after birth, at 11 months after birth or at 12 months after birth). In some aspects, the vectors, nucleic acids and/or pharmaceutiacal compositons are delivered at birth. In some aspects, the vectors, nucleic acids and/or pharmaceutical compositons are delivered 2 or 3 or 4 months after birth. In some aspects, the vectors, nucleic acids and/or pharmaceutical compositions are delivered 1, 2, 3, 4, 5, 6, 7, 8, 9, 10, 11, 12, 13, 14, 15, 16, 17, or 18 years after birth. In some aspects, the vectors, nucleic acids and/or pharmaceutical compositons are delivered to an adult subject. In some aspects, the vectors, nucleic acids and/or pharmaceutical compositions can be delivered at multiple points during the subject's life (e.g., during a neonatal/infant period or during childhood and again as an adult).

Delivery to a target tissue can also be achieved by delivering a depot comprising the virus vector(s) and/or capsid(s). In representative embodiments, a depot comprising the vector and/or capsid is implanted into skeletal, cardiac and/or diaphragm muscle tissue or the tissue can be contacted with a film or other matrix comprising the virus vector and/or capsid.

In those embodiments where the nucleic acids, vectors, compositions and/or pharmaceutical compositions are separate (i.e., the G6PC and CRISPR/Cas9 are not in a single mixture/formulation), then the first nucleic acid, vector, composition and/or pharmaceutical composition is administered prior to the second nucleic acid, vector, composition and/or pharmaceutical composition. In another embodiment, the first nucleic acid, vector, composition and/or pharmaceutical composition and the second nucleic acid, vector, composition and/or pharmaceutical composition are administered concurrently. In yet other embodiments, the first nucleic acid, vector, composition and/or pharmaceutical composition is administered after the second nucleic acid, vector, composition and/or pharmaceutical composition.

According to various aspects herein, the methods provided herein provide for administering (e.g, to a subject) the first nucleic acid or vector in a ratio with the second nucleic acid vector. As described herein, the first nucleic acid or vector can be referred to herein as the “Donor Vector” and the second nucleic acid or vector can be referred to herein as the “CRISPR vector”. Therefore, the disclosure further provides for different ratios of Donor vs CRISPR administration. In some aspects, a ratio of the first vector to the second vector js from about 10:1 to about 1:1, from about 9:1 to about 1:1, from about 8:1 to about 1:1, from about 7:1 to about 1:1, from about 6:1 to about 1:1, from about 5:1 to about 1:1, from about 4:1 to about 1:1, from about 3:1 to about 1:1, from about 2:1 to about 1:1. In some aspects, a ratio of the first vector to the second vector js from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1. For example, in some aspects, the ratio of the first vector to the second vector is about 10:1, about 9:1, about 8:1, about 7:1, about 6:1, about 5:1, about 4:1, about 3:1, about 2:1, or about 1:1. In other aspects, the ratio of the first vector to the second vector is 10:1, 9:1, 8:1, 7:1, 6:1, 5:1, 4:1, 3:1, 2:1, or 1:1. For example, in some aspects, the ratio of the first vector is about 4:1, about 2:1, or about 1:1. In further aspects, the ratio of the first vector to the second vector is 4:1, 2:1 or 1:1.

Combination therapies (e.g., with one or more additional therapeutic agent(s)) are also contemplated by the present disclosure. Combination as used herein includes both simultaneous treatment and sequential treatments (e.g., before or after administration of a nucleic acid cassette, vector/vector system, composition, or pharmaceutical composition thereof). Combinations of methods of the present disclosure with standard medical treatments are specifically contemplated, as are combinations with alternative vectors mentioned above, novel vectors that are engineered and generated to enhance the effect of therapy and novel therapies.

In some embodiments, the one or more additional therapeutic agent(s) comprises a small molecule drug. In some embodiments, the small molecule drug comprises an antilipemic agent. Examples of suitable antilipemic agents include, but are not limited to, bile acidresins/sequestrants such as cholestryramine, colesevelam, colestipol; Fibrates such as clofibrate, fenofibrate, gemfibrozil, benzafibrate; monoclonal antibodies, such as alirocumab, evinacumab, evolocumab; niacin; Omega-3 fatty acids such as icosapent ethyl, omega-3-acid ethyl esters, omega-3 carboxylic acids; statins, such as atorvastatin, Fluvastatin, lovastatin, pitavastatin, pravastatin, rosuvastatin, simvastatin, exetimibe, lomitapide, mipomoersen, and combinations thereof and the like. In some embodiments, the small molecule drug comprises an an mTOR inhibitor (e.g., an mTOR inhibitor that induces autophagy). In some aspects, the mTOR inhibitor that induces autophagy can comprise resveratrol, rapamycin, CC1-779, RAD001, Torin 1, KU-0063794, WYE-354, AZD8055, metformin or any combination thereof.

In accordance with the foregoin, the one or more additional therapeutic agents can comprise cholestryramine, colesevelam, colestipol, clofibrate, fenofibrate, gemfibrozil, benzafibrate, alirocumab, evinacumab, evolocumab, niacin, icosapent theyl, omedga-3-acid ethyl esters, omega-3 carboxylic acids, atorvastatin, fluvastatin, lovastatin, pitavastatin, pravastatin, rosuvastatin, simvastatin, exetimibe, lomitapide, mipomoersen, resveratrol, rapamycin, CC1-779, RAD001, Torin 1, KU-0063794, WYE-354, AZD8055, metformin or any combination thereof. In one embodiment, the one or more additional therapeutic agent(s) comprises benzafibrate, rapamycin or a rapamycin analog. In other aspects, the one or more additional therapeutic agent can comprise a gene replacement vector (e.g., such as one provided herein as SEQ ID NO: 49). In various aspects, the gene replacement vector can comprise a G6PC transgene operably linked to a promoter, such that the gene replacement vector is expressed episomally in a cell of the subject (i.e., is not integrated into the genome). In various aspects, the gene replacement vector can be an AAV vector.

In an aspect, a disclosed method can comprise measuring and/or determining one or more liver enzymes and/or metabolites. Liver enzyme and/or metabolites can comprise Alanine transaminase (ALT), Aspartate transaminase (AST), Alkaline phosphatase (ALP), Albumin and total protein, Bilirubin, Gamma-glutamyltransferase (GGT), L-lactate dehydrogenase (LD), Prothrombin time (PT), or any combination thereof. In an aspect, a disclosed method can comprise measuring and/or determining one or more urine enzymes and/or metabolites (such as, for example, glucotetrasaccharides (HEX4)).

### V. Kits

The present disclosure further provides kits comprising the compositions provided herein and for carrying out the subject methods as provided herein. For example, in one embodiment, a subject kit can comprise, consist of, or consist essentially of one or more of the following: (i) nucleic acid cassettes as provided herein; (ii) a vector(s) and/or vector systems as provided herein; (iii) delivery systems comprising a nucleic acid cassettes and/or vector(s) and/or vector systems as provided herein; (iv) cells comprising a nucleic acid cassette(s) and/or vector(s), and/or vector systems and/or delivery system comprising a nucleic acid cassettes and/or vector(s), vector systems, compositions as provided herein; and/or (v) pharmaceutical compositions as provided herein.

In other embodiments, a kit can further include other components. Such components can be provided individually or in combination and can provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents and the like, (ii) one or more control expression vectors or RNA polynucleotides; (iii) one or more reagents for in vitro production and/or maintenance of the of the molecules, cells, delivery systems etc. provided herein; and the like. Components (e.g., reagents) can also be provided in a form that is usable in a particular assay, or in a form that requires addition of one or more other components before use (e.g., in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In an aspect, a disclosed kit can be used to measure and/or determine one or more liver enzymes and/or metabolites. Liver enzyme and/or metabolites can comprise Alanine transaminase (ALT), Aspartate transaminase (AST), Alkaline phosphatase (ALP), Albumin and total protein, Bilirubin, Gamma-glutamyltransferase (GGT), L-lactate dehydrogenase (LD), Prothrombin time (PT), or any combination thereof. In an aspect, a disclosed kit can comprise measure and/or determine one or more urine enzymes and/or metabolites (such as, for example, glucotetrasaccharides (HEX4)).

In addition to above-mentioned components, a subject kit can further include instructions for using the components of the kit to practice the subject methods. The instructions for practicing the subject methods are generally recorded on a suitable recording medium. For example, the instructions can be printed on a substrate, such as paper or plastic, etc. As such, the instructions can be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

Glycogen storage disease type Ia (GSD Ia) is a rare inherited disease caused by mutations in the G6PC gene, which encodes glucose-6-phosphatase (G6Pase). Absence of G6Pase causes life-threatening hypoglycemia and long-term complications including renal failure, nephrolithiasis, hepatocellular adenomas (HCA), and a significant risk for hepatocellular carcinoma (HCC). The complications occur due to the accumulations of metabolic intermediates including glycogen and triglycerides in the liver, kidney, and small intestine. The canine GSD Ia model mimics the human disease more accurately than mouse models, given the longer lifespan and outbred genetics of dogs. Specifically, the GSD Ia model has a p1211 (n.t.G450C) missense mutation in Exon 3. Affected puppies have significantly increased glycogen content and decreased G6Pase activity in the liver and decreased G6Pase activity in the kidney (P. S. Kishnani Vet Pathol 2001. P. S Kishnani Biochemical and Molecular Mediicne 1997 and A. E Brix Vet Pathol 1995).

AAV vectors that deliver the G6Pase gene for exogenous expression have been developed for treatment of GSD Ia and shown effective at correcting hypoglycemia and greatly prolonging lifespan; however, these vectors have not prevented all long-term complications. AAV vector genomes remain almost exclusively in an episomal state in the cells, and therefore AAV derived transgene expression has diminished over time.

In the following examples, experiments are described using a CRISPR/Cas9 system that cleaves the G6PC exon 1/intron 1 boundary and delivers a repair template to induce homologous recombination (HR) and to integrate a functional G6PC gene. The data presented herein below supports use of the CRISPR/Cas9 gene editing system in vivo.

### Example 1

AAV Vectors were Developed and Tested in Vitro.

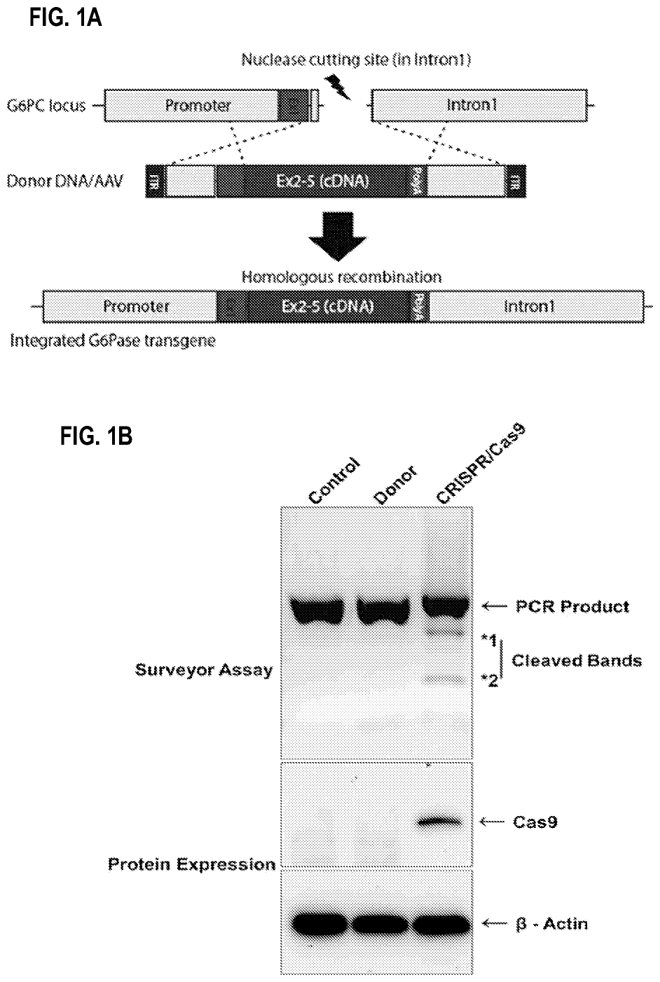

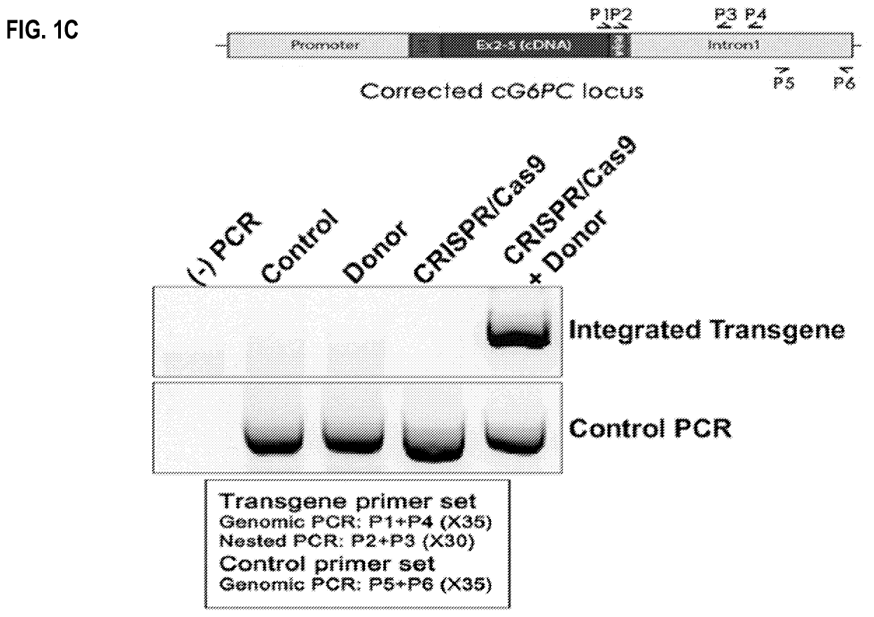

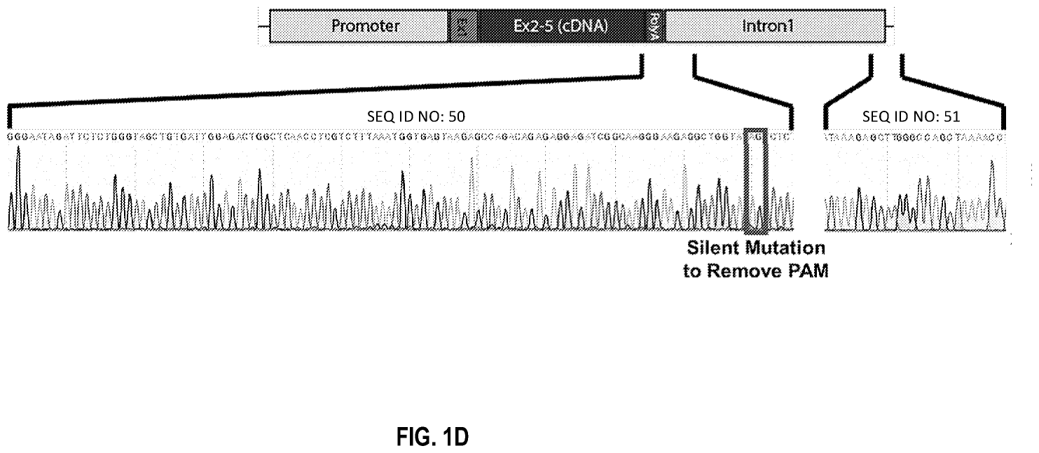

In this example, genome editing was evaluated in cultured GSD Ia dog fibroblasts, transfected with plasmids containing the vector transgenes. FIG. 1A shows a schematic of CRISPR/Cas9 cutting at the exon 1/intron 1 boundary of the dog G6PC gene, followed by HDR to achieve integration of a canine G6PC cDNA downstream of the G6PC promoter. Specifically, one vector delivered the S. aureus Cas9 endonuclease (AAV-SaCas9) and an sgRNA expression cassette that directs SaCas9 to cleave the G6PC gene at exon 1/intron 1 boundary, while a second vector (AAV-cG6PC) delivered a repair template (Donor) to induce HDR and to integrate a functional G6PC gene. To avoid potential problems caused by the limited DNA packaging capacity of AAV, the S. aureus Cas9 protein was used, instead of Streptococcus pyogenes Cas9, which is more commonly used. The S. aureus Cas9 open reading frame (ORF) is 3162 base pairs (bp) in length, substantially smaller than the 4107 bp S. pyogenes Cas9 ORF, yet S. aureus Cas9 shows an similar level of genome editing activity in mammalian cells. 16

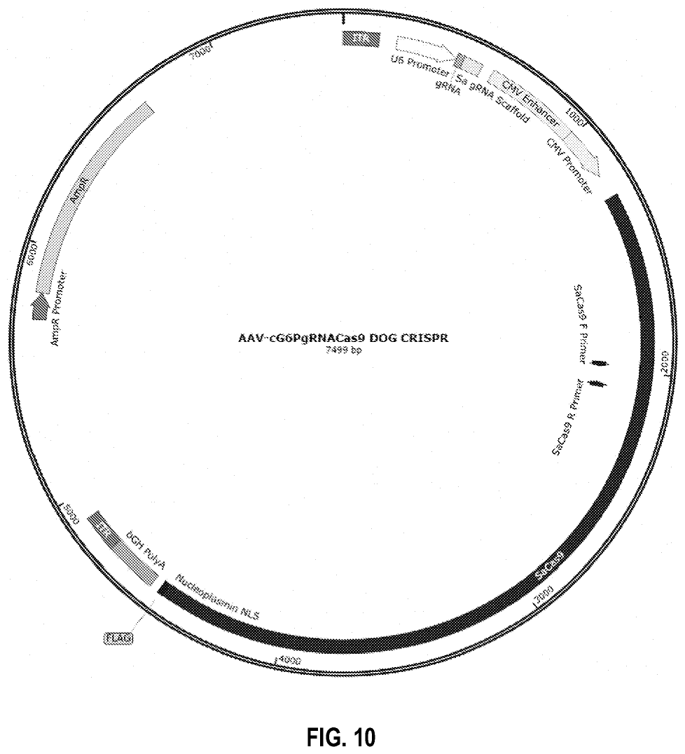

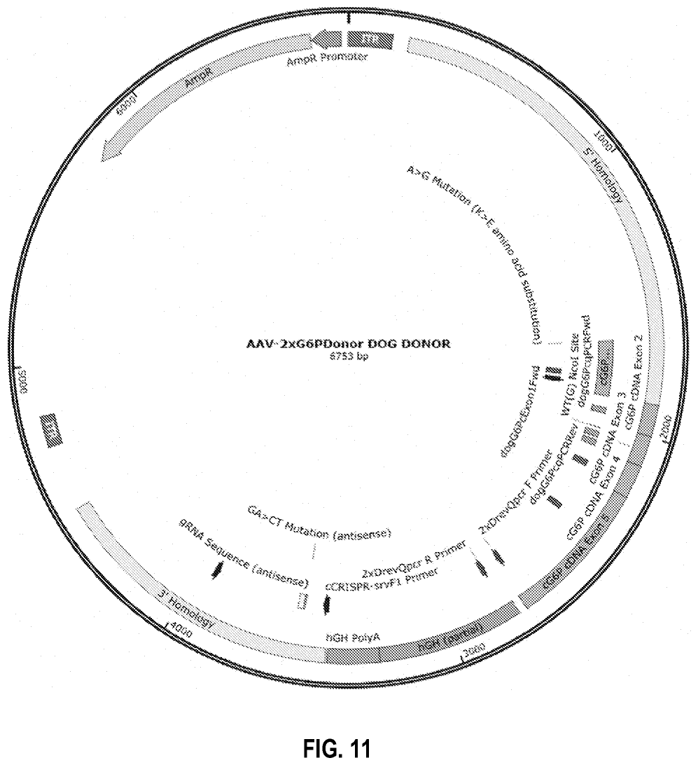

The AAV vector plasmid pAAV-saCas9 (SEQ ID NO: 47, FIG. 10) contained the AAV vector gene comprised of two inverted terminal repeats (ITRs) flanking two transgenes: (1) the U6 promoter expressing a gRNA targeting SEQ ID NO: 1) and (2) a minimal CMV promoter expressing Cas9 from S. aureus (SEQ ID NO: 56) with a FLAG tag and bovine growth hormone genomic polyadenylation sequence. The second AAV vector plasmid, AAV-cG6PC (SEQ ID NO: 43, FIG. 11), contained two ITRs flanking the transgene consisting of the canine G6Pc cDNA (SEQ ID NO: 19). The cDNA was flanked upstream by a 5′ homology arm (the 5′ UTR genomic sequence of canine G6PC, including a 1361 bp canine G6PC promoter), SEQ ID NO: 29 or 30. Downstream of the cDNA was the human growth hormone genomic polyadenylation sequence followed by a 3′ homology arm (the Intron 1 genomic sequence of canine G6PC) (SEQ ID NO: 31). The 3′ homology arm further comprised a GA>CT mutation in the antisense direction that removed the PAM site when integrated into the genome (see bolded and underlined section in Table 7). Vectors were purified and quantified by Southern blot as described in Demaster, A., et al. (2012). Hum Gene Ther. 23, 407-418, which is incorporated herein by reference in its entirety.

Primary canine fibroblasts were transfected with AAV-SaCas9 (SEQ ID NO: 47) and AAV-cG6PC (SEQ ID NO: 43) plasmids using Lipofectamine 3000 (Thermo-Fisher Scientific, Waltham, MA; #L3000015) according to manufacturer's protocol. Donor vector integration and nuclease activity (level of indels) was detected as described below.

To quantify nuclease activity, a Surveyor assay was performed. Cultured dog skin fibroblasts were transfected with the pAAV-CRISPR/Cas9 plasmid, pAAV-cG6PC (Donor) plasmid, or untransfected (control) and incubated for 72 hours before DNA was extracted. Using the extracted DNA, the canine G6PC locus was amplified using one round of PCR following the conditions described below except using the primers: dogsurveyorFwd (5′-GCCTTCTATGTCCTCTTTCCC-3′, SEQ ID NO: 57) and dogsurveyorRev (5′-TTAGAGCCCAGTTCTCTGGGTTAC-3′, SEQ ID NO: 58). The PCR product was analyzed using the Surveyor Mutation Detection Kit (Integrated DNA Technologies, Coralville, IA) according to manufacturer's instructions. The PCR products were also sequenced using Sanger sequencing methods (Eton Biosciences, Durham, NC). The Surveyor assay revealed the expected bands reflecting indels from NHEJ (FIG. 1B).

Western blotting was also performed to detect Cas9 protein expression in transfected fibroblasts. Briefly, fibroblasts from cell culture were homogenized in radioimmunoprecipitation assay (RIPA) lysis buffer (Thermo-Fisher Scientific, Waltham, MA), and protein concentration was determined via BCA Assay (Thermo-Fisher Scientific, Waltham, MA). Laemmli sample buffer was added (250 mmol/L Tris [pH 7.4], 2% w/v SDS, 25% v/v glycerol, 10% v/v 2-mercaptoethanol, 0.01% w/v bromophenol blue), and gel samples were boiled for 10 min and stored at −20 C until SDS-PAGE was performed. Samples were run on a SDS-polyacrylamide gel and transferred to a polyvinylidene difluoride membrane (Bio-Rad Laboratories, Hercules, CA). Washing, blocking, and antibody solutions were prepared in PBS with 0.1% Tween 20 (PBST). Following washing, membranes were blocked for an hour in 5% skim milk, incubated overnight at 4° C. with the primary antibody (Santa Cruz HA-Tag Antibody #sc-7392), washed, and reincubated for an hour with the secondary antibody (Sigma Chemical Co., St. Louis, MO, mouse-HRP #12-249). After a final wash, enhanced chemiluminescence (ECL) detection reagents (Thermo-Fisher Scientific, Waltham, MA) were added to the membrane, and protein signal was read using a ChemiDoc imaging system (Bio-Rad Laboratories, Hercules, CA). Membranes were also imaged for β-actin control signal after stripping and re-blocking the membrane. As shown in FIG. 1B, a single band was detected corresponding to Cas9 protein.

Next, a nested PCR reaction was performed to detect levels of DNA integration in genomic DNA in the transfected fibroblasts. Fibroblast DNA were extracted using the Wizard Genomic DNA Purification Kit (Promega, Madison, WI, USA). The canine G6Pc locus was amplified by Q5 Taq Polymerase (NEB, Ipswich, MA, USA) with the following reagents: 5 μL of Q5 buffer, 5 μL of high GC enhancer solution, 2 μL of 2.5 mM dNTP mix, 1.25 μL of 10 uM primer P1 (5′-GCCAGACAAGAAGTCTTTGTAAGGC-3′, SEQ ID NO: 59)), 1.25 μL of 10 uM primer P4 (5′-GCTGTTGAATAGGGGACATTACAGACG-3′, SEQ ID NO: 62)), 9.25 μL of water, 1 μL (100 ng) of genomic DNA, and 0.25 μL of Q5 Taq Polymerase. Cycling conditions were 35 cycles of denaturation at 95° C. for 30 s, annealing at 59° C. for 30 s, extension at 72° C. for 2 min, followed by incubation at 4° C. One microliter of first-round PCR products was used in a nested reaction with the same conditions except primers were P2 (5′-GGACATGGACAAGGTCGAGACATTCC-3′ (SEQ ID NO: 60)) and P3 (5′-CCAAAGAATATTAGAGCTAGAAG-3′ (SEQ ID NO: 61)) and cycling was 30 cycles. Control primers were P5 (5′-CGTCTGTAATGTCCCCTATTCAACAGC-3′ (SEQ ID NO: 63)) and P6 (5′-AAGTACCTAGAACAGTGTCTGGCACAG-3′ (SEQ ID NO: 64)). This integration PCR revealed the presence of the band expected from the junction between dog G6PC gene and vector transgene by HDR (FIG. 1C).

Sequencing of the integration PCR product (total sequence: SEQ ID NO: 52) confirmed the donor sequence was inserted in the dog G6PC gene at the exon 1/intron 1 boundary. FIG. 1D depicts a select fragment the total PCR product showing the transition from the poly A sequence to intron 1 containing a silent mutation that removes the PAM sequence (SEQ ID NO: 50). The transition from the end of the vector's right homology arm into the dog G6PC genomic sequence is also shown as SEQ ID NO: 51.

Therefore, the data in this example demonstrate that the two vector plasmids were functional in vitro, as demonstrated by the generation of indels detected in the Surveyor assay (FIG. 1B) and transgene integration in canine GSD Ia fibroblasts (FIG. 1C). Integration was dependent upon the presence of CRISPR/Cas9, because transfection with Donor alone resulted in no detectable integration events. Sequencing of transgene integration events confirmed its location in the dog G6PC exon 1/intron 1 boundary in the genome (FIG. 1D).

### Example 2

### Gene Editing Vectors Successfully Integrated and Resulted in Persistent GSD Ia Expression in Dogs Treated as Adults.

This example describes experiments showing successful delivery and integration of the gene editing vectors described in Example 1 in adult animals in a canine model of GSD Ia.

Three dogs were treated between birth and three months with three gene replacement AAV vectors (AAV-G6Pase/AAV9, 2×10¹³ vp/kg at birth, AAV-G6Pase/AAV10, 5×10¹² vp/kg at 2 months, and AAV-G6Pase/8, 2×10¹³ vp/kg at 3 months). The vector sequence for each of these (AAV-G6Pase) is provided herein as SEQ ID NO: 49. These “gene replacement” AAV vectors, referred to herein as “AAV-G6Pase”, were designed using different AAV serotypes than the gene editing AAV7 vectors described in Example 1. They delivered the human G6Pase cDNA under the control of a human G6Pase minimal promoter and were intended for episomal gene expression, not genomic integration as they lacked any CRISPR machinery. These gene replacement vectors are described in more detail in Luo, X., et al., (2011). Hepatorenal correction in murine glycogen storage disease type I with a double-stranded adeno-associated virus vector. Mol Ther. 19, 1961-1970 which is herein incorporated by reference in its entirety. In addition, the dogs were also treated with a brief course of rapamycin (Resverotrol at 5 mg/kg/day between months 2 and 3) to stimulate autophagy as described (Ding, S., et al., (2017). PLOS One. 12, e0183541.)

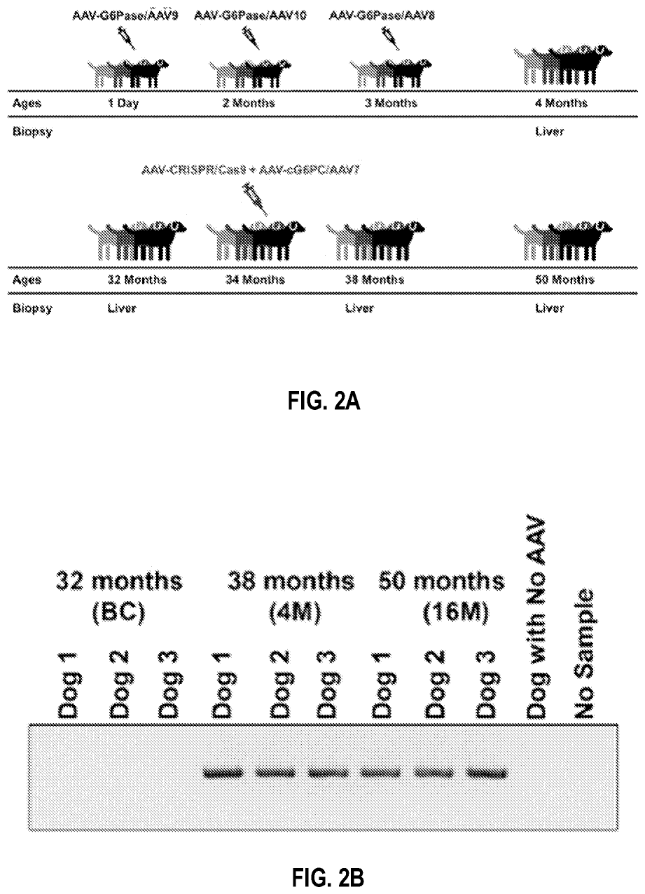

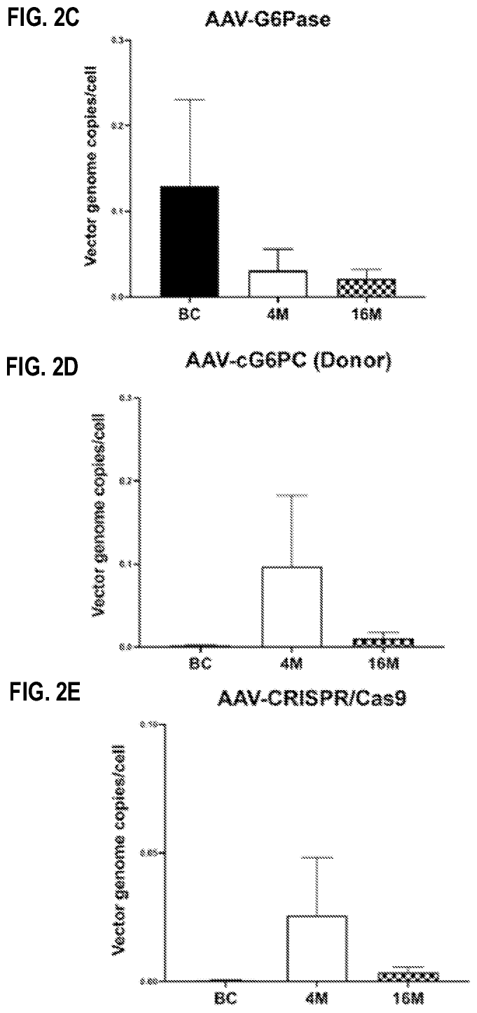

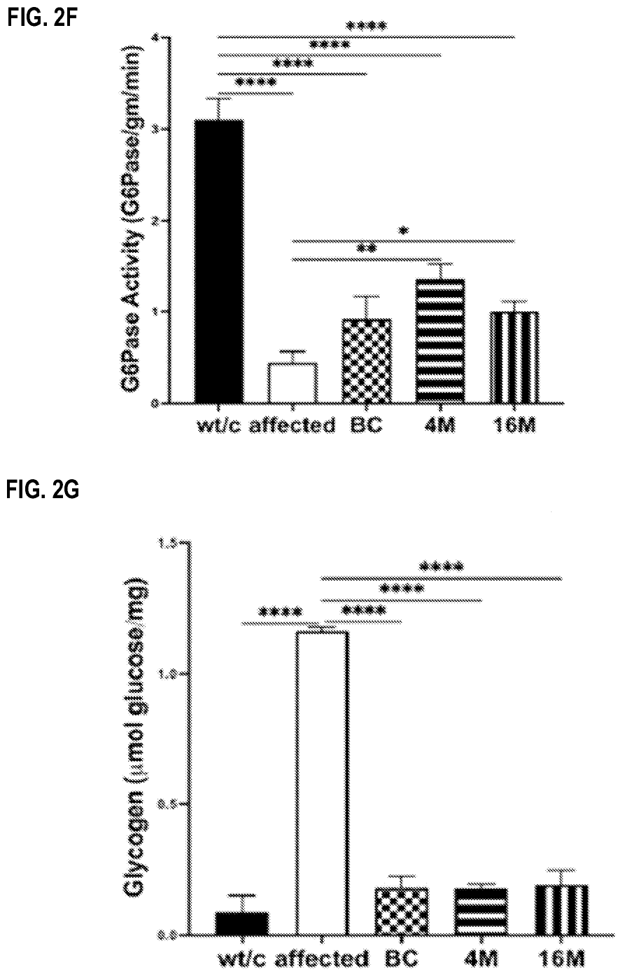

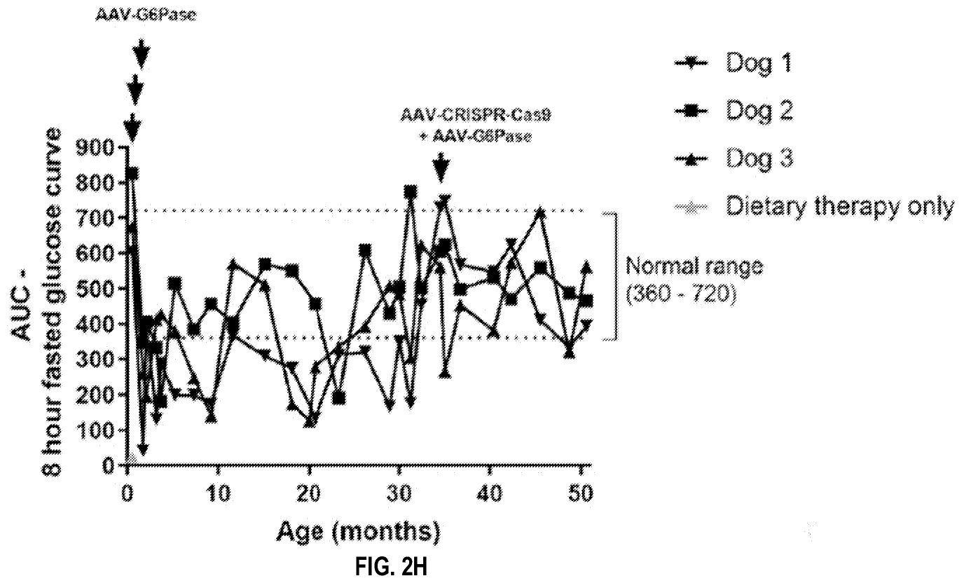

Once the three dogs reached adulthood, the dogs were treated with bezofibrate (4 mg/kg/day between 29 and 30 months and then treated at 34 months of age using relatively high vector dosages of the gene editing vectors of Example 1:1×10¹³ vp/kg of the pAAV-saCas9/AAV7 vector expressing SaCas9 and an sgRNA (SEQ ID NO: 47) and 2×10¹² vp/kg of the pAAV-G6Pase/AAV7 vector expressing a G6Pase transgene (SEQ ID NO: 43) (FIG. 2A). Table 2 below describes all treatment regimens for the animals in this Example.

TABLE 2
Vector Administration Protocols for Animals Treated as Adults

| | | Age when | | | | |
|---|---|---|---|---|---|---|
| | | vector/medication | | | Vector/Medication | |
| Dog | Sex | administered | | AAV vector/medication | Dose | |
| Dog 1 | M | 1 | day | AAV2/9 G6Pase | 2 × 10¹³ | vp/kg |
| | | 2 | months | AAV2/10 G6PASE | 5 × 10¹² | vp/kg |
| | | 2-3 | months | Resveratrol^{A} | 5 | mg/kg/day |
| | | 3 | months | AAV2/8 G6Pase | 2 × 10¹³ | vp/kg |
| | | 29-32 | months | Bezafibrate^{B} | 4 | mg/kg/day |
| | | 34 | months | AAV2/7 G6Pase + AAV2/7 | 1 × 10¹³ vp/kg + 2 × | |
| | | | | CRISPR | 10¹² vp/kg | |
| Dog 2 | M | 1 | day | AAV2/9 G6Pase | 2 × 10¹³ | vp/kg |
| | | 2 | months | AAV2/10 G6PASE | 5 × 10¹² | vp/kg |
| | | 2-3 | months | Resveratrol^{A} | 5 | mg/kg/day |
| | | 3 | months | AAV2/8 G6Pase | 7 × 10¹² | vp/kg |
| | | 29-32 | months | Bezafibrate^{B} | 4 | mg/kg/day |
| | | 34 | months | AAV2/7 G6Pase + AAV2/7 | 1 × 10¹³ vp/kg + 2 × | |
| | | | | CRISPR | 10¹² vp/kg | |
| Dog 3 | M | 1 | day | AAV2/9 G6Pase | 2 × 10¹³ | vp/kg |
| | | 2 | months | AAV2/10 G6PASE | 5 × 10¹² | vp/kg |
| | | 2-3 | months | Resveratrol^{A} | 5 | mg/kg/day |
| | | 3 | months | AAV2/8 G6Pase | 2 × 10¹² | vp/kg |
| | | 30-32 | months | Bezafibrate^{B} | 4 | mg/kg/day |

| | | | |
|---|---|---|---|
| 34 | months | AAV2/7 G6Pase + AAV2/7 | 1 × 10¹³ vp/kg + 2 × |
| | | CRISPR | 10¹² vp/kg |

^{A}Resveratrol was administered to stimulate autophagy as described in Ding et al., PLoS One. 12, e0183541.
^{B}Description and results of bezafibrate treatment described in Waskowicz et al., Hum Mol Genet. 28, 143-154.

All three dogs had a liver biopsy 2 months before treatment with editing vectors and 4 and 16 months after receiving gene editing vectors to use for analysis described in this Example and Example 3. Transgene integration was evaluated as described above in Example 1. As shown in FIG. 2B, integration PCR revealed the presence of integrated Donor at 36 months and 50 months of age or 4- and 16-months following administration of genome editing, respectively. In contrast, no integration was detected at 32 months of age, before CRISPR vector administration (“BC”).

Vector genomes in liver were quantified with qPCR. Briefly, AAV vector genome copy number was measured by quantitative real-time PCR with liver genomic DNA and normalized to β-actin. Plasmid DNA corresponding to 0.01 to 100 copies of canine G6Pase gene (in 500 ng genomic DNA) was used in a standard curve. qPCR was performed on a Lightcycler 480 (Roche Diagnostics, Basel, Switzerland) using SYBR Green mix (Thermo-Fisher Scientific, Waltham, MA) and the following primers: cG6Pc Fwd (5′-TCTTCGACCAGCCAGACAAG-3′, SEQ ID NO: 65), cG6Pc Rev (5′-GGTCCTTTAGGAGGTCATAG-3′, SEQ ID NO: 66), hG6PC Fwd (5′-GCAGTTCCCTGTAACCTGTGAG-3′, SEQ ID NO: 67), hG6PC Rev (5′-GGTCGGCTTTATCTTTCCCTG-3′, SEQ ID NO: 68), saCas9 Fwd (5′-GTTGGTATACACGGTGTGCCTG-3′, SEQ ID NO: 69), saCas9 Rev (5′-CTGACGCCAGCGTCAATCAC-3′, SEQ ID NO: 70), cB-actin Fwd (5′-ATGGAATCCTGCGGCATCCATG-3′, SEQ ID NO: 71), cB-actin Rev (5′-CAGGGTACATGGTGGTTCCAC-3′, SEQ ID NO: 72). Cycling conditions were 95° C. for 5 min, followed by 45 cycles of 95° C. for 10 s, 60° C. for 10 s, and 72° C. for 20 s followed by acquisition. FIG. 2C, FIG. 2D and FIG. 2E show that the original gene replacement vector, AAV-G6Pase, as well as the gene editing vectors AAV-cG6PC and AAV-saCas9 (“AAV-CRISPR/Cas9”) were all detected at months 4 and 16 following delivery of the gene editing vectors. As shown in FIG. 2C, the original gene replacement vector, AAV-G6Pase, was also detected before CRISPR vector administration (“BC”) but has a low copy number (<0.1 vg/nucleus) at 4 months of age following gene editing (4M). Higher copy numbers of the gene editing vectors (AAV-cG6Pc and AAV-CRISPR/Cas9) were observed 4 months following gene editing (FIG. 2D and FIG. 2E, respectively). The latter vectors trended toward greater copy number 4 months following genome editing (p=0.09).

### Example 3

Treatment of Adult Dogs with GSD Ia with Gene Editing Vectors after Treatment with Gene Replacement Therapy as Neonates Resulted in Biochemical Correction in Tissue Samples.

This example describes levels of biochemical correction following gene editing and gene replacement vectors in the dog population described in Example 2. Specifically, G6Pase activity, glycogen levels and glucose tolerance were all measured to evaluate the effect of gene editing and/or gene replacement vectors on GSD Ia phenotypes in affected animals.

G6Pase activity and glycogen levels were analyzed at 4- and 16-months following genome editing at 34 months of age as described in (Koeberl, D. et al., (2006). Early, sustained efficacy of adeno-associated virus vector-mediated gene therapy in glycogen storage disease type Ia. Gene Therapy. 13, 1281-1289). Both assays reflected corrections of the biochemical abnormalities in comparison with untreated affected controls. Normal activity was measured in a group of three unaffected dogs (two carriers and one wildtype; both genotypes are accepted as normal controls in published studies of animals with GSD Ia). Briefly, liver biopsy tissues, obtained as described in Example 2, were flash-frozen and stored at −70° C. Glycogen content was measured by complete digestion of polysaccharide using amyloglucosidase (Sigma Chemical Co., St. Louis, MO). The structure of the polysaccharide was inferred by using phosphorylase free of the debranching enzyme to measure the yield of glucose-1-phosphate. Specific G6Pase activity was measured by using glucose-6-phosphate as substrate after subtraction of nonspecific phosphatase activity as estimated by β-glycerophosphate. FIG. 2F shows that significant increased G6Pase activity was detected in treated dogs in comparison with untreated dogs with GSD Ia. At 32 months of age, the dogs had 29±8% of normal G6Pase activity in liver, which increased to 43±5% at 38 months of age, 4 months after receiving both Donor and CRISPR vectors. However, the difference in G6Pase at 4 months was not statistically significant, in comparison with baseline (4M versus BC; FIG. 2F). Liver G6Pase activity declined to 32+/−3% at 50 months of age, indicating that the majority of transgene expression was from episomal AAV vector genomes that were lost over the intervening 12 months. Likewise, liver glycogen content was significantly decreased in comparison with untreated dogs with GSD Ia (FIG. 2G) and remained stably low following genome editing. However, there was no decrease in glycogen content following vector administration (4M versus BC; FIG. 2G).

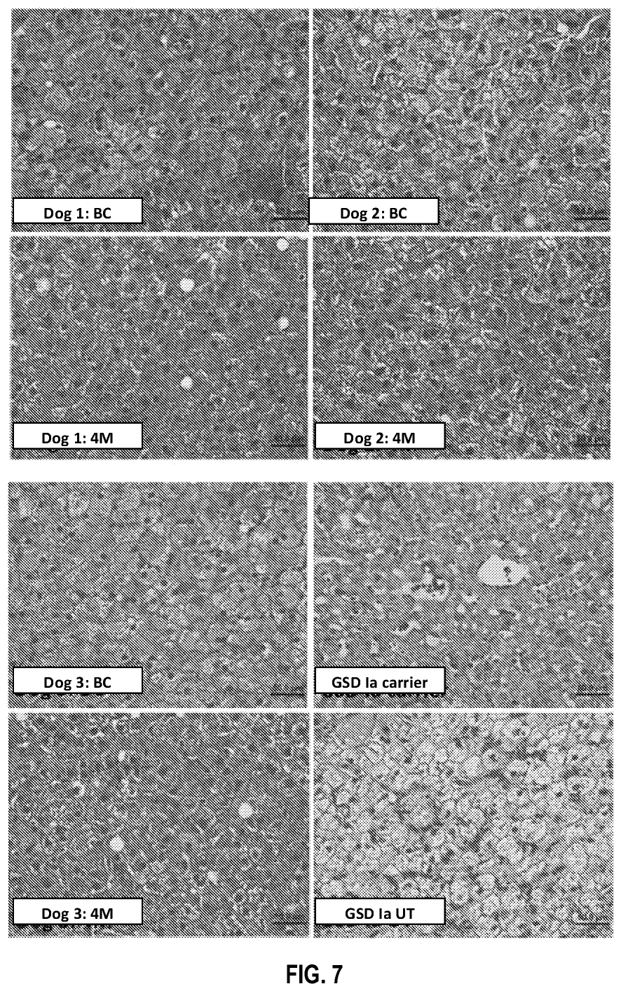

Histopathology was performed on liver biopsy samples. Briefly, liver biopsies were fixed in 10% neutral-buffered formalin and stored at 4° C. until embedded in paraffin and sectioned at 5 μm. Histologic stains included hematoxylin and eosin (H&E) and Periodic acid-Schiff (PAS) on selected sections. Microscopic examination of liver biopsy samples revealed similar histopathological features in all three treated dogs both pre- and post-treatment with genome editing (FIG. 7). Specifically, photomicrographs of hepatic sections of Dogs 1-3 pre-treatment (BC) reveal mosaic pattern of diffuse hepatocyte hypertrophy with vacuolar and glycogen changes and inconspicuous hepatic sinusoids relative to that of the GSD-la carrier liver. There is minimal (Dog 2) to mild (Dog 1 and 3) glycogen depletion noted in the post-treatment hepatic sections (4M). However, these changes were markedly decreased in comparison with an untreated adult dog with GSD Ia and were consistent with stable correction from G6PC transgene expression. For example, in comparison with an untreated adult dog (GSD Ia UT), vacuolar changes and glycogen accumulations were markedly decreased for Dogs 1-3 (FIG. 7). The photomicrograph of the liver from GSD Ia UT also shows marked diffuse vacuolar change with maintenance of prominent hepatic sinusoids congested with erythrocytes (FIG. 7). Magnification 400×.

Finally, levels of hypoglycemia in treated animals were tested using a glucose tolerance test (GTT). The GTT was performed at regular intervals from birth to month 50 and so measured levels of hypoglycemia before and after administration of CRISPR vectors. In brief, glucose curves for monitoring hypoglycemia were performed by fasting the dogs for up to 8 hours and monitoring blood glucose every 2 hours. If blood glucose dropped below 50-60 mg/dL or clinical signs of hypoglycemia occurred, the curve was stopped, and dogs were given dextrose therapy as needed and fed. Blood glucose was measured by a point of care glucometer, either the AlphaTRAK or AlphaTRAK2 (Zoetis, Parsippany, NJ).

As shown in FIG. 2H, genome editing was accompanied by correction of hypoglycemia during fasting, as demonstrated by stably increased blood glucose into the normal range of unaffected controls (see dotted lines in FIG. 2H). Specifically, the three treated animals had normal blood glucose (139+/−13 mg/dl) after a two hour fast (data not shown) at two weeks of age following administration of the first gene replacement AAV vector (AAV-G6Pase/AAV9) but untreated, affected puppies could not fast longer than two hours and had low blood glucose (9+/−9.5 mg/dl; age-matched normal range 110+/−23 mg/dl) (FIG. 2H). The group of treated animals also had normal area under the curve (AUC) blood glucose during the 8-hour fasting test at two weeks of age following gene therapy (701+/−113 mg/dl; normal 360-720 mg/dl) (FIG. 2H). However, AUC eventually declined prior to genome editing, and normalized following the administration of genome editing vectors (FIG. 2H).

### Example 4

An Immune Response to CRISPR Vectors was Detected in Animals Treated with Gene Editing Vectors as Adults.

In this example, antibody responses were evaluated in animals treated with CRISPR vectors as adults to assess any effect from immune responses on genome editing. A puppy treated as a neonate with the editing vectors (described below in Example 5) was included as a control.

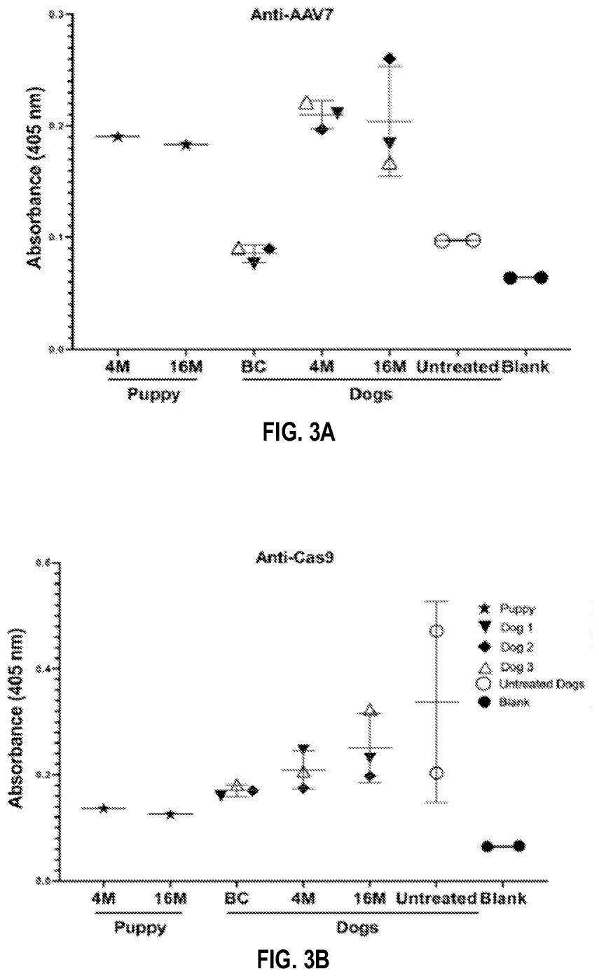

IgG responses were determined by ELISA for anti-AAV7 and anti-SaCas9. In brief, MAxisorp 96-well plates (Thermo Fisher) were coated with Cap7 or SasCas9 protein in carbonate buffer at 4° C. overnight. A standard curve of IgG isotype (Sigma Chemical Co., St. Louis, MO) was coated to the wells in seven 2-fold dilution starting from 1 μg/mL. After blocking, plasma samples diluted at 1:100 were added to plates and incubated for 1 hr at 37° C. Isotype-specific secondary antibodies coupled to HRP were used for detection (Southern Biotech, Birmingham, AL). Then 3,3′,5,5′-tetramethylbenzidine substrate (BD Biosciences, San Jose, CA) was added to the wells and color development was measures at 450 and 570 nm (for background subtraction) on an Enspire plate reader (Perkin Elmer, Waltham, MA) after blocking the reaction with H2SO4. IgG response measured at different time points after vector administration (e.g., at 4M or 16M) are shown in FIGS. 3A and 3B for anti-AAV7 and anti-Cas9 antibodies, respectively. Anti-AAV7 IgG antibodies were positive following vector administration, demonstrating the expected response to AAV7 vector administration in all dogs (FIG. 3A). In contrast to the neonatally treated dog that maintained low anti-SaCas9, anti-SaCas9 was positive for adult dogs treated with genome editing at baseline, and at Months 4 and 16 following editing (FIG. 3B) indicating that adult dogs were exposed to S. aureus prior to receiving gene editing vectors.

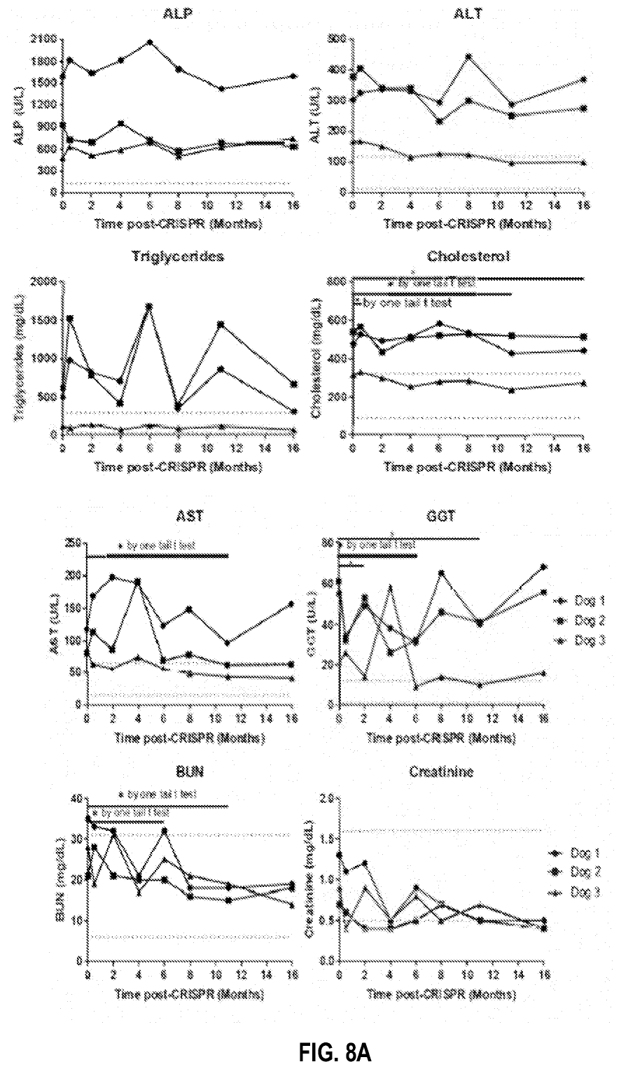

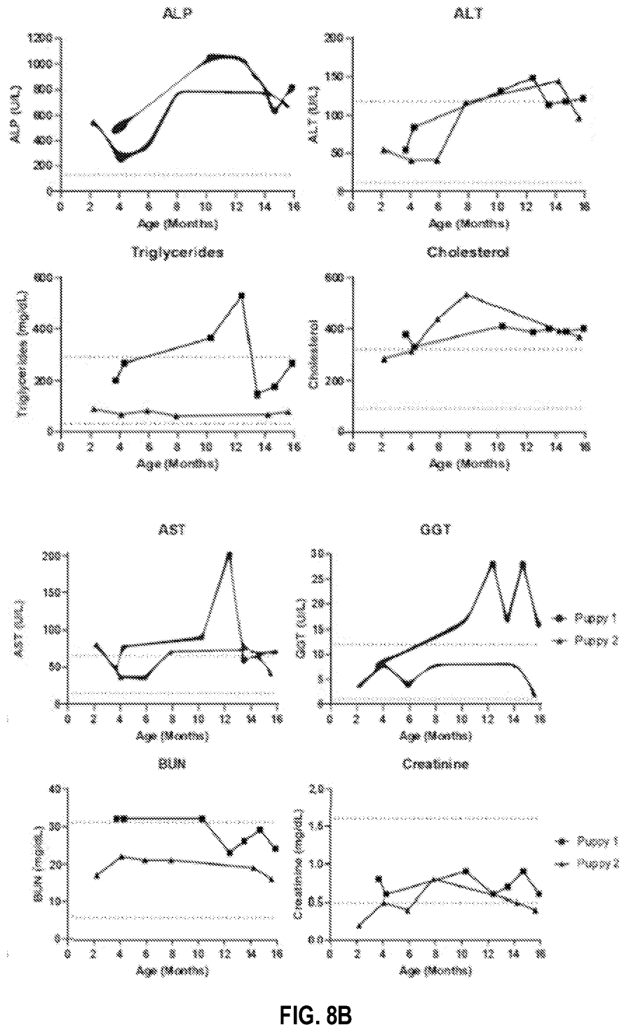

A blood chemistry analysis was also performed on these animals with standard methods. Specifically, blood analyses were either performed in house at the Duke University Division of Laboratory Animal Services clinical pathology lab or sent out to a commercial laboratory, Antech Diagnostics (Antech, Diagnostic Laboratories, Cary, NC). FIG. 8A shows levels of ALP, ALT, AST, GGT, triglycerides, cholesterol, BUN and creatine immediately before (T=0) and up to 16 months after CRISPR treatment in adult GSD Ia dogs. FIG. 8B shows levels of the same analytes in neonatal animals treated with CRISPR as described below in Example 5. Elevated transaminases, both alanine aminotransferase (ALT) and aspartate aminotransferase, were variably elevated prior to and following genome editing (FIG. 8A-8B), which were attributed to the liver effects of GSD Ia.

### Example 5

### Gene Editing Vectors Successfully Integrated and Resulted in Persistent GSD Ia Expression in Dogs Treated as Neonates

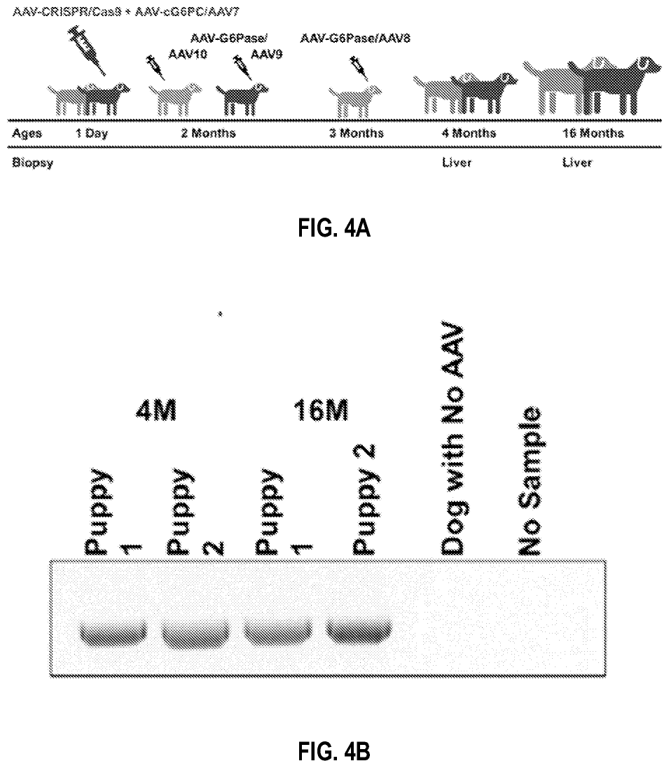

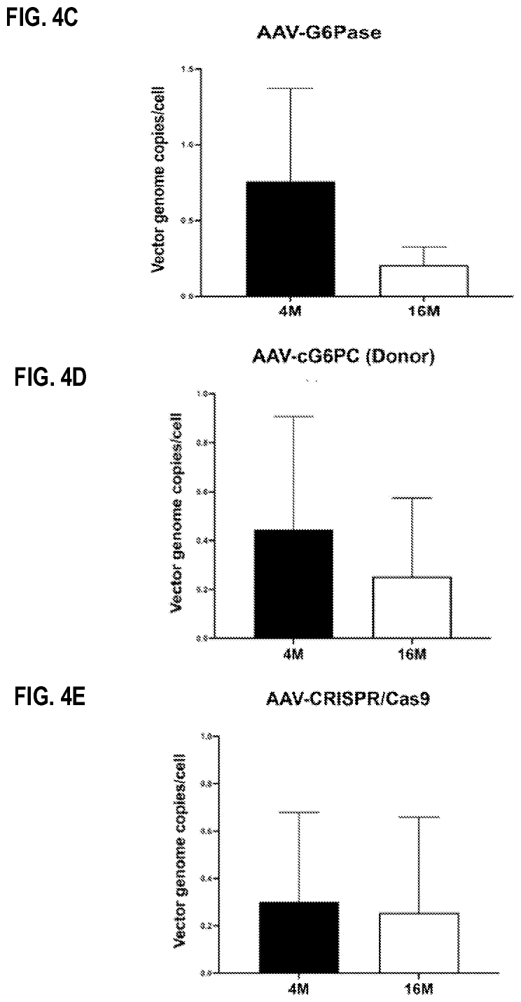

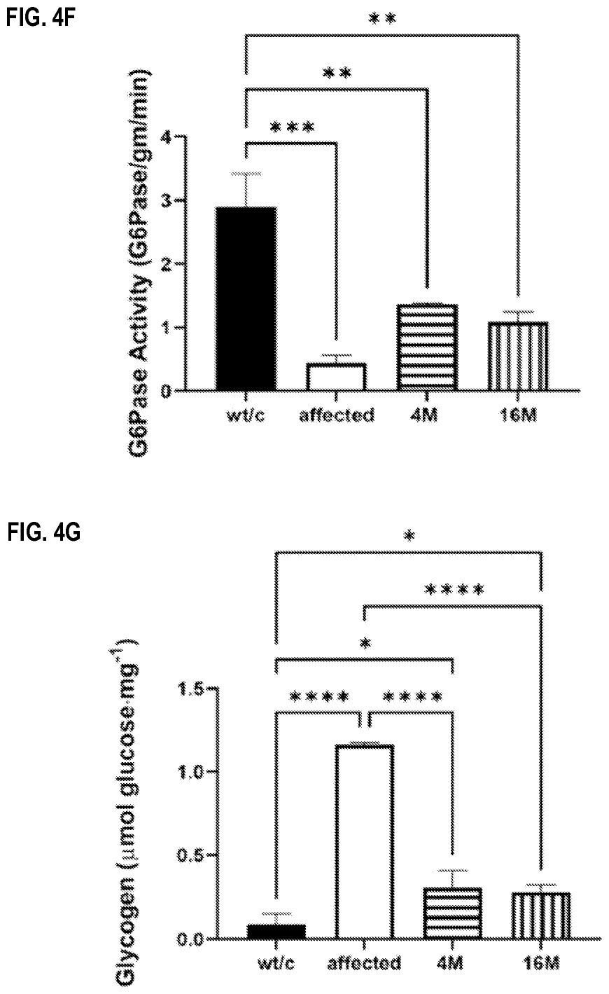

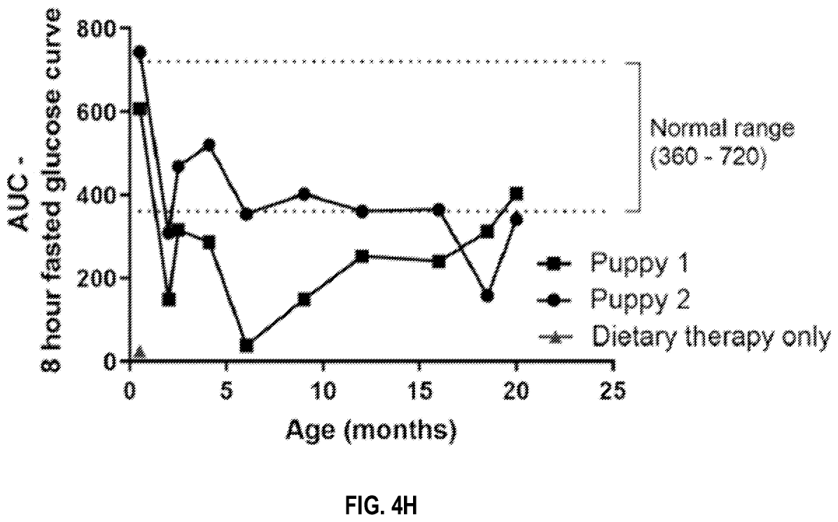

In this example, puppies with GSD Ia were initially treated with AAV-CRISPR/Cas9 and AAV-cG6PC (Donor) to perform neonatal genome editing at 2 days of age, followed by gene replacement therapy with one or more alternative serotypes of AAV to control symptoms of GSD Ia at the indicated ages for the individual puppies (Puppy 1 shown in green, Puppy 2 in purple in FIG. 4A, see Table 3 below for details). The control group of dogs were those described in Example 2 that received 3 doses of gene replacement therapy during infancy.

As shown in FIG. 4A and described in Table 3 below, two puppies with GSD Ia were treated with genome editing as neonates at a 5:1 ratio of AAV7-cG6PC and AAV7-SaCas9. One puppy was dosed at 4×10¹³ vp/kg AAV7-cG6PC and 8×10¹² vp/kg AAV7-SaCas9. The other was dosed with 5×10¹³ vp/kg AAV7-cG6PC and 1×10¹² vp/kg AAV7-SaCas9. As described further below, the donor vector was efficacious in preventing hypoglycemia and improving survival of GSD Ia puppies in the first two months of life, especially since affected puppies have previously demonstrated severe hypoglycemia and very high mortality in the first two months of life when treated with diet therapy alone (Koeberl et al., AAV vector mediated reversal of hypoglycemia in canine and murine glycogen storage disease type Ia” Molecular Therapy, 16, 665-672). However, the CRISPR/Cas9 treated puppies subsequently developed recurrent hypoglycemia and so were treated with gene replacement vector as described below, which reversed their symptoms (FIG. 4A, FIG. 4H). Specifically, one puppy (the first described above) received two doses of gene replacement therapy (e.g., AAV10 G6Pase at 3×10¹² vp/kg and AAV8 G6Pase at 1×10¹³ vp/kg) at ages 2 and 3 months, respectively. The second puppy received only one gene replacement therapy at 2 months of age (e.g., AAV9 G6Pase at 3×10¹³ vp/kg). Table 3 below details the treatment protocols used for both puppies.

TABLE 3
Vector Administration Protocols for Animals Treated as Neonates

| | | Age when | | | |
|---|---|---|---|---|---|
| | | vector/medication | | | Vector/Medication |
| Puppy | Sex | administered | | AAV vector/medication | Dose |
| #1 | M | 2 | days | AAV2/7 G6Pase + AAV2/7 | 4 × 10¹³ vp/kg + 8 × |
| | | | | CRISPR | 10¹² vp/kg |

| | | | | |
|---|---|---|---|---|
| 2 | months | AAV2/10 G6PASE | 3 × 10¹² | vp/kg |
| 3 | months | AAV2/8 G6Pase | 1 × 10¹³ | vp/kg |
| 12-16 | months | Bezafibrate^{B} | 4 | mg/kg/day |

| | | | | | |
|---|---|---|---|---|---|
| #2 | M | 2 | days | AAV2/7 G6Pase + AAV2/7 | 5 × 10¹³ vp/kg + 1 × |
| | | | | CRISPR | 10¹³ vp/kg |

| | | | | |
|---|---|---|---|---|
| 2 | months | AAV2/9 G6Pase | 3 × 10¹³ | vp/kg |

^{B}Description and results of bezafibrate treatment described in Waskowicz et al., Hum Mol Genet. 28, 143-154.

Liver biopsies from treated puppies were taken at both 4 and 16 months after treatment with gene editing vectors. The liver biopsies were analyzed for integration of donor transgene and vector copy number as described above in Example 2. Specifically, integration of the Donor transgene was detected in both puppies' liver biopsies at Months 4 and 16 following administration of the editing vectors (FIG. 4B). Both the editing vector genomes (AAV-cG6PC and AAV-SaCas9) and the gene replacement vector genome (AAV-G6Pase) were detected at Months 4 and 16 (FIG. 4C-FIG. 4E).

Biochemical effects of the vector treatment (e.g., G6Pase activity and glycogen content) were also assessed as described in Example 2 above. Specifically, in comparison with untreated GSD Ia dog liver, treated animals had increased G6Pase activity (FIG. 4F) and decreased glycogen content (FIG. 4G) that was stable. It is noted that both assays reflected corrections of the biochemical abnormalities in comparison with untreated affected controls. Furthermore, when the treated animals were tested using a glucose tolerance test, as detailed in Example 2 above, blood glucose during fasting decreased in the first months of life and stabilized thereafter near the normal lower limit (FIG. 4H). Specifically, genome editing treated puppies had normal blood glucose at two weeks of age following AAV vector administration (155+/−28 mg/dl, data not shown) after a two hour fast, which was markedly higher than for untreated, affected puppies that had low blood glucose (9+/−9.5 mg/dl; age-matched normal range 110+/−23 mg/dl, FIG. 4H). The genome editing treated puppies had normal area AUC for blood glucose during the 8-hour fasting test at two weeks of age (676+/−95 mg/dl; normal 360-720 mg/dl, shown in FIG. 4H), which subsequently decreased to below the normal range before recovering upon administration of additional gene replacement vectors (FIG. 4H).

### Example 6

cG6PC Transgene was Successfully Integrated in all Animals Treated as Neonates or Adults with Gene Editing Vectors.

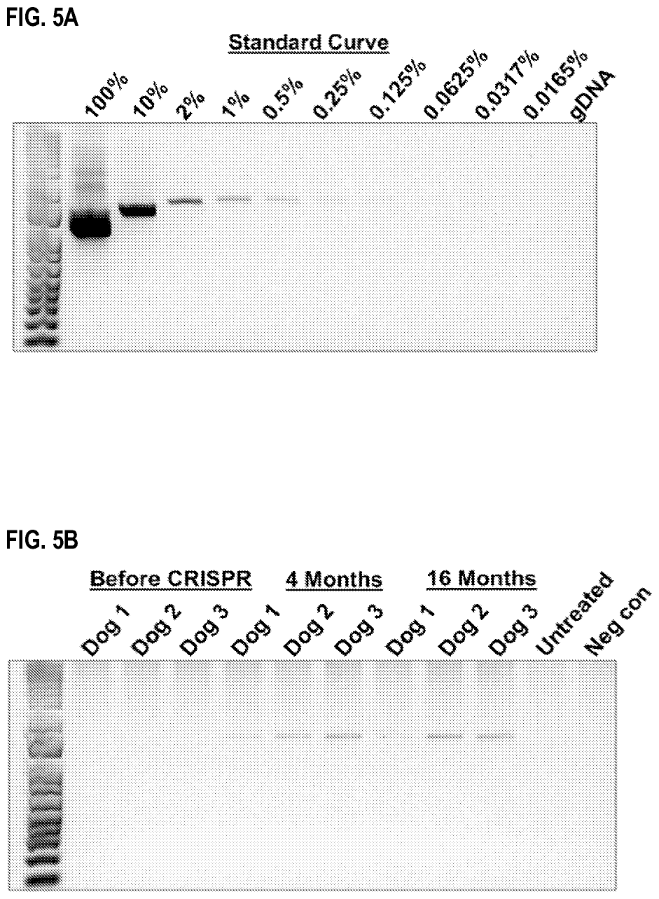

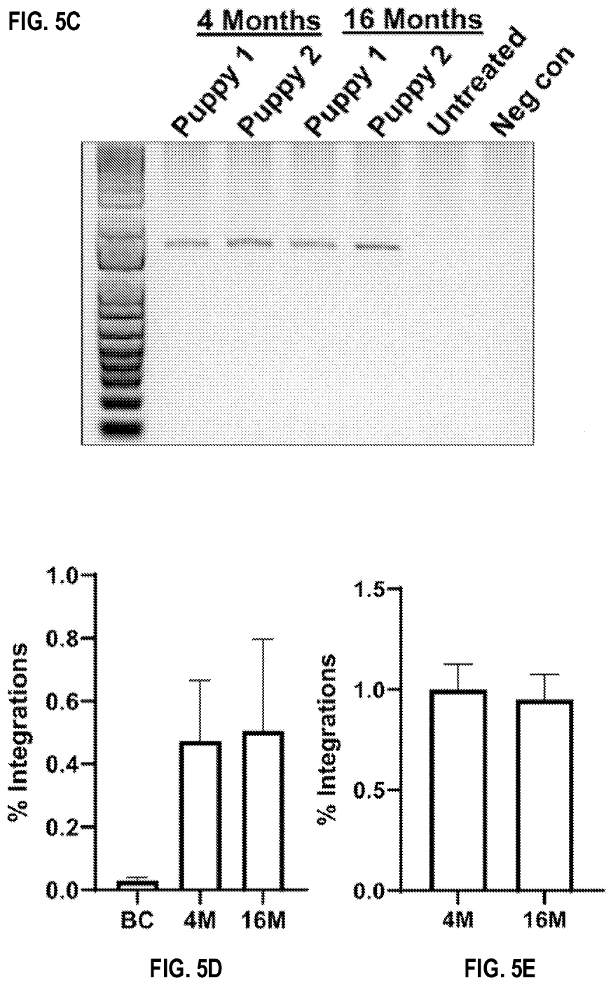

Integration of the therapeutic cG6PC transgene in all five treated animals from Examples 2 and 5 was quantified using a long-range nested PCR and compared with a standard curve using a synthetic DNA template containing the transgene flanked by canine G6PC genomic DNA (FIG. 5A-C). Specifically, a synthetic DNA fragment was generated by PCR with primers PI (SEQ ID NO: 59) and P4 (SEQ ID NO: 62) in the first round of PCR, followed by primers P2 (SEQ ID NO: 60 and P3 (SEQ ID NO: 61) using the integration PCR conditions detailed above in Example 2, which contained the junction fragment from the 3′ end of the canine G6PC cDNA in the transgene to the intron 1 G6PC sequence in dog genomic DNA. Serial dilutions of the synthetic DNA templates were made and used as the starting template for each PCR reaction to generate the standard curve. A standard curve was generated using serial dilutions of a starting template, which consisted of the purified junction fragment from integrated vector in intron 1 of G6PC that was generated by the integration PCR (FIG. 5A). The amount of starting template in the standard curve was calculated to represent 0.0165% to 100% modification of intron 1. Dog genomic DNA was amplified simultaneously to measure the level of integrated transgene and the G6PC locus.

FIG. 5B and FIG. 5C show integration PCR products for liver samples taken from dogs treated with CRISPR vectors as adults (FIG. 5B) or puppies (FIG. 5C). The relative intensities of the integration PCR products for liver DNA samples from dogs and puppies were compared with the standard curve to quantify the frequency of integration for each sample and averaged in FIG. 5D and FIG. 5E, respectively.

All three dogs treated as adults contained the integrated transgene at Months 4 and 16 (0.47%±0.19% and 0.51%±0.29%) with the transgene appearing to be stable (FIG. 5D). Both dogs treated as infants also contained detectable transgene integrations that remained stable from Months 4 to 16 (1.00%±0.13% and 0.95%±0.13%) (FIG. 5E).

### Example 7

### CRISPR Vector Treatment Resulted in Integrated Transgene Expression and Indel Formation at the Dog G6PC Locus in Animals Treated as Adults or Neonates.

In this example, expression of the integrated transgene was measured by next generation sequencing of canine G6PC transcripts. Transcripts expressed from the integrated vector were detected by unique SNPs in the sequence and compared with the total cG6PC transcripts expressed including those from the endogenous locus and episomal vectors. In brief, RNA was isolated from dog liver biopsies and converted to cDNA using RevertAid First Strand cDNA Synthesis Kit (Thermo-Fisher Scientific, Waltham, MA). G6PC transcripts were PCR using a forward primer in the 5′ UTR (5′-amplified by TGATAGCAGAGCAATCGCCAAGTC-3′, SEQ ID NO: 73) and the reverse primer in exon 2 (5′-AGGGTAGATGTGACCATCACGTAG-3′, SEQ ID NO: 74). The PCR products were purified with the Qiagen PCR Purification Kit (Quiagen, Germantown, MD, #28104). The DNA was sequenced using Illumina Mi-Seq and analyzed (performed by Azenta Lifesciences, South Plainfield, NJ). The donor AAV vector contains an BamHI restriction site −5 to −10 bp upstream of the transcription start site and the wild type base at position 363 that is mutated in GSD Ia dogs. Transcripts without the BamHI site but with the correction at position 363 were considered to be expressed off the integration transgene and quantified with a ChemiDoc imaging system (Bio-Rad Laboratories, Hercules, CA).

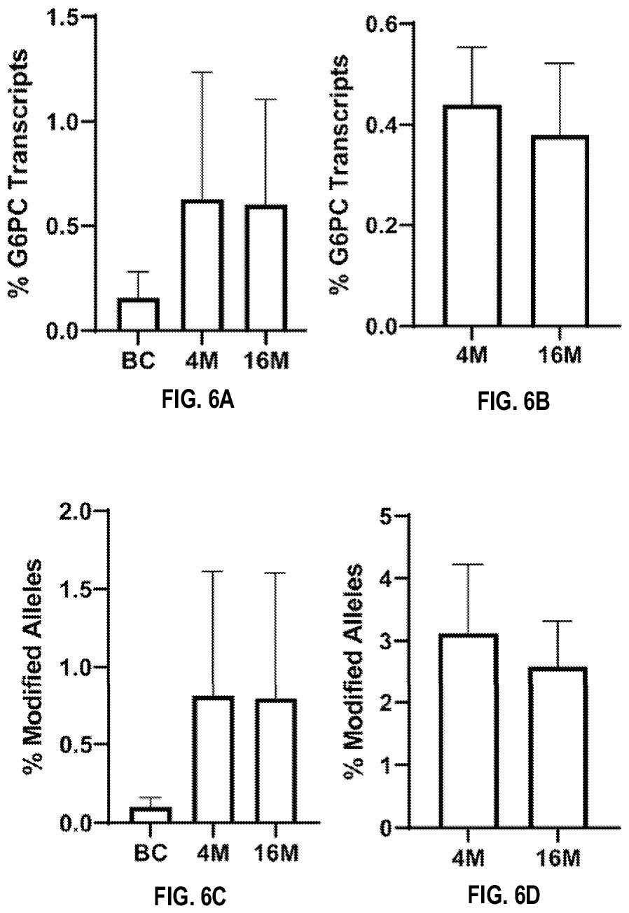

As described above, dog and puppy mRNA were extracted and converted to cDNA. Next generation sequencing was performed to determine the percentage of canine G6PC transcripts expressed from the integrated cG6PC transgene. CRISPR/Cas9 activity was measured by detecting indels generated at the G6PC locus in dogs. The target region of the G6PC locus was amplified by PCR followed by next gen sequencing of the amplicon. As shown in FIG. 6A, for the three dogs treated as adults, integrated transcripts were detected at Months 4 and 16 (0.63%±0.61% and 0.60%±0.50%). As shown in FIG. 6B, the two dogs treated as infants also had detectable transcripts expressed from the integrated transgene at Months 4 and 16 (0.44%±0.11% and 0.38%±0.14%).

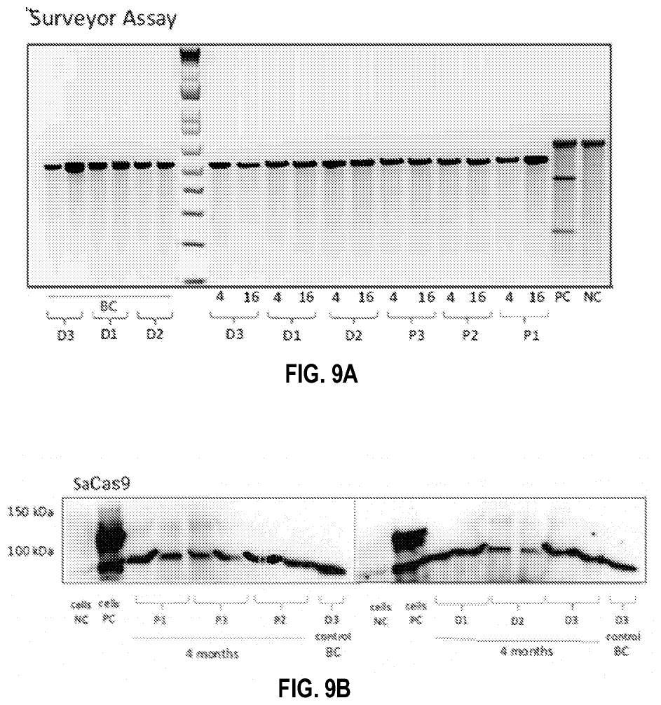

For all dogs the transcript expression remains stable 16 months after treatment. CRISPR/Cas9 activity at the target site at the exon 1/intron 1 boundary in G6PC was evaluated by Surveyor assay (see Example 2), which detected no detectable indel formation following vector administration that would indicate NHEJ at double-stranded breaks created by CRISPR/Cas9 (FIG. 9A-9B). Specifically, no on-target cleavage was detected on dog and puppy liver samples after 4 and 16 months of AAV vector administration (FIG. 9A). FIG. 9B shows representative western blots indicating the presence of SaCas9 protein with 128 kDa size on 4 months live samples.

To quantify percentage of modified G6PC alleles, next generation amplicon sequencing was used to detect small indels generated at the locus-indicating DNA cleavage repaired by NHEJ instead of HDR following CRISPR/Cas9 administration. The 3 adult dogs had indel rates of 0.81%±0.78% and 0.80%±0.76% at Months 4 and 16 (FIG. 6C). One of the adult dogs had extremely low indel formation (less than 0.1% at Months 4 and 16) indicating low nuclease activity and accounting for the wide variability. Both dogs treated as puppies had higher rates of indel formation at Months 4 and 16 (3.13%±1.10% and 2.59%±0.73%) (FIG. 6D).

To assess specificity of the CRISPR/Cas9 vector, the 10 most similar sites for potential off-target activity were analyzed. The software CRISPOR was used to determine potential off target sites. Those sites were amplified using gene specific primers (Table 4, below). PCR products were purified with the Qiagen PCR Purification Kit (#28104). The DNA was sequenced using Illumina Mi-Seq and analyzed (performed by Azenta Lifesciences, South Plainfield, NJ).

TABLE 4
Primers for Off-target CRISPR sites

| | | Forward | SEQ | Reverse | SEQ |
|---|---|---|---|---|---|
| | | Primer | ID | Primer | ID |
| Target Gene: | Location: | Sequence | NO: | Sequence | NO: |
| G6PC | 9: 20143274- | TGTCCGTGATTG | 75 | AGATGATCCA | 76 |
| | 20143299 | CAGACCTCAG | | GAGTCAGGAG | |
| | | | | GG | |
| U6- | 25: 22315002- | CTGTTCGAAGA | 77 | GAGGGGTTTT | 78 |
| ENSCAFG000 | 22315027 | GTCTGCATGTA | | AGGAAGTGCT | |
| 00007798 | | AG | | GG | |
| ST6GAL1 | 34: 19506977- | CAGCTTGTTCAC | 79 | CAGTTTTCTCC | 80 |
| | 195070002 | AACCAGACTC | | AGGCTCCTAA | |
| | | | | C | |
| C1ORf95 | 7: 38396252- | TGGGACATTTTA | 81 | CATTCTCGTCT | 82 |
| | 38396277 | GGGAGCCAAG | | TGGAACGTGT | |
| | | | | G | |
| RSPRY1 | 2: 59172272- | GCAAGATAAGA | 83 | ACACTAGCAG | 84 |
| | 59172297 | GTCGGTCTTCGT | | TCATACGGAC | |
| | | G | | TC | |
| ZNF536 | 1: 121194596- | ACTGGGGCTCG | 85 | CAAGGGAGAG | 86 |
| | 121194621 | TATCTCGG | | GCTTCAGAAT | |
| | | | | CG | |
| FANCC | 1: 71473173- | TACAGGGAGCC | 87 | CACAATCCCTT | 88 |
| | 71473198 | TGCTTCTGCC | | GTGGGAAGGG | |
| | | | | C | |
| CCDC170 | 1: 41875925- | AGCACATCATT | 89 | CCAGTTCACTT | 90 |
| | 41875950 | CATTCCTGGGC | | CGAGCGTCTT | |
| | | | | C | |
| TG | 13: 29383324- | GCATCCCTGGG | 91 | TAGCTGAGCG | 92 |
| | 29383349 | ATCCTTAGG | | TGAGAAGGGG | |
| DIXDC1 | 5: 21154057- | CTGTTTAGCTGG | 93 | GCTTTTGTCTG | 94 |
| | 21154082 | CATTGACTCC | | GTGTCATCCCC | |
| Pak7 | 24: 12671044- | CTCTGGAAGAG | 95 | GTACCATGAA | 96 |
| | 12671069 | CTACATAGTCAT | | CTTTTAGGAC | |
| | | C | | GGTC | |

For 9 sites, there was no significant increase in the rates of indel formation compared with an untreated dog control. Indel formation at the CCDC170 locus was increased 1.8-2.9-fold for the treated dogs compared with the untreated control dog (Table 5, below). Specifically, in Table 5 below, columns 1 and 2 contain the gene names and location within the gene where the gRNA targets. Columns 3 and 4 contain the target sequence and adjacent PAM for each site analyzed and differences with the G6PC target sequence (Row 3). Columns 8 contains the genomic location of the next generation sequencing of amplicons. All 10 off target sites were analyzed for the adult dog and puppy that had the highest on target indel formation and transgene integration (dog 3 and Puppy 1) and an untreated control dog. The percentage of indels for each site is shown in columns 5-7. The percentage of indels was equal for the treated dogs compared with the control and typically less than 1%. Next gen sequencing did reveal some natural genetic variation in the dog genome as the high rates of indels is ST6GAL1 and PAK7 is likely not due to CRISPR/Cas9 because it was detected in the Control.

TABLE 5
% Indels at Off-Target CRISPR locationsA

| | gRNA | gRNA Target Sequence | | Dog 3 | Puppy 1 | Control | |
|---|---|---|---|---|---|---|---|
| Gene | Location | (SEQ ID NO:) | PAM | Indel % | Indel % | Indel % | Genomic Location |
| G6PC | Intron | GATGGTCAGACATTTCCAGA | GAGAAT | 0.712 | 3.900 | 0.057 | 9: 20143274-20143299 |
| | | (SEQ ID NO: 1) | | | | | |
| U6- | Intergenic | AATGGTCAGACATTTCCAAA | GTGGAG | 0.024 | 0.032 | 0.025 | 25: 22315002-22315027 |
| ENSCAFGO | | (SEQ ID NO: 97) | | | | | |
| 0000007798 | | | | | | | |
| ST6GALI^{B} | Intron | AATGGTGAGAGATTTCCAGA | GAGAGA | 20.500 | 21.200 | 20.600 | 34: 19506977-19507002 |
| | | (SEQ ID NO: 98) | | | | | |
| Clorf95 | Intron | GATGCTCAGACATTTCAAAA | CAGAAT | 0.064 | 0.098 | 0.096 | 7: 38396252-38396277 |
| | | (SEQ ID NO: 99) | | | | | |
| RSPRY1 | Intron | AATGTTTAGACATTTCCAGA | TAGGAG | 0.098 | 0.145 | 0.119 | 2: 59172272-59172297 |
| | | (SEQ ID NO: 100) | | | | | |
| ZNF536 | Intron | GATGGGCTGGCATTTCCAGA | ACGGGG | 0.604 | 0.572 | 0.546 | 1: 121194596-121194621 |
| | | (SEQ ID NO: 101) | | | | | |
| FANCC | Intron | GAGTGTCAGACACTTCCAGA | GAGAAA | 0.133 | 0.127 | 0.109 | 1: 71473173-71473198 |
| | | (SEQ ID NO: 102) | | | | | |
| CCDC170 | Exon | GATGGTCATAAATTTCCTGA | CAGGGT | 1.120 | 1.790 | 0.625 | 1: 41875925-41875950 |
| | | (SEQ ID NO: 103) | | | | | |
| TG | Intron | TATAGTCAGACTTTTCCAGA | AGGAGG | 0.129 | 0.118 | 0.124 | 13: 29383324-29383349 |
| | | (SEQ ID NO: 104) | | | | | |
| DIXDC1 | Intron | GATTTTCAGACATTTCCACA | ATGAAT | 0.096 | 0.062 | 0.088 | 5: 21154057-21154082 |
| | | (SEQ ID NO: 105) | | | | | |
| Pak7^{B} | Exon | CATGGTGAGACATTTCCAGT | TTGGGG | 46.600 | 46.700 | 47.700 | 24: 12671044-12671069 |
| | | (SEQ ID NO: 106) | | | | | |

^{A}To measure off target nuclease activity by the CRISPR/Cas9 vector, 10 sites in the genome were analyzed for indel formation. The software CRISPOR was used to determine the 10 most likely off target sites based on the gRNA used in the AAV-CRISPR/Cas9 vector targeting the canine G6PC locus. See Example 7.
^{B}Next gen sequencing did reveal natural genetic variation in the dog genome as the high rates of indels is ST6GALI and PAK7 is likely not due to CRISPR/Cas9, because it was detected in the Control.

### Example 8

### Gene Editing Vectors Successfully Integrated and Resulted in Persistent GSD Ia Expression in a Mouse Model of GSD Ia

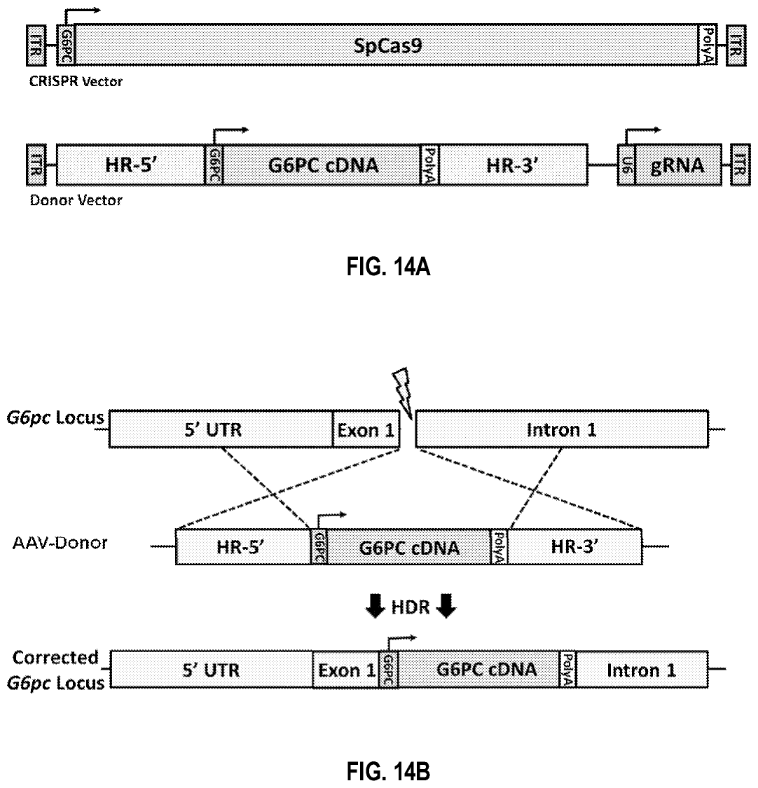

This example describes data from gene editing study in a neonatal G6pc KO mouse model. The design was similar to the strategy for the canine study described in Examples 1 to 7, with the exception that the “CRISPR” vector delivers SpCas9 and the gRNA is delivered by the “Donor” vector (FIG. 14A). The first vector named CRISPR (FIG. 14A, top) contains the S. pyogenes Cas9 gene (SEQ ID NO: 55) driven by a 303 bp minimal G6PC promoter (SEQ ID NO: 21). The second vector named mouse Donor (Donor) contains a human G6PC transgene (SEQ ID NO: 16) with 297 bp minimal G6PC promoter (SEQ ID NO: 20) flanked by mouse G6pc exon 1 sequence upstream and mouse G6pc intron 1 sequence downstream. The Donor vector also contained a U6 promoter expressing a gRNA targeting the exon 1/intron 1 boundary of the endogenous mouse G6pc gene (FIG. 14A, bottom). FIG. 14B shows a schematic of CRISPR/Cas9 cutting at the exon 1/intron 1 boundary of the mouse G6PC gene, followed by HDR to achieve integration of a human G6PC cDNA under control of its own promoter (human minimal G6PC promoter). In contrast to examples 1-7 which described integration of a G6PC transgene into a G6PC locus under control of an endogenous promoter, the Donor vector described above contains its own exogenous promoter. Therefore, it is capable of expression on its own (i.e., without integration into the G6PC locus). In this way it mirrors current gene therapy strategies for treating GSD Ia—where an exogenous AAV vector is delivered for episomal expression of the therapeutic protein. A goal of this example was to demonstrate whether inclusion of a CRISPR/Cas9 editing vector (e.g., pAAV-CRISPR) would increase efficacy of this Donor G6PC transgene vector. Data shown herein show that CRISPR/Cas9 based genome editing increases transgene integration and expression. Additionally, G6Pase activity and glycogen content are improved following genome editing. The combination of treatments resulted in improved blood glucose levels in GSD Ia mice as well as stable transgene integration and expression.

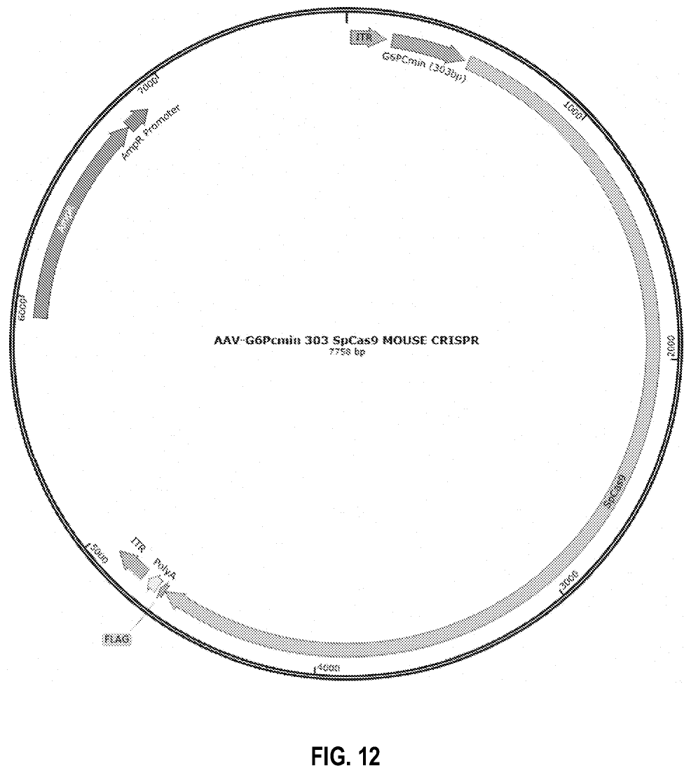

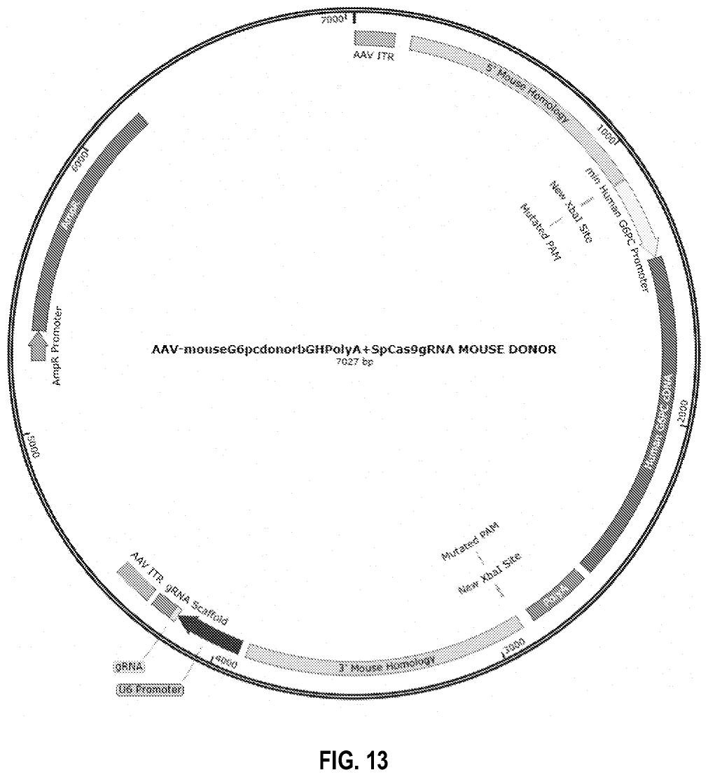

AAV vectors were prepared as described above using previously described AAV serotypes (see Gao et al., Proc Natl Acad Sci USA. 2002; 99 (18): 11854-9, incorporated herein by reference in its entirety). The AAV vector plasmid pAAV-CRISPR (SEQ ID NO: 46, FIG. 12) contained the vector gene comprised of an inverted terminal repeat (ITR) at each end flanking a 303 bp minimal G6PC promoter (SEQ ID NO: 21) expressing Cas9 from S. pyogenes with a FLAG tag and bovine growth hormone genomic polyadenylation sequence. The second AAV vector plasmid, pAAV-Donor (SEQ ID NO: 41, FIG. 13), contained an ITR at each end flanking the two transgenes 1) the human G6PC cDNA and 2) the U6 promoter expressing a gRNA. The transgene of (1) was flanked upstream by a 5′ homology arm (5′ UTR genomic sequence of mouse G6pc, including a 297 bp minimal G6PC promoter, SEQ ID NO: 25) and downstream by the human growth hormone genomic polyadenylation sequence followed by a 3′ homology arm (the intron I genomic sequence of mouse G6pc, SEQ ID NO: 26). Vectors were purified and quantified by Southern blot as described (Demaster A. et al., Human Gene Therapy. 2012 Apr. 1 2011; 23 (4): 407-418).

### Cohort 1

A first cohort of GSD Ia mice were treated at twelve days old with three different dosages of vector: low dose (Donor, 2×10¹² vg/kg; +/−CRISPR 4×10¹¹ vg/kg), medium dose (Donor 8×10¹² vg/kg; +/−CRISPR 1.6×10¹² vg/kg), and high dose (Donor, 3.2×10¹³ vg/kg; +/−CRISPR 6.4×10¹² vg/kg). Both donor and CRISPR editing vectors were delivered together 10 or separately. Mice were then evaluated 2 weeks and 4 weeks post treatment for blood glucose concentrations, glucose metabolism (e.g., glucose tolerance test), G6Pase activity, and liver glycogen content. Each of these tests were performed using methods and protocols similar to those in Examples 1-7 but are described further below.

Eight Hour Fast and Glucose Tolerance Test. Eight hour fasts for monitoring hypoglycemia were performed by fasting the mice for up to 8 hours and monitoring blood glucose. Blood glucose was measured by a point of care glucometer, either the AlphaTRAK or AlphaTRAK2 (Zoetis, Parsippany, NJ). The glucose tolerance test was performed by fasting the mice for 4 hours, checking blood glucose, and then injecting 10 μL/g of 10% dextrose prior to monitoring blood glucose 30, 60, 90, and 120 minutes later.

Monitoring G6Pase Activity and Glycogen Content in Liver. Enzyme analysis was performed as described (Koeberl D D et al. Gene Therapy. 2006 Sep. 1 2006; 13 (17): 1281-1289). Briefly, tissues were flash-frozen and stored at −70° C. Glycogen content was measured by complete digestion of polysaccharide using amyloglucosidase (Sigma Chemical Co., St. Louis, MO). The structure of the polysaccharide was inferred by using phosphorylase free of the debranching enzyme to measure the yield of glucose-1-phosphate. Specific G6Pase activity was measured by using glucose-6-phosphate as substrate after subtraction of nonspecific phosphatase activity as estimated by β-glycerophosphate.

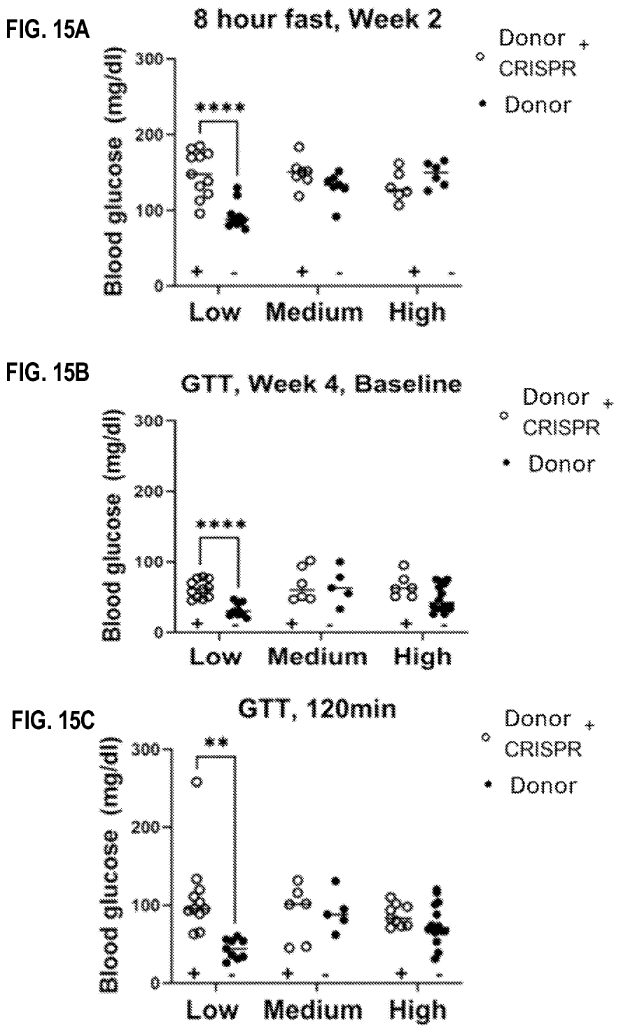

Two weeks after vector administration GSD Ia mice receiving both low dose Donor+CRISPR vectors had increased blood glucose concentrations measured after fasting for 8 hours (FIG. 15A), in comparison with mice treated with Donor alone. Glucose tolerance test was performed four weeks after treatment to further evaluate glucose metabolism. During the glucose tolerance test (GTT) mice were fasted for 2 hours then injected with dextrose. Blood glucose levels were measured at the start (baseline) and every 30 minutes for 120 minutes. In the glucose tolerance test, low dose Donor+CRISPR vector administration improved blood glucose at Baseline following 4 hours fasting (FIG. 15B) and at 120 minutes following glucose administration (FIG. 15C).

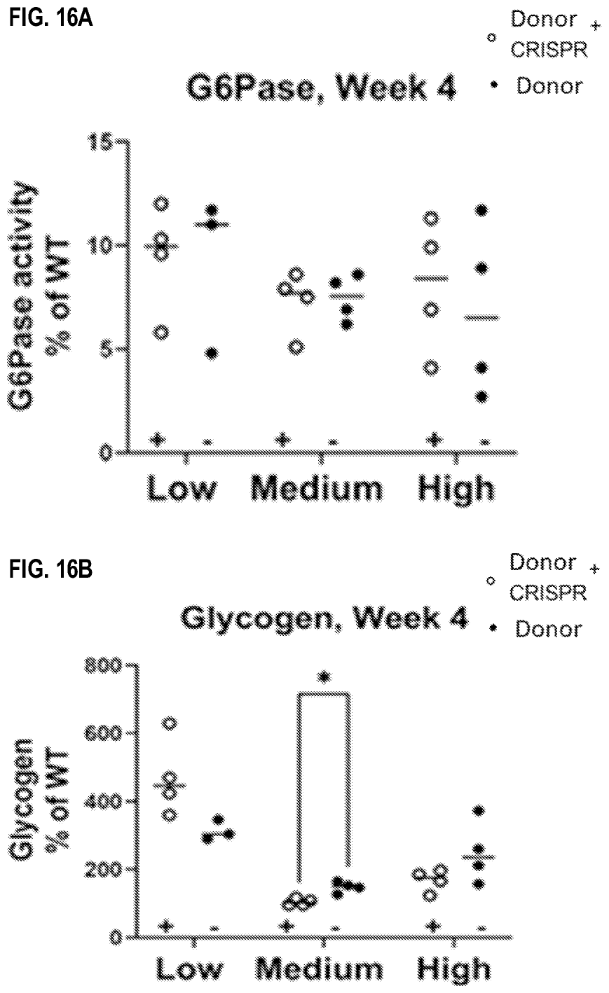

Biochemical correction of the GSD Ia mice livers was evaluated four weeks after treatment by analyzing G6Pase activity and glycogen content as described above. G6Pase activity was similar between groups (FIG. 16A). However, liver glycogen was significantly decreased for GSD Ia mice receiving both medium dose Donor+CRISPR vectors, in comparison with mice receiving Donor vector only (FIG. 16B). Additionally, CRISPR treated mice had hepatic glycogen content similar to wildtype mice (not shown).

Copy numbers of the donor vector and transgene expression was also evaluated 4 weeks post treatment. AAV vector genome copy number was measured by quantitative real-time PCR with liver genomic DNA and normalized to β-actin. Quantification of donor transcripts was evaluated using qPCR as a measure of transgene expression. Plasmid DNA corresponding to 0.01 to 100 copies of the murine G6pc gene (in 500 ng genomic DNA) was used in a standard curve. qPCR was performed on a Lightcycler 480 (Roche Diagnostics, Basel, Switzerland) using SYBR Green mix (Thermo-Fisher Scientific, Waltham, MA) and the following primers: hG6PC Fwd (5′-GCAGTTCCCTGTAACCTGTGAG-3′, SEQ ID NO: 67), hG6PC Rev (5′-GGTCGGCTTTATCTTTCCCTG-3′, SEQ ID NO: 68), SpCas9 Fwd (5′-AGTACAGCATCGGCCTGGAC-3′, SEQ ID NO: 107), SpCas9 Rev (5′-GGGCTCCGATCAGGTTCTTC-3′, SEQ ID NO: 108), mB-actin Fwd (5′-GGCTGTATTCCCCTCCATCG-3′, SEQ ID NO: 109), mB-actin Rev (5′-CCAGTTGGTAACAATGCCATGT-3′, SEQ ID NO: 110). Cycling conditions were 95° C. for 5 min, followed by 45 cycles of 95° C. for 10 s, 60° C. for 10 s, and 72° C. for 20 s followed by acquisition).

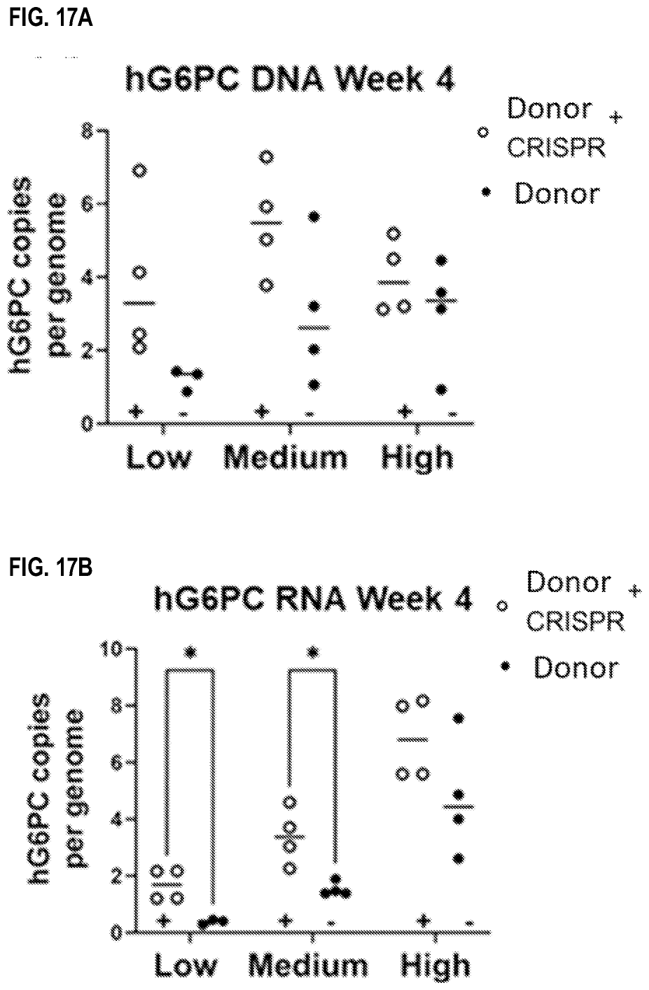

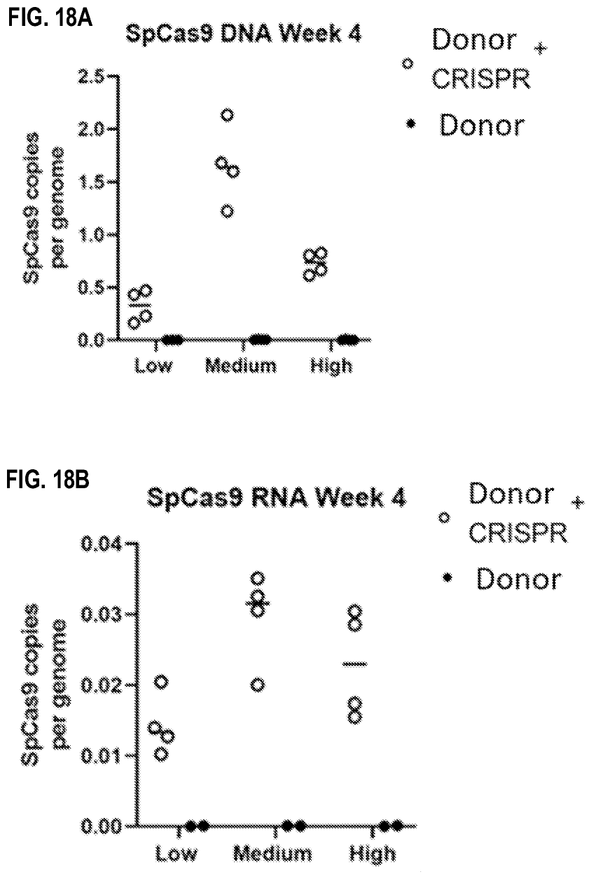

It was found that copies of the Donor vector trended higher in the liver for the CRISPR+Donor treated group, in comparison with the Donor treated mice (FIG. 17A). Expression of the Donor transgene in the liver was higher in both the low and medium Donor+CRISPR treated groups compared with mice receiving Donor vector only (FIG. 17B). As expected, the CRISPR transgene was elevated in the liver of mice treated with Donor+CRISPR vectors, but it was present at much lower copy number than the Donor vector containing the therapeutic transgene (FIG. 18A-18B).

### Cohort 2

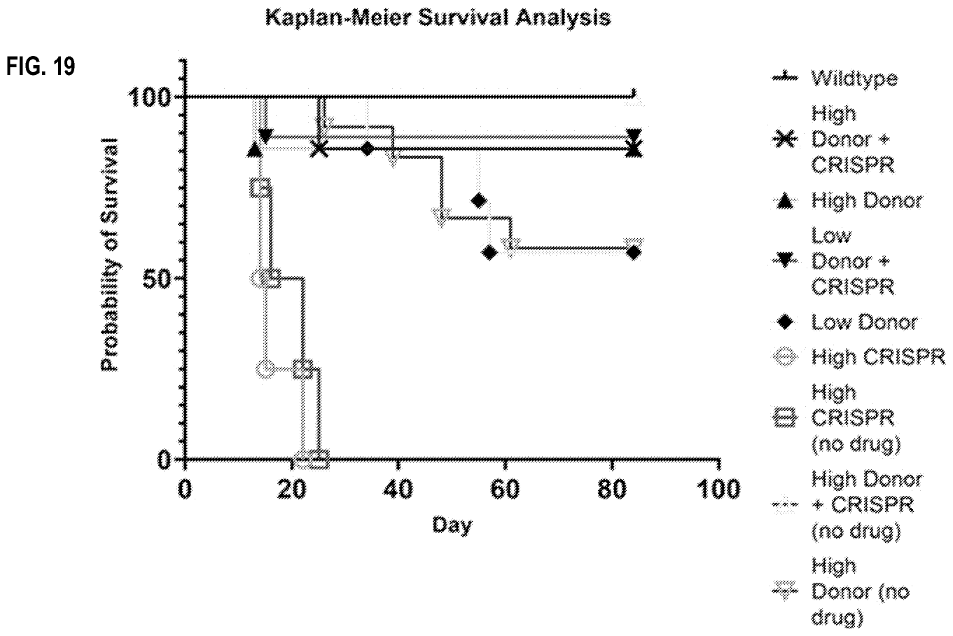

After observing some benefits with the addition of a CRISPR editing vector to the gene therapy in Cohort 1, the study was expanded to include more groups to find the most efficacious treatment. First, the length of treatment was increased to twelve weeks. Also, the drug bezafibrate, a pan-agonist of peroxisome proliferator-activated receptors (PPARs), which enhances the expression of genes involved in lipid homeostasis and energy metabolism, was included in addition to the viral vectors (Waskowicz L R et al. Human Molecular Genetics. 2019; 28 (1): 143-154). Bezafibrate has previously been shown to lower liver triglycerides and glycogen in GSD Ia mice while also increasing the transduction of AAV and expression of transgenes (Kang H-R et al., Molecular Therapy-Methods & Clinical Development. 2019; 13:265-273). Survival analysis was performed, revealing increased survival for GSD Ia mice receiving both Donor+CRISPR vectors at low or high dose with bezafibrate, in comparison with mice treated with Donor alone (FIG. 19). Specifically, as shown in FIG. 19, mice receiving CRISPR only (with or without bezafibrate) did not survive more than 4 weeks (orange and red lines). Mice receiving high dose Donor alone (solid black line, gray triangle) or low dose Donor with bezafibrate (teal line, black diamond) had improved survival rate of 60%. A combination of at least two of three following treatments (high dose Donor, CRISPR, and/or bezafibrate) resulted in survival rate between 80 and 100%. Additionally, all mice treated with CRISPR only did not survive past 30 days, indicating the need for a therapeutic transgene in GSD Ia mice early in life.

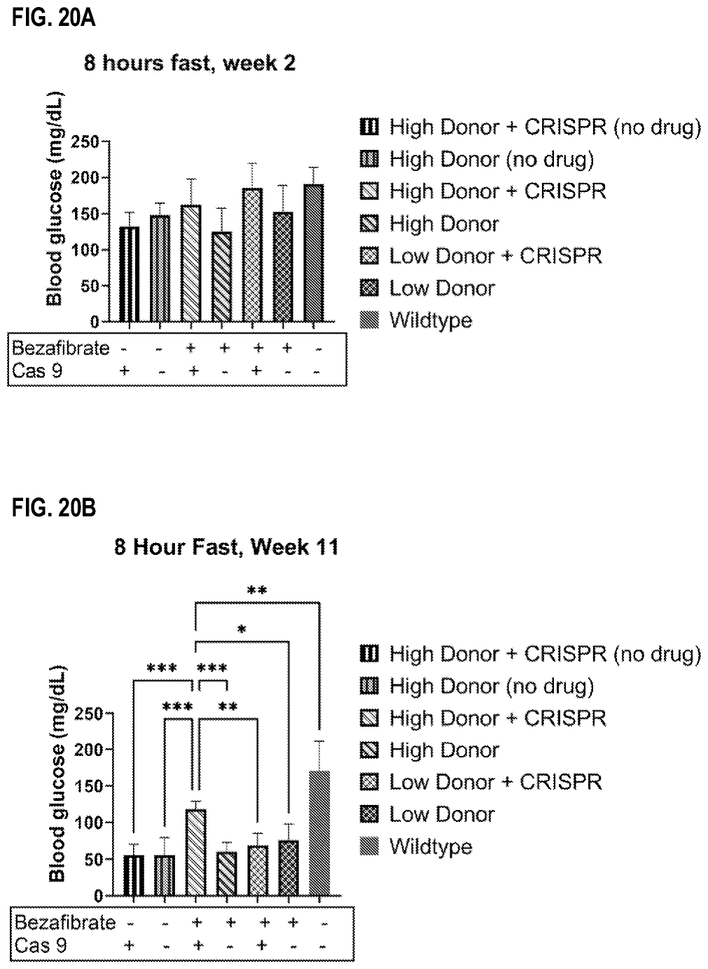

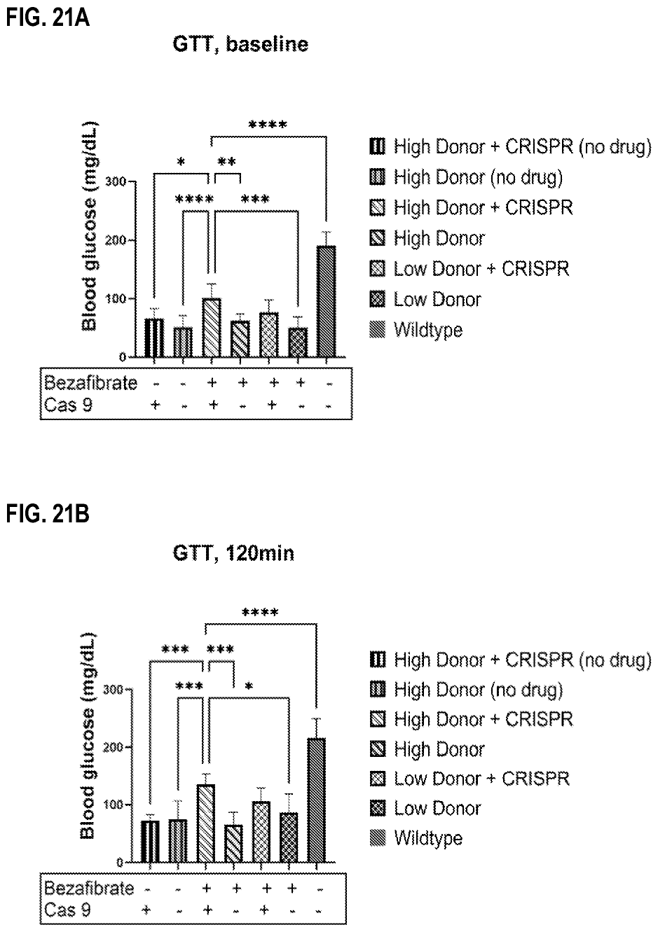

Blood glucose concentrations during fasting were measured two weeks after administration of the drug and vectors in this second cohort. No differences were observed among treatment groups and blood glucose was not significantly lower in any group compared with wild type mice (FIG. 20A). However, eleven weeks after treatment, the fasting blood glucose of mice receiving bezafibrate and high dose Donor+CRISPR vectors was significantly higher compared with all other treatment groups (FIG. 20B). Additionally, four weeks after treatment a glucose tolerance test revealed that the bezafibrate plus high dose Donor+CRISPR mice had elevated blood glucose at baseline and 120 minutes following glucose administration compared with all other treatment groups except bezafibrate plus low dose Donor+CRISPR vectors (FIG. 21A-21B). As above, mice were fasted for 2 hours before administering dextrose (glucose) in the glucose tolerance test. Baseline measurements were taken immediately before dextrose administration.

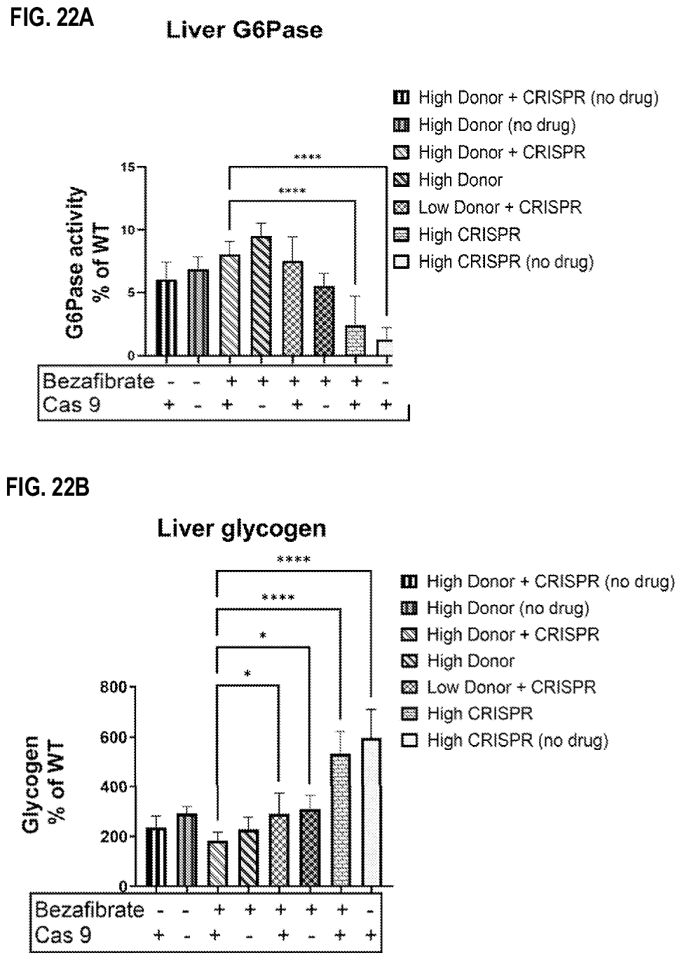

Levels of G6Pase activity and glycogen content were also measured in mice 12 weeks after treatment. It was found that all treatment groups had lower levels of G6Pase activity compared with wildtype levels. However, mice receiving bezafibrate plus Donor+CRISPR vectors had 8.0%±1.1% of WT G6Pase activity 12 weeks after administration compared with 1.3%±0.96% in mice receiving CRISPR vector only. (FIG. 22A). Adding bezafibrate with the Donor+CRISPR vectors significantly increased G6Pase activity compared with mice receiving the CRISPR vector only. Mice receiving bezafibrate plus high dose Donor+CRISPR vectors had the lowest liver glycogen content and was significantly lower than mice receiving low dose Donor+CRISPR and bezafibrate and mice receiving the CRISPR vector only with bezafibrate (FIG. 22B).

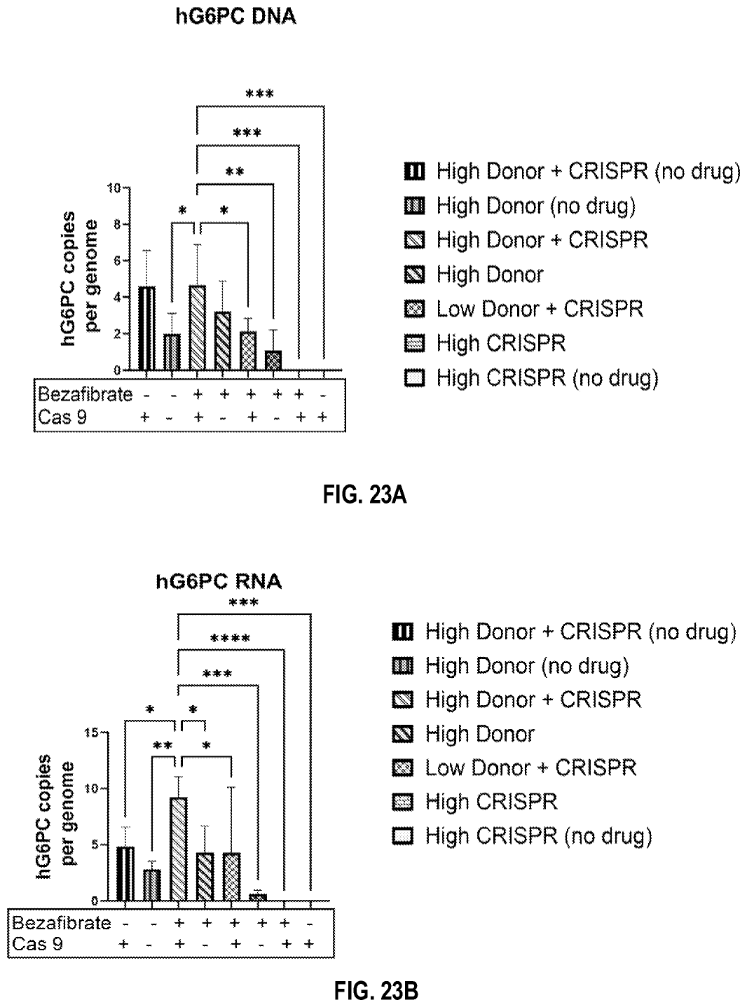

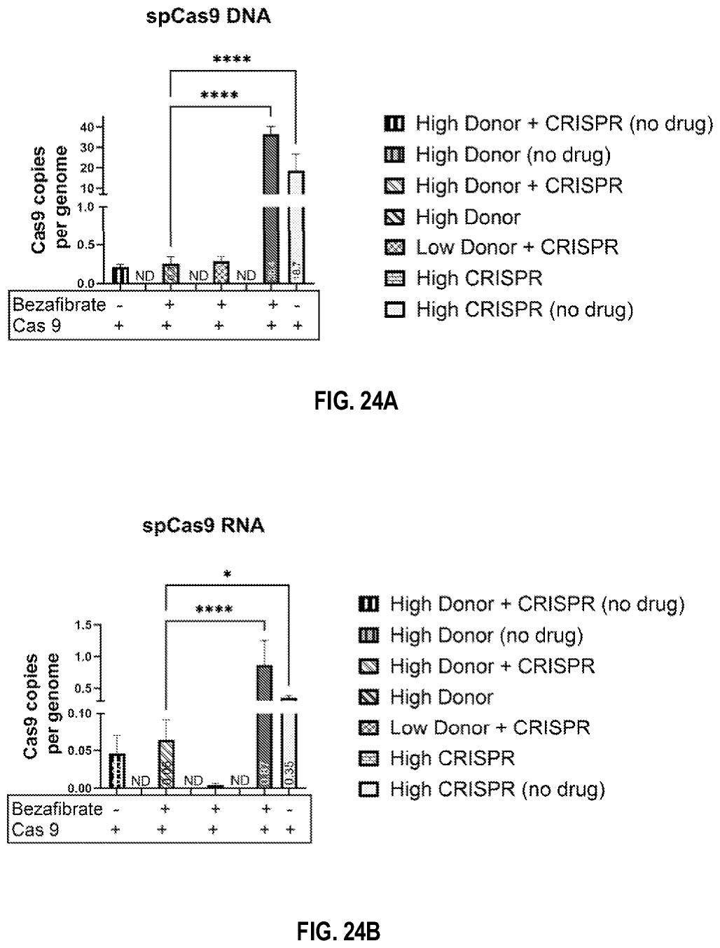

Copies of donor and CRISPR vectors were quantified in treated animals 12 weeks after treatment using methods described above. Copies of the Donor vector were highest in the livers of high dose Donor+CRISPR plus bezafibrate treated mice (FIG. 23A). The Donor vector copy number in the liver was significantly higher than all other groups expect for mice receiving high dose Donor+CRISPR and high dose Donor plus bezafibrate. The Donor transgene RNA expression was also highest in the high dose Donor+CRISPR with bezafibrate and significantly increased compared with all other treatment groups (FIG. 23B). CRISPR vector genomes were present at low levels in mice receiving both vectors (FIG. 24A). Expression of SpCas9 was minimal at 12 weeks and only detectable in mice receiving both vectors (FIG. 24B). Note that in FIG. 24A-24B, the far-right bars have significantly higher values because they represent levels of CRISPR vector and transcript copy number in mice treated only with CRISPR (no donor) and so were measured at an earlier timepoint (2 weeks instead of 12 weeks).

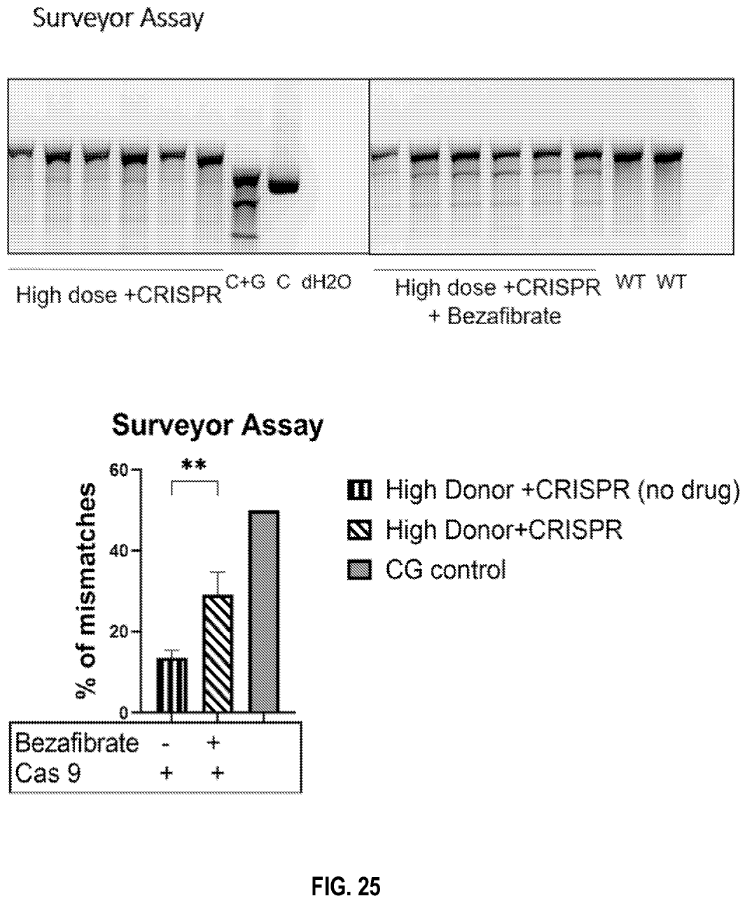

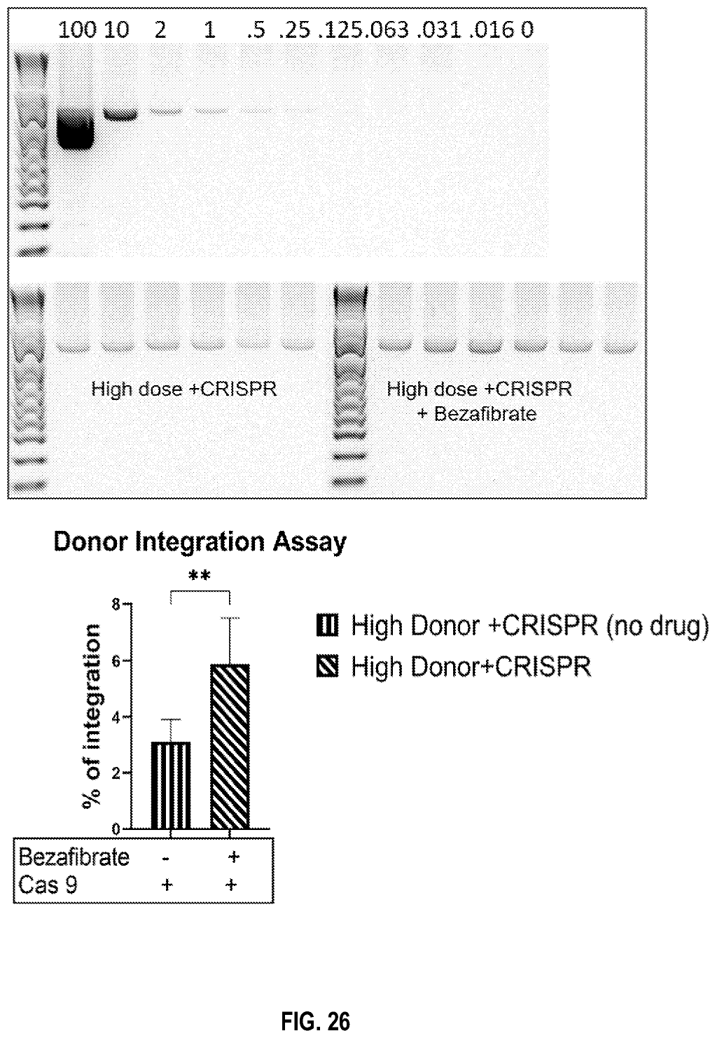

To measure nuclease activity at the mouse Gopc locus, the site was PCR amplified and analyzed by the Surveyor assay. The Surveyor assay denatures the double stranded PCR product then slowly reanneals the single strands. The single strands do not always reanneal to their original complimentary strand. Any indels generated by cleavage and NHEJ will form bulges in the reannealed DNA. The Surveyor nuclease will recognize the bulges in DNA and cleave it. The amount of indel formation is calculated but the volume of the two lower bands in each lane (the cleaved PCR product resulting from indel formation) compared to the total volume of all three bands in each lane. The Surveyor assay were performed as follows. Using purified DNA, the murine Gopc locus was amplified using one round of PCR following the conditions mentioned above in Example 1 except for the primers mousesurveyorFwd (5′-TGACCTACAGACTGAATCCAGG-3′, SEQ ID NO: 111) and mousesurveyorRev (5′-TAACATCTGTGCTCAGGAGCTG-3′, SEQ ID NO: 112). The PCR product was analyzed using the Surveyor Mutation Detection Kit (Integrated DNA Technologies, Coralville, IA) according to manufacturer's instructions. The PCR products were also sequenced using Sanger sequencing methods (Eton Biosciences, Durham, NC). The Surveyor assay detected increased indels in the high dose Donor+CRISPR with bezafibrate treated mice (30%±5.6%) compared with high dose Donor+CRISPR alone (16%±2.4%; FIG. 25). Furthermore, the integrated transgene could be detected only in mice treated with Donor+CRISPR and no integrated transgene was observed in mice receiving the Donor vector only. This was determined using a quantitative PCR integration assay. Briefly, a synthetic DNA fragment was generated by PCR with primers M1 (5′-CAGCCGCACAAGAAGTCGTTG-3′, SEQ ID NO: 113) and M4 (5′-TCTGGGAATCAGGGACTGGG-3′, SEQ ID NO: 116) in the first round of PCR, followed by primers M2 (5′-CCACTCCCACTGTCCTTTCC-3′, SEQ ID NO: 114) and M3 (5′-GGCTCAGTAGATCAAGTGCCTGC-3′, SEQ ID NO: 115) using the integration PCR conditions detailed above in Example 6, which contained the junction fragment from the 3′ end of the human G6PC cDNA in the transgene to the intron 1 G6pc sequence in mouse genomic DNA. Serial dilutions of the synthetic DNA templates were made and used as the starting template for each PCR reaction to generate the standard curve. The amount of starting template was calculated to represent 0.016% to 100% modification of intron 1. The band intensity was quantified for both groups and compared with the standard curve to quantify the frequency of integration for each sample. Mouse genomic DNA was amplified simultaneously to measure the level of integrated transgene and the G6pc locus. Data from this quantitative PCR integration assay revealed mice in the high dose Donor+CRISPR with bezafibrate group had 5.9% #1.7% of alleles containing the integrated transgene. This was significantly more than the high dose Donor+CRISPR without drug (3.1±0.8%; FIG. 26). These data confirmed the activity of CRISPR/Cas9 in the genome editing mouse liver. Adding bezafibrate increased expression of the transgenes resulting in increased nuclease activity and transgene integration.

In general, the data in this example shows that CRISPR/Cas9 based genome editing can integrate a full-length therapeutic transgene in the liver of GSD Ia mice. Administering the CRISPR vector that delivered Cas9 to activate the CRISPR/Cas9 nuclease, along with a functional Donor transgene improved the therapeutic effect in young mice. This demonstrated the benefit of targeted nuclease dependent genome editing as CRISPR/Cas9 improved efficacy of the Donor transgene but had no effect on its own. Additionally, the Donor transgene never integrated in the G6pc locus without CRISPR/Cas9. This indicates that nuclease activity increases the rate of HDR mediated integration despite claims that Donor templates can integrate spontaneously or independent of nuclease activity. Furthermore, adding bezafibrate, a drug known to increase transgene expression and editing efficacy, improved integration frequency and biochemical correction in mice long term. In the canine study described in Examples 1-7, transgene integration was observed, and the transgene persisted, but the biochemical corrections were minimal and attributable to the remaining episomal vector genomes. Even the dogs treated as neonates developed hypoglycemia and required rescue doses of gene replacement therapy. Combining the dual vector treatment with bezafibrate resulted in the most efficacious outcome, which was a stand-alone treatment in mice with GSD Ia. This will help when designing future preclinical studies with large animal models or patients to achieve a more robust therapy.

### Example 9

Alternative Mouse Vectors for Alignment of G6PC Transgene with Native Promoter Were Designed

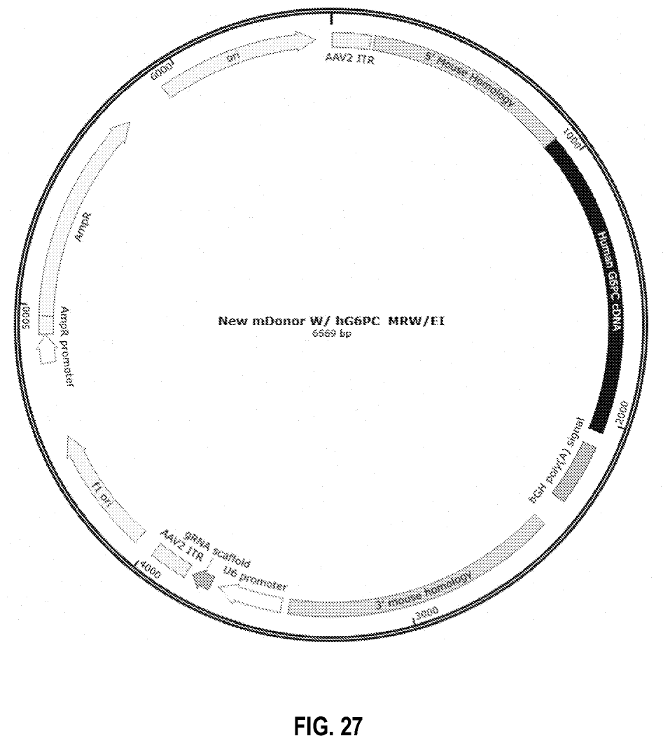

The murine vectors described in Example 8 contain a G6PC transgene operably linked to an exogenous promoter so that the exogenous promoter (e.g., minimal human G6PC promoter) controlled expression of the G6PC transgene (e.g., human G6PC) once integrated into the mouse genome. This is in contrast with the canine vectors described in Examples 1 to 7 which incorporate a G6PC transgene in frame with endogenous canine G6PC promoters, allowing for endogenous control of G6PC expression in edited cells. To achieve this in the mouse model, a different “donor” vector was prepared where the start codon of the human G6PC transgene is integrated at the position of the mouse G6PC start codon. This vector (FIG. 27, SEQ ID NO: 42) comprises two ITRs flanking two genes: (1) the transgene consisting of human G6Pc cDNA (SEQ ID NO: 17) and (2) the U6 promoter expressing a gRNA targeting SEQ ID NO: 9, where (1) was flanked upstream by a 5′ homology arm and 3′ homology arm aligning to the portion of the mouse genome surrounding the start codon of the mouse G6PC gene. This vector is provided as SEQ ID NO: 42. It is delivered to mice as described in Example 8 along with the CRISPR vector described in that Example (e.g., SEQ ID NO: 46). Phenotypic effect of the transgene insertion is measured as described above and include biochemical assays such as G6Pase activity and glycogen content in liver, glucose tolerance, and fasting glucose levels as well as survival curves. Further, genetic analysis is performed to track copy numbers of the vectors as well as donor integration and CRISPR activity.

### Example 10

### Human Vectors for Gene Editing in GSD Ia Patients

Based on data and results in earlier Examples 1 to 9, it was determined that SaCas9 and SpCas9 gene editing systems each had unique advantages. SaCas9 is smaller and therefore ideal for delivery in AAV vectors and SpCas9 may be more efficient at editing. To this end, two different human gene editing vector systems were designed similarly to those used in canine and murine models above. Specifically, the SaCas9 vectors included a donor vector that delivered only the transgene flanked by homology arms and the CRISPR vector delivers the SaCas9 transgene and the guide RNA. In the SpCas9 vector system, the donor vector delivers the transgene and the guide RNA and the CRISPR vector only delivers spCas9 due to size constraints. The combination of these two gene editing systems allows for both flexibility and adaptability to effectively treat GSD Ia in patients.

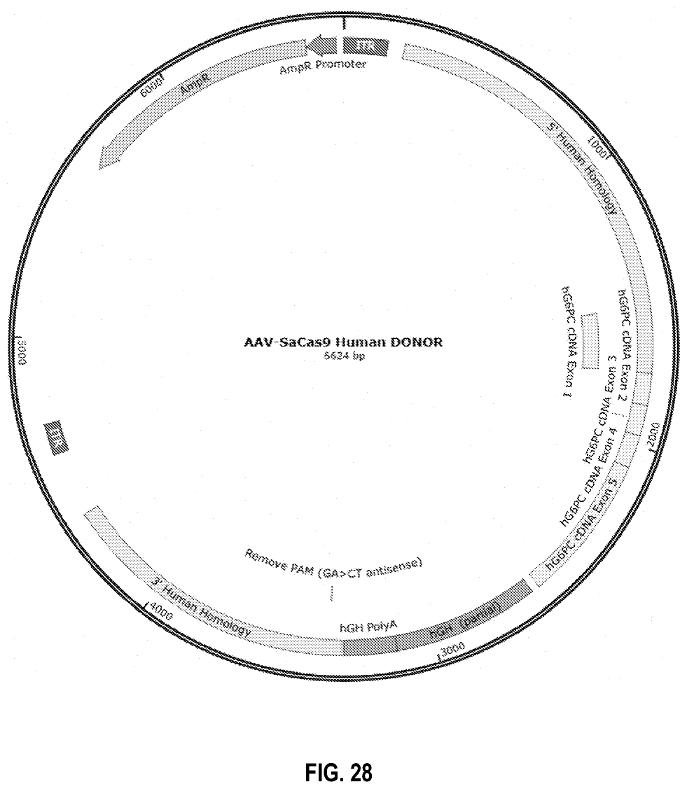

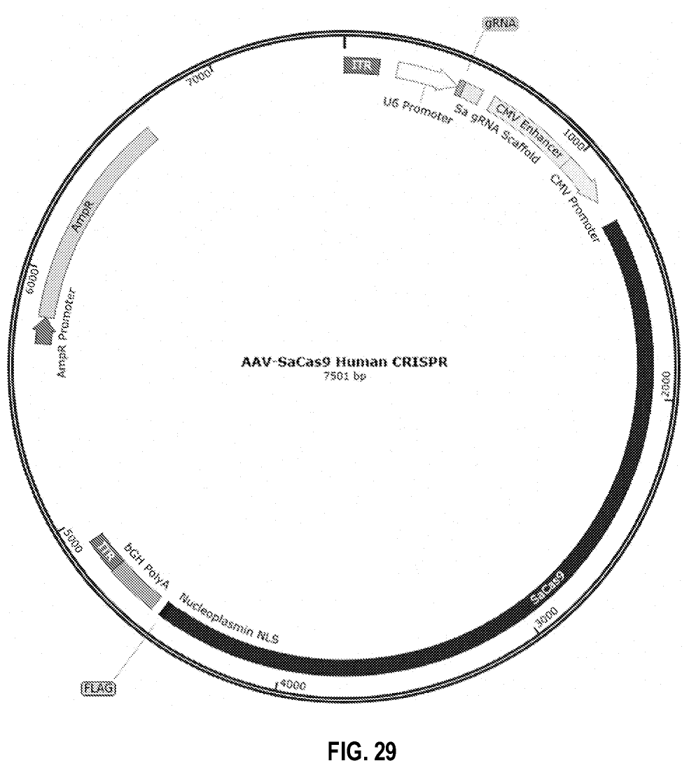

SaCas9 vector system. The human SaCas9 donor vector (FIG. 28, SEQ ID NO: 44) contains two ITRs flanking a human G6Pc cDNA transgene (SEQ ID NO: 18). The cDNA is flanked upstream by a 5′ homology arm (SEQ ID NO: 33) containing the 5′ UTR genomic sequence of human G6PC, including a 1284 bp human G6PC promoter (SEQ ID NO: 23). It is noted that the human G6Pc cDNA transgene and 5′ homology arm can overlap such that exon 1 of the human G6Pc cDNA transgene is considered part of the 5′ homology arm. In this aspect, the homology arm is provided as SEQ ID NO 32, and the human G6Pc cDNA transgene flanked by the 5′ homology arm lacks exon 1. Table 7, below, provides annotated sequences for SEQ ID NOs 32 and 33 that indicate exon 1. Downstream of the human G6Pc cDNA transgene is the human growth hormone genomic polyadenylation sequence followed by a 3′ homology arm (SEQ ID NO: 35) containing the Intron 1 genomic sequence of human G6PC. Since in this vector system, a representative gRNA target sequence is located in intron 1 (e.g., see SEQ ID NO: 5) The 3′ homology arm further comprises a GA>CT mutation in the antisense direction that removes the PAM site when integrated into the genome (see bolded and underlined section in Table 7). This full SaCas9 donor vector is provided as SEQ ID NO: 44. The human SaCas9 CRISPR vector (FIG. 29, SEQ ID NO: 48) contains the AAV vector gene comprised of two inverted terminal repeats (ITRs) flanking two transgenes: (1) the U6 promoter expressing a gRNA targeting SEQ ID NO: 5 and (2) a minimal CMV promoter expressing Cas9 from S. aureus (SEQ ID NO: 56) with a FLAG tag and bovine growth hormone genomic polyadenylation sequence. This vector is provided as SEQ ID NO: 48.

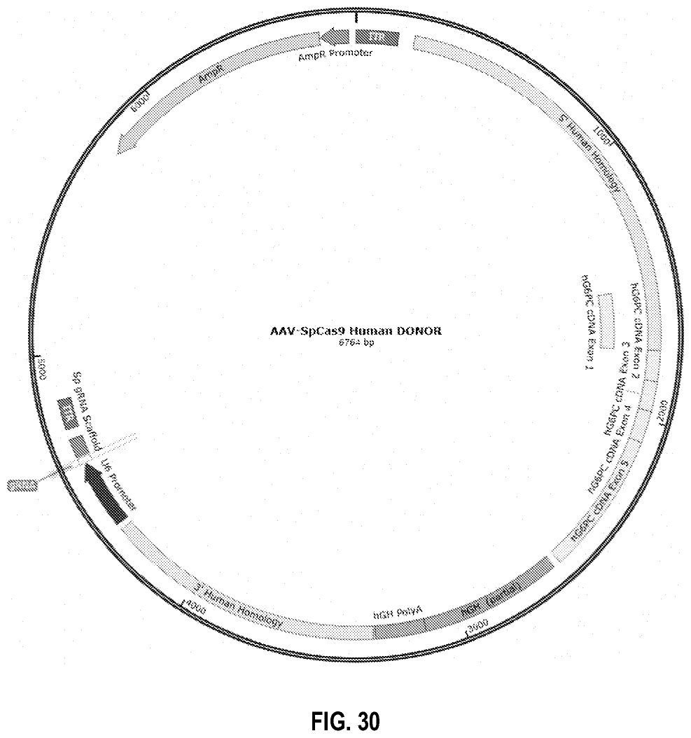

SpCas9 vector system. The human SpCas9 donor vector (FIG. 30, SEQ ID NO: 45) contains two ITRs flanking two genes: (1) the transgene consisting of human G6Pc cDNA (SEQ ID NO: 18) and (2) the U6 promoter expressing a gRNA targeting SEQ ID NO: 10, where (1) is flanked upstream by a 5′ homology arm (SEQ ID NO: 33 or 32, described above) and 3′ homology arm (SEQ ID NO: 34) aligning to the portion of the human genome surrounding the start codon of the human G6PC gene. This vector is provided as SEQ ID NO: 45. The human SpCas9 CRISPR vector (FIG. 12, SEQ ID NO: 46) is as described previously in Example 8 and contains two ITRS flanking a minimal hG6PC promoter expressing Cas9 from S. pyogenes (SEQ ID NO: 55) with a FLAG tag and bovine growth hormone genomic polyadenylation sequence. This vector is provided as SEQ ID NO: 46.

In addition to the gRNAs encoded by the vectors above, other gRNA target sequences are provided in the following tables that would be expected to work with the SaCas9 or SpCas9 vector systems above. Accordingly, other human SaCas9 CRISPR vectors (e.g., FIG. 29) will be prepared incorporating a nucleic acid sequence encoding any of the gRNAs targeting a sequence in the G6PC gene corresponding to any of SEQ ID NOs: 5-8. Likewise, other human SpCas9 DONOR vectors (e.g., FIG. 30) will be prepared incorporating a nucleic acid sequence encoding any of the gRNAs targeting a sequence in the G6PC gene corresponding to any of SEQ ID NOs: 10 to 15. These gRNAs target sequences, along with their PAMs are described in the Tables 6A and 6B below which also show the location of the PAM within the G6PC gene locus. When the gRNAs target sequences with PAMs in an intron of the G6PC gene locus (overlapping with either the 3′ or 5′ homology arms described above), modified homology arms (and donor nucleic acids thereof) will be prepared incorporating mutations to prevent additional editing after transgene insertion, according to standard methods in the art. These modified PAMs are also included in the Tables 6A and 6B below.

TABLE 6A
SaCas9 guides for editing human G6PC

| gRNA target | | | | |
|---|---|---|---|---|
| sequence | | Mutant | | |
| (SEQ ID NO) | PAM | PAM | Strand | Location |
| TGATGGACAGA | GCGAAT | GCctAT | minus | Intron 1 |
| CATTGCGAGA | | | | |
| (SEQ ID NO 5) | | | | |
| GCATGAGTAGC | CTGAAT | CTctAT | minus | Intron 1 |
| CCGTGGCTTC | | | | |
| (SEQ ID NO: 6) | | | | |
| TCAGAGAGAGG | ATGAGT | ATctGT | minus | Intron 1 |
| GGTTGGAAGC | | | | |
| (SEQ ID NO: 7) | | | | |
| AGATGATCCAA | AGGGGT | AGccGT | minus | Intron 1 |
| AGTCAGAGAG | | | | |
| (SEQ ID NO: 8) | | | | |

TABLE 6B
SpCas9 guides for editing human G6PC

| gRNA target sequence | | Mutant | | |
|---|---|---|---|---|
| (SEQ ID NO) | PAM | PAM | Strand | Location |
| TCTACGTCCTCTTCCCCATC | TGG | | Plus | Exon 1 |
| (SEQ ID NO: 10) | | | | |
| TTGCTGATCTCCTCTCTATA | TGG | TaG | Minus | Intron 1 |
| (SEQ ID NO: 11) | | | | |
| GTGGTAAGAACCATATAGAG | AGG | AaG | Plus | Intron 1 |
| (SEQ ID NO: 12) | | | | |
| GAGGAGATCAGCAAGAAAAG | AGG | AaG | Plus | Intron 1 |
| (SEQ ID NO: 13) | | | | |
| AGATCAGCAAGAAAAGAGGC | TGG | TaG | Plus | Intron 1 |
| (SEQ ID NO: 14) | | | | |
| CTGGGGAAAGCAACTTCTGA | TGG | TaG | Minus | Intron 1 |
| (SEQ ID NO: 15) | | | | |

Following preparation of the gene editing vectors, patients (e.g., patients with a GSD type 1 disease) are administered the gene editing vectors according to methods standard in the art. Phenotypic effect of the transgene insertion is measured as described above and includes biochemical assays such as G6Pase activity and glycogen content in liver, glucose tolerance, and fasting glucose levels. Further, genetic analysis is performed to track copy numbers of the vectors as well as donor integration and CRISPR activity in cells of the patients. In addition, patients are evaluated at regular points for clinical measurements of glycogen storage disease. Patients are treated early in life (neonatally) or in early childhood and are also be treated as adults. In some experiments, patients are further be treated with a gene replacement AAV vector containing a G6PC transgene under control of an exogenous promoter (e.g., a human G6PC promoter provided herein) but without homology arms. Patients receiving gene editing vectors alone are analyzed alone and compared to patients receiving only a gene replacement AAV vector or a combination of the gene editing vectors and gene replacement vectors. Positive outcomes in all groups are measured as improved glucose tolerance, improved fasting glucose levels, increased G6Pase activity, and reduced glycogen content and any other measure of improvement in progression of the glycogen storage disease.

### SUMMARY OF EXAMPLES

As detailed in Examples 1-9, the disclosed systems and methods demonstrate the successful integration of a therapeutic G6PC transgene into a mutant and/or dysfunctional G6PC gene. As detailed supra, the insertion of a functional G6PC cDNA downstream of the G6PC gene promoter provides numerous advantages. First, regardless of the underlying mutation in the G6PC gene, any patient can be treated. Second, the safe integration of the transgene into the mutant G6PC locus avoids inactivating any other gene. Third, the use of the endogenous G6PC promoter to drive expression avoids over-expression of G6Pase (which could otherwise cause a pre-diabetic state). Fourth, the gene editing methods disclosed herein can be combined with early transgene expression from an episomal Donor vector (e.g., as done with SEQ ID NO: 49 above), to prevent mortality or increase benefit of the disclosed gene editing methods.

Moreover, the Examples demonstrated that the methods and vectors disclosed herein achieved a significantly higher degree of transgene integration than seen in other models (e.g., models achieving only 0.5%-1% transgene integration). Alternative strategies of editing the G6PC gene locus have also not been as successful. Conversely, the vectors used here for the mouse GSD Ia genome editing achieved transgene integration in up to 6% of G6pc alleles in liver, which was further enhanced from 3.5% by adding bezafibrate treatment. Accordingly, the disclosed method achieved well above a threshold of 3% of normal G6Pase activity (up to 8% of normal) that prevents tumor formation in the GSD Ia liver.

In addition, the Cas9 transgene was almost completely lost following editing, based upon comparing two groups that were both administered high dose CRISPR: Cas9 DNA decreased 120-fold between Day 3 and Week 12. Loss of the Cas9 transgene increased safety by decreasing the potential risks of prolonged nuclease activity.

In summary, the best treatment had multiple benefits including a high rate of survival and higher blood glucose during fasting, and safe transgene integration that likely persists for the lifetime of the treated subject (see e.g., Example 8). It is predicted that a combination of treating mice during early infancy, but not in the neonatal period as done in dogs, and using appropriately high dose of the AAV vectors along with bezafibrate (or equivalent) treatment may be important to optimize genome editing for GSD Ia. Adding bezafibrate (or an equivalent) to a protocol optimized for delivery time and delivery dose strengthens the disclosed GSD Ia genome editing approach.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

TABLE 7
List of Sequences

| DESCRIPTION | SEQUENCE | SEQ ID NO |
|---|---|---|
| SaCas9 gRNA | gatggtcagacatttccaga | 1 |
| (canine target) | | |
| SaCas9 gRNA | ctggctcaacctcgtctttaaatg | 2 |
| (canine target) | | |
| SaCas9 gRNA | ttctgatggtcagacatttccaga | 3 |
| (canine target) | | |
| SaCas9 gRNA | aggtagatgatccagagtcaggag | 4 |
| (canine target) | | |
| SaCas9 gRNA | tgatggacagacattgcgaga | 5 |
| (human target) | | |
| SaCas9 gRNA | gcatgagtagcccgtggcttc | 6 |
| (human target) | | |
| SaCas9 gRNA | tcagagagaggggttggaagc | 7 |
| (human target) | | |
| SaCas9 gRNA | agatgatccaaagtcagagag | 8 |
| (human target) | | |
| SaCas9 gRNA | tctatgtcctctttcccatc | 9 |
| (mouse target) | | |
| SaCas9 gRNA | tctacgtcctcttccccatc | 10 |
| (human target) | | |
| SaCas9 gRNA | ttgctgatctcctctctata | 11 |
| (human target) | | |
| SaCas9 gRNA | gtggtaagaaccatatagag | 12 |
| (human target) | | |
| SaCas9 gRNA | gaggagatcagcaagaaaag | 13 |
| (human target) | | |
| SaCas9 gRNA | agatcagcaagaaaagaggc | 14 |
| (human target) | | |
| SaCas9 gRNA | ctggggaaagcaacttctga | 15 |
| (human target) | | |

| DESCRIPTION | SEQUENCE |
|---|---|
| Human G6PC | tagcagagcaatcaccaccaagcctggaataactgcaagggctctgctgacatcttcctgaggtgccaaggaaatgaggatggaggaaggaatgaatgttctccatgactttgggat |
| transgene (1) | ccagtcaacacattacctccaggtgaattaccaagactcccaggactggttcatcttggtgtccgtgatcgcagacctcaggaatgccttctacgtcctcttccccatctggttccatctt |
| (SEQ ID NO: 16) | caggaagctgtgggcattaaactcctttgggtagctgtgattggagactggctcaacctcgtctttaagtggattctctttggacagcgtccatactggtgggttttggatactgactacta |
| | cagcaacacttccgtgcccctgataaagcagttccctgtaacctgtgagactggaccagggagcccctctggccatgccatgggcacagcaggtgtatactacgtgatggtcacatc |
| | tactctttccatctttcagggaaagataaagccgacctacagatttcggtgcttgaatgtcattttgtggttgggattctgggctgtgcagctgaatgtctgtctgtcacgaatctaccttgct |
| | gctcattttcctcatcaagttgttgctggagtcctgtcaggcattgctgttacagaaactttcagccacatccacagcatctataatgccagcctcaagaaatattttctcattaccttcttcct |
| | gttcagcttcgccatcggattttatctgctgctcaagggactgggtgtagacctcctgtggactctggagaaagcccagaggtggtgcgagcagccagaatgggtccacattgacac |
| | cacaccctttgccagcctcctcaagaacctgggcacgctctttggcctggggctggctctcaactccagcatgtacagggagagctgcaaggggaaactcagcaagtggctcccat |
| | tccgcctcagctctattgtagcctccctcgtcctcctgcacgtctttgactccttgaaacccccatcccaagtcgagctggtcttctacgtcttgtccttctgcaagagtgcggtagtgccc |
| | ctggcatccgtcagtgtcatcccctactgcctcgcccaggtcctgggccagccgcacaagaagtcgttgtaagagatgtggagtcttcggtgtttaaagtc |
| Human G6PC | tcgaggtgccaaggaaatgaggatggaggaaggaatgaatgttctccatgactttgggatccagtcaacacattacctccaggtgaattaccaagactcccaggactggttcatcttg |
| transgene (2) | gtgtccgtgatcgcagacctcaggaatgccttctacgtcctcttccccatctggttccatcttcaggaagctgtgggcattaaactcctttgggtagctgtgattggagactggctcaacc |
| (SEQ ID NO: 17) | tcgtctttaagtggattctctttggacagcgtccatactggtgggttttggatactgactactacagcaacacttccgtgcccctgataaagcagttccctgtaacctgtgagactggacc |
| | agggagcccctctggccatgccatgggcacagcaggtgtatactacgtgatggtcacatctactctttccatctttcagggaaagataaagccgacctacagatttcggtgcttgaatg |
| | tcattttgtggttgggattctgggctgtgcagctgaatgtctgtctgtcacgaatctaccttgctgctcattttcctcatcaagttgttgctggagtcctgtcaggcattgctgttacagaaact |
| | ttcagccacatccacagcatctataatgccagcctcaagaaatattttctcattaccttcttcctgttcagcttcgccatcggattttatctgctgctcaagggactgggtgtagacctcctgt |
| | ggactctggagaaagcccagaggtggtgcgagcagccagaatgggtccacattgacaccacaccctttgccagcctcctcaagaacctgggcacgctctttggcctggggctggc |
| | tctcaactccagcatgtacagggagagctgcaaggggaaactcagcaagtggctcccattccgcctcagctctattgtagcctccctcgtcctcctgcacgtctttgactccttgaaac |
| | ccccatcccaagtcgagctggtcttctacgtcttgtccttctgcaagagtgcggtagtgcccctggcatccgtcagtgtcatcccctactgcctcgcccaggtcctgggccagccgca |
| | caagaagtcgttgtaagagatgtggagtcttcggtgtttaaagtc |
| Human G6PC | atggaggaaggaatgaatgttctccatgactttgggatccagtcaacacattacctccaggtgaattaccaagactcccaggactggttcatcttggtgtccgtgatcgcagacctcag |
| transgene (3) | gaatgccttctacgtcctcttccccatctggttccatcttcaggaagctgtgggcattaaactcctttgggtagctgtgattggagactggctcaacctcgtctttaagtggattctctttgga |
| (SEQ ID NO: 18) | cagcgtccatactggtgggttttggatactgactactacagcaacacttccgtgcccctgataaagcagttccctgtaacctgtgagactggaccagggagcccctctggccatgcca |
| | tgggcacagcaggtgtatactacgtgatggtcacatctactctttccatctttcagggaaagataaagccgacctacagatttcggtgcttgaatgtcattttgtggttgggattctgggct |
| | gtgcagctgaatgtctgtctgtcacgaatctaccttgctgctcattttcctcatcaagttgttgctggagtcctgtcaggcattgctgttgcagaaactttcagccacatccacagcatctat |
| | aatgccagcctcaagaaatattttctcattaccttcttcctgttcagcttcgccatcggattttatctgctgctcaagggactgggtgtagacctcctgtggactctggagaaagcccagag |
| | gtggtgcgagcagccagaatgggtccacattgacaccacaccctttgccagcctcctcaagaacctgggcacgctctttggcctggggctggctctcaactccagcatgtacaggg |
| | agagctgcaaggggaaactcagcaagtggctcccattccgcctcagctctattgtagcctccctcgtcctcctgcacgtctttgactccttgaaacccccatcccaagtcgagctggtc |
| | ttctacgtcttgtccttctgcaagagtgcggtagtgcccctggcatccgtcagtgtcatcccctactgcctcgcccaggtcctgggccagccgcacaagaagtcgttgtaa |
| Canine G6PC | atggaggaaggaatggatgtgctccatgactttggaatccagtcaacgcactacctccaggtgaattaccaggactctcaggattggttcatcttggtgtccgtgattgcagacctcag |
| transgene with | gaatgccttctatgtcctctttcccatctggttccatctgcgtgaagctgtgggcatcaaacttctctgggtagctgtgattggagactggctcaacctcgtctttaaatggattctgtttgga |
| A > G mutation | cagcgtccatactggtgggtcatggacaccgactactatagcaacacctctgtgccactgataaagcaatttccagttacctgtgaaactggaccagggagtccctctggtcatgccat |
| (G) | gggtacagcaggtgtatactacgtgatggtcacatctaccctttctatctttcgggggagaaaaaggccaacctacagatttcggtgcttgaatatccttctgtggttgggattctgggct |
| (SEQ ID NO: 19) | gtgcagctgaacgtctgtctgtcccgaatctaccttgctgctcatttcccccatcaggttgttgctggagtcctgtcaggcattgctgttgctgaaactttccgccacatccagagcatcta |
| | caatgccagcctcaagaaatattttctcattactttcttcctgttcagttttgccattggattttacctgctgctcaaggggctgggtgtggacctcctgtggacactggaaaaagccagga |
| | gatggtgtgagcggccggaatgggttcacattgacaccacaccctttgccagccttctcaagaacgtggggaccctctttggcctgggggtggctctcaactccagcatgtacaggg |
| | aaagctgcaagggcaagcttagcaagtggttcccattccgcctcagctgcattgtggtgtctctcatcctcctgcacctctttgactctttgaaacccccatcccaaactgagctgatctt |
| | ctacaccttgtccttctgcaagagtgcagcagtgcccctggcatctgtcagcctcatcccctactgccttgcccgggtcttcgaccagccagacaagaagtctttgtaa |
| Human min | cctgtgtctctggcctggtttcggggaccaggagggcagacccttgcactgccaagaagcatgccaaagttaatcattggccctgctgagtacatggccgatcaggctgtttttgtgtg |
| G6PC promoter | cctgtttttctattttacgtaaatcaccctgaacatgtttgcatcaacctactggtgatgcacctttgatcaatacattttagacaaacgtggtttttgagtccaaagatcagggctgggttgac |
| (297 bp) | ctgaatactggatacagggcatataaaacaggggcaaggcacagactca |
| (SEQ ID NO: 20) | |
| Human min | ctctgtcctgtgtctctggcctggtttcggggaccaggagggcagacccttgcactgccaagaagcatgccaaagttaatcattggccctgctgagtacatggccgatcaggctgtttt |
| G6PC promoter | tgtgtgcctgtttttctattttacgtaaatcaccctgaacatgtttgcatcaacctactggtgatgcacctttgatcaatacattttagacaaacgtggtttttgagtccaaagatcagggctgg |
| (303 bp) | gttgacctgaatactggatacagggcatataaaacaggggcaaggcacagactcatagc |
| (SEQ ID NO: 21) | |
| Human min | ctctgtcctgtgtctctggcctggtttcggggaccaggagggcagacccttgcactgccaagaagcatgccaaagttaatcattggccctgctgagtacatggccgatcaggctgtttt |
| G6PC promoter | tgtgtgcctgtttttctattttacgtaaatcaccctgaacatgtttgcatcaacctactggtgatgcacctttgatcaatacattttagacaaacgtggtttttgagtccaaagatcagggctgg |
| (356 bp) | gttgacctgaatactggatacagggcatataaaacaggggcaaggcacagactcatagcagagcaatcaccaccaagcctggaataactgcaagggctctgctgacatcttc |
| (SEQ ID NO: 22) | |
| Full Human | agctcaggaattcaagaccagcctgggcaacatggaaaaaccccatctctacaaaagatagaaaaattagccaggcatggtggcgtgtgcctgtggtcccagctactcaggaggct |
| G6PC promoter | gaggtgggaggatcacattagcccaggaggttgaggctgcagtgagccgtgattatgccactgcactccagcctgggagacagagtgagaccctgtttcaaaaaaaagagagag |
| (1284 bp) | aaaatttaaaaaagaaaacaacaccaagggctgtaactttaaggtcattaaatgaattaatcactgcattcaaaaacgattactttctggccctaagagacatgaggccaataccagga |
| (SEQ ID NO: 23) | agggggttgatctcccaaaccagaggcagaccctagactctaatacagttaaggaaagaccagcaagatgatagtccccaatacaatagaagttactatattttatttgttgtttttctttt |
| | gttttgttttgttttgttttgttttgttttagagactggggtcttgctcgattgcccaggctgtagtgcagcggtgggacaatagctcactgcagactccaactcctgggctcaagcaatcctc |
| | ctgcctcagcctcctgaatagctgggactacaagggtacaccatcacacacaccaaaacaattttttaaatttttgtgtagaaacgagggtcttgctttgttgcccaggctggtctccaac |
| | tcctggcttcaagggatcctcccacctcagcctcccaaattgctgggattacaggtgtgagccaccacaaccagccagaactttactaattttaaaattaagaacttaaaacttgaatag |
| | ctagagcaccaagatttttctttgtccccaaataagtgcagttgcaggcatagaaaatctgacatctttgcaagaatcatcgtggatgtagactctgtcctgtgtctctggcctggtttcgg |
| | ggaccaggagggcagacccttgcactgccaagaagcatgccaaagttaatcattggccctgctgagtacatggccgatcaggctgtttttgtgtgcctgtttttctattttacgtaaatca |
| | ccctgaacatgtttgcatcaacctactggtgatgcacctttgatcaatacattttagacaaacgtggtttttgagtccaaagatcagggctgggttgacctgaatactggatacagggcat |
| | ataaaacaggggcaaggcacagactcatagcagagcaatcaccaccaagcctggaataactgcaagggctctgctgacatcttc |
| Human G6PC | cctgtgtctctggcctggtttcggggaccaggagggcagacccttgcactgccaagaagcatgccaaagttaatcattggccctgctgagtacatggccgatcaggctgtttttgtgtg |
| transgene with | cctgtttttctattttacgtaaatcaccctgaacatgtttgcatcaacctactggtgatgcacctttgatcaatacattttagacaaacgtggtttttgagtccaaagatcagggctgggttgac |
| human G6PC | ctgaatactggatacagggcatataaaacaggggcaaggcacagactcatagcagagcaatcaccaccaagcctggaataactgcaagggctctgctgacatcttcctgaggtgc |
| promoter (297 | caaggaaatgaggatggaggaaggaatgaatgttctccatgactttgggatccagtcaacacattacctccaggtgaattaccaagactcccaggactggttcatcttggtgtccgtg |
| bp) | atcgcagacctcaggaatgccttctacgtcctcttccccatctggttccatcttcaggaagctgtgggcattaaactcctttgggtagctgtgattggagactggctcaacctcgtctttaa |
| (SEQ ID NO: 24) | gtggattctctttggacagcgtccatactggtgggttttggatactgactactacagcaacacttccgtgcccctgataaagcagttccctgtaacctgtgagactggaccagggagcc |
| | cctctggccatgccatgggcacagcaggtgtatactacgtgatggtcacatctactctttccatctttcagggaaagataaagccgacctacagatttcggtgcttgaatgtcattttgtg |
| | gttgggattctgggctgtgcagctgaatgtctgtctgtcacgaatctaccttgctgctcattttcctcatcaagttgttgctggagtcctgtcaggcattgctgttacagaaactttcagcca |
| | catccacagcatctataatgccagcctcaagaaatattttctcattaccttcttcctgttcagcttcgccatcggattttatctgctgctcaagggactgggtgtagacctcctgtggactctg |
| | gagaaagcccagaggtggtgcgagcagccagaatgggtccacattgacaccacaccctttgccagcctcctcaagaacctgggcacgctctttggcctggggctggctctcaact |
| | ccagcatgtacagggagagctgcaaggggaaactcagcaagtggctcccattccgcctcagctctattgtagcctccctcgtcctcctgcacgtctttgactccttgaaacccccatc |
| | ccaagtcgagctggtcttctacgtcttgtccttctgcaagagtgcggtagtgcccctggcatccgtcagtgtcatcccctactgcctcgcccaggtcctgggccagccgcacaagaag |
| | tcgttgtaagagatgtggagtcttcggtgtttaaagtc |
| 5′ homology arm | cactcagtttgttcccaaatcacttttattctgtgtgtggagagaaacaggaggtcagaggtatcccttcctcttcgtcagagcaaatggttctattacactagggctctgtctttatggtctc |
| (mouse 1) with | ctttaagttatacttttaacttcttttggcttaaagtacctatctccaactgtgatccacgtgaacttggtgaaagtccaagagagtgagcatttccaggacaacaaagccctactgctgtgt |
| mutated PAM | attttgtatattttaaattaataacttaaaaggtcacttccggcagtagcaaacttccctttgtcttcaaatagatataattgcagacttcaaaaaacagatgcccttgcaagagtcatggttg |
| (G > A mutation), | aaacagactctgccctgggcctctggcctggcttcaaggaccaggaaggagggcagcctctagcactgtcaagcagtgtgcccaagttaataattggctctgccaatggcgatcag |
| (A). | gctgtttttgtgtgcctgttttgctattttacgtaaatcaccctgaacatgtttgcatcaacctactgatgatgcacctttgatcaatagattttagacaaaagtggttttttgagtccaaagatca |
| (SEQ ID NO: 25) | gggctgggttgacctacagactgaatccagggcatataaaacaggggcaaggcacagactgatagcagagggatcaagccaaccgggcttggactcactgcacgggctctgctg |
| | gcagcttcctgaggtaccaagggaggaagctcgagggaaggaatgaacattctccatgactttgggatccagtcgactcgctatctccaagtgaattaccaagactcccaggactg |
| | gttcatccttgtgtctgtgattgctgacctgaggaacgccttctatgtcgactttcccatctagatc |
| 3′ homology arm | tctagatcacgcgtaaagagactgtgggcatcaatctcctctgggtggcagtggtcggagactggttcaacctcgtcttcaagtggtaagaaccatcccgaggggtgatgggtagag |
| (mouse 1) with | aaatggctgtcctccagcaggggtaccccatactgttcaccaggctacaggtaagtccccattggcttgagagtctctacagaatcccacctgtggctggcaatgaggaatggctattt |
| mutated PAM | tgtcagacctgagggatggaaagacagctgagtttaatgggaagtctgagtgtagcctttactctggtttatttgactttattatttagagttagggtcttgtgtagcccaggctggcctca |
| (G > A mutation), | aacttacaaattaagagacaacgaccttgaattgctcaacttctgagtgctgagattacaggtgtgtgttcccacgtgtagtttagcatgctagggattgaacctggggcttggagcatg |
| (A) | agagcaaggactcccccatgaggctatatcccagctcctgagcacagatgttaaaaaaacccttttactactatttctatgggtgctatagaaacagatgtgggaaagaaggggggg |
| (SEQ ID NO: 26) | gggagtgatttctaaccttttggttaagcggtgaaaattgatacggatagccaggaccttccaactggtgaattacataaactcactattctagttttagaatctataaactttggttctctcc |
| | aggatcttaataatatgcacaatcaacgtttattgatcagctactatgtgccaggctcgcactgtctctccctgtgtctgtctcagtctgtgtgtgtgtttgtgtgtgtgcgcacgtgttcatct |
| | gcgtacatgcatgtggctgtgtttgtggaagccagtgcagtcattcctcaaaacttattaagataaagtctctgattgaactggagctccggagtaaatttgaccaatgagcactggagg |
| | tctacctgcctgtattcccatagctatatgccaccatg |
| 5′ homology arm | cactcagtttgttcccaaatcacttttattctgtgtgtggagagaaacaggaggtcagaggtatcccttcctcttcgtcagagcaaatggttctattacactagggctctgtctttatggtctc |
| (mouse 2) | ctttaagttatacttttaacttcttttggcttaaagtacctatctccaactgtgatccacgtgaacttggtgaaagtccaagagagtgagcatttccaggacaacaaagccctactgctgtgt |
| (SEQ ID NO: 27) | attttgtatattttaaattaataacttaaaaggtcacttccggcagtagcaaacttccctttgtcttcaaatagatataattgcagacttcaaaaaacagatgcccttgcaagagtcatggttg |
| | aaacagactctgccctgggcctctggcctggcttcaaggaccaggaaggagggcagcctctagcactgtcaagcagtgtgcccaagttaataattggctctgccaatggcgatcag |
| | gctgtttttgtgtgcctgttttgctattttacgtaaatcaccctgaacatgtttgcatcaacctactgatgatgcacctttgatcaatagattttagacaaaagtggttttttgagtccaaagatca |
| | gggctgggttgacctacagactgaatccagggcatataaaacaggggcaaggcacagactgatagcagagggatcaagccaaccgggcttggactcactgcacgggctctgctg |
| | gcagcttcctgaggtaccaagggaggaagc |
| 3′ homology arm | ctttcccatctagatccatcttaaagagactgtgggcatcaatctcctctgggtggcagtggtcggagactggttcaacctcgtcttcaagtggtaagaaccatcccgaggggtgatgg |
| (mouse 2) | gtagagaaatggctgtcctccagcaggggtaccccatactgttcaccaggctacaggtaagtccccattggcttgagagtctctacagaatcccacctgtggctggcaatgaggaat |
| (SEQ ID NO: 28) | ggctattttgtcagacctgagggatggaaagacagctgagtttaatgggaagtctgagtgtagcctttactctggtttatttgactttattatttagagttagggtcttgtgtagcccaggct |
| | ggcctcaaacttacaaattaagagacaacgaccttgaattgctcaacttctgagtgctgagattacaggtgtgtgttcccacgtgtagtttagcatgctagggattgaacctggggcttg |
| | gagcatgagagcaaggactcccccatgaggctatatcccagctcctgagcacagatgttaaaaaaacccttttactactatttctatgggtgctatagaaacagatgtgggaaagaag |
| | gggggggggagtgatttctaaccttttggttaagcggtgaaaattgatacggatagccaggaccttccaactggtgaattacataaactcactattctagttttagaatctataaactttgg |
| | ttctctccaggatcttaataatatgcacaatcaacgtttattgatcagctactatgtgccaggctcgcactgtctctccctgtgtctgtctcagtctgtgtgtgtgtttgtgtgtgtgcgcacgt |
| | gttcatctgcgtacatgcatgtggctgtgtttgtggaagccagtgcagtcattcctcaaaacttattaagataaagtctctgattgaactggagctccggagtaaatttgaccaatgagca |
| | ctggaggtctacctgcctgtattcccatagctatatgccaccatg |
| 5′ homology arm | atgctaagtgaaataagacagaaaaagacaaatatcatatggtttcacttatatgtagggtctaaaaagcgaaacaaatgagtaaacaaacacaaaaagcagactcaaacctactatg |
| (canine) with | tggagaacaaattgatacttgcccaagaggaggagcagtgagaggatggggaaaatgggtgaaggggaatgagagatacaggcttccagacctcatggaatgagtaagtcactg |
| exon 1 cf G6PC | gaatgaaaggcacagcacagggaatacagtcaatggtattgtaacagtgttgtacagtgacaggtggtagctatactgtggtgaacgaagcataatgcacagagatgttgaataact |
| cDNA bolded | atgttgtacacctgaaactaatgttagattagtttcattatgtaacatcagtttcagctacattctaatacactttaaaataatacagtagtcctttccagagtccccagtcaccaatttgctcgt |
| and A > G | gtttttatttagaaccccatattcattacattgtttttgcatgaacacacattcattctactgcttaaacttatgtgttaaaaccccaaaggtcctaacttggaggacattgcatgaattaatcagt |
| Mutation | gcattcaaagatgattacttcctggccctaagatatatagaatcaacactaggaagaaggtttgatctcccaaaccagaggcacttcacagacactatgcagttaagaatacacagcaa |
| underlined and | ggtaggtgatggttcccagtacaatagaacttagcgaatatgtttttttgagattttatttatgagagacacacagagagaggcagagacacaggtagaaggagaagcaggctccctg |
| bolded (G) | aagggagcccgatgccggactcaatcccaggaccctgagatcacaacctgagccaaaggtagatgctcatccactgagccatgcaggtaccccgaacttagcatattttaaaagta |
| (SEQ ID NO: 29) | atatgtttaaaacatgctcactagggtaccaagacttttctttgtcctcaaataagtgcacttgcagacagaaaatcaggatctttgcaagaaccatggcggatttagactctgtcctgtgt |
| | ctccggcccagtttcggagccaggagggcagcccttgcactatcaagtagcatgccaaagttaatcattggccctgccaagtacatggcagatcaggctgtttttgtgtgcctgtttttc |
| | tattttacgtaaatcaccctgaacatgtttgcatcaacctcctggtgatgtacctttgatcaatagattttagacaaaagtggtttttgagtccaaagatcagggctgggttgacctgcagac |
| | tggatacagagtgtataaaaacaggggcaaggcgcaggctgatagcagagcaatcgccaagtcggtaccgagctcggaaagggcgaattccagcacactggcggccgttacta |
| | gtggatccaaggatggaggaaggaatggatgtgctccatgactttggaatccagtcaacgcactacctccaggtgaattaccaggactctcaggattggttcatcttggtgt |
| | ccgtgattgcagacctcaggaatgccttctatgtcctctttcccatctggttccatctgcgtgaagctgtgggcatcaaacttctctgggtagctgtgattggagactggctca |
| | acctcgtctttaaatg |
| 5′ homology arm | atgctaagtgaaataagacagaaaaagacaaatatcatatggtttcacttatatgtagggtctaaaaagcgaaacaaatgagtaaacaaacacaaaaagcagactcaaacctactatg |
| (canine) without | tggagaacaaattgatacttgcccaagaggaggagcagtgagaggatggggaaaatgggtgaaggggaatgagagatacaggcttccagacctcatggaatgagtaagtcactg |
| exon 1 | gaatgaaaggcacagcacagggaatacagtcaatggtattgtaacagtgttgtacagtgacaggtggtagctatactgtggtgaacgaagcataatgcacagagatgttgaataact |
| (SEQ ID NO: 30) | atgttgtacacctgaaactaatgttagattagtttcattatgtaacatcagtttcagctacattctaatacactttaaaataatacagtagtcctttccagagtccccagtcaccaatttgctcgt |
| | gtttttatttagaaccccatattcattacattgtttttgcatgaacacacattcattctactgcttaaacttatgtgttaaaaccccaaaggtcctaacttggaggacattgcatgaattaatcagt |
| | gcattcaaagatgattacttcctggccctaagatatatagaatcaacactaggaagaaggtttgatctcccaaaccagaggcacttcacagacactatgcagttaagaatacacagcaa |
| | ggtaggtgatggttcccagtacaatagaacttagcgaatatgtttttttgagattttatttatgagagacacacagagagaggcagagacacaggtagaaggagaagcaggctccctg |
| | aagggagcccgatgccggactcaatcccaggaccctgagatcacaacctgagccaaaggtagatgctcatccactgagccatgcaggtaccccgaacttagcatattttaaaagta |
| | atatgtttaaaacatgctcactagggtaccaagacttttctttgtcctcaaataagtgcacttgcagacagaaaatcaggatctttgcaagaaccatggcggatttagactctgtcctgtgt |
| | ctccggcccagtttcggagccaggagggcagcccttgcactatcaagtagcatgccaaagttaatcattggccctgccaagtacatggcagatcaggctgtttttgtgtgcctgtttttc |
| | tattttacgtaaatcaccctgaacatgtttgcatcaacctcctggtgatgtacctttgatcaatagattttagacaaaagtggtttttgagtccaaagatcagggctgggttgacctgcagac |
| | tggatacagagtgtataaaaacaggggcaaggcgcaggctgatagcagagcaatcgccaagtcggtaccgagctcggaaagggcgaattccagcacactggcggccgttacta |
| | gtggatccaagg |
| 3′ homology arm | ttctctgggtagctgtgattggagactggctcaacctcgtctttaaatggtgagtaagagccagacagagaggagatcggcaagggaagaggctggtatagtctctggaaatgtctg |
| (canine) with | acatcagaaattgccttctccgtgctagtcgggaagccacaggttactcccctcctgactctggatcatctacctaaagagggaagacagcgaaaatcttgttgatgaattgaagatac |
| GA > CT | aggcaaggctatttcatatggatggaaggccagccggacttagtgaaaagtctaaagtatataggctataagcaaagaagcacagttgtattactttgaagcagaataggtaacataa |
| mutation | agataggtgtatgaaagatgatgtatgaaaacaggaaacaagtatataatttaaaaacccactgcatggagaaagaagaagtaacccagagaactgggctctaacccttgttcggcc |
| (antisense) | acctaccagctgtatgtcctgggaaagtcagagcaactctctgagtttctgtcttacaatctgtaaacttaaattatccttgggagcttaataataattctagttaacatttattgaacatttagt |
| bolded and | aggtactaggccatgtgccaagtgcttttctgtgaggtagttactactgtattatagatgaagagacagagccattacgataagggaccagaactagatccacatggtggtcaaaggc |
| underlined. | agaggcagcattttaaggccagacaggctagtttcagaggcaaagctcttaaggattatgctgtattcattgctgacaaattgactttttcagggagcaggaggaagccaaattttaatg |
| (AG) | tttgtcaatttctctggtgtatatattcctattggggccacttttgatgggtcaagcttgcaaaaatcctgaatattttacaattgtcagccaataccatatgattctagctcattagtggcttgg |
| (SEQ ID NO: 31) | cctctatttcctcatctagaaaataaagagcttgggccagctaaaacctttaccatccctaaactatctatg |
| 5′ homology arm | gagctcaggaattcaagaccagcctgggcaacatggaaaaaccccatctctacaaaagatagaaaaattagccaggcatggtggcgtgtgcctgtggtcccagctactcaggagg |
| spCas9 Donor | ctgaggtgggaggatcacattagcccaggaggttgaggctgcagtgagccgtgattatgccactgcactccagcctgggagacagagtgagaccctgtttcaaaaaaaagagaga |
| (Human) with | gaaaatttaaaaaagaaaacaacaccaagggctgtaactttaaggtcattaaatgaattaatcactgcattcaaaaacgattactttctggccctaagagacatgaggccaataccagg |
| exon 1 cf G6PC | aagggggttgatctcccaaaccagaggcagaccctagactctaatacagttaaggaaagaccagcaagatgatagtccccaatacaatagaagttactatattttatttgttgtttttcttt |
| cDNA bolded | tgttttgttttgttttgttttgttttgttttagagactggggtcttgctcgattgcccaggctgtagtgcagcggtgggacaatagctcactgcagactccaactcctgggctcaagcaatcct |
| (SEQ ID NO: 32) | cctgcctcagcctcctgaatagctgggactacaagggtacaccatcacacacaccaaaacaattttttaaatttttgtgtagaaacgagggtcttgctttgttgcccaggctggtctccaa |
| | ctcctggcttcaagggatcctcccacctcagcctcccaaattgctgggattacaggtgtgagccaccacaaccagccagaactttactaattttaaaattaagaacttaaaacttgaata |
| | gctagagcaccaagatttttctttgtccccaaataagtgcagttgcaggcatagaaaatctgacatctttgcaagaatcatcgtggatgtagactctgtcctgtgtctctggcctggtttcg |
| | gggaccaggagggcagacccttgcactgccaagaagcatgccaaagttaatcattggccctgctgagtacatggccgatcaggctgtttttgtgtgcctgtttttctattttacgtaaatc |
| | accctgaacatgtttgcatcaacctactggtgatgcacctttgatcaatacattttagacaaacgtggtttttgagtccaaagatcagggctgggttgacctgaatactggatacagggca |
| | tataaaacaggggcaaggcacagactcatagcagagcaatcaccaccaagcctggaataactgcaagggctctgctgacatcttcctgaggtgccaaggaaatgaggatggagg |
| | aaggaatgaatgttctccatgactttgggatccagtcaacacattacctccaggtgaattaccaagactcccaggactggttcatcttggtgtccgtgatcgcagacctcag |
| | gaatgccttctacgtcctcttccccatctggttccatcttcaggaagctgtgggcattaaactcctttgggtagctgtgattggagactggctcaacctcgtctttaagtg |
| 5′ homology arm | gagctcaggaattcaagaccagcctgggcaacatggaaaaaccccatctctacaaaagatagaaaaattagccaggcatggtggcgtgtgcctgtggtcccagctactcaggagg |
| spCas9 Donor | ctgaggtgggaggatcacattagcccaggaggttgaggctgcagtgagccgtgattatgccactgcactccagcctgggagacagagtgagaccctgtttcaaaaaaaagagaga |
| (Human) without | gaaaatttaaaaaagaaaacaacaccaagggctgtaactttaaggtcattaaatgaattaatcactgcattcaaaaacgattactttctggccctaagagacatgaggccaataccagg |
| exon 1 | aagggggttgatctcccaaaccagaggcagaccctagactctaatacagttaaggaaagaccagcaagatgatagtccccaatacaatagaagttactatattttatttgttgtttttcttt |
| (SEQ ID NO: 33) | tgttttgttttgttttgttttgttttgttttagagactggggtcttgctcgattgcccaggctgtagtgcagcggtgggacaatagctcactgcagactccaactcctgggctcaagcaatcct |
| | cctgcctcagcctcctgaatagctgggactacaagggtacaccatcacacacaccaaaacaattttttaaatttttgtgtagaaacgagggtcttgctttgttgcccaggctggtctccaa |
| | ctcctggcttcaagggatcctcccacctcagcctcccaaattgctgggattacaggtgtgagccaccacaaccagccagaactttactaattttaaaattaagaacttaaaacttgaata |
| | gctagagcaccaagatttttctttgtccccaaataagtgcagttgcaggcatagaaaatctgacatctttgcaagaatcatcgtggatgtagactctgtcctgtgtctctggcctggtttcg |
| | gggaccaggagggcagacccttgcactgccaagaagcatgccaaagttaatcattggccctgctgagtacatggccgatcaggctgtttttgtgtgcctgtttttctattttacgtaaatc |
| | accctgaacatgtttgcatcaacctactggtgatgcacctttgatcaatacattttagacaaacgtggtttttgagtccaaagatcagggctgggttgacctgaatactggatacagggca |
| | tataaaacaggggcaaggcacagactcatagcagagcaatcaccaccaagcctggaataactgcaagggctctgctgacatcttcctgaggtgccaaggaaatgagg |
| 3′ homology arm | gtaagaaccatatagagaggagatcagcaagaaaagaggctggcattcgctctcgcaatgtctgtccatcagaagttgctttccccaggctattcaggaagccacgggctactcatg |
| (spCas9 donor - | cttccaacccctctctctgactttggatcatctacataaagggggaagacagaaaaaatcctaccagtgagttgaaaatacaggaaagcctatttcatatgggttaaagggtaggacag |
| human) | ttgaatttcgtgaaaagtctgagttatataggctttgagcaaagagttttattagtatgaagcagaagaggtaacataaagaaagatgtatggggccaggcatggtggctcacacctgta |
| (SEQ ID NO: 34) | atcccagcactttgggaggccgaggtgggcgaatcactcctgggtgaactcaggagttcaagaccagcctgggcaacatggcgaaactccatctctacaaaaacattacgaaaatt |
| | agctgggcgtgttggtgctgtagtcccagctactcaggaggctgaggtgagaggcggaggaggttgcagtgagtcaagatcatgccactgcactccagcctgggcaacagagta |
| | agaccctgtctcaaaaaaaaaaaaaagatagatgatgtatgctgtatgaaaaaaggaaacacacagatgattcaacagcctgttttgtggggtaatgaaaagtcaccctgggaactg |
| | ggctccagccctcgttctgccacccaccaactacatgtccttggcaagtcatatcaattatctgagtttctgttttataatctacaaataggttatctctggcagcttaataataatcagggtt |
| | aacatttattaaacagtgtgtgccagtccatgtgctatgtgcttttctgtgaggtagttactgctatttacagaaacagtagatgcagagaccaaggtgctgagttaaatgattaggccaac |
| | aaggttagtacatgccgagccaggatggaagcccaggtaggcaggctggcttccgcgg |
| 3′ homology arm | gtaagaaccatatagagaggagatcagcaagaaaagaggctggcataggctctcgcaatgtctgtccatcagaagttgctttccccaggctattcaggaagccacgggctactcat |
| (SaCas9 donor) | gcttccaacccctctctctgactttggatcatctacataaagggggaagacagaaaaaatcctaccagtgagttgaaaatacaggaaagcctatttcatatgggttaaagggtaggaca |
| with GA > CT | gttgaatttcgtgaaaagtctgagttatataggctttgagcaaagagttttattagtatgaagcagaagaggtaacataaagaaagatgtatggggccaggcatggtggctcacacctgt |
| mutation | aatcccagcactttgggaggccgaggtgggcgaatcactcctgggtgaactcaggagttcaagaccagcctgggcaacatggcgaaactccatctctacaaaaacattacgaaaat |
| (antisense) | tagctgggcgtgttggtgctgtagtcccagctactcaggaggctgaggtgagaggcggaggaggttgcagtgagtcaagatcatgccactgcactccagcctgggcaacagagta |
| bolded and | agaccctgtctcaaaaaaaaaaaaaagatagatgatgtatgctgtatgaaaaaaggaaacacacagatgattcaacagcctgttttgtggggtaatgaaaagtcaccctgggaactg |
| underlined. | ggctccagccctcgttctgccacccaccaactacatgtccttggcaagtcatatcaattatctgagtttctgttttataatctacaaataggttatctctggcagcttaataataatcagggtt |
| (SEQ ID NO: 35) | aacatttattaaacagtgtgtgccagtccatgtgctatgtgcttttctgtgaggtagttactgctatttacagaaacagtagatgcagagaccaaggtgctgagttaaatgattaggccaac |
| | aaggttagtacatgccgagccaggatggaagcccaggtaggcaggctggcttccgcggcaatgctcttatgaactatgttacgtccagtgctg |
| Donor Nucleic | cactcagtttgttcccaaatcacttttattctgtgtgtggagagaaacaggaggtcagaggtatcccttcctcttcgtcagagcaaatggttctattacactagggctctgtctttatggtctc |
| Acid (murine 1) | ctttaagttatacttttaacttcttttggcttaaagtacctatctccaactgtgatccacgtgaacttggtgaaagtccaagagagtgagcatttccaggacaacaaagccctactgctgtgt |
| (SEQ ID NO: 36) | attttgtatattttaaattaataacttaaaaggtcacttccggcagtagcaaacttccctttgtcttcaaatagatataattgcagacttcaaaaaacagatgcccttgcaagagtcatggttg |
| | aaacagactctgccctgggcctctggcctggcttcaaggaccaggaaggagggcagcctctagcactgtcaagcagtgtgcccaagttaataattggctctgccaatggcgatcag |
| | gctgtttttgtgtgcctgttttgctattttacgtaaatcaccctgaacatgtttgcatcaacctactgatgatgcacctttgatcaatagattttagacaaaagtggttttttgagtccaaagatca |
| | gggctgggttgacctacagactgaatccagggcatataaaacaggggcaaggcacagactgatagcagagggatcaagccaaccgggcttggactcactgcacgggctctgctg |
| | gcagcttcctgaggtaccaagggaggaagctcgagggaaggaatgaacattctccatgactttgggatccagtcgactcgctatctccaagtgaattaccaagactcccaggactg |
| | gttcatccttgtgtctgtgattgctgacctgaggaacgccttctatgtcgactttcccatctagatcacgcgtcctgtgtctctggcctggtttcggggaccaggagggcagacccttgca |
| | ctgccaagaagcatgccaaagttaatcattggccctgctgagtacatggccgatcaggctgtttttgtgtgcctgtttttctattttacgtaaatcaccctgaacatgtttgcatcaacctact |
| | ggtgatgcacctttgatcaatacattttagacaaacgtggtttttgagtccaaagatcagggctgggttgacctgaatactggatacagggcatataaaacaggggcaaggcacagac |
| | tcatagcagagcaatcaccaccaagcctggaataactgcaagggctctgctgacatcttcctgaggtgccaaggaaatgaggatggaggaaggaatgaatgttctccatgactttgg |
| | gatccagtcaacacattacctccaggtgaattaccaagactcccaggactggttcatcttggtgtccgtgatcgcagacctcaggaatgccttctacgtcctcttccccatctggttccat |
| | cttcaggaagctgtgggcattaaactcctttgggtagctgtgattggagactggctcaacctcgtctttaagtggattctctttggacagcgtccatactggtgggttttggatactgacta |
| | ctacagcaacacttccgtgcccctgataaagcagttccctgtaacctgtgagactggaccagggagcccctctggccatgccatgggcacagcaggtgtatactacgtgatggtcac |
| | atctactctttccatctttcagggaaagataaagccgacctacagatttcggtgcttgaatgtcattttgtggttgggattctgggctgtgcagctgaatgtctgtctgtcacgaatctacctt |
| | gctgctcattttcctcatcaagttgttgctggagtcctgtcaggcattgctgttacagaaactttcagccacatccacagcatctataatgccagcctcaagaaatattttctcattaccttctt |
| | cctgttcagcttcgccatcggattttatctgctgctcaagggactgggtgtagacctcctgtggactctggagaaagcccagaggtggtgcgagcagccagaatgggtccacattga |
| | caccacaccctttgccagcctcctcaagaacctgggcacgctctttggcctggggctggctctcaactccagcatgtacagggagagctgcaaggggaaactcagcaagtggctc |
| | ccattccgcctcagctctattgtagcctccctcgtcctcctgcacgtctttgactccttgaaacccccatcccaagtcgagctggtcttctacgtcttgtccttctgcaagagtgcggtagt |
| | gcccctggcatccgtcagtgtcatcccctactgcctcgcccaggtcctgggccagccgcacaagaagtcgttgtaagagatgtggagtcttcggtgtttaaagtcgaattcgtttaaac |
| | ccgctgatcagcctcgactgtgccttctagttgccagccatctgttgtttgcccctcccccgtgccttccttgaccctggaaggtgccactcccactgtcctttcctaataaaatgaggaa |
| | attgcatcgcattgtctgagtaggtgtcattctattctggggggtggggggggcaggacagcaagggggaggattgggaagacaatagcaggcatgctggggatgcggtgggct |
| | ctatggcttctgaggcggaaagaaccagctggaattctgcagatctcgagtctagatcacgcgtaaagagactgtgggcatcaatctcctctgggtggcagtggtcggagactggtt |
| | caacctcgtcttcaagtggtaagaaccatcccgaggggtgatgggtagagaaatggctgtcctccagcaggggtaccccatactgttcaccaggctacaggtaagtccccattggct |
| | tgagagtctctacagaatcccacctgtggctggcaatgaggaatggctattttgtcagacctgagggatggaaagacagctgagtttaatgggaagtctgagtgtagcctttactctgg |
| | tttatttgactttattatttagagttagggtcttgtgtagcccaggctggcctcaaacttacaaattaagagacaacgaccttgaattgctcaacttctgagtgctgagattacaggtgtgtgt |
| | tcccacgtgtagtttagcatgctagggattgaacctggggcttggagcatgagagcaaggactcccccatgaggctatatcccagctcctgagcacagatgttaaaaaaaccctttta |
| | ctactatttctatgggtgctatagaaacagatgtgggaaagaaggggggggggagtgatttctaaccttttggttaagcggtgaaaattgatacggatagccaggaccttccaactggt |
| | gaattacataaactcactattctagttttagaatctataaactttggttctctccaggatcttaataatatgcacaatcaacgtttattgatcagctactatgtgccaggctcgcactgtctctcc |
| | ctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtc |
| | tctgattgaactggagctccggagtaaatttgaccaatgagcactggaggtctacctgcctgtattcccatagctatatgccaccatg |
| Donor Nucleic | cactcagtttgttcccaaatcacttttattctgtgtgtggagagaaacaggaggtcagaggtatcccttcctcttcgtcagagcaaatggttctattacactagggctctgtctttatggtctc |
| Acid (murine 2) | ctttaagttatacttttaacttcttttggcttaaagtacctatctccaactgtgatccacgtgaacttggtgaaagtccaagagagtgagcatttccaggacaacaaagccctactgctgtgt |
| (SEQ ID NO: 37) | attttgtatattttaaattaataacttaaaaggtcacttccggcagtagcaaacttccctttgtcttcaaatagatataattgcagacttcaaaaaacagatgcccttgcaagagtcatggttg |
| | aaacagactctgccctgggcctctggcctggcttcaaggaccaggaaggagggcagcctctagcactgtcaagcagtgtgcccaagttaataattggctctgccaatggcgatcag |
| | gctgtttttgtgtgcctgttttgctattttacgtaaatcaccctgaacatgtttgcatcaacctactgatgatgcacctttgatcaatagattttagacaaaagtggttttttgagtccaaagatca |
| | gggctgggttgacctacagactgaatccagggcatataaaacaggggcaaggcacagactgatagcagagggatcaagccaaccgggcttggactcactgcacgggctctgctg |
| | gcagcttcctgaggtaccaagggaggaagctcgaggtgccaaggaaatgaggatggaggaaggaatgaatgttctccatgactttgggatccagtcaacacattacctccaggtga |
| | attaccaagactcccaggactggttcatcttggtgtccgtgatcgcagacctcaggaatgccttctacgtcctcttccccatctggttccatcttcaggaagctgtgggcattaaactcctt |
| | tgggtagctgtgattggagactggctcaacctcgtctttaagtggattctctttggacagcgtccatactggtgggttttggatactgactactacagcaacacttccgtgcccctgataa |
| | agcagttccctgtaacctgtgagactggaccagggagcccctctggccatgccatgggcacagcaggtgtatactacgtgatggtcacatctactctttccatctttcagggaaagata |
| | aagccgacctacagatttcggtgcttgaatgtcattttgtggttgggattctgggctgtgcagctgaatgtctgtctgtcacgaatctaccttgctgctcattttcctcatcaagttgttgctg |
| | gagtcctgtcaggcattgctgttacagaaactttcagccacatccacagcatctataatgccagcctcaagaaatattttctcattaccttcttcctgttcagcttcgccatcggattttatct |
| | gctgctcaagggactgggtgtagacctcctgtggactctggagaaagcccagaggtggtgcgagcagccagaatgggtccacattgacaccacaccctttgccagcctcctcaag |
| | aacctgggcacgctctttggcctggggctggctctcaactccagcatgtacagggagagctgcaaggggaaactcagcaagtggctcccattccgcctcagctctattgtagcctcc |
| | ctcgtcctcctgcacgtctttgactccttgaaacccccatcccaagtcgagctggtcttctacgtcttgtccttctgcaagagtgcggtagtgcccctggcatccgtcagtgtcatcccct |
| | actgcctcgcccaggtcctgggccagccgcacaagaagtcgttgtaagagatgtggagtcttcggtgtttaaagtcgaattctgcagatctcgagtctagagggcccgtttaaacccg |
| | ctgatcagcctcgactgtgccttctagttgccagccatctgttgtttgcccctcccccgtgccttccttgaccctggaaggtgccactcccactgtcctttcctaataaaatgaggaaattg |
| | catcgcattgtctgagtaggtgtcattctattctggggggtggggggggcaggacagcaagggggaggattgggaagacaatagcaggcatgctggggatgcggtgggctctat |
| | ggcttctgaggcggaaagaaccagctggggctctagggggtatccccacgcgccctgtagcggcgcattaagcgcggcgggtgtggtggttacgcgcaaaaacgtcgactttcc |
| | catctagatccatcttaaagagactgtgggcatcaatctcctctgggtggcagtggtcggagactggttcaacctcgtcttcaagtggtaagaaccatcccgaggggtgatgggtaga |
| | gaaatggctgtcctccagcaggggtaccccatactgttcaccaggctacaggtaagtccccattggcttgagagtctctacagaatcccacctgtggctggcaatgaggaatggctat |
| | tttgtcagacctgagggatggaaagacagctgagtttaatgggaagtctgagtgtagcctttactctggtttatttgactttattatttagagttagggtcttgtgtagcccaggctggcctc |
| | aaacttacaaattaagagacaacgaccttgaattgctcaacttctgagtgctgagattacaggtgtgtgttcccacgtgtagtttagcatgctagggattgaacctggggcttggagcat |
| | gagagcaaggactcccccatgaggctatatcccagctcctgagcacagatgttaaaaaaacccttttactactatttctatgggtgctatagaaacagatgtgggaaagaagggggg |
| | ggggagtgatttctaaccttttggttaagcggtgaaaattgatacggatagccaggaccttccaactggtgaattacataaactcactattctagttttagaatctataaactttggttctctc |
| | caggatcttaataatatgcacaatcaacgtttattgatcagctactatgtgccaggctcgcactgtctctccctgtgtctgtctcagtctgtgtgtgtgtttgtgtgtgtgcgcacgtgttcatc |
| | tgcgtacatgcatgtggctgtgtttgtggaagccagtgcagtcattcctcaaaacttattaagataaagtctctgattgaactggagctccggagtaaatttgaccaatgagcactggag |
| | gtctacctgcctgtattcccatagctatatgccaccatg |
| Donor Nucleic | atgctaagtgaaataagacagaaaaagacaaatatcatatggtttcacttatatgtagggtctaaaaagcgaaacaaatgagtaaacaaacacaaaaagcagactcaaacctactatg |
| Acid (canine) | tggagaacaaattgatacttgcccaagaggaggagcagtgagaggatggggaaaatgggtgaaggggaatgagagatacaggcttccagacctcatggaatgagtaagtcactg |
| with | gaatgaaaggcacagcacagggaatacagtcaatggtattgtaacagtgttgtacagtgacaggtggtagctatactgtggtgaacgaagcataatgcacagagatgttgaataact |
| GA > CT | atgttgtacacctgaaactaatgttagattagtttcattatgtaacatcagtttcagctacattctaatacactttaaaataatacagtagtcctttccagagtccccagtcaccaatttgctcgt |
| antisense | gtttttatttagaaccccatattcattacattgtttttgcatgaacacacattcattctactgcttaaacttatgtgttaaaaccccaaaggtcctaacttggaggacattgcatgaattaatcagt |
| mutation tc | gcattcaaagatgattacttcctggccctaagatatatagaatcaacactaggaagaaggtttgatctcccaaaccagaggcacttcacagacactatgcagttaagaatacacagcaa |
| remove PAM in | ggtaggtgatggttcccagtacaatagaacttagcgaatatgtttttttgagattttatttatgagagacacacagagagaggcagagacacaggtagaaggagaagcaggctccctg |
| 3′ homology arm | aagggagcccgatgccggactcaatcccaggaccctgagatcacaacctgagccaaaggtagatgctcatccactgagccatgcaggtaccccgaacttagcatattttaaaagta |
| bolded and | atatgtttaaaacatgctcactagggtaccaagacttttctttgtcctcaaataagtgcacttgcagacagaaaatcaggatctttgcaagaaccatggcggatttagactctgtcctgtgt |
| underlined | ctccggcccagtttcggagccaggagggcagcccttgcactatcaagtagcatgccaaagttaatcattggccctgccaagtacatggcagatcaggctgtttttgtgtgcctgtttttc |
| (GA) | tattttacgtaaatcaccctgaacatgtttgcatcaacctcctggtgatgtacctttgatcaatagattttagacaaaagtggtttttgagtccaaagatcagggctgggttgacctgcagac |
| (SEQ ID NO: 38) | tggatacagagtgtataaaaacaggggcaaggcgcaggctgatagcagagcaatcgccaagtcggtaccgagctcggaaagggcgaattccagcacactggcggccgttacta |
| | gtggatccaaggatggaggaaggaatggatgtgctccatgactttggaatccagtcaacgcactacctccaggtgaattaccaggactctcaggattggttcatcttggtgtccgtgat |
| | tgcagacctcaggaatgccttctatgtcctctttcccatctggttccatctgcgtgaagctgtgggcatcaaacttctctgggtagctgtgattggagactggctcaacctcgtctttaaat |
| | ggattctgtttggacagcgtccatactggtgggtcatggacaccgactactatagcaacacctctgtgccactgataaagcaatttccagttacctgtgaaactggaccagggagtccc |
| | tctggtcatgccatgggtacagcaggtgtatactacgtgatggtcacatctaccctttctatctttcgggggagaaaaaggccaacctacagatttcggtgcttgaatatccttctgtggtt |
| | gggattctgggctgtgcagctgaacgtctgtctgtcccgaatctaccttgctgctcatttcccccatcaggttgttgctggagtcctgtcaggcattgctgttgctgaaactttccgccaca |
| | tccagagcatctacaatgccagcctcaagaaatattttctcattactttcttcctgttcagttttgccattggattttacctgctgctcaaggggctgggtgtggacctcctgtggacactgg |
| | aaaaagccaggagatggtgtgagcggccggaatgggttcacattgacaccacaccctttgccagccttctcaagaacgtggggaccctctttggcctgggggtggctctcaactcc |
| | agcatgtacagggaaagctgcaagggcaagcttagcaagtggttcccattccgcctcagctgcattgtggtgtctctcatcctcctgcacctctttgactctttgaaacccccatcccaa |
| | actgagctgatcttctacaccttgtccttctgcaagagtgcagcagtgcccctggcatctgtcagcctcatcccctactgccttgcccgggtcttcgaccagccagacaagaagtctttg |
| | taaggcgaattctgcagatatccatcacactggcggccgctcgacagcaacgtctatgacctcctaaaggacctagaggaaggcatccaaacgctgatgggggtgagggtggcgc |
| | caggggtccccaatcctggagccccactgactttgagagctgtgttagagaaacactgctgccctctttttagcagtcaggccctgacccaagagaactcaccttattcttcatttcccc |
| | tcgtgaatcctccaggcctttctctacaccctgaaggggagggaggaaaatgaatgaatgagaaagggagggaacagtacccaagcgcttggcctctccttctcttccttcactttgc |
| | agaggctggaagatggcagcccccggactgggcagatcttcaagcagacctacagcaagttcgacacaaactcacacaacgatgacgcactactcaagaactacgggctgctct |
| | actgcttcaggaaggacatggacaaggtcgagacattcctgcgcatcgtgcagtgccgctctgtggagggcagctgtggcttctagctgcccgggggcatccctgtgacccctcc |
| | ccagtgcctctcctggccctggaagttgccactccagtgcccaccagtcttgtcctaataaaattaagttgcatcattttgtctgactaggtgtccttctataatattatggggtggagggg |
| | ggtggtatggagcaagggaatagattctctgggtagctgtgattggagactggctcaacctcgtctttaaatggtgagtaagagccagacagagaggagatcggcaagggaagag |
| | gctggtatAGtctctggaaatgtctgacatcagaaattgccttctccgtgctagtcgggaagccacaggttactcccctcctgactctggatcatctacctaaagagggaagacagc |
| | gaaaatcttgttgatgaattgaagatacaggcaaggctatttcatatggatggaaggccagccggacttagtgaaaagtctaaagtatataggctataagcaaagaagcacagttgtat |
| | tactttgaagcagaataggtaacataaagataggtgtatgaaagatgatgtatgaaaacaggaaacaagtatataatttaaaaacccactgcatggagaaagaagaagtaacccaga |
| | gaactgggctctaacccttgttcggccacctaccagctgtatgtcctgggaaagtcagagcaactctctgagtttctgtcttacaatctgtaaacttaaattatccttgggagcttaataata |
| | attctagttaacatttattgaacatttagtaggtactaggccatgtgccaagtgcttttctgtgaggtagttactactgtattatagatgaagagacagagccattacgataagggaccaga |
| | actagatccacatggtggtcaaaggcagaggcagcattttaaggccagacaggctagtttcagaggcaaagctcttaaggattatgctgtattcattgctgacaaattgactttttcagg |
| | gagcaggaggaagccaaattttaatgtttgtcaatttctctggtgtatatattcctattggggccacttttgatgggtcaagcttgcaaaaatcctgaatattttacaattgtcagccaatacc |
| | atatgattctagctcattagtggcttggcctctatttcctcatctagaaaataaagagcttgggccagctaaaacctttaccatccctaaactatctatg |
| Donor Nucleic | gagctcaggaattcaagaccagcctgggcaacatggaaaaaccccatctctacaaaagatagaaaaattagccaggcatggtggcgtgtgcctgtggtcccagctactcaggagg |
| Acid (Human | ctgaggtgggaggatcacattagcccaggaggttgaggctgcagtgagccgtgattatgccactgcactccagcctgggagacagagtgagaccctgtttcaaaaaaaagagaga |
| SaCas9) with | gaaaatttaaaaaagaaaacaacaccaagggctgtaactttaaggtcattaaatgaattaatcactgcattcaaaaacgattactttctggccctaagagacatgaggccaataccagg |
| GA > CT | aagggggttgatctcccaaaccagaggcagaccctagactctaatacagttaaggaaagaccagcaagatgatagtccccaatacaatagaagttactatattttatttgttgtttttcttt |
| antisense | tgttttgttttgttttgttttgttttgttttagagactggggtcttgctcgattgcccaggctgtagtgcagcggtgggacaatagctcactgcagactccaactcctgggctcaagcaatcct |
| mutation tc | cctgcctcagcctcctgaatagctgggactacaagggtacaccatcacacacaccaaaacaattttttaaatttttgtgtagaaacgagggtcttgctttgttgcccaggctggtctccaa |
| remove PAM in | ctcctggcttcaagggatcctcccacctcagcctcccaaattgctgggattacaggtgtgagccaccacaaccagccagaactttactaattttaaaattaagaacttaaaacttgaata |
| 3′ homology arm | gctagagcaccaagatttttctttgtccccaaataagtgcagttgcaggcatagaaaatctgacatctttgcaagaatcatcgtggatgtagactctgtcctgtgtctctggcctggtttcg |
| bolded and | gggaccaggagggcagacccttgcactgccaagaagcatgccaaagttaatcattggccctgctgagtacatggccgatcaggctgtttttgtgtgcctgtttttctattttacgtaaatc |
| underlined | accctgaacatgtttgcatcaacctactggtgatgcacctttgatcaatacattttagacaaacgtggtttttgagtccaaagatcagggctgggttgacctgaatactggatacagggca |
| (AG) | tataaaacaggggcaaggcacagactcatagcagagcaatcaccaccaagcctggaataactgcaagggctctgctgacatcttcctgaggtgccaaggaaatgaggatggagg |
| (SEQ ID NO: 39) | aaggaatgaatgttctccatgactttgggatccagtcaacacattacctccaggtgaattaccaagactcccaggactggttcatcttggtgtccgtgatcgcagacctcaggaatgcct |
| | tctacgtcctcttccccatctggttccatcttcaggaagctgtgggcattaaactcctttgggtagctgtgattggagactggctcaacctcgtctttaagtggattctctttggacagcgtc |
| | catactggtgggttttggatactgactactacagcaacacttccgtgcccctgataaagcagttccctgtaacctgtgagactggaccagggagcccctctggccatgccatgggcac |
| | agcaggtgtatactacgtgatggtcacatctactctttccatctttcagggaaagataaagccgacctacagatttcggtgcttgaatgtcattttgtggttgggattctgggctgtgcagc |
| | tgaatgtctgtctgtcacgaatctaccttgctgctcattttcctcatcaagttgttgctggagtcctgtcaggcattgctgttgcagaaactttcagccacatccacagcatctataatgcca |
| | gcctcaagaaatattttctcattaccttcttcctgttcagcttcgccatcggattttatctgctgctcaagggactgggtgtagacctcctgtggactctggagaaagcccagaggtggtgc |
| | gagcagccagaatgggtccacattgacaccacaccctttgccagcctcctcaagaacctgggcacgctctttggcctggggctggctctcaactccagcatgtacagggagagctg |
| | caaggggaaactcagcaagtggctcccattccgcctcagctctattgtagcctccctcgtcctcctgcacgtctttgactccttgaaacccccatcccaagtcgagctggtcttctacgt |
| | cttgtccttctgcaagagtgcggtagtgcccctggcatccgtcagtgtcatcccctactgcctcgcccaggtcctgggccagccgcacaagaagtcgttgtaaggcgaattctgcaga |
| | tatccatcacactggcggccgctcgacagcaacgtctatgacctcctaaaggacctagaggaaggcatccaaacgctgatgggggtgagggtggcgccaggggtccccaatcct |
| | ggagccccactgactttgagagctgtgttagagaaacactgctgccctctttttagcagtcaggccctgacccaagagaactcaccttattcttcatttcccctcgtgaatcctccaggc |
| | ctttctctacaccctgaaggggagggaggaaaatgaatgaatgagaaagggagggaacagtacccaagcgcttggcctctccttctcttccttcactttgcagaggctggaagatgg |
| | cagcccccggactgggcagatcttcaagcagacctacagcaagttcgacacaaactcacacaacgatgacgcactactcaagaactacgggctgctctactgcttcaggaaggac |
| | atggacaaggtcgagacattcctgcgcatcgtgcagtgccgctctgtggagggcagctgtggcttctagctgcccgggtggcatccctgtgacccctccccagtgcctctcctggcc |
| | ctggaagttgccactccagtgcccaccagtcttgtcctaataaaattaagttgcatcattttgtctgactaggtgtccttctataatattatggggtggaggggggtggtatggagcaagg |
| | gaatagagtaagaaccatatagagaggagatcagcaagaaaagaggctggcatAGgctctcgcaatgtctgtccatcagaagttgctttccccaggctattcaggaagccacgg |
| | gctactcatgcttccaacccctctctctgactttggatcatctacataaagggggaagacagaaaaaatcctaccagtgagttgaaaatacaggaaagcctatttcatatgggttaaagg |
| | gtaggacagttgaatttcgtgaaaagtctgagttatataggctttgagcaaagagttttattagtatgaagcagaagaggtaacataaagaaagatgtatggggccaggcatggtggct |
| | cacacctgtaatcccagcactttgggaggccgaggtgggcgaatcactcctgggtgaactcaggagttcaagaccagcctgggcaacatggcgaaactccatctctacaaaaacat |
| | tacgaaaattagctgggcgtgttggtgctgtagtcccagctactcaggaggctgaggtgagaggcggaggaggttgcagtgagtcaagatcatgccactgcactccagcctgggc |
| | aacagagtaagaccctgtctcaaaaaaaaaaaaaagatagatgatgtatgctgtatgaaaaaaggaaacacacagatgattcaacagcctgttttgtggggtaatgaaaagtcaccct |
| | gggaactgggctccagccctcgttctgccacccaccaactacatgtccttggcaagtcatatcaattatctgagtttctgttttataatctacaaataggttatctctggcagcttaataata |
| | atcagggttaacatttattaaacagtgtgtgccagtccatgtgctatgtgcttttctgtgaggtagttactgctatttacagaaacagtagatgcagagaccaaggtgctgagttaaatgatt |
| | aggccaacaaggttagtacatgccgagccaggatggaagcccaggtaggcaggctggcttccgcggcaatgctcttatgaactatgttacgtccagtgctg |
| Donor Nucleic | gagctcaggaattcaagaccagcctgggcaacatggaaaaaccccatctctacaaaagatagaaaaattagccaggcatggtggcgtgtgcctgtggtcccagctactcaggagg |
| Acid (Human | ctgaggtgggaggatcacattagcccaggaggttgaggctgcagtgagccgtgattatgccactgcactccagcctgggagacagagtgagaccctgtttcaaaaaaaagagaga |
| SpCas9) | gaaaatttaaaaaagaaaacaacaccaagggctgtaactttaaggtcattaaatgaattaatcactgcattcaaaaacgattactttctggccctaagagacatgaggccaataccagg |
| (SEQ ID NO: 40) | aagggggttgatctcccaaaccagaggcagaccctagactctaatacagttaaggaaagaccagcaagatgatagtccccaatacaatagaagttactatattttatttgttgtttttcttt |
| | tgttttgttttgttttgttttgttttgttttagagactggggtcttgctcgattgcccaggctgtagtgcagcggtgggacaatagctcactgcagactccaactcctgggctcaagcaatcct |
| | cctgcctcagcctcctgaatagctgggactacaagggtacaccatcacacacaccaaaacaattttttaaatttttgtgtagaaacgagggtcttgctttgttgcccaggctggtctccaa |
| | ctcctggcttcaagggatcctcccacctcagcctcccaaattgctgggattacaggtgtgagccaccacaaccagccagaactttactaattttaaaattaagaacttaaaacttgaata |
| | gctagagcaccaagatttttctttgtccccaaataagtgcagttgcaggcatagaaaatctgacatctttgcaagaatcatcgtggatgtagactctgtcctgtgtctctggcctggtttcg |
| | gggaccaggagggcagacccttgcactgccaagaagcatgccaaagttaatcattggccctgctgagtacatggccgatcaggctgtttttgtgtgcctgtttttctattttacgtaaatc |
| | accctgaacatgtttgcatcaacctactggtgatgcacctttgatcaatacattttagacaaacgtggtttttgagtccaaagatcagggctgggttgacctgaatactggatacagggca |
| | tataaaacaggggcaaggcacagactcatagcagagcaatcaccaccaagcctggaataactgcaagggctctgctgacatcttcctgaggtgccaaggaaatgaggatggagg |
| | aaggaatgaatgttctccatgactttgggatccagtcaacacattacctccaggtgaattaccaagactcccaggactggttcatcttggtgtccgtgatcgcagacctcaggaatgcct |
| | tctacgtcctcttccccatctggttccatcttcaggaagctgtgggcattaaactcctttgggtagctgtgattggagactggctcaacctcgtctttaagtggattctctttggacagcgtc |
| | catactggtgggttttggatactgactactacagcaacacttccgtgcccctgataaagcagttccctgtaacctgtgagactggaccagggagcccctctggccatgccatgggcac |
| | agcaggtgtatactacgtgatggtcacatctactctttccatctttcagggaaagataaagccgacctacagatttcggtgcttgaatgtcattttgtggttgggattctgggctgtgcagc |
| | tgaatgtctgtctgtcacgaatctaccttgctgctcattttcctcatcaagttgttgctggagtcctgtcaggcattgctgttgcagaaactttcagccacatccacagcatctataatgcca |
| | gcctcaagaaatattttctcattaccttcttcctgttcagcttcgccatcggattttatctgctgctcaagggactgggtgtagacctcctgtggactctggagaaagcccagaggtggtgc |
| | gagcagccagaatgggtccacattgacaccacaccctttgccagcctcctcaagaacctgggcacgctctttggcctggggctggctctcaactccagcatgtacagggagagctg |
| | caaggggaaactcagcaagtggctcccattccgcctcagctctattgtagcctccctcgtcctcctgcacgtctttgactccttgaaacccccatcccaagtcgagctggtcttctacgt |
| | cttgtccttctgcaagagtgcggtagtgcccctggcatccgtcagtgtcatcccctactgcctcgcccaggtcctgggccagccgcacaagaagtcgttgtaaggcgaattctgcaga |
| | tatccatcacactggcggccgctcgacagcaacgtctatgacctcctaaaggacctagaggaaggcatccaaacgctgatgggggtgagggtggcgccaggggtccccaatcct |
| | ggagccccactgactttgagagctgtgttagagaaacactgctgccctctttttagcagtcaggccctgacccaagagaactcaccttattcttcatttcccctcgtgaatcctccaggc |
| | ctttctctacaccctgaaggggagggaggaaaatgaatgaatgagaaagggagggaacagtacccaagcgcttggcctctccttctcttccttcactttgcagaggctggaagatgg |
| | cagcccccggactgggcagatcttcaagcagacctacagcaagttcgacacaaactcacacaacgatgacgcactactcaagaactacgggctgctctactgcttcaggaaggac |
| | atggacaaggtcgagacattcctgcgcatcgtgcagtgccgctctgtggagggcagctgtggcttctagctgcccgggtggcatccctgtgacccctccccagtgcctctcctggcc |
| | ctggaagttgccactccagtgcccaccagtcttgtcctaataaaattaagttgcatcattttgtctgactaggtgtccttctataatattatggggtggaggggggtggtatggagcaagg |
| | gaatagagtaagaaccatatagagaggagatcagcaagaaaagaggctggcattcgctctcgcaatgtctgtccatcagaagttgctttccccaggctattcaggaagccacgggct |
| | actcatgcttccaacccctctctctgactttggatcatctacataaagggggaagacagaaaaaatcctaccagtgagttgaaaatacaggaaagcctatttcatatgggttaaagggta |
| | ggacagttgaatttcgtgaaaagtctgagttatataggctttgagcaaagagttttattagtatgaagcagaagaggtaacataaagaaagatgtatggggccaggcatggtggctcac |
| | acctgtaatcccagcactttgggaggccgaggtgggcgaatcactcctgggtgaactcaggagttcaagaccagcctgggcaacatggcgaaactccatctctacaaaaacattac |
| | gaaaattagctgggcgtgttggtgctgtagtcccagctactcaggaggctgaggtgagaggcggaggaggttgcagtgagtcaagatcatgccactgcactccagcctgggcaac |
| | agagtaagaccctgtctcaaaaaaaaaaaaaagatagatgatgtatgctgtatgaaaaaaggaaacacacagatgattcaacagcctgttttgtggggtaatgaaaagtcaccctgg |
| | gaactgggctccagccctcgttctgccacccaccaactacatgtccttggcaagtcatatcaattatctgagtttctgttttataatctacaaataggttatctctggcagcttaataataatc |
| | agggttaacatttattaaacagtgtgtgccagtccatgtgctatgtgcttttctgtgaggtagttactgctatttacagaaacagtagatgcagagaccaaggtgctgagttaaatgattag |
| | gccaacaaggttagtacatgccgagccaggatggaagcccaggtaggcaggctggcttccgcgg |
| First vector | atgtcctgcaggcagctgcgcgctcgctcgctcactgaggccgcccgggcaaagcccgggcgtcgggcgacctttggtcgcccggcctcagtgagcgagcgagcgcgcagag |
| (Vector Set A) | agggagtggccaactccatcactaggggttcctgcggccctgaaaacacaaatggcgtgttttggttggcgtaaggcgcctgtcagttaaccactcagtttgttcccaaatcacttttat |
| (AAV- | tctgtgtgtggagagaaacaggaggtcagaggtatcccttcctcttcgtcagagcaaatggttctattacactagggctctgtctttatggtctcctttaagttatacttttaacttcttttggct |
| mouseG6PCdon | taaagtacctatctccaactgtgatccacgtgaacttggtgaaagtccaagagagtgagcatttccaggacaacaaagccctactgctgtgtattttgtatattttaaattaataacttaaaa |
| orbGHPolyA + | ggtcacttccggcagtagcaaacttccctttgtcttcaaatagatataattgcagacttcaaaaaacagatgcccttgcaagagtcatggttgaaacagactctgccctgggcctctggc |
| SpCas9gRNA Final | ctggcttcaaggaccaggaaggagggcagcctctagcactgtcaagcagtgtgcccaagttaataattggctctgccaatggcgatcaggctgtttttgtgtgcctgttttgctattttac |
| Mouse DONOR, | gtaaatcaccctgaacatgtttgcatcaacctactgatgatgcacctttgatcaatagattttagacaaaagtggttttttgagtccaaagatcagggctgggttgacctacagactgaatc |
| see FIG. 13) | cagggcatataaaacaggggcaaggcacagactgatagcagagggatcaagccaaccgggcttggactcactgcacgggctctgctggcagcttcctgaggtaccaagggagg |
| (SEQ ID NO: 41) | aagctcgagggaaggaatgaacattctccatgactttgggatccagtcgactcgctatctccaagtgaattaccaagactcccaggactggttcatccttgtgtctgtgattgctgacct |
| | gaggaacgccttctatgtcgactttcccatctagatcacgcgtcctgtgtctctggcctggtttcggggaccaggagggcagacccttgcactgccaagaagcatgccaaagttaatc |
| | attggccctgctgagtacatggccgatcaggctgtttttgtgtgcctgtttttctattttacgtaaatcaccctgaacatgtttgcatcaacctactggtgatgcacctttgatcaatacatttta |
| | gacaaacgtggtttttgagtccaaagatcagggctgggttgacctgaatactggatacagggcatataaaacaggggcaaggcacagactcatagcagagcaatcaccaccaagc |
| | ctggaataactgcaagggctctgctgacatcttcctgaggtgccaaggaaatgaggatggaggaaggaatgaatgttctccatgactttgggatccagtcaacacattacctccaggt |
| | gaattaccaagactcccaggactggttcatcttggtgtccgtgatcgcagacctcaggaatgccttctacgtcctcttccccatctggttccatcttcaggaagctgtgggcattaaactc |
| | ctttgggtagctgtgattggagactggctcaacctcgtctttaagtggattctctttggacagcgtccatactggtgggttttggatactgactactacagcaacacttccgtgcccctgat |
| | aaagcagttccctgtaacctgtgagactggaccagggagcccctctggccatgccatgggcacagcaggtgtatactacgtgatggtcacatctactctttccatctttcagggaaag |
| | ataaagccgacctacagatttcggtgcttgaatgtcattttgtggttgggattctgggctgtgcagctgaatgtctgtctgtcacgaatctaccttgctgctcattttcctcatcaagttgttgc |
| | tggagtcctgtcaggcattgctgttacagaaactttcagccacatccacagcatctataatgccagcctcaagaaatattttctcattaccttcttcctgttcagcttcgccatcggattttat |
| | ctgctgctcaagggactgggtgtagacctcctgtggactctggagaaagcccagaggtggtgcgagcagccagaatgggtccacattgacaccacaccctttgccagcctcctcaa |
| | gaacctgggcacgctctttggcctggggctggctctcaactccagcatgtacagggagagctgcaaggggaaactcagcaagtggctcccattccgcctcagctctattgtagcctc |
| | cctcgtcctcctgcacgtctttgactccttgaaacccccatcccaagtcgagctggtcttctacgtcttgtccttctgcaagagtgcggtagtgcccctggcatccgtcagtgtcatcccc |
| | tactgcctcgcccaggtcctgggccagccgcacaagaagtcgttgtaagagatgtggagtcttcggtgtttaaagtcgaattcgtttaaacccgctgatcagcctcgactgtgccttcta |
| | gttgccagccatctgttgtttgcccctcccccgtgccttccttgaccctggaaggtgccactcccactgtcctttcctaataaaatgaggaaattgcatcgcattgtctgagtaggtgtcat |
| | tctattctggggggtggggggggcaggacagcaagggggaggattgggaagacaatagcaggcatgctggggatgcggtgggctctatggcttctgaggcggaaagaaccag |
| | ctggaattctgcagatctcgagtctagatcacgcgtaaagagactgtgggcatcaatctcctctgggtggcagtggtcggagactggttcaacctcgtcttcaagtggtaagaaccatc |
| | ccgaggggtgatgggtagagaaatggctgtcctccagcaggggtaccccatactgttcaccaggctacaggtaagtccccattggcttgagagtctctacagaatcccacctgtggc |
| | tggcaatgaggaatggctattttgtcagacctgagggatggaaagacagctgagtttaatgggaagtctgagtgtagcctttactctggtttatttgactttattatttagagttagggtctt |
| | gtgtagcccaggctggcctcaaacttacaaattaagagacaacgaccttgaattgctcaacttctgagtgctgagattacaggtgtgtgttcccacgtgtagtttagcatgctagggatt |
| | gaacctggggcttggagcatgagagcaaggactcccccatgaggctatatcccagctcctgagcacagatgttaaaaaaacccttttactactatttctatgggtgctatagaaacaga |
| | tgtgggaaagaaggggggggggagtgatttctaaccttttggttaagcggtgaaaattgatacggatagccaggaccttccaactggtgaattacataaactcactattctagttttaga |
| | atctataaactttggttctctccaggatcttaataatatgcacaatcaacgtttattgatcagctactatgtgccaggctcgcactgtctctccctgtgtctgtctcagtctgtgtgtgtgtttgt |
| | gtgtgtgcgcacgtgttcatctgcgtacatgcatgtggctgtgtttgtggaagccagtgcagtcattcctcaaaacttattaagataaagtctctgattgaactggagctccggagtaaatt |
| | tgaccaatgagcactggaggtctacctgcctgtattcccatagctatatgccaccatgccgcggcagtttggttaattaaggtaccgagggcctatttcccatgattccttcatatttgcat |
| | atacgatacaaggctgttagagagataattagaattaatttgactgtaaacacaaagatattagtacaaaatacgtgacgtagaaagtaataatttcttgggtagtttgcagttttaaaattat |
| | gttttaaaatggactatcatatgcttaccgtaacttgaaagtatttcgatttcttggctttatatatcttgtggaaaggacgaaacaccgtctatgtcctctttcccatcgttttagagctagaaa |
| | tagcaagttaaaataaggctagtccgttatcaacttgaaaaagtggcaccgagtcggtgcttttttgccgcggtggggccgcaggaacccctagtgatggagttggccactccctctct |
| | gcgcgctcgctcgctcactgaggccgggcgaccaaaggtcgcccgacgcccgggctttgcccgggcggcctcagtgagcgagcgagcgcgcagctgcctgcaggggcgcct |
| | gatgcggtattttctccttacgcatctgtgcggtatttcacaccgcatacgtcaaagcaaccatagtacgcgccctgtagcggcgcattaagcgcggcgggtgtggtggttacgcgca |
| | gcgtgaccgctacacttgccagcgccctagcgcccgctcctttcgctttcttcccttcctttctcgccacgttcgccggctttccccgtcaagctctaaatcgggggctccctttagggtt |
| | ccgatttagtgctttacggcacctcgaccccaaaaaacttgatttgggtgatggttcacgtagtgggccatcgccctgatagacggtttttcgccctttgacgttggagtccacgttcttta |
| | atagtggactcttgttccaaactggaacaacactcaaccctatctcgggctattcttttgatttataagggattttgccgatttcggcctattggttaaaaaatgagctgatttaacaaaaattt |
| | aacgcgaattttaacaaaatattaacgtttacaattttatggtgcactctcagtacaatctgctctgatgccgcatagttaagccagccccgacacccgccaacacccgctgacgcgcc |
| | ctgacgggcttgtctgctcccggcatccgcttacagacaagctgtgaccgtctccgggagctgcatgtgtcagaggttttcaccgtcatcaccgaaacgcgcgagacgaaagggcc |
| | tcgtgatacgcctatttttataggttaatgtcatgataataatggtttcttagacgtcaggtggcacttttcggggaaatgtgcgcggaacccctatttgtttatttttctaaatacattcaaatat |
| | gtatccgctcatgagacaataaccctgataaatgcttcaataatattgaaaaaggaagagtatgagtattcaacatttccgtgtcgcccttattcccttttttgcggcattttgccttcctgtttt |
| | tgctcacccagaaacgctggtgaaagtaaaagatgctgaagatcagttgggtgcacgagtgggttacatcgaactggatctcaacagcggtaagatccttgagagttttcgccccga |
| | agaacgttttccaatgatgagcacttttaaagttctgctatgtggcgcggtattatcccgtattgacgccgggcaagagcaactcggtcgccgcatacactattctcagaatgacttggtt |
| | gagtactcaccagtcacagaaaagcatcttacggatggcatgacagtaagagaattatgcagtgctgccataaccatgagtgataacactgcggccaacttacttctgacaacgatcg |
| | gaggaccgaaggagctaaccgcttttttgcacaacatgggggatcatgtaactcgccttgatcgttgggaaccggagctgaatgaagccataccaaacgacgagcgtgacaccac |
| | gatgcctgtagcaatggcaacaacgttgcgcaaactattaactggcgaactacttactctagcttcccggcaacaattaatagactggatggaggcggataaagttgcaggaccactt |
| | ctgcgctcggcccttccggctggctggtttattgctgataaatctggagccggtgagcgtgggtctcgcggtatcattgcagcactggggccagatggtaagccctcccgtatcgtag |
| | ttatctacacgacggggagtcaggcaactatggatgaacgaaatagacagatcgctgagataggtgcctcactgattaagcattggtaactgtcagaccaagtttactcatatatacttt |
| | agattgatttaaaacttcatttttaatttaaaaggatctaggtgaagatcctttttgataatctcatgaccaaaatcccttaacgtgagttttcgttccactgagcgtcagaccccgtagaaaa |
| | gatcaaaggatcttcttgagatcctttttttctgcgcgtaatctgctgcttgcaaacaaaaaaaccaccgctaccagcggtggtttgtttgccggatcaagagctaccaactctttttccga |
| | aggtaactggcttcagcagagcgcagataccaaatactgtccttctagtgtagccgtagttaggccaccacttcaagaactctgtagcaccgcctacatacctcgctctgctaatcctgt |
| | taccagtggctgctgccagtggcgataagtcgtgtcttaccgggttggactcaagacgatagttaccggataaggcgcagcggtcgggctgaacggggggttcgtgcacacagcc |
| | cagcttggagcgaacgacctacaccgaactgagatacctacagcgtgagctatgagaaagcgccacgcttcccgaagggagaaaggcggacaggtatccggtaagcggcagg |
| | gtcggaacaggagagcgcacgagggagcttccagggggaaacgcctggtatctttatagtcctgtcgggtttcgccacctctgacttgagcgtcgatttttgtgatgctcgtcagggg |
| | ggcggagcctatggaaaaacgccagcaacgcggcctttttacggttcctggccttttgctggccttttgctcac |
| First Vector | atgtcctgcaggcagctgcgcgctcgctcgctcactgaggccgcccgggcaaagcccgggcgtcgggcgacctttggtcgcccggcctcagtgagcgagcgagcgcgcagag |
| (Vector Set B) | agggagtggccaactccatcactaggggttcctgcggcccagttaaccactcagtttgttcccaaatcacttttattctgtgtgtggagagaaacaggaggtcagaggtatcccttcctc |
| (New Mouse | ttcgtcagagcaaatggttctattacactagggctctgtctttatggtctcctttaagttatacttttaacttcttttggcttaaagtacctatctccaactgtgatccacgtgaacttggtgaaag |
| Donor w/ | tccaagagagtgagcatttccaggacaacaaagccctactgctgtgtattttgtatattttaaattaataacttaaaaggtcacttccggcagtagcaaacttccctttgtcttcaaatagata |
| hG6PC, see FIG. | taattgcagacttcaaaaaacagatgcccttgcaagagtcatggttgaaacagactctgccctgggcctctggcctggcttcaaggaccaggaaggagggcagcctctagcactgtc |
| 27) | aagcagtgtgcccaagttaataattggctctgccaatggcgatcaggctgtttttgtgtgcctgttttgctattttacgtaaatcaccctgaacatgtttgcatcaacctactgatgatgcacc |
| (SEQ ID NO: 42) | tttgatcaatagattttagacaaaagtggttttttgagtccaaagatcagggctgggttgacctacagactgaatccagggcatataaaacaggggcaaggcacagactgatagcaga |
| | gggatcaagccaaccgggcttggactcactgcacgggctctgctggcagcttcctgaggtaccaagggaggaagctcgaggtgccaaggaaatgaggatggaggaaggaatga |
| | atgttctccatgactttgggatccagtcaacacattacctccaggtgaattaccaagactcccaggactggttcatcttggtgtccgtgatcgcagacctcaggaatgccttctacgtcct |
| | cttccccatctggttccatcttcaggaagctgtgggcattaaactcctttgggtagctgtgattggagactggctcaacctcgtctttaagtggattctctttggacagcgtccatactggtg |
| | ggttttggatactgactactacagcaacacttccgtgcccctgataaagcagttccctgtaacctgtgagactggaccagggagcccctctggccatgccatgggcacagcaggtgt |
| | atactacgtgatggtcacatctactctttccatctttcagggaaagataaagccgacctacagatttcggtgcttgaatgtcattttgtggttgggattctgggctgtgcagctgaatgtctg |
| | tctgtcacgaatctaccttgctgctcattttcctcatcaagttgttgctggagtcctgtcaggcattgctgttacagaaactttcagccacatccacagcatctataatgccagcctcaagaa |
| | atattttctcattaccttcttcctgttcagcttcgccatcggattttatctgctgctcaagggactgggtgtagacctcctgtggactctggagaaagcccagaggtggtgcgagcagcca |
| | gaatgggtccacattgacaccacaccctttgccagcctcctcaagaacctgggcacgctctttggcctggggctggctctcaactccagcatgtacagggagagctgcaaggggaa |
| | actcagcaagtggctcccattccgcctcagctctattgtagcctccctcgtcctcctgcacgtctttgactccttgaaacccccatcccaagtcgagctggtcttctacgtcttgtccttctg |
| | caagagtgcggtagtgcccctggcatccgtcagtgtcatcccctactgcctcgcccaggtcctgggccagccgcacaagaagtcgttgtaagagatgtggagtcttcggtgtttaaa |
| | gtcgaattctgcagatctcgagtctagagggcccgtttaaacccgctgatcagcctcgactgtgccttctagttgccagccatctgttgtttgcccctcccccgtgccttccttgaccctg |
| | gaaggtgccactcccactgtcctttcctaataaaatgaggaaattgcatcgcattgtctgagtaggtgtcattctattctggggggtggggggggcaggacagcaagggggaggatt |
| | gggaagacaatagcaggcatgctggggatgcggtgggctctatggcttctgaggcggaaagaaccagctggggctctagggggtatccccacgcgccctgtagcggcgcattaa |
| | gcgcggcgggtgtggtggttacgcgcaaaaacgtcgactttcccatctagatccatcttaaagagactgtgggcatcaatctcctctgggtggcagtggtcggagactggttcaacct |
| | cgtcttcaagtggtaagaaccatcccgaggggtgatgggtagagaaatggctgtcctccagcaggggtaccccatactgttcaccaggctacaggtaagtccccattggcttgaga |
| | gtctctacagaatcccacctgtggctggcaatgaggaatggctattttgtcagacctgagggatggaaagacagctgagtttaatgggaagtctgagtgtagcctttactctggtttattt |
| | gactttattatttagagttagggtcttgtgtagcccaggctggcctcaaacttacaaattaagagacaacgaccttgaattgctcaacttctgagtgctgagattacaggtgtgtgttccca |
| | cgtgtagtttagcatgctagggattgaacctggggcttggagcatgagagcaaggactcccccatgaggctatatcccagctcctgagcacagatgttaaaaaaacccttttactacta |
| | tttctatgggtgctatagaaacagatgtgggaaagaaggggggggggagtgatttctaaccttttggttaagcggtgaaaattgatacggatagccaggaccttccaactggtgaatta |
| | cataaactcactattctagttttagaatctataaactttggttctctccaggatcttaataatatgcacaatcaacgtttattgatcagctactatgtgccaggctcgcactgtctctccctgtgt |
| | ctgtctcagtctgtgtgtgtgtttgtgtgtgtgcgcacgtgttcatctgcgtacatgcatgtggctgtgtttgtggaagccagtgcagtcattcctcaaaacttattaagataaagtctctgatt |
| | gaactggagctccggagtaaatttgaccaatgagcactggaggtctacctgcctgtattcccatagctatatgccaccatgccgcggcagtttggttaattaaggtaccgagggcctat |
| | ttcccatgattccttcatatttgcatatacgatacaaggctgttagagagataattagaattaatttgactgtaaacacaaagatattagtacaaaatacgtgacgtagaaagtaataatttct |
| | tgggtagtttgcagttttaaaattatgttttaaaatggactatcatatgcttaccgtaacttgaaagtatttcgatttcttggctttatatatcttgtggaaaggacgaaacaccgtctatgtcctc |
| | tttcccatcgttttagagctagaaatagcaagttaaaataaggctagtccgttatcaacttgaaaaagtggcaccgagtcggtgcttttttgccgcggtggggccgcaggaacccctagt |
| | gatggagttggccactccctctctgcgcgctcgctcgctcactgaggccgggcgaccaaaggtcgcccgacgcccgggctttgcccgggggcctcagtgagcgagcgagcgc |
| | gcagctgcctgcaggggcgcctgatgcggtattttctccttacgcatctgtgcggtatttcacaccgcatacgtcaaagcaaccatagtacgcgccctgtagcggcgcattaagcgcg |
| | gcgggtgtggtggttacgcgcagcgtgaccgctacacttgccagcgccctagcgcccgctcctttcgctttcttcccttcctttctcgccacgttcgccggctttccccgtcaagctcta |
| | aatcgggggctccctttagggttccgatttagtgctttacggcacctcgaccccaaaaaacttgatttgggtgatggttcacgtagtgggccatcgccctgatagacggtttttcgccctt |
| | tgacgttggagtccacgttctttaatagtggactcttgttccaaactggaacaacactcaaccctatctcgggctattcttttgatttataagggattttgccgatttcggcctattggttaaaa |
| | aatgagctgatttaacaaaaatttaacgcgaattttaacaaaatattaacgtttacaattttatggtgcactctcagtacaatctgctctgatgccgcatagttaagccagccccgacaccc |
| | gccaacacccgctgacgcgccctgacgggcttgtctgctcccggcatccgcttacagacaagctgtgaccgtctccgggagctgcatgtgtcagaggttttcaccgtcatcaccgaa |
| | acgcgcgagacgaaagggcctcgtgatacgcctatttttataggttaatgtcatgataataatggtttcttagacgtcaggtggcacttttcggggaaatgtgcgcggaacccctatttgt |
| | ttatttttctaaatacattcaaatatgtatccgctcatgagacaataaccctgataaatgcttcaataatattgaaaaaggaagagtatgagtattcaacatttccgtgtcgcccttattcccttt |
| | tttgcggcattttgccttcctgtttttgctcacccagaaacgctggtgaaagtaaaagatgctgaagatcagttgggtgcacgagtgggttacatcgaactggatctcaacagcggtaag |
| | atccttgagagttttcgccccgaagaacgttttccaatgatgagcacttttaaagttctgctatgtggcgcggtattatcccgtattgacgccgggcaagagcaactcggtcgccgcata |
| | cactattctcagaatgacttggttgagtactcaccagtcacagaaaagcatcttacggatggcatgacagtaagagaattatgcagtgctgccataaccatgagtgataacactgcggc |
| | caacttacttctgacaacgatcggaggaccgaaggagctaaccgcttttttgcacaacatgggggatcatgtaactcgccttgatcgttgggaaccggagctgaatgaagccatacc |
| | aaacgacgagcgtgacaccacgatgcctgtagcaatggcaacaacgttgcgcaaactattaactggcgaactacttactctagcttcccggcaacaattaatagactggatggaggc |
| | ggataaagttgcaggaccacttctgcgctcggcccttccggctggctggtttattgctgataaatctggagccggtgagcgtgggtctcgcggtatcattgcagcactggggccagat |
| | ggtaagccccccgtatcgtagttatctacacgacggggagtcaggcaactatggatgaacgaaatagacagatcgctgagataggtgcctcactgattaagcattggtaactgtcag |
| | accaagtttactcatatatactttagattgatttaaaacttcatttttaatttaaaaggatctaggtgaagatcctttttgataatctcatgaccaaaatcccttaacgtgagttttcgttccactga |
| | gcgtcagaccccgtagaaaagatcaaaggatcttcttgagatcctttttttctgcgcgtaatctgctgcttgcaaacaaaaaaaccaccgctaccagcggtggtttgtttgccggatcaa |
| | gagctaccaactctttttccgaaggtaactggcttcagcagagcgcagataccaaatactgtccttctagtgtagccgtagttaggccaccacttcaagaactctgtagcaccgcctac |
| | atacctcgctctgctaatcctgttaccagtggctgctgccagtggcgataagtcgtgtcttaccgggttggactcaagacgatagttaccggataaggcgcagcggtcgggctgaac |
| | ggggggttcgtgcacacagcccagcttggagcgaacgacctacaccgaactgagatacctacagcgtgagctatgagaaagcgccacgcttcccgaagggagaaaggcggac |
| | aggtatccggtaagcggcagggtcggaacaggagagcgcacgagggagcttccagggggaaacgcctggtatctttatagtcctgtcgggtttcgccacctctgacttgagcgtc |
| | gatttttgtgatgctcgtcaggggggggagcctatggaaaaacgccagcaacgcggcctttttacggttcctggccttttgctggccttttgctcac |
| First Vector | cagatctttggccactccctctctgcgcgctcgctcgctcactgaggccgggcgaccaaaggtcgcccgacgcccgggctttgcccgggcggcctcagtgagcgagcgagcgc |
| (Vector Set C) | gcagagagggagtggccaactccatcactaggggttcctggaggggtggagtcgtgacgtgaattacgtcatagggttagggaggtcggccgctctagaatgctaagtgaaataa |
| (AAV- | gacagaaaaagacaaatatcatatggtttcacttatatgtagggtctaaaaagcgaaacaaatgagtaaacaaacacaaaaagcagactcaaacctactatgtggagaacaaattgat |
| 2xG6PDonor | acttgcccaagaggaggagcagtgagaggatggggaaaatgggtgaaggggaatgagagatacaggcttccagacctcatggaatgagtaagtcactggaatgaaaggcacag |
| DOG DONOR, | cacagggaatacagtcaatggtattgtaacagtgttgtacagtgacaggtggtagctatactgtggtgaacgaagcataatgcacagagatgttgaataactatgttgtacacctgaaa |
| see FIG. 11) | ctaatgttagattagtttcattatgtaacatcagtttcagctacattctaatacactttaaaataatacagtagtcctttccagagtccccagtcaccaatttgctcgtgtttttatttagaacccc |
| (SEQ ID NO: 43) | atattcattacattgtttttgcatgaacacacattcattctactgcttaaacttatgtgttaaaaccccaaaggtcctaacttggaggacattgcatgaattaatcagtgcattcaaagatgatt |
| | acttcctggccctaagatatatagaatcaacactaggaagaaggtttgatctcccaaaccagaggcacttcacagacactatgcagttaagaatacacagcaaggtaggtgatggttc |
| | ccagtacaatagaacttagcgaatatgtttttttgagattttatttatgagagacacacagagagaggcagagacacaggtagaaggagaagcaggctccctgaagggagcccgatg |
| | ccggactcaatcccaggaccctgagatcacaacctgagccaaaggtagatgctcatccactgagccatgcaggtaccccgaacttagcatattttaaaagtaatatgtttaaaacatg |
| | ctcactagggtaccaagacttttctttgtcctcaaataagtgcacttgcagacagaaaatcaggatctttgcaagaaccatggcggatttagactctgtcctgtgtctccggcccagtttc |
| | ggagccaggagggcagcccttgcactatcaagtagcatgccaaagttaatcattggccctgccaagtacatggcagatcaggctgtttttgtgtgcctgtttttctattttacgtaaatca |
| | ccctgaacatgtttgcatcaacctcctggtgatgtacctttgatcaatagattttagacaaaagtggtttttgagtccaaagatcagggctgggttgacctgcagactggatacagagtgt |
| | ataaaaacaggggcaaggcgcaggctgatagcagagcaatcgccaagtcggtaccgagctcggaaagggcgaattccagcacactggcggccgttactagtggatccaaggat |
| | ggaggaaggaatggatgtgctccatgactttggaatccagtcaacgcactacctccaggtgaattaccaggactctcaggattggttcatcttggtgtccgtgattgcagacctcagga |
| | atgccttctatgtcctctttcccatctggttccatctgcgtgaagctgtgggcatcaaacttctctgggtagctgtgattggagactggctcaacctcgtctttaaatggattctgtttggaca |
| | gcgtccatactggtgggtcatggacaccgactactatagcaacacctctgtgccactgataaagcaatttccagttacctgtgaaactggaccagggagtccctctggtcatgccatg |
| | ggtacagcaggtgtatactacgtgatggtcacatctaccctttctatctttcgggggagaaaaaggccaacctacagatttcggtgcttgaatatccttctgtggttgggattctgggctgt |
| | gcagctgaacgtctgtctgtcccgaatctaccttgctgctcatttcccccatcaggttgttgctggagtcctgtcaggcattgctgttgctgaaactttccgccacatccagagcatctaca |
| | atgccagcctcaagaaatattttctcattactttcttcctgttcagttttgccattggattttacctgctgctcaaggggctgggtgtggacctcctgtggacactggaaaaagccaggaga |
| | tggtgtgagcggccggaatgggttcacattgacaccacaccctttgccagccttctcaagaacgtggggaccctctttggcctgggggtggctctcaactccagcatgtacagggaa |
| | agctgcaagggcaagcttagcaagtggttcccattccgcctcagctgcattgtggtgtctctcatcctcctgcacctctttgactctttgaaacccccatcccaaactgagctgatcttcta |
| | caccttgtccttctgcaagagtgcagcagtgcccctggcatctgtcagcctcatcccctactgccttgcccgggtcttcgaccagccagacaagaagtctttgtaaggcgaattctgca |
| | gatatccatcacactggcggccgctcgacagcaacgtctatgacctcctaaaggacctagaggaaggcatccaaacgctgatgggggtgagggtggcgccaggggtccccaatc |
| | ctggagccccactgactttgagagctgtgttagagaaacactgctgccctctttttagcagtcaggccctgacccaagagaactcaccttattcttcatttcccctcgtgaatcctccagg |
| | cctttctctacaccctgaaggggagggaggaaaatgaatgaatgagaaagggagggaacagtacccaagcgcttggcctctccttctcttccttcactttgcagaggctggaagatg |
| | gcagcccccggactgggcagatcttcaagcagacctacagcaagttcgacacaaactcacacaacgatgacgcactactcaagaactacgggctgctctactgcttcaggaagga |
| | catggacaaggtcgagacattcctgcgcatcgtgcagtgccgctctgtggagggcagctgtggcttctagctgcccgggtggcatccctgtgacccctccccagtgcctctcctggc |
| | cctggaagttgccactccagtgcccaccagtcttgtcctaataaaattaagttgcatcattttgtctgactaggtgtccttctataatattatggggggaggggggtggtatggagcaag |
| | ggaatagattctctgggtagctgtgattggagactggctcaacctcgtctttaaatggtgagtaagagccagacagagaggagatcggcaagggaagaggctggtatagtctctgga |
| | aatgtctgacatcagaaattgccttctccgtgctagtcgggaagccacaggttactcccctcctgactctggatcatctacctaaagagggaagacagcgaaaatcttgttgatgaattg |
| | aagatacaggcaaggctatttcatatggatggaaggccagccggacttagtgaaaagtctaaagtatataggctataagcaaagaagcacagttgtattactttgaagcagaataggt |
| | aacataaagataggtgtatgaaagatgatgtatgaaaacaggaaacaagtatataatttaaaaacccactgcatggagaaagaagaagtaacccagagaactgggctctaacccttg |
| | ttcggccacctaccagctgtatgtcctgggaaagtcagagcaactctctgagtttctgtcttacaatctgtaaacttaaattatccttgggagcttaataataattctagttaacatttattgaa |
| | catttagtaggtactaggccatgtgccaagtgcttttctgtgaggtagttactactgtattatagatgaagagacagagccattacgataagggaccagaactagatccacatggtggtc |
| | aaaggcagaggcagcattttaaggccagacaggctagtttcagaggcaaagctcttaaggattatgctgtattcattgctgacaaattgactttttcagggagcaggaggaagccaaa |
| | ttttaatgtttgtcaatttctctggtgtatatattcctattggggccacttttgatgggtcaagcttgcaaaaatcctgaatattttacaattgtcagccaataccatatgattctagctcattagtg |
| | gcttggcctctatttcctcatctagaaaataaagagcttgggccagctaaaacctttaccatccctaaactatctatggaagggtctctcctgtagggcctgcggggtctattgggaacc |
| | aagctggagtgcagtggcacaatcttggctcactgcaatctccgcctcctgggttcaagcgattctcctgcctcagcctcccgagttgttgggattccaggcatcgagtagataagtag |
| | catggcgggttaatcattaactacaaggaacccctagtgatggagttggccactccctctctgcgcgctcgctcgctcactgaggccgggcgaccaaaggtcgcccgacgcccgg |
| | gctttgcccgggcggcctcagtgagcgagcgagcgcgcagagagggagtggccaaagatctgcgacgcgaggctggatggccttccccattatgattcttctcgcttccggcggc |
| | atcgggatgcccgcgttgcaggccatgctgtccaggcaggtagatgacgaccatcagggacagcttcagcaaaaggccagcaaaaggccaggaaccgtaaaaaggccgcgttg |
| | ctggcgtttttccataggctccgcccccctgacgagcatcacaaaaatcgacgctcaagtcagaggtggcgaaacccgacaggactataaagataccaggcgtttccccctggaag |
| | ctccctcgtgcgctctcctgttccgaccctgccgcttaccggatacctgtccgcctttctcccttcgggaagcgtggcgctttctcatagctcacgctgtaggtatctcagttcggtgtag |
| | gtcgttcgctccaagctgggctgtgtgcacgaaccccccgttcagcccgaccgctgcgccttatccggtaactatcgtcttgagtccaacccggtaagacacgacttatcgccactgg |
| | cagcagccactggtaacaggattagcagagcgaggtatgtaggcggtgctacagagttcttgaagtggtggcctaactacggctacactagaaggacagtatttggtatctgcgctc |
| | tgctgaagccagttaccttcggaaaaagagttggtagctcttgatccggcaaacaaaccaccgctggtagcggtggtttttttgtttgcaagcagcagattacgcgcagaaaaaaagg |
| | atctcaagaagatcctttgatcttttctacggggtctgacgctcagtggaacgaaaactcacgttaagggattttggtcatgagattatcaaaaaggatcttcacctagatccttttaaatta |
| | aaaatgaagttttaaatcaatctaaagtatatatgagtaaacttggtctgacagttaccaatgcttaatcagtgaggcacctatctcagcgatctgtctatttcgttcatccatagttgcctga |
| | ctccccgtcgtgtagataactacgatacgggagggcttaccatctggccccagtgctgcaatgataccgcgagacccacgctcaccggctccagatttatcagcaataaaccagcc |
| | agccggaagggccgagcgcagaagtggtcctgcaactttatccgcctccatccagtctattaattgttgccgggaagctagagtaagtagttcgccagttaatagtttgcgcaacgttg |
| | ttgccattgctgcaggcatcgtggtgtcacgctcgtcgtttggtatggcttcattcagctccggttcccaacgatcaaggcgagttacatgatcccccatgttgtgcaaaaaagcggtta |
| | gctccttcggtcctccgatcgttgtcagaagtaagttggccgcagtgttatcactcatggttatggcagcactgcataattctcttactgtcatgccatccgtaagatgcttttctgtgactg |
| | gtgagtactcaaccaagtcattctgagaatagtgtatgcggcgaccgagttgctcttgcccggcgtcaacacgggataataccgcgccacatagcagaactttaaaagtgctcatcat |
| | tggaaaacgttcttcggggcgaaaactctcaaggatcttaccgctgttgagatccagttcgatgtaacccactcgtgcacccaactgatcttcagcatcttttactttcaccagcgtttctg |
| | ggtgagcaaaaacaggaaggcaaaatgccgcaaaaaagggaataagggcgacacggaaatgttgaatactcatactcttcctttttcaatattattgaagcatttatcagggttattgtc |
| | tcatgagcggatacatatttgaatgtatttagaaaaataaacaaataggggttccgcgcacatttccccgaaaagtgccacctg |
| First Vector | cagatctttggccactccctctctgcgcgctcgctcgctcactgaggccgggcgaccaaaggtcgcccgacgcccgggctttgcccgggcggcctcagtgagcgagcgagcgc |
| (Vector Set D) | gcagagagggagtggccaactccatcactaggggttcctggaggggtggagtcgtgacgtgaattacgtcatagggttagggaggtcggccgctctagagagctcaggaattcaa |
| (AAV-SaCas9 | gaccagcctgggcaacatggaaaaaccccatctctacaaaagatagaaaaattagccaggcatggtggcgtgtgcctgtggtcccagctactcaggaggctgaggtgggaggatc |
| Human | acattagcccaggaggttgaggctgcagtgagccgtgattatgccactgcactccagcctgggagacagagtgagaccctgtttcaaaaaaaagagagagaaaatttaaaaaaga |
| DONOR, see | aaacaacaccaagggctgtaactttaaggtcattaaatgaattaatcactgcattcaaaaacgattactttctggccctaagagacatgaggccaataccaggaagggggttgatctcc |
| FIG. 28) | caaaccagaggcagaccctagactctaatacagttaaggaaagaccagcaagatgatagtccccaatacaatagaagttactatattttatttgttgtttttcttttgttttgttttgttttgtttt |
| (SEQ ID NO: 44) | gttttgttttagagactggggtcttgctcgattgcccaggctgtagtgcagcggtgggacaatagctcactgcagactccaactcctgggctcaagcaatcctcctgcctcagcctcct |
| | gaatagctgggactacaagggtacaccatcacacacaccaaaacaattttttaaatttttgtgtagaaacgagggtcttgctttgttgcccaggctggtctccaactcctggcttcaagg |
| | gatcctcccacctcagcctcccaaattgctgggattacaggtgtgagccaccacaaccagccagaactttactaattttaaaattaagaacttaaaacttgaatagctagagcaccaag |
| | atttttctttgtccccaaataagtgcagttgcaggcatagaaaatctgacatctttgcaagaatcatcgtggatgtagactctgtcctgtgtctctggcctggtttcggggaccaggaggg |
| | cagacccttgcactgccaagaagcatgccaaagttaatcattggccctgctgagtacatggccgatcaggctgtttttgtgtgcctgtttttctattttacgtaaatcaccctgaacatgttt |
| | gcatcaacctactggtgatgcacctttgatcaatacattttagacaaacgtggtttttgagtccaaagatcagggctgggttgacctgaatactggatacagggcatataaaacagggg |
| | caaggcacagactcatagcagagcaatcaccaccaagcctggaataactgcaagggctctgctgacatcttcctgaggtgccaaggaaatgaggatggaggaaggaatgaatgtt |
| | ctccatgactttgggatccagtcaacacattacctccaggtgaattaccaagactcccaggactggttcatcttggtgtccgtgatcgcagacctcaggaatgccttctacgtcctcttcc |
| | ccatctggttccatcttcaggaagctgtgggcattaaactcctttgggtagctgtgattggagactggctcaacctcgtctttaagtggattctctttggacagcgtccatactggtgggttt |
| | tggatactgactactacagcaacacttccgtgcccctgataaagcagttccctgtaacctgtgagactggaccagggagcccctctggccatgccatgggcacagcaggtgtatact |
| | acgtgatggtcacatctactctttccatctttcagggaaagataaagccgacctacagatttcggtgcttgaatgtcattttgtggttgggattctgggctgtgcagctgaatgtctgtctgt |
| | cacgaatctaccttgctgctcattttcctcatcaagttgttgctggagtcctgtcaggcattgctgttgcagaaactttcagccacatccacagcatctataatgccagcctcaagaaatatt |
| | ttctcattaccttcttcctgttcagcttcgccatcggattttatctgctgctcaagggactgggtgtagacctcctgtggactctggagaaagcccagaggtggtgcgagcagccagaat |
| | gggtccacattgacaccacaccctttgccagcctcctcaagaacctgggcacgctctttggcctggggctggctctcaactccagcatgtacagggagagctgcaaggggaaactc |
| | agcaagtggctcccattccgcctcagctctattgtagcctccctcgtcctcctgcacgtctttgactccttgaaacccccatcccaagtcgagctggtcttctacgtcttgtccttctgcaa |
| | gagtgcggtagtgcccctggcatccgtcagtgtcatcccctactgcctcgcccaggtcctgggccagccgcacaagaagtcgttgtaaggcgaattctgcagatatccatcacactg |
| | gcggccgctcgacagcaacgtctatgacctcctaaaggacctagaggaaggcatccaaacgctgatgggggtgagggtggcgccaggggtccccaatcctggagccccactga |
| | ctttgagagctgtgttagagaaacactgctgccctctttttagcagtcaggccctgacccaagagaactcaccttattcttcatttcccctcgtgaatcctccaggcctttctctacaccctg |
| | aaggggagggaggaaaatgaatgaatgagaaagggagggaacagtacccaagcgcttggcctctccttctcttccttcactttgcagaggctggaagatggcagcccccggactg |
| | ggcagatcttcaagcagacctacagcaagttcgacacaaactcacacaacgatgacgcactactcaagaactacgggctgctctactgcttcaggaaggacatggacaaggtcga |
| | gacattcctgcgcatcgtgcagtgccgctctgtggagggcagctgtggcttctagctgcccgggtggcatccctgtgacccctccccagtgcctctcctggccctggaagttgccact |
| | ccagtgcccaccagtcttgtcctaataaaattaagttgcatcattttgtctgactaggtgtccttctataatattatggggtggaggggggtggtatggagcaagggaatagagtaagaa |
| | ccatatagagaggagatcagcaagaaaagaggctggcataggctctcgcaatgtctgtccatcagaagttgctttccccaggctattcaggaagccacgggctactcatgcttccaa |
| | cccctctctctgactttggatcatctacataaagggggaagacagaaaaaatcctaccagtgagttgaaaatacaggaaagcctatttcatatgggttaaagggtaggacagttgaattt |
| | cgtgaaaagtctgagttatataggctttgagcaaagagttttattagtatgaagcagaagaggtaacataaagaaagatgtatggggccaggcatggtggctcacacctgtaatccca |
| | gcactttgggaggccgaggtgggcgaatcactcctgggtgaactcaggagttcaagaccagcctgggcaacatggcgaaactccatctctacaaaaacattacgaaaattagctgg |
| | gcgtgttggtgctgtagtcccagctactcaggaggctgaggtgagaggcggaggaggttgcagtgagtcaagatcatgccactgcactccagcctgggcaacagagtaagaccct |
| | gtctcaaaaaaaaaaaaaagatagatgatgtatgctgtatgaaaaaaggaaacacacagatgattcaacagcctgttttgtggggtaatgaaaagtcaccctgggaactgggctcca |
| | gccctcgttctgccacccaccaactacatgtccttggcaagtcatatcaattatctgagtttctgttttataatctacaaataggttatctctggcagcttaataataatcagggttaacatttat |
| | taaacagtgtgtgccagtccatgtgctatgtgcttttctgtgaggtagttactgctatttacagaaacagtagatgcagagaccaaggtgctgagttaaatgattaggccaacaaggttag |
| | tacatgccgagccaggatggaagcccaggtaggcaggctggcttccgcggcaatgctcttatgaactatgttacgtccagtgctggaagggtctctcctgtagggcctgcggggtct |
| | attgggaaccaagctggagtgcagtggcacaatcttggctcactgcaatctccgcctcctgggttcaagcgattctcctgcctcagcctcccgagttgttgggattccaggcatcgagt |
| | agataagtagcatggcgggttaatcattaactacaaggaacccctagtgatggagttggccactccctctctgcgcgctcgctcgctcactgaggccgggcgaccaaaggtcgccc |
| | gacgcccgggctttgcccgggcggcctcagtgagcgagcgagcgcgcagagagggagtggccaaagatctgcgacgcgaggctggatggccttccccattatgattcttctcgc |
| | ttccggcggcatcgggatgcccgcgttgcaggccatgctgtccaggcaggtagatgacgaccatcagggacagcttcagcaaaaggccagcaaaaggccaggaaccgtaaaaa |
| | ggccgcgttgctggcgtttttccataggctccgcccccctgacgagcatcacaaaaatcgacgctcaagtcagaggtggcgaaacccgacaggactataaagataccaggcgtttc |
| | cccctggaagctccctcgtgcgctctcctgttccgaccctgccgcttaccggatacctgtccgcctttctcccttcgggaagcgtggcgctttctcatagctcacgctgtaggtatctca |
| | gttcggtgtaggtcgttcgctccaagctgggctgtgtgcacgaaccccccgttcagcccgaccgctgcgccttatccggtaactatcgtcttgagtccaacccggtaagacacgactt |
| | atcgccactggcagcagccactggtaacaggattagcagagcgaggtatgtaggcggtgctacagagttcttgaagtggtggcctaactacggctacactagaaggacagtatttg |
| | gtatctgcgctctgctgaagccagttaccttcggaaaaagagttggtagctcttgatccggcaaacaaaccaccgctggtagcggtggtttttttgtttgcaagcagcagattacgcgc |
| | agaaaaaaaggatctcaagaagatcctttgatcttttctacggggtctgacgctcagtggaacgaaaactcacgttaagggattttggtcatgagattatcaaaaaggatcttcacctag |
| | atccttttaaattaaaaatgaagttttaaatcaatctaaagtatatatgagtaaacttggtctgacagttaccaatgcttaatcagtgaggcacctatctcagcgatctgtctatttcgttcatcc |
| | atagttgcctgactccccgtcgtgtagataactacgatacgggagggcttaccatctggccccagtgctgcaatgataccgcgagacccacgctcaccggctccagatttatcagca |
| | ataaaccagccagccggaagggccgagcgcagaagtggtcctgcaactttatccgcctccatccagtctattaattgttgccgggaagctagagtaagtagttcgccagttaatagttt |
| | gcgcaacgttgttgccattgctgcaggcatcgtggtgtcacgctcgtcgtttggtatggcttcattcagctccggttcccaacgatcaaggcgagttacatgatcccccatgttgtgcaa |
| | aaaagcggttagctccttcggtcctccgatcgttgtcagaagtaagttggccgcagtgttatcactcatggttatggcagcactgcataattctcttactgtcatgccatccgtaagatgct |
| | tttctgtgactggtgagtactcaaccaagtcattctgagaatagtgtatgcggcgaccgagttgctcttgcccggcgtcaacacgggataataccgcgccacatagcagaactttaaaa |
| | gtgctcatcattggaaaacgttcttcggggcgaaaactctcaaggatcttaccgctgttgagatccagttcgatgtaacccactcgtgcacccaactgatcttcagcatcttttactttcac |
| | cagcgtttctgggtgagcaaaaacaggaaggcaaaatgccgcaaaaaagggaataagggcgacacggaaatgttgaatactcatactcttcctttttcaatattattgaagcatttatc |
| | agggttattgtctcatgagcggatacatatttgaatgtatttagaaaaataaacaaataggggttccgcgcacatttccccgaaaagtgccacctg |
| First Vector | cagatctttggccactccctctctgcgcgctcgctcgctcactgaggccgggcgaccaaaggtcgcccgacgcccgggctttgcccgggggcctcagtgagcgagcgagcgc |
| (Vector Set E) | gcagagagggagtggccaactccatcactaggggttcctggaggggtggagtcgtgacgtgaattacgtcatagggttagggaggtcggccgctctagagagctcaggaattcaa |
| (AAV-SpCas9 | gaccagcctgggcaacatggaaaaaccccatctctacaaaagatagaaaaattagccaggcatggtggcgtgtgcctgtggtcccagctactcaggaggctgaggtgggaggatc |
| Human | acattagcccaggaggttgaggctgcagtgagccgtgattatgccactgcactccagcctgggagacagagtgagaccctgtttcaaaaaaaagagagagaaaatttaaaaaaga |
| DONOR, see | aaacaacaccaagggctgtaactttaaggtcattaaatgaattaatcactgcattcaaaaacgattactttctggccctaagagacatgaggccaataccaggaagggggttgatctcc |
| FIG. 30) | caaaccagaggcagaccctagactctaatacagttaaggaaagaccagcaagatgatagtccccaatacaatagaagttactatattttatttgttgtttttcttttgttttgttttgttttgtttt |
| (SEQ ID NO: 45) | gttttgttttagagactggggtcttgctcgattgcccaggctgtagtgcagcggtgggacaatagctcactgcagactccaactcctgggctcaagcaatcctcctgcctcagcctcct |
| | gaatagctgggactacaagggtacaccatcacacacaccaaaacaattttttaaatttttgtgtagaaacgagggtcttgctttgttgcccaggctggtctccaactcctggcttcaagg |
| | gatcctcccacctcagcctcccaaattgctgggattacaggtgtgagccaccacaaccagccagaactttactaattttaaaattaagaacttaaaacttgaatagctagagcaccaag |
| | atttttctttgtccccaaataagtgcagttgcaggcatagaaaatctgacatctttgcaagaatcatcgtggatgtagactctgtcctgtgtctctggcctggtttcggggaccaggaggg |
| | cagacccttgcactgccaagaagcatgccaaagttaatcattggccctgctgagtacatggccgatcaggctgtttttgtgtgcctgtttttctattttacgtaaatcaccctgaacatgttt |
| | gcatcaacctactggtgatgcacctttgatcaatacattttagacaaacgtggtttttgagtccaaagatcagggctgggttgacctgaatactggatacagggcatataaaacagggg |
| | caaggcacagactcatagcagagcaatcaccaccaagcctggaataactgcaagggctctgctgacatcttcctgaggtgccaaggaaatgaggatggaggaaggaatgaatgtt |
| | ctccatgactttgggatccagtcaacacattacctccaggtgaattaccaagactcccaggactggttcatcttggtgtccgtgatcgcagacctcaggaatgccttctacgtcctcttcc |
| | ccatctggttccatcttcaggaagctgtgggcattaaactcctttgggtagctgtgattggagactggctcaacctcgtctttaagtggattctctttggacagcgtccatactggtgggttt |
| | tggatactgactactacagcaacacttccgtgcccctgataaagcagttccctgtaacctgtgagactggaccagggagcccctctggccatgccatgggcacagcaggtgtatact |
| | acgtgatggtcacatctactctttccatctttcagggaaagataaagccgacctacagatttcggtgcttgaatgtcattttgtggttgggattctgggctgtgcagctgaatgtctgtctgt |
| | cacgaatctaccttgctgctcattttcctcatcaagttgttgctggagtcctgtcaggcattgctgttgcagaaactttcagccacatccacagcatctataatgccagcctcaagaaatatt |
| | ttctcattaccttcttcctgttcagcttcgccatcggattttatctgctgctcaagggactgggtgtagacctcctgtggactctggagaaagcccagaggtggtgcgagcagccagaat |
| | gggtccacattgacaccacaccctttgccagcctcctcaagaacctgggcacgctctttggcctggggctggctctcaactccagcatgtacagggagagctgcaaggggaaactc |
| | agcaagtggctcccattccgcctcagctctattgtagcctccctcgtcctcctgcacgtctttgactccttgaaacccccatcccaagtcgagctggtcttctacgtcttgtccttctgcaa |
| | gagtgcggtagtgcccctggcatccgtcagtgtcatcccctactgcctcgcccaggtcctgggccagccgcacaagaagtcgttgtaaggcgaattctgcagatatccatcacactg |
| | gcggccgctcgacagcaacgtctatgacctcctaaaggacctagaggaaggcatccaaacgctgatgggggtgagggtggcgccaggggtccccaatcctggagccccactga |
| | ctttgagagctgtgttagagaaacactgctgccctctttttagcagtcaggccctgacccaagagaactcaccttattcttcatttcccctcgtgaatcctccaggcctttctctacaccctg |
| | aaggggagggaggaaaatgaatgaatgagaaagggagggaacagtacccaagcgcttggcctctccttctcttccttcactttgcagaggctggaagatggcagcccccggactg |
| | ggcagatcttcaagcagacctacagcaagttcgacacaaactcacacaacgatgacgcactactcaagaactacgggctgctctactgcttcaggaaggacatggacaaggtcga |
| | gacattcctgcgcatcgtgcagtgccgctctgtggagggcagctgtggcttctagctgcccgggtggcatccctgtgacccctccccagtgcctctcctggccctggaagttgccact |
| | ccagtgcccaccagtcttgtcctaataaaattaagttgcatcattttgtctgactaggtgtccttctataatattatggggtggaggggggtggtatggagcaagggaatagagtaagaa |
| | ccatatagagaggagatcagcaagaaaagaggctggcattcgctctcgcaatgtctgtccatcagaagttgctttccccaggctattcaggaagccacgggctactcatgcttccaac |
| | ccctctctctgactttggatcatctacataaagggggaagacagaaaaaatcctaccagtgagttgaaaatacaggaaagcctatttcatatgggttaaagggtaggacagttgaatttc |
| | gtgaaaagtctgagttatataggctttgagcaaagagttttattagtatgaagcagaagaggtaacataaagaaagatgtatggggccaggcatggtggctcacacctgtaatcccag |
| | cactttgggaggccgaggtgggcgaatcactcctgggtgaactcaggagttcaagaccagcctgggcaacatggcgaaactccatctctacaaaaacattacgaaaattagctggg |
| | cgtgttggtgctgtagtcccagctactcaggaggctgaggtgagaggcggaggaggttgcagtgagtcaagatcatgccactgcactccagcctgggcaacagagtaagaccctg |
| | tctcaaaaaaaaaaaaaagatagatgatgtatgctgtatgaaaaaaggaaacacacagatgattcaacagcctgttttgtggggtaatgaaaagtcaccctgggaactgggctccag |
| | ccctcgttctgccacccaccaactacatgtccttggcaagtcatatcaattatctgagtttctgttttataatctacaaataggttatctctggcagcttaataataatcagggttaacatttatt |
| | aaacagtgtgtgccagtccatgtgctatgtgcttttctgtgaggtagttactgctatttacagaaacagtagatgcagagaccaaggtgctgagttaaatgattaggccaacaaggttagt |
| | acatgccgagccaggatggaagcccaggtaggcaggctggcttccgcggcagtttggttaattaaggtaccgagggcctatttcccatgattccttcatatttgcatatacgatacaag |
| | gctgttagagagataattagaattaatttgactgtaaacacaaagatattagtacaaaatacgtgacgtagaaagtaataatttcttgggtagtttgcagttttaaaattatgttttaaaatgga |
| | ctatcatatgcttaccgtaacttgaaagtatttcgatttcttggctttatatatcttgtggaaaggacgaaacaccgtctacgtcctcttccccatcgttttagagctagaaatagcaagttaaa |
| | ataaggctagtccgttatcaacttgaaaaagtggcaccgagtcggtgcttttttgccgcgggaagggtctctcctgtagggccttcgctcgctcactgaggccgggcgaccaaaggt |
| | cgcccgacgcccgggctttgcccgggcggcctcagtgagcgagcgagcgcgcagagagggagtggccaaagatctgcgacgcgaggctggatggccttccccattatgattct |
| | tctcgcttccggcggcatcgggatgcccgcgttgcaggccatgctgtccaggcaggtagatgacgaccatcagggacagcttcagcaaaaggccagcaaaaggccaggaaccg |
| | taaaaaggccgcgttgctggcgtttttccataggctccgcccccctgacgagcatcacaaaaatcgacgctcaagtcagaggtggcgaaacccgacaggactataaagataccag |
| | gcgtttccccctggaagctccctcgtgcgctctcctgttccgaccctgccgcttaccggatacctgtccgcctttctcccttcgggaagcgtggcgctttctcatagctcacgctgtaggt |
| | atctcagttcggtgtaggtcgttcgctccaagctgggctgtgtgcacgaaccccccgttcagcccgaccgctgcgccttatccggtaactatcgtcttgagtccaacccggtaagaca |
| | cgacttatcgccactggcagcagccactggtaacaggattagcagagcgaggtatgtaggcggtgctacagagttcttgaagtggtggcctaactacggctacactagaaggacag |
| | tatttggtatctgcgctctgctgaagccagttaccttcggaaaaagagttggtagctcttgatccggcaaacaaaccaccgctggtagcggtggtttttttgtttgcaagcagcagattac |
| | gcgcagaaaaaaaggatctcaagaagatcctttgatcttttctacggggtctgacgctcagtggaacgaaaactcacgttaagggattttggtcatgagattatcaaaaaggatcttcac |
| | ctagatccttttaaattaaaaatgaagttttaaatcaatctaaagtatatatgagtaaacttggtctgacagttaccaatgcttaatcagtgaggcacctatctcagcgatctgtctatttcgttc |
| | atccatagttgcctgactccccgtcgtgtagataactacgatacgggagggcttaccatctggccccagtgctgcaatgataccgcgagacccacgctcaccggctccagatttatca |
| | gcaataaaccagccagccggaagggccgagcgcagaagtggtcctgcaactttatccgcctccatccagtctattaattgttgccgggaagctagagtaagtagttcgccagttaat |
| | agtttgcgcaacgttgttgccattgctgcaggcatcgtggtgtcacgctcgtcgtttggtatggcttcattcagctccggttcccaacgatcaaggcgagttacatgatcccccatgttgt |
| | gcaaaaaagcggttagctccttcggtcctccgatcgttgtcagaagtaagttggccgcagtgttatcactcatggttatggcagcactgcataattctcttactgtcatgccatccgtaag |
| | atgcttttctgtgactggtgagtactcaaccaagtcattctgagaatagtgtatgcggcgaccgagttgctcttgcccggcgtcaacacgggataataccgcgccacatagcagaactt |
| | taaaagtgctcatcattggaaaacgttcttcggggcgaaaactctcaaggatcttaccgctgttgagatccagttcgatgtaacccactcgtgcacccaactgatcttcagcatcttttact |
| | ttcaccagcgtttctgggtgagcaaaaacaggaaggcaaaatgccgcaaaaaagggaataagggcgacacggaaatgttgaatactcatactcttcctttttcaatattattgaagcat |
| | ttatcagggttattgtctcatgagcggatacatatttgaatgtatttagaaaaataaacaaataggggttccgcgcacatttccccgaaaagtgccacctg |
| Second Vector | ggggggggggggggggggttggccactccctctctgcgcgctcgctcgctcactgaggccgggcgaccaaaggtcgcccgacgcccgggctttgcccgggcggcctcagtg |
| (Vector Set | agcgagcgagcgcgcagagagggagtggccaactccatcactaggggttcctgcggccaattcgagctcggtaccctctgtcctgtgtctctggcctggtttcggggaccaggag |
| A/B/E) | ggcagacccttgcactgccaagaagcatgccaaagttaatcattggccctgctgagtacatggccgatcaggctgtttttgtgtgcctgtttttctattttacgtaaatcaccctgaacatg |
| (AAV-G6Pcmin | tttgcatcaacctactggtgatgcacctttgatcaatacattttagacaaacgtggtttttgagtccaaagatcagggctgggttgacctgaatactggatacagggcatataaaacaggg |
| 303 SpCas9 | gcaaggcacagactcatagctctagagcgctgccaccatggacaagaagtacagcatcggcctggacatcggcaccaactctgtgggctgggccgtgatcaccgacgagtacaa |
| Final MOUSE | ggtgcccagcaagaaattcaaggtgctgggcaacaccgaccggcacagcatcaagaagaacctgatcggagccctgctgttcgacagcggcgaaacagccgaggccacccgg |
| CRISPR, see | ctgaagagaaccgccagaagaagatacaccagacggaagaaccggatctgctatctgcaagagatcttcagcaacgagatggccaaggtggacgacagcttcttccacagactg |
| FIG. 12) | gaagagtccttcctggtggaagaggataagaagcacgagcggcaccccatcttcggcaacatcgtggacgaggtggcctaccacgagaagtaccccaccatctaccacctgaga |
| (SEQ ID NO: 46) | aagaaactggtggacagcaccgacaaggccgacctgcggctgatctatctggccctggcccacatgatcaagttccggggccacttcctgatcgagggcgacctgaaccccgac |
| | aacagcgacgtggacaagctgttcatccagctggtgcagacctacaaccagctgttcgaggaaaaccccatcaacgccagcggcgtggacgccaaggccatcctgtctgccaga |
| | ctgagcaagagcagacggctggaaaatctgatcgcccagctgcccggcgagaagaagaatggcctgttcggaaacctgattgccctgagcctgggcctgacccccaacttcaag |
| | agcaacttcgacctggccgaggatgccaaactgcagctgagcaaggacacctacgacgacgacctggacaacctgctggcccagatcggcgaccagtacgccgacctgtttctg |
| | gccgccaagaacctgtccgacgccatcctgctgagcgacatcctgagagtgaacaccgagatcaccaaggcccccctgagcgcctctatgatcaagagatacgacgagcaccac |
| | caggacctgaccctgctgaaagctctcgtgcggcagcagctgcctgagaagtacaaagagattttcttcgaccagagcaagaacggctacgccggctacattgacggcggagcc |
| | agccaggaagagttctacaagttcatcaagcccatcctggaaaagatggacggcaccgaggaactgctcgtgaagctgaacagagaggacctgctgcggaagcagcggaccttc |
| | gacaacggcagcatcccccaccagatccacctgggagagctgcacgccattctgcggcggcaggaagatttttacccattcctgaaggacaaccgggaaaagatcgagaagatc |
| | ctgaccttccgcatcccctactacgtgggccctctggccaggggaaacagcagattcgcctggatgaccagaaagagcgaggaaaccatcaccccctggaacttcgaggaagtg |
| | gtggacaagggcgcttccgcccagagcttcatcgagcggatgaccaacttcgataagaacctgcccaacgagaaggtgctgcccaagcacagcctgctgtacgagtacttcaccg |
| | tgtataacgagctgaccaaagtgaaatacgtgaccgagggaatgagaaagcccgccttcctgagcggcgagcagaaaaaggccatcgtggacctgctgttcaagaccaaccgga |
| | aagtgaccgtgaagcagctgaaagaggactacttcaagaaaatcgagtgcttcgactccgtggaaatctccggcgtggaagatcggttcaacgcctccctgggcacataccacgat |
| | ctgctgaaaattatcaaggacaaggacttcctggacaatgaggaaaacgaggacattctggaagatatcgtgctgaccctgacactgtttgaggacagagagatgatcgaggaacg |
| | gctgaaaacctatgcccacctgttcgacgacaaagtgatgaagcagctgaagcggcggagatacaccggctggggcaggctgagccggaagctgatcaacggcatccgggac |
| | aagcagtccggcaagacaatcctggatttcctgaagtccgacggcttcgccaacagaaacttcatgcagctgatccacgacgacagcctgacctttaaagaggacatccagaaagc |
| | ccaggtgtccggccagggcgatagcctgcacgagcacattgccaatctggccggcagccccgccattaagaagggcatcctgcagacagtgaaggtggtggacgagctcgtga |
| | aagtgatgggccggcacaagcccgagaacatcgtgatcgaaatggccagagagaaccagaccacccagaagggacagaagaacagccgcgagagaatgaagcggatcgaa |
| | gagggcatcaaagagctgggcagccagatcctgaaagaacaccccgtggaaaacacccagctgcagaacgagaagctgtacctgtactacctgcagaatggggggatatgta |
| | cgtggaccaggaactggacatcaaccggctgtccgactacgatgtggaccatatcgtgcctcagagctttctgaaggacgactccatcgacaacaaggtgctgaccagaagcgac |
| | aagaaccggggcaagagcgacaacgtgccctccgaagaggtcgtgaagaagatgaagaactactggcggcagctgctgaacgccaagctgattacccagagaaagttcgaca |
| | atctgaccaaggccgagagaggcggcctgagcgaactggataaggccggcttcatcaagagacagctggtggaaacccggcagatcacaaagcacgtggcacagatcctgga |
| | ctcccggatgaacactaagtacgacgagaatgacaagctgatccgggaagtgaaagtgatcaccctgaagtccaagctggtgtccgatttccggaaggatttccagttttacaaagt |
| | gcgcgagatcaacaactaccaccacgcccacgacgcctacctgaacgccgtcgtgggaaccgccctgatcaaaaagtaccctaagctggaaagcgagttcgtgtacggcgacta |
| | caaggtgtacgacgtgcggaagatgatcgccaagagcgagcaggaaatcggcaaggctaccgccaagtacttcttctacagcaacatcatgaactttttcaagaccgagattaccct |
| | ggccaacggcgagatccggaagcggcctctgatcgagacaaacggcgaaaccggggagatcgtgtgggataagggccgggattttgccaccgtgcggaaagtgctgagcatg |
| | ccccaagtgaatatcgtgaaaaagaccgaggtgcagacaggcggcttcagcaaagagtctatcctgcccaagaggaacagcgataagctgatcgccagaaagaaggactggga |
| | ccctaagaagtacggcggcttcgacagccccaccgtggcctattctgtgctggtggtggccaaagtggaaaagggcaagtccaagaaactgaagagtgtgaaagagctgctggg |
| | gatcaccatcatggaaagaagcagcttcgagaagaatcccatcgactttctggaagccaagggctacaaagaagtgaaaaaggacctgatcatcaagctgcctaagtactccctgtt |
| | cgagctggaaaacggccggaagagaatgctggcctctgccggcgaactgcagaagggaaacgaactggccctgccctccaaatatgtgaacttcctgtacctggccagccactat |
| | gagaagctgaagggctcccccgaggataatgagcagaaacagctgtttgtggaacagcacaagcactacctggacgagatcatcgagcagatcagcgagttctccaagagagtg |
| | atcctggccgacgctaatctggacaaagtgctgtccgcctacaacaagcaccgggataagcccatcagagagcaggccgagaatatcatccacctgtttaccctgaccaatctggg |
| | agcccctgccgccttcaagtactttgacaccaccatcgaccggaagaggtacaccagcaccaaagaggtgctggacgccaccctgatccaccagagcatcaccggcctgtacga |
| | gacacggatcgacctgtctcagctgggaggcgacaagcgacctgccgccacaaagaaggctggacaggctaagaagaagaaagattacaaagacgatgacgataagtgagtc |
| | gacaataaaatatctttattttcattacatctgtgtgttggttttttgtgtgtgcatgctggggacggccgcaggaacccctagtgatggagttggccactccctctctgcgcgctcgctcg |
| | ctcactgaggccgcccgggcaaagcccgggcgtcgggcgacctttggtcgcccggcctcagtgagcgagcgagcgcgcagagagggagtggccaaccccccccccccccc |
| | ccctgcagccctgcattaatgaatcggccaacgcgcggggagaggcggtttgcgtattgggcgctcttccgcttcctcgctcactgactcgctgcgctcggtcgttcggctgcggcg |
| | agcggtatcagctcactcaaaggcggtaatacggttatccacagaatcaggggataacgcaggaaagaacatgtgagcaaaaggccagcaaaaggccaggaaccgtaaaaagg |
| | ccgcgttgctggcgtttttccataggctccgcccccctgacgagcatcacaaaaatcgacgctcaagtcagaggtggcgaaacccgacaggactataaagataccaggcgtttccc |
| | cctggaagctccctcgtgcgctctcctgttccgaccctgccgcttaccggatacctgtccgcctttctcccttcgggaagcgtggcgctttctcaatgctcacgctgtaggtatctcagtt |
| | cggtgtaggtcgttcgctccaagctgggctgtgtgcacgaaccccccgttcagcccgaccgctgcgccttatccggtaactatcgtcttgagtccaacccggtaagacacgacttatc |
| | gccactggcagcagccactggtaacaggattagcagagcgaggtatgtaggcggtgctacagagttcttgaagtggtggcctaactacggctacactagaaggacagtatttggtat |
| | ctgcgctctgctgaagccagttaccttcggaaaaagagttggtagctcttgatccggcaaacaaaccaccgctggtagcggtggtttttttgtttgcaagcagcagattacgcgcagaa |
| | aaaaaggatctcaagaagatcctttgatcttttctacggggtctgacgctcagtggaacgaaaactcacgttaagggattttggtcatgagattatcaaaaaggatcttcacctagatcct |
| | tttaaattaaaaatgaagttttaaatcaatctaaagtatatatgagtaaacttggtctgacagttaccaatgcttaatcagtgaggcacctatctcagcgatctgtctatttcgttcatccatagt |
| | tgcctgactccccgtcgtgtagataactacgatacgggagggcttaccatctggccccagtgctgcaatgataccgcgagacccacgctcaccggctccagatttatcagcaataaa |
| | ccagccagccggaagggccgagcgcagaagtggtcctgcaactttatccgcctccatccagtctattaattgttgccgggaagctagagtaagtagttcgccagttaatagtttgcgc |
| | aacgttgttgccattgctacaggcatcgtggtgtcacgctcgtcgtttggtatggcttcattcagctccggttcccaacgatcaaggcgagttacatgatcccccatgttgtgcaaaaaa |
| | gcggttagctccttcggtcctccgatcgttgtcagaagtaagttggccgcagtgttatcactcatggttatggcagcactgcataattctcttactgtcatgccatccgtaagatgcttttct |
| | gtgactggtgagtactcaaccaagtcattctgagaatagtgtatgcggcgaccgagttgctcttgcccggcgtcaatacgggataataccgcgccacatagcagaactttaaaagtgc |
| | tcatcattggaaaacgttcttcggggcgaaaactctcaaggatcttaccgctgttgagatccagttcgatgtaacccactcgtgcacccaactgatcttcagcatcttttactttcaccagc |
| | gtttctgggtgagcaaaaacaggaaggcaaaatgccgcaaaaaagggaataagggcgacacggaaatgttgaatactcatactcttcctttttcaatattattgaagcatttatcagggt |
| | tattgtctcatgagcggatacatatttgaatgtatttagaaaaataaacaaataggggttccgcgcacatttccccgaaaagtgccacctgacgtctaagaaaccattattatcatgacatt |
| | aacctataaaaataggcgtatcacgaggccctttcgtctcgcgcgtttcggtgatgacggtgaaaacctctgacacatgcagctcccggagacggtcacagcttgtctgtaagcggat |
| | gccgggagcagacaagcccgtcagggcgcgtcagcgggtgttggcgggtgtcggggctggcttaactatgcggcatcagagcagattgtactgagagtgcaccatatgcggtgt |
| | gaaataccgcacagatgcgtaaggagaaaataccgcatcaggaaattgtaaacgttaatattttgttaaaattcgcgttaaatttttgttaaatcagctcattttttaaccaataggccgaaa |
| | tcggcaaaatcccttataaatcaaaagaatagaccgagatagggttgagtgttgttccagtttggaacaagagtccactattaaagaacgtggactccaacgtcaaagggcgaaaaa |
| | ccgtctatcagggcgatggcccactacgtgaaccatcaccctaatcaagttttttggggtcgaggtgccgtaaagcactaaatcggaaccctaaagggagcccccgatttagagctt |
| | gacggggaaagccggcgaacgtggcgagaaaggaagggaagaaagcgaaaggagcgggcgctagggcgctggcaagtgtagcggtcacgctgcgcgtaaccaccacacc |
| | cgccgcgcttaatgcgccgctacagggcgcgtcgcgccattcgccattcaggctacgcaactgttgggaagggcgatcggtgcgggcctcttcgctattacgccaggctgca |
| Second Vector | cctgcaggcagctgcgcgctcgctcgctcactgaggccgcccgggcaaagcccgggcgtcgggcgacctttggtcgcccggcctcagtgagcgagcgagcgcgcagagagg |
| (Vector Set C) | gagtggccaactccatcactaggggttcctgcggccgcctcgaggatccgaattcgagctcggtaccaagcttgccgagctcaggtacccgctaatctagagagggcctatttccc |
| (AAV- | atgattccttcatatttgcatatacgatacaaggctgttagagagataattggaattaatttgactgtaaacacaaagatattagtacaaaatacgtgacgtagaaagtaataatttcttgggt |
| cG6PgRNACas9 | agtttgcagttttaaaattatgttttaaaatggactatcatatgcttaccgtaacttgaaagtatttcgatttcttggctttatatatcttgtggaaaggacgaaacaccgatggtcagacatttc |
| DOG CRISPR, | cagagttttagtactctggaaacagaatctactaaaacaaggcaaaatgccgtgtttatctcgtcaacttgttggcgagattttttctcgaggtcggaggatccacaaaggctagccggt |
| see FIG. 10) | ctatgcatacattgattattgactagttattaatagtaatcaattacggggtcattagttcatagcccatatatggagttccgcgttacataacttacggtaaatggcccgcctggctgaccg |
| (SEQ ID NO: 47) | cccaacgacccccgcccattgacgtcaataatgacgtatgttcccatagtaacgccaatagggactttccattgacgtcaatgggtggagtatttacggtaaactgcccacttggcagt |
| | acatcaagtgtatcatatgccaagtacgccccctattgacgtcaatgacggtaaatggcccgcctggcattatgcccagtacatgaccttatgggactttcctacttggcagtacatcta |
| | cgtattagtcatcgctattaccatggtgatgcggttttggcagtacatcaatgggcgtggatagcggtttgactcacggggatttccaagtctccaccccattgacgtcaatgggagtttg |
| | ttttggcaccaaaatcaacgggactttccaaaatgtcgtaacaactccgccccattgacgcaaatgggcggtaggcgtgtacggtgggaggtctatataagcagagctcgtttagtga |
| | accgtcagatcgcctggagacaccatccacgctgttttgacctccatagaagacaccgggaccgatccagcctaagcttgccaccatgaagaggaattacatcctcggactggaca |
| | tcggcattaccagcgtcggctacggcatcatcgattacgagacaagggacgtgattgacgctggcgtcaggctgttcaaggaggccaacgtggagaataatgagggcaggagaa |
| | gcaagaggggcgccaggaggctgaagaggaggagaaggcacagaatccagagggtcaagaagctcctgttcgactacaacctgctgaccgaccatagcgagctgagcggca |
| | ttaacccctacgaggctagggtcaaaggcctctcccagaaactgtccgaggaggagtttagcgctgctctcctgcacctggccaagaggaggggagtgcacaatgtgaacgaggt |
| | cgaagaggacaccggcaatgagctcagcaccaaggagcagattagcagaaacagcaaagccctggaggaaaaatatgtcgccgagctacagctggagagactcaagaaggat |
| | ggcgaggtcagaggctccattaacaggttcaagacctccgactacgtgaaagaagccaagcagctgctgaaggtgcaaaaggcctatcatcagctggaccagagcttcattgata |
| | cctatatcgacctgctcgaaaccaggagaacctactatgagggccctggagaaggaagccccttcggatggaaagacattaaggagtggtacgaaatgctgatgggacactgcac |
| | ctacttccccgaggagctgaggagcgtgaaatatgcctacaacgccgatctctacaacgccctgaacgacctgaacaatctcgtgattacaagggatgagaacgaaaagctggaat |
| | attacgaaaaatttcagatcattgaaaacgtgtttaagcaaaagaagaagcccaccctgaagcagattgccaaggagatcctggtgaacgaagaggacattaaaggctacagagtg |
| | acctccacaggcaaacccgaatttaccaatctgaaggtgtatcacgatatcaaagatatcacagctagaaaggagatcatcgagaatgctgagctgctcgaccaaatcgccaagatt |
| | ctcaccatctaccagagcagcgaggacatccaggaagaactgacaaacctgaactccgagctcacccaggaagaaatcgagcagatttccaacctgaaaggctacaccggcacc |
| | cacaacctgtccctgaaggccatcaacctgatcctcgatgaactctggcacaccaatgacaatcaaatcgctatcttcaacagactgaagctcgtgcccaagaaagtggacctgagc |
| | cagcagaaggaaatccctacaaccctcgtggacgatttcatcctcagccccgtcgtgaagaggagcttcatccagtccatcaaggtgattaacgctatcatcaagaagtatggcctcc |
| | ccaacgacatcatcatcgagctcgctagggaaaaaaactccaaggatgctcagaaaatgatcaacgagatgcagaaaaggaacagacaaaccaacgaaaggattgaggagatta |
| | tcaggaccaccggcaaggagaatgctaagtatctgattgaaaaaattaaactccacgatatgcaggagggcaagtgtctctatagcctggaagccatccctctggaggatctcctga |
| | acaatccttttaactacgaagtggaccacattatccccaggagcgtgtccttcgataactcctttaataataaggtcctggtcaaacaggaggagaactccaagaagggaaacaggac |
| | acccttccaatatctctccagcagcgactccaaaataagctacgaaacctttaaaaaacacattctgaacctggccaagggcaaaggcaggatcagcaaaaccaaaaaggaatacc |
| | tcctggaggagagagacatcaacaggttctccgtgcagaaagacttcatcaacaggaacctggtggacaccaggtatgccaccaggggcctgatgaacctcctgaggtcctatttta |
| | gagtcaataatctcgacgtgaaagtcaaatccattaatggcggctttaccagcttcctgaggagaaaatggaagttcaagaaggagagaaacaaaggctacaaacaccacgccgaa |
| | gatgccctcatcattgctaacgccgacttcatctttaaggagtggaagaagctcgataaggccaagaaagtcatggagaatcaaatgttcgaggagaaacaagccgaatccatgccc |
| | gagattgaaacggagcaagagtacaaggaaatattcatcacccctcaccagatcaaacacatcaaggacttcaaagactacaaatatagccatagagtcgataagaagcccaacag |
| | ggagctgatcaacgacacactctattccaccagaaaggatgataagggcaacaccctgattgtgaacaacctgaacggcctctacgataaggataacgacaagctgaagaagctg |
| | attaacaagagccccgagaaactgctcatgtaccaccacgacccccaaacataccagaaactgaagctgatcatggaacagtatggcgacgagaaaaaccccctgtataagtacta |
| | tgaagaaaccggcaactacctcaccaagtacagcaaaaaggataacggacccgtgatcaaaaagattaagtattacggcaataagctgaatgcccacctcgatatcaccgatgatta |
| | ccccaactccagaaataaagtcgtgaaactgtccctgaaaccttacaggtttgacgtgtacctggacaatggcgtgtacaagttcgtgaccgtgaagaacctcgacgtgattaagaag |
| | gagaattactatgaggtcaacagcaaatgctatgaggaagccaagaaactgaagaagatttccaatcaggccgagttcatcgccagcttttataacaacgacctgattaagatcaatg |
| | gcgaactgtatagggtgatcggcgtcaacaacgacctcctgaacaggattgaggtcaatatgatcgatattacctacagagagtacctcgaaaacatgaatgacaagaggcccccc |
| | aggattatcaaaaccatcgccagcaagacccagagcattaagaaatacagcaccgacatcctcggcaacctgtacgaggtcaagtccaaaaagcacccccagattatcaaaaagg |
| | gaaagcgacctgccgccacaaagaaggctggacaggctaagaagaagaaagattacaaagacgatgacgataagtgagtcgaccgactagagctcgctgatcagcctcgactg |
| | tgccttctagttgccagccatctgttgtttgcccctcccccgtgccttccttgaccctggaaggtgccactcccactgtcctttcctaataaaatgaggaaattgcatcgcattgtctgagta |
| | ggtgtcattctattctggggggtggggggggcaggacagcaagggggaggattgggaagacaatagcaggcatgctggggacgcaggaacccctagtgatggagttggccac |
| | tccctctctgcgcgctcgctcgctcactgaggccgggcgaccaaaggtcgcccgacgcccgggctttgcccgggggcctcagtgagcgagcgagcgcgcagctgcctgcag |
| | gggcgcctgatgcggtattttctccttacgcatctgtgcggtatttcacaccgcatacgtcaaagcaaccatagtacgcgccctgtagcggcgcattaagcgcggcgggtgtggtggt |
| | tacgcgcagcgtgaccgctacacttgccagcgccctagcgcccgctcctttcgctttcttcccttcctttctcgccacgttcgccggctttccccgtcaagctctaaatcgggggctccc |
| | tttagggttccgatttagtgctttacggcacctcgaccccaaaaaacttgatttgggtgatggttcacgtagtgggccatcgccctgatagacggtttttcgccctttgacgttggagtcca |
| | cgttctttaatagtggactcttgttccaaactggaacaacactcaaccctatctcgggctattcttttgatttataagggattttgccgatttcggcctattggttaaaaaatgagctgatttaa |
| | caaaaatttaacgcgaattttaacaaaatattaacgtttacaattttatggtgcactctcagtacaatctgctctgatgccgcatagttaagccagccccgacacccgccaacacccgctg |
| | acgcgccctgacgggcttgtctgctcccggcatccgcttacagacaagctgtgaccgtctccgggagctgcatgtgtcagaggttttcaccgtcatcaccgaaacgcgcgagacga |
| | aagggcctcgtgatacgcctatttttataggttaatgtcatgataataatggtttcttagacgtcaggtggcacttttcggggaaatgtgcgcggaacccctatttgtttatttttctaaataca |
| | ttcaaatatgtatccgctcatgagacaataaccctgataaatgcttcaataatattgaaaaaggaagagtatgagtattcaacatttccgtgtcgcccttattcccttttttgcggcattttgcc |
| | ttcctgtttttgctcacccagaaacgctggtgaaagtaaaagatgctgaagatcagttgggtgcacgagtgggttacatcgaactggatctcaacagcggtaagatccttgagagttttc |
| | gccccgaagaacgttttccaatgatgagcacttttaaagttctgctatgtggcgcggtattatcccgtattgacgccgggcaagagcaactcggtcgccgcatacactattctcagaat |
| | gacttggttgagtactcaccagtcacagaaaagcatcttacggatggcatgacagtaagagaattatgcagtgctgccataaccatgagtgataacactgcggccaacttacttctgac |
| | aacgatcggaggaccgaaggagctaaccgcttttttgcacaacatgggggatcatgtaactcgccttgatcgttgggaaccggagctgaatgaagccataccaaacgacgagcgt |
| | gacaccacgatgcctgtagcaatggcaacaacgttgcgcaaactattaactggcgaactacttactctagcttcccggcaacaattaatagactggatggaggcggataaagttgca |
| | ggaccacttctgcgctcggcccttccggctggctggtttattgctgataaatctggagccggtgagcgtgggtctcgcggtatcattgcagcactggggccagatggtaagccctccc |
| | gtatcgtagttatctacacgacggggagtcaggcaactatggatgaacgaaatagacagatcgctgagataggtgcctcactgattaagcattggtaactgtcagaccaagtttactca |
| | tatatactttagattgatttaaaacttcatttttaatttaaaaggatctaggtgaagatcctttttgataatctcatgaccaaaatcccttaacgtgagttttcgttccactgagcgtcagacccc |
| | gtagaaaagatcaaaggatcttcttgagatcctttttttctgcgcgtaatctgctgcttgcaaacaaaaaaaccaccgctaccagcggtggtttgtttgccggatcaagagctaccaactc |
| | tttttccgaaggtaactggcttcagcagagcgcagataccaaatactgtccttctagtgtagccgtagttaggccaccacttcaagaactctgtagcaccgcctacatacctcgctctgc |
| | taatcctgttaccagtggctgctgccagtggcgataagtcgtgtcttaccgggttggactcaagacgatagttaccggataaggcgcagcggtcgggctgaacggggggttcgtgc |
| | acacagcccagcttggagcgaacgacctacaccgaactgagatacctacagcgtgagctatgagaaagcgccacgcttcccgaagggagaaaggcggacaggtatccggtaag |
| | cggcagggtcggaacaggagagcgcacgagggagcttccagggggaaacgcctggtatctttatagtcctgtcgggtttcgccacctctgacttgagcgtcgatttttgtgatgctc |
| | gtcaggggggcggagcctatggaaaaacgccagcaacgcggcctttttacggttcctggccttttgctggccttttgctcacatgt |
| Second Vector | cctgcaggcagctgcgcgctcgctcgctcactgaggccgcccgggcaaagcccgggcgtcgggcgacctttggtcgcccggcctcagtgagcgagcgagcgcgcagagagg |
| Set D | gagtggccaactccatcactaggggttcctgcggccgcctcgaggatccgaattcgagctcggtaccaagcttgccgagctcaggtacccgctaatctagagagggcctatttccc |
| (AAV-SaCas9 | atgattccttcatatttgcatatacgatacaaggctgttagagagataattggaattaatttgactgtaaacacaaagatattagtacaaaatacgtgacgtagaaagtaataatttcttgggt |
| Human CRISPR, | agtttgcagttttaaaattatgttttaaaatggactatcatatgcttaccgtaacttgaaagtatttcgatttcttggctttatatatcttgtggaaaggacgaaacaccgtgatggacagacatt |
| see FIG. 29) | gcgagagttttagtactctggaaacagaatctactaaaacaaggcaaaatgccgtgtttatctcgtcaacttgttggcgagattttttctcgaggtcggaggatccacaaaggctagccg |
| (SEQ ID NO: 48) | gtctatgcatacattgattattgactagttattaatagtaatcaattacggggtcattagttcatagcccatatatggagttccgcgttacataacttacggtaaatggcccgcctggctgac |
| | cgcccaacgacccccgcccattgacgtcaataatgacgtatgttcccatagtaacgccaatagggactttccattgacgtcaatgggtggagtatttacggtaaactgcccacttggca |
| | gtacatcaagtgtatcatatgccaagtacgccccctattgacgtcaatgacggtaaatggcccgcctggcattatgcccagtacatgaccttatgggactttcctacttggcagtacatct |
| | acgtattagtcatcgctattaccatggtgatgcggttttggcagtacatcaatgggcgtggatagcggtttgactcacggggatttccaagtctccaccccattgacgtcaatgggagttt |
| | gttttggcaccaaaatcaacgggactttccaaaatgtcgtaacaactccgccccattgacgcaaatgggcggtaggcgtgtacggtgggaggtctatataagcagagctcgtttagtg |
| | aaccgtcagatcgcctggagacaccatccacgctgttttgacctccatagaagacaccgggaccgatccagcctaagcttgccaccatgaagaggaattacatcctcggactggac |
| | atcggcattaccagcgtcggctacggcatcatcgattacgagacaagggacgtgattgacgctggcgtcaggctgttcaaggaggccaacgtggagaataatgagggcaggaga |
| | agcaagaggggcgccaggaggctgaagaggaggagaaggcacagaatccagagggtcaagaagctcctgttcgactacaacctgctgaccgaccatagcgagctgagcggc |
| | attaacccctacgaggctagggtcaaaggcctctcccagaaactgtccgaggaggagtttagcgctgctctcctgcacctggccaagaggaggggagtgcacaatgtgaacgag |
| | gtcgaagaggacaccggcaatgagctcagcaccaaggagcagattagcagaaacagcaaagccctggaggaaaaatatgtcgccgagctacagctggagagactcaagaagg |
| | atggcgaggtcagaggctccattaacaggttcaagacctccgactacgtgaaagaagccaagcagctgctgaaggtgcaaaaggcctatcatcagctggaccagagcttcattgat |
| | acctatatcgacctgctcgaaaccaggagaacctactatgagggccctggagaaggaagccccttcggatggaaagacattaaggagtggtacgaaatgctgatgggacactgca |
| | cctacttccccgaggagctgaggagcgtgaaatatgcctacaacgccgatctctacaacgccctgaacgacctgaacaatctcgtgattacaagggatgagaacgaaaagctgga |
| | atattacgaaaaatttcagatcattgaaaacgtgtttaagcaaaagaagaagcccaccctgaagcagattgccaaggagatcctggtgaacgaagaggacattaaaggctacagagt |
| | gacctccacaggcaaacccgaatttaccaatctgaaggtgtatcacgatatcaaagatatcacagctagaaaggagatcatcgagaatgctgagctgctcgaccaaatcgccaagat |
| | tctcaccatctaccagagcagcgaggacatccaggaagaactgacaaacctgaactccgagctcacccaggaagaaatcgagcagatttccaacctgaaaggctacaccggcac |
| | ccacaacctgtccctgaaggccatcaacctgatcctcgatgaactctggcacaccaatgacaatcaaatcgctatcttcaacagactgaagctcgtgcccaagaaagtggacctgag |
| | ccagcagaaggaaatccctacaaccctcgtggacgatttcatcctcagccccgtcgtgaagaggagcttcatccagtccatcaaggtgattaacgctatcatcaagaagtatggcctc |
| | cccaacgacatcatcatcgagctcgctagggaaaaaaactccaaggatgctcagaaaatgatcaacgagatgcagaaaaggaacagacaaaccaacgaaaggattgaggagatt |
| | atcaggaccaccggcaaggagaatgctaagtatctgattgaaaaaattaaactccacgatatgcaggagggcaagtgtctctatagcctggaagccatccctctggaggatctcctg |
| | aacaatccttttaactacgaagtggaccacattatccccaggagcgtgtccttcgataactcctttaataataaggtcctggtcaaacaggaggagaactccaagaagggaaacagga |
| | cacccttccaatatctctccagcagcgactccaaaataagctacgaaacctttaaaaaacacattctgaacctggccaagggcaaaggcaggatcagcaaaaccaaaaaggaatac |
| | ctcctggaggagagagacatcaacaggttctccgtgcagaaagacttcatcaacaggaacctggtggacaccaggtatgccaccaggggcctgatgaacctcctgaggtcctatttt |
| | agagtcaataatctcgacgtgaaagtcaaatccattaatggcggctttaccagcttcctgaggagaaaatggaagttcaagaaggagagaaacaaaggctacaaacaccacgccga |
| | agatgccctcatcattgctaacgccgacttcatctttaaggagtggaagaagctcgataaggccaagaaagtcatggagaatcaaatgttcgaggagaaacaagccgaatccatgcc |
| | cgagattgaaacggagcaagagtacaaggaaatattcatcacccctcaccagatcaaacacatcaaggacttcaaagactacaaatatagccatagagtcgataagaagcccaaca |
| | gggagctgatcaacgacacactctattccaccagaaaggatgataagggcaacaccctgattgtgaacaacctgaacggcctctacgataaggataacgacaagctgaagaagct |
| | gattaacaagagccccgagaaactgctcatgtaccaccacgacccccaaacataccagaaactgaagctgatcatggaacagtatggcgacgagaaaaaccccctgtataagtac |
| | tatgaagaaaccggcaactacctcaccaagtacagcaaaaaggataacggacccgtgatcaaaaagattaagtattacggcaataagctgaatgcccacctcgatatcaccgatgat |
| | taccccaactccagaaataaagtcgtgaaactgtccctgaaaccttacaggtttgacgtgtacctggacaatggcgtgtacaagttcgtgaccgtgaagaacctcgacgtgattaaga |
| | aggagaattactatgaggtcaacagcaaatgctatgaggaagccaagaaactgaagaagatttccaatcaggccgagttcatcgccagcttttataacaacgacctgattaagatcaa |
| | tggcgaactgtatagggtgatcggcgtcaacaacgacctcctgaacaggattgaggtcaatatgatcgatattacctacagagagtacctcgaaaacatgaatgacaagaggcccc |
| | ccaggattatcaaaaccatcgccagcaagacccagagcattaagaaatacagcaccgacatcctcggcaacctgtacgaggtcaagtccaaaaagcacccccagattatcaaaaa |
| | gggaaagcgacctgccgccacaaagaaggctggacaggctaagaagaagaaagattacaaagacgatgacgataagtgagtcgaccgactagagctcgctgatcagcctcgac |
| | tgtgccttctagttgccagccatctgttgtttgcccctcccccgtgccttccttgaccctggaaggtgccactcccactgtcctttcctaataaaatgaggaaattgcatcgcattgtctgag |
| | taggtgtcattctattctggggggtggggggggcaggacagcaagggggaggattgggaagacaatagcaggcatgctggggacgcaggaacccctagtgatggagttggcc |
| | actccctctctgcgcgctcgctcgctcactgaggccgggcgaccaaaggtcgcccgacgcccgggctttgcccgggcggcctcagtgagcgagcgagcgcgcagctgcctgca |
| | ggggcgcctgatgcggtattttctccttacgcatctgtgcggtatttcacaccgcatacgtcaaagcaaccatagtacgcgccctgtagcggcgcattaagcgcggcgggtgtggtg |
| | gttacgcgcagcgtgaccgctacacttgccagcgccctagcgcccgctcctttcgctttcttcccttcctttctcgccacgttcgccggctttccccgtcaagctctaaatcgggggctc |
| | cctttagggttccgatttagtgctttacggcacctcgaccccaaaaaacttgatttgggtgatggttcacgtagtgggccatcgccctgatagacggtttttcgccctttgacgttggagtc |
| | cacgttctttaatagtggactcttgttccaaactggaacaacactcaaccctatctcgggctattcttttgatttataagggattttgccgatttcggcctattggttaaaaaatgagctgattt |
| | aacaaaaatttaacgcgaattttaacaaaatattaacgtttacaattttatggtgcactctcagtacaatctgctctgatgccgcatagttaagccagccccgacacccgccaacacccgc |
| | tgacgcgccctgacgggcttgtctgctcccggcatccgcttacagacaagctgtgaccgtctccgggagctgcatgtgtcagaggttttcaccgtcatcaccgaaacgcgcgagac |
| | gaaagggcctcgtgatacgcctatttttataggttaatgtcatgataataatggtttcttagacgtcaggtggcacttttcggggaaatgtgcgcggaacccctatttgtttatttttctaaata |
| | cattcaaatatgtatccgctcatgagacaataaccctgataaatgcttcaataatattgaaaaaggaagagtatgagtattcaacatttccgtgtcgcccttattcccttttttgcggcattttg |
| | ccttcctgtttttgctcacccagaaacgctggtgaaagtaaaagatgctgaagatcagttgggtgcacgagtgggttacatcgaactggatctcaacagcggtaagatccttgagagtt |
| | ttcgccccgaagaacgttttccaatgatgagcacttttaaagttctgctatgtggcgcggtattatcccgtattgacgccgggcaagagcaactcggtcgccgcatacactattctcaga |
| | atgacttggttgagtactcaccagtcacagaaaagcatcttacggatggcatgacagtaagagaattatgcagtgctgccataaccatgagtgataacactgcggccaacttacttctg |
| | acaacgatcggaggaccgaaggagctaaccgcttttttgcacaacatgggggatcatgtaactcgccttgatcgttgggaaccggagctgaatgaagccataccaaacgacgagc |
| | gtgacaccacgatgcctgtagcaatggcaacaacgttgcgcaaactattaactggcgaactacttactctagcttcccggcaacaattaatagactggatggaggcggataaagttgc |
| | aggaccacttctgcgctcggcccttccggctggctggtttattgctgataaatctggagccggtgagcgtgggtctcgcggtatcattgcagcactggggccagatggtaagccctcc |
| | cgtatcgtagttatctacacgacggggagtcaggcaactatggatgaacgaaatagacagatcgctgagataggtgcctcactgattaagcattggtaactgtcagaccaagtttactc |
| | atatatactttagattgatttaaaacttcatttttaatttaaaaggatctaggtgaagatcctttttgataatctcatgaccaaaatcccttaacgtgagttttcgttccactgagcgtcagaccc |
| | cgtagaaaagatcaaaggatcttcttgagatcctttttttctgcgcgtaatctgctgcttgcaaacaaaaaaaccaccgctaccagcggtggtttgtttgccggatcaagagctaccaact |
| | ctttttccgaaggtaactggcttcagcagagcgcagataccaaatactgtccttctagtgtagccgtagttaggccaccacttcaagaactctgtagcaccgcctacatacctcgctctg |
| | ctaatcctgttaccagtggctgctgccagtggcgataagtcgtgtcttaccgggttggactcaagacgatagttaccggataaggcgcagcggtcgggctgaacggggggttcgtg |
| | cacacagcccagcttggagcgaacgacctacaccgaactgagatacctacagcgtgagctatgagaaagcgccacgcttcccgaagggagaaaggcggacaggtatccggtaa |
| | gcggcagggtcggaacaggagagcgcacgagggagcttccagggggaaacgcctggtatctttatagtcctgtcgggtttcgccacctctgacttgagcgtcgatttttgtgatgct |
| | cgtcaggggggcggagcctatggaaaaacgccagcaacgcggcctttttacggttcctggccttttgctggccttttgctcacatgt |
| AAV-G6Pasc | cagatctttggccactccctctctgcgcgctcgctcgctcactgaggccgggcgaccaaaggtcgcccgacgcccgggctttgcccgggcggcctcagtgagcgagcgagcgc |
| vector | gcagagagggagtggccaactccatcactaggggttcctggaggggtggagtcgtgacgtgaattacgtcatagggttagggaggtcggccgtctagactctgtcctgtgtctctg |
| (SEQ ID NO: 49) | gcctggtttcggggaccaggagggcagacccttgcactgccaagaagcatgccaaagttaatcattggccctgctgagtacatggccgatcaggctgtttttgtgtgcctgtttttctat |
| | tttacgtaaatcaccctgaacatgtttgcatcaacctactggtgatgcacctttgatcaatacattttagacaaacgtggtttttgagtccaaagatcagggctgggttgacctgaatactg |
| | gatacagggcatataaaacaggggcaaggcacagactcatagcagagcaatcaccaccaagcctggaataactgcaagggctctgctgacatcttcctgaggtgccaaggaaat |
| | gaggatggaggaaggaatgaatgttctccatgactttgggatccagtcaacacattacctccaggtgaattaccaagactcccaggactggttcatcttggtgtccgtgatcgcagac |
| | ctcaggaatgccttctacgtcctcttccccatctggttccatcttcaggaagctgtgggcattaaactcctttgggtagctgtgattggagactggctcaacctcgtctttaagtggattctc |
| | tttggacagcgtccatactggtgggttttggatactgactactacagcaacacttccgtgcccctgataaagcagttccctgtaacctgtgagactggaccagggagcccctctggcca |
| | tgccatgggcacagcaggtgtatactacgtgatggtcacatctactctttccatctttcagggaaagataaagccgacctacagatttcggtgcttgaatgtcattttgtggttgggattct |
| | gggctgtgcagctgaatgtctgtctgtcacgaatctaccttgctgctcattttcctcatcaagttgttgctggagtcctgtcaggcattgctgttacagaaactttcagccacatccacagc |
| | atctataatgccagcctcaagaaatattttctcattaccttcttcctgttcagcttcgccatcggattttatctgctgctcaagggactgggtgtagacctcctgtggactctggagaaagcc |
| | cagaggtggtgcgagcagccagaatgggtccacattgacaccacaccctttgccagcctcctcaagaacctgggcacgctctttggcctggggctggctctcaactccagcatgta |
| | cagggagagctgcaaggggaaactcagcaagtggctcccattccgcctcagctctattgtagcctccctcgtcctcctgcacgtctttgactccttgaaacccccatcccaagtcgag |
| | ctggtcttctacgtcttgtccttctgcaagagtgcggtagtgcccctggcatccgtcagtgtcatcccctactgcctcgcccaggtcctgggccagccgcacaagaagtcgttgtaaga |
| | gatgtggagtcttcggtgtttaaagtcgaattctgcagatctgtggcttctagctgcccgggtggcatccctgtgacccctccccagtgcctctcctggccttggaagttgccactccag |
| | tgcccaccagccttgtcctaataaaattaagttgcatcattttgtctgactaggtgtcctctataatattatggggtggaggggggtggtttggagcaaggggcccaagttgggaagaca |
| | caccatattggtcgagtagataagtagcatggcgggttaatcattaactacaaggaacccctagtgatggagttggccactccctctctgcgcgctcgctcgctcactgaggccgggc |
| | gaccaaaggtcgcccgacgcccgggctttgcccgggcggcctcagtgagcgagcgagcgcgcagagagggagtggccaaagatctgcgacgcgaggctggatggccttccc |
| | cattatgattcttctcgcttccggcggcatcgggatgcccgcgttgcaggccatgctgtccaggcaggtagatgacgaccatcagggacagcttcagcaaaaggccagcaaaagg |
| | ccaggaaccgtaaaaaggccgcgttgctggcgtttttccataggctccgcccccctgacgagcatcacaaaaatcgacgctcaagtcagaggtggcgaaacccgacaggactata |
| | aagataccaggcgtttccccctggaagctccctcgtgcgctctcctgttccgaccctgccgcttaccggatacctgtccgcctttctcccttcgggaagcgtggcgctttctcatagctc |
| | acgctgtaggtatctcagttcggtgtaggtcgttcgctccaagctgggctgtgtgcacgaaccccccgttcagcccgaccgctgcgccttatccggtaactatcgtcttgagtccaacc |
| | cggtaagacacgacttatcgccactggcagcagccactggtaacaggattagcagagcgaggtatgtaggcggtgctacagagttcttgaagtggtggcctaactacggctacact |
| | agaaggacagtatttggtatctgcgctctgctgaagccagttaccttcggaaaaagagttggtagctcttgatccggcaaacaaaccaccgctggtagcggtggtttttttgtttgcaag |
| | cagcagattacgcgcagaaaaaaaggatctcaagaagatcctttgatcttttctacggggtctgacgctcagtggaacgaaaactcacgttaagggattttggtcatgagattatcaaa |
| | aaggatcttcacctagatccttttaaattaaaaatgaagttttaaatcaatctaaagtatatatgagtaaacttggtctgacagttaccaatgcttaatcagtgaggcacctatctcagcgatc |
| | tgtctatttcgttcatccatagttgcctgactccccgtcgtgtagataactacgatacgggagggcttaccatctggccccagtgctgcaatgataccgcgagacccacgctcaccggc |
| | tccagatttatcagcaataaaccagccagccggaagggccgagcgcagaagtggtcctgcaactttatccgcctccatccagtctattaattgttgccgggaagctagagtaagtagt |
| | tcgccagttaatagtttgcgcaacgttgttgccattgctgcaggcatcgtggtgtcacgctcgtcgtttggtatggcttcattcagctccggttcccaacgatcaaggcgagttacatgat |
| | cccccatgttgtgcaaaaaagcggttagctccttcggtcctccgatcgttgtcagaagtaagttggccgcagtgttatcactcatggttatggcagcactgcataattctcttactgtcatg |
| | ccatccgtaagatgcttttctgtgactggtgagtactcaaccaagtcattctgagaatagtgtatgcggcgaccgagttgctcttgcccggcgtcaacacgggataataccgcgccaca |
| | tagcagaactttaaaagtgctcatcattggaaaacgttcttcggggcgaaaactctcaaggatcttaccgctgttgagatccagttcgatgtaacccactcgtgcacccaactgatcttc |
| | agcatcttttactttcaccagcgtttctgggtgagcaaaaacaggaaggcaaaatgccgcaaaaaagggaataagggcgacacggaaatgttgaatactcatactcttcctttttcaata |
| | ttattgaagcatttatcagggttattgtctcatgagcggatacatatttgaatgtatttagaaaaataaacaaataggggttccgcgcacatttccccgaaaagtgccacctg |
| First excerpt of | gggaatagattctctgggtagctgtgattggagactggctcaacctcgtctttaaatggtgagtaagagccagacagagaggagatcggcaagggaagaggctggtatAGtctc |
| sequence from | |
| edited dog G6PC | |
| gene sequenced | |
| and depicted in | |
| FIG. 1D with | |
| AG mutation to | |
| remove PAM | |
| site underlined | |
| and bolded (AG) | |
| (SEQ ID NO: 50) | |
| Second excerpt | ataaagagcttgggccagctaaaacc |
| of sequence from | |
| edited dog G6PC | |
| gene sequenced | |
| and depicted in | |
| FIG. 1D | |
| (SEQ ID NO: 51) | |
| Full sequence | ctcctggccctggaagttgccactccagtgcccaccagtcttgtcctaataaaattaagttgcatcattttgtctgactaggtgtccttctataatattatggggtggaggggggtggtatg |
| from edited dog | gagcaagggaatagattctctgggtagctgtgattggagactggctcaacctcgtctttaaatggtgagtaagagccagacagagaggagatcggcaagggaagaggctggtatA |
| G6PC sequenced | Gtctctggaaatgtctgacatcagaaattgccttctccgtgctagtcgggaagccacaggttactcccctcctgactctggatcatctacctaaagagggaagacagcgaaaatcttgt |
| in FIG. 1D with | tgatgaattgaagatacaggcaaggctatttcatatggatggaaggccagccggacttagtgaaaagtctaaagtatataggctataagcaaagaagcacagttgtattactttgaagc |
| primers used | agaataggtaacataaagataggtgtatgaaagatgatgtatgaaaacaggaaacaagtatataatttaaaaacccactgcatggagaaagaagaagtaacccagagaactgggct |
| underlined and | ctaacccttgttcggccacctaccagctgtatgtcctgggaaagtcagagcaactctctgagtttctgtcttacaatctgtaaacttaaattatccttgggagcttaataataattctagttaa |
| AG mutation to | catttattgaacatttagtaggtactaggccatgtgccaagtgcttttctgtgaggtagttactactgtattatagatgaagagacagagccattacgataagggaccagaactagatcca |
| remove PAM | catggtggtcaaaggcagaggcagcattttaaggccagacaggctagtttcagaggcaaagctcttaaggattatgctgtattcattgctgacaaattgactttttcagggagcaggag |
| site underlined | gaagccaaattttaatgtttgtcaatttctctggtgtatatattcctattggggccacttttgatgggtcaagcttgcaaaaatcctgaatattttacaattgtcagccaataccatatgattcta |
| and bolded (AG) | gctcattagtggcttggcctctatttcctcatctagaaaataaagagcttgggccagctaaaacctttaccatccctaaactatctatgtctactgtcaagtgattctgaagacccttctagct |
| (SEQ ID NO: 52) | c |
| Cas9 protein | MDKKYSIGLDIGTNSVGWAVITDEYKVPSKKFKVLGNTDRHSIKKNLIGALLFDSGETAEATRLKRTARRRYTRR |
| Streptococcus | KNRICYLQEIFSNEMAKVDDSFFHRLEESFLVEEDKKHERHPIFGNIVDEVAYHEKYPTIYHLRKKLVDSTDKADL |
| pyogenes | RLIYLALAHMIKFRGHFLIEGDLNPDNSDVDKLFIQLVQTYNQLFEENPINASGVDAKAILSARLSKSRRLENLIAQL |
| (SEQ ID NO: 53) | PGEKKNGLFGNLIALSLGLTPNFKSNFDLAEDAKLQLSKDTYDDDLDNLLAQIGDQYADLFLAAKNLSDAILLSDI |
| | LRVNTEITKAPLSASMIKRYDEHHQDLTLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPFLKDNREKIEKILTFRIPYYVGPLARGNS |
| | RFAWMTRKSEETITPWNFEEVVDKGASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVEDRFNASLGTYHDLLKIIKDKDFLDNEE |
| | NEDILEDIVLTLTLFEDREMIEERLKTYAHLFDDKVMKQLKRRRYTGWGRLSRKLINGIRDKQSGKTILDFLKSDG |
| | FANRNFMQLIHDDSLTFKEDIQKAQVSGQGDSLHEHIANLAGSPAIKKGILQTVKVVDELVKVMGRHKPENIVIEM |
| | ARENQTTQKGQKNSRERMKRIEEGIKELGSQILKEHPVENTQLQNEKLYLYYLQNGRDMYVDQELDINRLSDYDV |
| | DHIVPQSFLKDDSIDNKVLTRSDKNRGKSDNVPSEEVVKKMKNYWRQLLNAKLITQRKFDNLTKAERGGLSELD |
| | KAGFIKRQLVETRQITKHVAQILDSRMNTKYDENDKLIREVKVITLKSKLVSDFRKDFQFYKVREINNYHHAHDA |
| | YLNAVVGTALIKKYPKLESEFVYGDYKVYDVRKMIAKSEQEIGKATAKYFFYSNIMNFFKTEITLANGEIRKRPLIE |
| | TNGETGEIVWDKGRDFATVRKVLSMPQVNIVKKTEVQTGGFSKESILPKRNSDKLIARKKDWDPKKYGGFDSPTV |
| | AYSVLVVAKVEKGKSKKLKSVKELLGITIMERSSFEKNPIDFLEAKGYKEVKKDLIIKLPKYSLFELENGRKRMLAS |
| | AGELQKGNELALPSKYVNFLYLASHYEKLKGSPEDNEQKQLFVEQHKHYLDEIIEQISEFSKRVILADANLDKVLS |
| | AYNKHRDKPIREQAENIIHLFTLTNLGAPAAFKYFDTTIDRKRYTSTKEVLDATLIHQSITGLYETRIDLSQLGGD |
| Cas9 prctcin | MKRNYILGLDIGITSVGYGIIDYETRDVIDAGVRLFKEANVENNEGRRSKRGARRLKRRRRHRIQRVKKLLFDYNL |
| Staphylococcus | LTDHSELSGINPYEARVKGLSQKLSEEEFSAALLHLAKRRGVHNVNEVEEDTGNELSTKEQISRNSKALEEKYVAE |
| aureus | LQLERLKKDGEVRGSINRFKTSDYVKEAKQLLKVQKAYHQLDQSFIDTYIDLLETRRTYYEGPGEGSPFGWKDIKE |
| (SEQ ID NO: 54) | WYEMLMGHCTYFPEELRSVKYAYNADLYNALNDLNNLVITRDENEKLEYYEKFQIIENVFKQKKKPTLKQIAKEI |
| | LVNEEDIKGYRVTSTGKPEFTNLKVYHDIKDITARKEIIENAELLDQIAKILTIYQSSEDIQEELTNLNSELTQEEIEQI |
| | SNLKGYTGTHNLSLKAINLILDELWHTNDNQIAIFNRLKLVPKKVDLSQQKEIPTTLVDDFILSPVVKRSFIQSIKVIN |
| | AIIKKYGLPNDIIIELAREKNSKDAQKMINEMQKRNRQTNERIEEIIRTTGKENAKYLIEKIKLHDMQEGKCLYSLEA |
| | IPLEDLLNNPFNYEVDHIIPRSVSFDNSFNNKVLVKQEENSKKGNRTPFQYLSSSDSKISYETFKKHILNLAKGKGRI |
| | SKTKKEYLLEERDINRFSVQKDFINRNLVDTRYATRGLMNLLRSYFRVNNLDVKVKSINGGFTSFLRRKWKFKKE |
| | RNKGYKHHAEDALIIANADFIFKEWKKLDKAKKVMENQMFEEKQAESMPEIETEQEYKEIFITPHQIKHIKDFKDY |
| | KYSHRVDKKPNRELINDTLYSTRKDDKGNTLIVNNLNGLYDKDNDKLKKLINKSPEKLLMYHHDPQTYQKLKLI |
| | MEQYGDEKNPLYKYYEETGNYLTKYSKKDNGPVIKKIKYYGNKLNAHLDITDDYPNSRNKVVKLSLKPYRFDVY |
| | LDNGVYKFVTVKNLDVIKKENYYEVNSKCYEEAKKLKKISNQAEFIASFYNNDLIKINGELYRVIGVNNDLLNRIE |
| | VNMIDITYREYLENMNDKRPPRIIKTIASKTQSIKKYSTDILGNLYEVKSKKHPQIIKKG |
| spCas9 nucleic | atggacaagaagtacagcatcggcctggacatcggcaccaactctgtgggctgggccgtgatcaccgacgagtacaaggtgcccagcaagaaattcaaggtgctgggcaacacc |
| acid (used in | gaccggcacagcatcaagaagaacctgatcggagccctgctgttcgacagcggcgaaacagccgaggccacccggctgaagagaaccgccagaagaagatacaccagacgg |
| mouse and | aagaaccggatctgctatctgcaagagatcttcagcaacgagatggccaaggtggacgacagcttcttccacagactggaagagtccttcctggtggaagaggataagaagcacg |
| human SpCas9 | agcggcaccccatcttcggcaacatcgtggacgaggtggcctaccacgagaagtaccccaccatctaccacctgagaaagaaactggtggacagcaccgacaaggccgacctg |
| CRISPR vectors) | cggctgatctatctggccctggcccacatgatcaagttccggggccacttcctgatcgagggcgacctgaaccccgacaacagcgacgtggacaagctgttcatccagctggtgca |
| (SEQ ID NO: 55) | gacctacaaccagctgttcgaggaaaaccccatcaacgccagcggcgtggacgccaaggccatcctgtctgccagactgagcaagagcagacggctggaaaatctgatcgccc |
| | agctgcccggcgagaagaagaatggcctgttcggaaacctgattgccctgagcctgggcctgacccccaacttcaagagcaacttcgacctggccgaggatgccaaactgcagct |
| | gagcaaggacacctacgacgacgacctggacaacctgctggcccagatcggcgaccagtacgccgacctgtttctggccgccaagaacctgtccgacgccatcctgctgagcga |
| | catcctgagagtgaacaccgagatcaccaaggcccccctgagcgcctctatgatcaagagatacgacgagcaccaccaggacctgaccctgctgaaagctctcgtgcggcagca |
| | gctgcctgagaagtacaaagagattttcttcgaccagagcaagaacggctacgccggctacattgacggcggagccagccaggaagagttctacaagttcatcaagcccatcctg |
| | gaaaagatggacggcaccgaggaactgctcgtgaagctgaacagagaggacctgctgcggaagcagcggaccttcgacaacggcagcatcccccaccagatccacctgggag |
| | agctgcacgccattctgcggcggcaggaagatttttacccattcctgaaggacaaccgggaaaagatcgagaagatcctgaccttccgcatcccctactacgtgggccctctggcc |
| | aggggaaacagcagattcgcctggatgaccagaaagagcgaggaaaccatcaccccctggaacttcgaggaagtggtggacaagggcgcttccgcccagagcttcatcgagcg |
| | gatgaccaacttcgataagaacctgcccaacgagaaggtgctgcccaagcacagcctgctgtacgagtacttcaccgtgtataacgagctgaccaaagtgaaatacgtgaccgag |
| | ggaatgagaaagcccgccttcctgagcggcgagcagaaaaaggccatcgtggacctgctgttcaagaccaaccggaaagtgaccgtgaagcagctgaaagaggactacttcaa |
| | gaaaatcgagtgcttcgactccgtggaaatctccggcgtggaagatcggttcaacgcctccctgggcacataccacgatctgctgaaaattatcaaggacaaggacttcctggacaa |
| | tgaggaaaacgaggacattctggaagatatcgtgctgaccctgacactgtttgaggacagagagatgatcgaggaacggctgaaaacctatgcccacctgttcgacgacaaagtga |
| | tgaagcagctgaagcggcggagatacaccggctggggcaggctgagccggaagctgatcaacggcatccgggacaagcagtccggcaagacaatcctggatttcctgaagtcc |
| | gacggcttcgccaacagaaacttcatgcagctgatccacgacgacagcctgacctttaaagaggacatccagaaagcccaggtgtccggccagggcgatagcctgcacgagcac |
| | attgccaatctggccggcagccccgccattaagaagggcatcctgcagacagtgaaggtggtggacgagctcgtgaaagtgatgggccggcacaagcccgagaacatcgtgatc |
| | gaaatggccagagagaaccagaccacccagaagggacagaagaacagccgcgagagaatgaagcggatcgaagagggcatcaaagagctgggcagccagatcctgaaaga |
| | acaccccgtggaaaacacccagctgcagaacgagaagctgtacctgtactacctgcagaatggggggatatgtacgtggaccaggaactggacatcaaccggctgtccgacta |
| | cgatgtggaccatatcgtgcctcagagctttctgaaggacgactccatcgacaacaaggtgctgaccagaagcgacaagaaccggggcaagagcgacaacgtgccctccgaag |
| | aggtcgtgaagaagatgaagaactactggcggcagctgctgaacgccaagctgattacccagagaaagttcgacaatctgaccaaggccgagagaggcggcctgagcgaactg |
| | gataaggccggcttcatcaagagacagctggtggaaacccggcagatcacaaagcacgtggcacagatcctggactcccggatgaacactaagtacgacgagaatgacaagct |
| | gatccgggaagtgaaagtgatcaccctgaagtccaagctggtgtccgatttccggaaggatttccagttttacaaagtgcgcgagatcaacaactaccaccacgcccacgacgccta |
| | cctgaacgccgtcgtgggaaccgccctgatcaaaaagtaccctaagctggaaagcgagttcgtgtacggcgactacaaggtgtacgacgtgcggaagatgatcgccaagagcga |
| | gcaggaaatcggcaaggctaccgccaagtacttcttctacagcaacatcatgaactttttcaagaccgagattaccctggccaacggcgagatccggaagcggcctctgatcgaga |
| | caaacggcgaaaccggggagatcgtgtgggataagggccgggattttgccaccgtgcggaaagtgctgagcatgccccaagtgaatatcgtgaaaaagaccgaggtgcagaca |
| | ggcggcttcagcaaagagtctatcctgcccaagaggaacagcgataagctgatcgccagaaagaaggactgggaccctaagaagtacggggcttcgacagccccaccgtggc |
| | ctattctgtgctggtggtggccaaagtggaaaagggcaagtccaagaaactgaagagtgtgaaagagctgctggggatcaccatcatggaaagaagcagcttcgagaagaatccc |
| | atcgactttctggaagccaagggctacaaagaagtgaaaaaggacctgatcatcaagctgcctaagtactccctgttcgagctggaaaacggccggaagagaatgctggcctctgc |
| | cggcgaactgcagaagggaaacgaactggccctgccctccaaatatgtgaacttcctgtacctggccagccactatgagaagctgaagggctcccccgaggataatgagcagaa |
| | acagctgtttgtggaacagcacaagcactacctggacgagatcatcgagcagatcagcgagttctccaagagagtgatcctggccgacgctaatctggacaaagtgctgtccgcct |
| | acaacaagcaccgggataagcccatcagagagcaggccgagaatatcatccacctgtttaccctgaccaatctgggagcccctgccgccttcaagtactttgacaccaccatcgac |
| | cggaagaggtacaccagcaccaaagaggtgctggacgccaccctgatccaccagagcatcaccggcctgtacgagacacggatcgacctgtctcagctgggaggcgacaagc |
| | gacctgccgccacaaagaaggctggacaggctaagaagaagaaa |
| saCs9 nucleic | atgaagaggaattacatcctcggactggacatcggcattaccagcgtcggctacggcatcatcgattacgagacaagggacgtgattgacgctggcgtcaggctgttcaaggaggc |
| acid (used in dog | caacgtggagaataatgagggcaggagaagcaagaggggcgccaggaggctgaagaggaggagaaggcacagaatccagagggtcaagaagctcctgttcgactacaacct |
| and human | gctgaccgaccatagcgagctgagcggcattaacccctacgaggctagggtcaaaggcctctcccagaaactgtccgaggaggagtttagcgctgctctcctgcacctggccaag |
| saCas9 - | aggaggggagtgcacaatgtgaacgaggtcgaagaggacaccggcaatgagctcagcaccaaggagcagattagcagaaacagcaaagccctggaggaaaaatatgtcgcc |
| CRISPR vectors) | gagctacagctggagagactcaagaaggatggcgaggtcagaggctccattaacaggttcaagacctccgactacgtgaaagaagccaagcagctgctgaaggtgcaaaaggc |
| (SEQ ID NO: 56) | ctatcatcagctggaccagagcttcattgatacctatatcgacctgctcgaaaccaggagaacctactatgagggccctggagaaggaagccccttcggatggaaagacattaagg |
| | agtggtacgaaatgctgatgggacactgcacctacttccccgaggagctgaggagcgtgaaatatgcctacaacgccgatctctacaacgccctgaacgacctgaacaatctcgtg |
| | attacaagggatgagaacgaaaagctggaatattacgaaaaatttcagatcattgaaaacgtgtttaagcaaaagaagaagcccaccctgaagcagattgccaaggagatcctggtg |
| | aacgaagaggacattaaaggctacagagtgacctccacaggcaaacccgaatttaccaatctgaaggtgtatcacgatatcaaagatatcacagctagaaaggagatcatcgagaa |
| | tgctgagctgctcgaccaaatcgccaagattctcaccatctaccagagcagcgaggacatccaggaagaactgacaaacctgaactccgagctcacccaggaagaaatcgagca |
| | gatttccaacctgaaaggctacaccggcacccacaacctgtccctgaaggccatcaacctgatcctcgatgaactctggcacaccaatgacaatcaaatcgctatcttcaacagactg |
| | aagctcgtgcccaagaaagtggacctgagccagcagaaggaaatccctacaaccctcgtggacgatttcatcctcagccccgtcgtgaagaggagcttcatccagtccatcaaggt |
| | gattaacgctatcatcaagaagtatggcctccccaacgacatcatcatcgagctcgctagggaaaaaaactccaaggatgctcagaaaatgatcaacgagatgcagaaaaggaaca |
| | gacaaaccaacgaaaggattgaggagattatcaggaccaccggcaaggagaatgctaagtatctgattgaaaaaattaaactccacgatatgcaggagggcaagtgtctctatagc |
| | ctggaagccatccctctggaggatctcctgaacaatccttttaactacgaagtggaccacattatccccaggagcgtgtccttcgataactcctttaataataaggtcctggtcaaacag |
| | gaggagaactccaagaagggaaacaggacacccttccaatatctctccagcagcgactccaaaataagctacgaaacctttaaaaaacacattctgaacctggccaagggcaaag |
| | gcaggatcagcaaaaccaaaaaggaatacctcctggaggagagagacatcaacaggttctccgtgcagaaagacttcatcaacaggaacctggtggacaccaggtatgccacca |
| | ggggcctgatgaacctcctgaggtcctattttagagtcaataatctcgacgtgaaagtcaaatccattaatggcggctttaccagcttcctgaggagaaaatggaagttcaagaagga |
| | gagaaacaaaggctacaaacaccacgccgaagatgccctcatcattgctaacgccgacttcatctttaaggagtggaagaagctcgataaggccaagaaagtcatggagaatcaa |
| | atgttcgaggagaaacaagccgaatccatgcccgagattgaaacggagcaagagtacaaggaaatattcatcacccctcaccagatcaaacacatcaaggacttcaaagactaca |
| | aatatagccatagagtcgataagaagcccaacagggagctgatcaacgacacactctattccaccagaaaggatgataagggcaacaccctgattgtgaacaacctgaacggcct |
| | ctacgataaggataacgacaagctgaagaagctgattaacaagagccccgagaaactgctcatgtaccaccacgacccccaaacataccagaaactgaagctgatcatggaacag |
| | tatggcgacgagaaaaaccccctgtataagtactatgaagaaaccggcaactacctcaccaagtacagcaaaaaggataacggacccgtgatcaaaaagattaagtattacggcaa |
| | taagctgaatgcccacctcgatatcaccgatgattaccccaactccagaaataaagtcgtgaaactgtccctgaaaccttacaggtttgacgtgtacctggacaatggcgtgtacaagt |
| | tcgtgaccgtgaagaacctcgacgtgattaagaaggagaattactatgaggtcaacagcaaatgctatgaggaagccaagaaactgaagaagatttccaatcaggccgagttcatc |
| | gccagcttttataacaacgacctgattaagatcaatggcgaactgtatagggtgatcggcgtcaacaacgacctcctgaacaggattgaggtcaatatgatcgatattacctacagaga |
| | gtacctcgaaaacatgaatgacaagaggccccccaggattatcaaaaccatcgccagcaagacccagagcattaagaaatacagcaccgacatcctcggcaacctgtacgaggtc |
| | aagtccaaaaagcacccccagattatcaaaaaggga |

| DESCRIPTION | SEQUENCE |
|---|---|
| dogsurveyorFwd | Gccttctatgtcctctttccc |
| | (SEQ ID NO: 57) |
| dogsurveyorRev | Ttagagcccagttctctgggttac |
| | (SEQ ID NO: 58) |
| primer P1 | Gccagacaagaagtctttgtaaggc |
| | (SEQ ID NO: 59) |
| primer P2 | Ggacatggacaaggtcgagacattcc |
| | (SEQ ID NO: 60) |
| primer P3 | Ccaaagaatattagagctagaag |
| | (SEQ ID NO: 61) |
| primer P4 | Gctgttgaataggggacattacagacg |
| | (SEQ ID NO: 62) |
| Control primer P5 | Cgtctgtaatgtcccctattcaacagc |
| | (SEQ ID NO: 63) |
| Control primer P6 | Aagtacctagaacagtgtctggcacag |
| | (SEQ ID NO: 64) |
| cG6Pc Fwd | Tcttcgaccagccagacaag |
| | (SEQ ID NO: 65) |
| cG6Pc Rev | Ggtcctttaggaggtcatag |
| | (SEQ ID NO: 66) |
| hG6PC Fwd | Gcagttccctgtaacctgtgag |
| | (SEQ ID NO: 67) |
| hG6PC Rev | Ggtcggctttatctttccctg |
| | (SEQ ID NO: 68) |
| saCas9 Fwd | Gttggtatacacggtgtgcctg |
| | (SEQ ID NO: 69) |
| saCas9 Rev | Ctgacgccagcgtcaatcac |
| | (SEQ ID NO: 70) |
| cB-actin Fwd | Atggaatcctgcggcatccatg |
| | (SEQ ID NO: 71) |
| CB-actin Rev | Cagggtacatggtggttccac |
| | (SEQ ID NO: 72) |
| forward primer in the | Tgatagcagagcaatcgccaagtc |
| 5′ UTR of G6PC | (SEQ ID NO: 73) |
| reverse primer in | Agggtagatgtgaccatcacgtag |
| exon 2 of G6PC | (SEQ ID NO: 74) |
| G6PC fwd primer | Tgtccgtgattgcagacctcag |
| | (SEQ ID NO: 75) |
| G6PC rev primer | Agatgatccagagtcaggaggg |
| | (SEQ ID NO: 76) |
| U6-ENSCAFG00000007798 | Ctgttcgaagagtctgcatgtaag |
| fwd primer | (SEQ ID NO: 77) |
| U6-ENSCAFG00000007798 | Gaggggttttaggaagtgctgg |
| rev primer | (SEQ ID NO: 78) |
| ST6GAL1 fwd primer | Cagcttgttcacaaccagactc |
| | (SEQ ID NO: 79) |
| ST6GAL1 rev primer | Cagttttctccaggctcctaac |
| | (SEQ ID NO: 80) |
| C1ORf95 fwd primer | Tgggacattttagggagccaag |
| | (SEQ ID NO: 81) |
| C1ORf95 fwd primer | Cattctcgtcttggaacgtgtg |
| | (SEQ ID NO: 82) |
| RSPRY1 fwd primer | Gcaagataagagtcggtcttcgtg |
| | (SEQ ID NO: 83) |
| RSPRY1 rev primer | Acactagcagtcatacggactc |
| | (SEQ ID NO: 84) |
| ZNF536 fwd primer | Actggggctcgtatctcgg |
| | (SEQ ID NO: 85) |
| ZNF536 rev primer | Caagggagaggcttcagaatcg |
| | (SEQ ID NO: 86) |
| FANCC fwd primer | Tacagggagcctgcttctgcc |
| | (SEQ ID NO: 87) |
| FANCC rev primer | Cacaatcccttgtgggaagggc |
| | (SEQ ID NO: 88) |
| CCDC170 fwd primer | Agcacatcattcattcctgggc |
| | (SEQ ID NO: 89) |
| CCDC170 rev primer | Ccagttcacttcgagegtcttc |
| | (SEQ ID NO: 90) |
| TG fwd primer | Gcatccctgggatccttagg |
| | (SEQ ID NO: 91) |
| TG rev primer | Tagctgagcgtgagaagggg |
| | (SEQ ID NO: 92) |
| DIXDC1 fwd primer | Ctgtttagctggcattgactcc |
| | (SEQ ID NO: 93) |
| DIXDC1 rev primer | Gcttttgtctggtgtcatcccc |
| | (SEQ ID NO: 94) |
| Pak7 fwd primer | Ctctggaagagctacatagtcatc |
| | (SEQ ID NO: 95) |
| Pak7 rev primer | Gtaccatgaacttttaggacggtc |
| | (SEQ ID NO: 96) |
| U6-ENSCAFG00000007798 | Aatggtcagacatttccaaa |
| gRNA | (SEQ ID NO: 97) |
| ST6GALI^{B} gRNA | Aatggtgagagatttccaga |
| | (SEQ ID NO: 98) |
| Clorf95 gRNA | Gatgctcagacatttcaaaa |
| | (SEQ ID NO: 99) |
| RSPRY1 gRNA | Aatgtttagacatttccaga |
| | (SEQ ID NO: 100) |
| ZNF536 gRNA | Gatgggctggcatttccaga |
| | (SEQ ID NO: 101) |
| FANCC gRNA | Gagtgtcagacacttccaga |
| | (SEQ ID NO: 102) |
| CCDC170 gRNA | Gatggtcataaatttcctga |
| | (SEQ ID NO: 103) |
| TG gRNA | Tatagtcagacttttccaga |
| | (SEQ ID NO: 104) |
| DIXDC1 gRNA | Gattttcagacatttccaca |
| | (SEQ ID NO: 105) |
| Pak7^{B} gRNA | Catggtgagacatttccagt |
| | (SEQ ID NO: 106) |
| SpCas9 Fwd Primer | Agtacagcatcggcctggac |
| | (SEQ ID NO: 107) |
| SpCas9 Rev Primer | Gggctccgatcaggttcttc |
| | (SEQ ID NO: 108) |
| mB-actin Fwd primer | Ggctgtattcccctccateg |
| | (SEQ ID NO: 109) |
| mB-actin Rev primer | Ccagttggtaacaatgccatgt |
| | (SEQ ID NO: 110) |
| mousesurveyor | Tgacctacagactgaatccagg |
| Fwd primer | (SEQ ID NO: 111) |
| mousesurveyor | Taacatctgtgctcaggagctg |
| Rev primer | (SEQ ID NO: 112) |
| M1 primer | Cagccgcacaagaagtogttg |
| | (SEQ ID NO: 113) |
| M2 primer | Ccactcccactgtcctttcc |
| | (SEQ ID NO: 114) |
| M3 primer | Ggctcagtagatcaagtgcctgc |
| | (SEQ ID NO: 115) |
| M4 primer | Tctgggaatcagggactggg |
| | (SEQ ID NO: 116) |
| SaCas9 gRNA | Gauggucagacauuuccaga |
| | (SEQ ID NO: 117) |
| SaCas9 gRNA | Cuggcucaaccucgucuuuaaaug |
| | (SEQ ID NO: 118) |
| SaCas9 gRNA | Uucugauggucagacauuuccaga |
| | (SEQ ID NO: 119) |
| SaCas9 gRNA | Agguagaugauccagagucaggag |
| | (SEQ ID NO: 120) |
| SaCas9 gRNA | Ugauggacagacauugcgaga |
| | (SEQ ID NO: 121) |
| SaCas9 gRNA | Gcaugaguagcccguggcuuc |
| | (SEQ ID NO: 122) |
| SaCas9 gRNA | Ucagagagagggguuggaagc |
| | (SEQ ID NO: 123) |
| SaCas9 gRNA | Agaugauccaaagucagagag |
| | (SEQ ID NO: 124) |
| SpCas9 gRNA | Ucuauguccucuuucccauc |
| | (SEQ ID NO: 125) |
| SpCas9 gRNA | Ucuacguccucuuccccauc |
| | (SEQ ID NO: 126) |
| SpCas9 gRNA | Uugcugaucuccucucuaua |
| | (SEQ ID NO: 127) |
| SpCas9 gRNA | Gugguaagaaccauauagag |
| | (SEQ ID NO: 128) |
| SpCas9 gRNA | Gaggagaucagcaagaaaag |
| | (SEQ ID NO: 129) |
| SpCas9 gRNA | Agaucagcaagaaaagaggc |
| | (SEQ ID NO: 130) |
| SpCas9 gRNA | Cuggggaaagcaacuucuga |
| | (SEQ ID NO: 131) |

1. An isolated nucleic acid, comprising:
(i) a nucleotide sequence encoding a human glucose-6-phosphatase,
(ii) a nucleotide sequence with homology with a region located 5′ of a target site in a G6PC gene locus, and
(iii) a nucleotide sequence with sequence homology with a region located 3′ of the target site in a G6PC gene locus,
wherein (i) is flanked by (ii) and (iii).

2. (canceled)

3. The isolated nucleic acid of claim 1, wherein the nucleotide sequence of (i) has at least 70%, at least 75%, at least 80%, at least 85%, at least 90%, at least 95%, at least 96%, at least 97%, at least 98%, at least 99%, or 100% sequence identity to any one of SEQ ID NO: 16 to SEQ ID NO: 18.

4.-8. (canceled)

9. The isolated nucleic acid of claim 1, wherein the nucleotide sequence of (i) further comprises a human G6PC promoter sequence operably linked to the nucleotide sequence encoding the human glucose-6-phosphatase.

10. The isolated nucleic acid of claim 9, wherein the human G6PC promoter sequence comprises the sequence set forth in any one of SEQ ID NO:20-SEQ ID NO: 22.

11.-14. (canceled)

15. The isolated nucleic acid of claim 1, wherein the nucleotide sequence of (ii) has at least 70%, at least 75%, at least 80%, at least 85%, at least 90%, at least 95%, at least 96%, at least 97%, at least 98%, at least 99%, or 100% sequence identity to SEQ ID NO: 32 or SEQ ID NO:33.

16.-20. (canceled)

21. The isolated nucleic acid of claim 1, wherein the nucleotide sequence of (iii) has at least 70%, at least 75%, at least 80%, at least 85%, at least 90%, at least 95%, at least 96%, at least 97%, at least 98%, at least 99%, or 100% sequence identity to SEQ ID NO: 34 or SEQ ID NO:35.

22.-26. (canceled)

27. A vector comprising the isolated nucleic acid of claim 1.

28. A vector system for stably integrating a therapeutic human G6PC transgene in a cell, the system comprising:
(a) a first vector comprising the nucleic acid of claim 1; and
(b) a second vector comprising a nucleotide sequence encoding a Cas9 endonuclease;
wherein either the first vector or the second vector further comprises a nucleotide sequence encoding a small guide RNA (gRNA) targeting the target site in the G6PC gene locus.

29. (canceled)

30. The vector system of claim 28, wherein the Cas9 endonuclease comprises a SaCas9 endonuclease and the target site in the G6PC gene locus comprises the sequence set forth in any one of SEQ ID NO:5 to SEQ ID NO:8.

31. (canceled)

32. The vector system of claim 28, wherein the Cas9 endonuclease comprises a SpCas9 endonuclease and the target site in the G6PC gene locus comprises the sequence set forth in any one of SEQ ID NO: 10 to SEQ ID NO:15.

33. (canceled)

34. The vector system of claim 28, wherein the nucleotide sequence encoding the gRNA is operably linked to an exogenous promoter and/or enhancer.

35. The vector system of claim 28, wherein the nucleotide sequence encoding the Cas9 endonuclease is operably linked to an exogenous promoter and/or enhancer.

36. The vector system of claim 34, wherein the exogenous promoter and/or enhancer is a U6 promoter, a CMV enhancer or a human G6PC promoter.

37.-38. (canceled)

39. The vector system of claim 28, wherein the first and second vectors are AAV vectors.

40.-47. (canceled)

48. A method of expressing a G6PC transgene in a subject having a glycogen storage disease (GSD), the method comprising: administering to the subject a therapeutically effective amount of the vector system of claim 28, wherein at least one cell of the subject stably integrates and expresses the G6PC transgene into its genomic DNA.

49. (canceled)

50. The method of claim 48, wherein stably integrating the human G6PC transgene comprises delivering one or more nucleic acid vectors to the subject, the nucleic acid vectors encoding for a site-directed endonuclease, a guide RNA targeting a target site in a G6PC gene locus, and the G6PC transgene.

51. The method of claim 50, wherein the site directed endonuclease generates a double stranded break at or near the target site in the G6PC gene locus and the G6PC transgene is integrated at the site of the double stranded break via homologous recombination.

52.-57. (canceled)

58. The method of claim 48, wherein a ratio of the first vector to the second vector delivered to the cell or administered to the subject is from about 10:1 to about 1:1, from about 8:1 to about 1:1, from about 5:1 to about 1:1, or from about 4:1 to about 1:1.

59.-73. (canceled)

74. The vector system of claim 35, wherein the exogenous promoter and/or enhancer is a U6 promoter, a CMV enhancer or a human G6PC promoter.

75. The method of claim 48, further comprising treating and/or slowing or preventing progression of the glycogen storage disease in the subject.
